(19) United States
(12) Patent Application Publication
(10) Pub. No.: US 2024/0218054 A1
(43) Pub. Date: Jul. 4, 2024

(54) ANTI-YELLOW FEVER VIRUS ANTIBODIES, AND METHODS OF THEIR GENERATION AND USE

(71) Applicant: Mabloc, LLC, Washington, DC (US)

(72) Inventors: Anna WEC, Lebanon, NH (US); Laura WALKER, Norwich, VT (US)

(21) Appl. No.: 18/485,163

(22) Filed: Oct. 11, 2023

Related U.S. Application Data
(63) Continuation of application No. 18/045,802, filed on Oct. 11, 2022, now abandoned.
Application No. 18/045,802 is a continuation of application No. 17/103,844, filed on Nov. 24, 2020, now Pat. No. 11,479,598.
(60) Provisional application No. 62/940,049, filed on Nov. 25, 2019.

(51) Int. Cl. C07K 16/10; A61K 39/42; A61P 31/14

(52) U.S. Cl. CPC C07K 16/10; A61K 39/42; A61P 31/14; C07K 2317/76; C07K 2317/92

(57) ABSTRACT

Antibodies and antigen-binding fragments thereof specific to the YFV E protein and with neutralizing potency against YFV are provided. These antibodies and antigen-binding fragments are useful in treating YFV.

### CROSS-REFERENCE TO RELATED APPLICATIONS

This application claims priority to U.S. patent application Ser. No. 18/045,802, filed Oct. 11, 2022, which is a continuation of U.S. patent application Ser. No. 17/103,844, filed Nov. 24, 2022, which issued as U.S. Pat. No. 11,479,598 on Oct. 25, 2022, and U.S. Provisional Application No. 62/940,049, filed Nov. 25, 2019, each of which is hereby incorporated by reference in their entirety.

### REFERENCE TO A “SEQUENCE LISTING,” A TABLE, OR A COMPUTER PROGRAM LISTING APPENDIX

The Sequence Listing written in file “059359-501C02US_SL_ST26.xml”, created Oct. 11, 2023, 1,323,730 bytes, machine format IBM-PC, MS Windows operating system, is hereby incorporated by reference.

### FIELD OF THE DISCLOSURE

The disclosure relates to anti-Yellow Fever Virus (YFV) antibodies and antigen-binding fragments thereof, and compositions containing such antibodies and antigen-binding fragments thereof, and therapeutic and diagnostic uses for the antibodies, antigen-binding fragments, and compositions.

### BACKGROUND OF THE DISCLOSURE

Yellow Fever Virus (YFV) is a mosquito-borne flavivirus found in tropical and subtropical areas of Africa and South America. It is transmitted to humans primarily through the bite of infected Aedes or Haemagogus mosquito species and has three distinct transmission cycles: 1) jungle or sylvatic cycle; 2) African savannah (intermediate) cycle; and 3) urban cycle. (www.cdc.gov/yellowfever/transmission/index.html). While many people infected with YFV are asymptomatic, others develop symptoms such as fever, chills, headache, low back pain, myalgia, loss of appetite, nausea, vomiting, and/or fatigue following an incubation period of 3-6 days. (www.who.int/news-room/fact-sheets/detail/yellow-fever). Roughly 15% of people infected develop a severe form of YFV that includes high fever, bleeding diatheses, abdominal pain, renal failure, cardiovascular instability, and liver failure; up to 50% of patients with the severe form of YFV will die. (McGuinness et al, Neurohospitalist 2017, 7(4); 157-158).

YFV has a RNA genome of 10,862 nucleotides that encode three structural and seven non-structural proteins. From the 5′ terminus, the order of the encoded proteins is: C; prM/M; E; NS1; NS2A; NS2B; NS3; NS4A; NS4B and NS5. The three structural proteins include the C (capsid) protein, the membrane protein, M, and the envelope protein, E. The envelope protein plays an important role in cell tropism, virulence, and immunity.

Live attenuated 17D vaccine is considered one of the safest and most efficacious vaccines developed to date. However, despite the availability of the vaccine, Yellow Fever remains a serious public health issue. There are some data suggesting immunity, though protective, may wane over time in certain populations. Additionally, YFV outbreaks in non-endemic countries (such as the 11 imported cases in China in 2016) and concurrent outbreaks exhausting stockpiles of 17D have underscored the importance of developing a treatment.

Indeed, to date there are currently no approved YFV treatments (the only course being supportive therapy) and, despite decades of research, the development of safe and effective therapeutic antibodies against YFV has remained elusive. The YFV E-specific serum antibody response has been shown to be overwhelmingly mediated by antibodies targeting domain I (DI) and/or domain II (DII) of the E protein, whereas antibodies targeting domain III (DIII) are absent or present at very low titers (DVratskikh et al. PLoS pathogens 9, e1003458 (2013)). Correspondingly, the six YFV E-specific human monoclonal antibodies described to date all target overlapping epitopes within DII of the E protein (Lu et al. Cell Reports 26, 438-446 e435 (2019); Daffis et al. Virology 337, 262-272 (2005)). Recently, the crystal structure of one of these mAbs (5A) in complex with a soluble YFV E dimer was determined, which showed that this mAb binds to a conserved neutralizing epitope within DII of one E monomer (Lu et al. Cell Reports 26, 438-446 e435 (2019)). Therefore, there remains a need for highly specific, high affinity, and highly potent neutralizing anti-YFV antibodies and antigen-binding fragments thereof.

### SUMMARY OF THE DISCLOSURE

The disclosure pertains to the discovery of antibodies and antigen-binding fragments thereof that bind to YFV protein and exhibit neutralizing potency, in particular antibodies binding to the domain III (DIII) of the E protein that exhibit high neutralization potency. The antibodies of the present disclosure may also cross-react with other flaviviruses, e.g., display binding reactivity to DENV-2, DENV-4, WNV, and/or ZIKV E proteins. An extensive panel of YFV-specific monoclonal antibodies is described. Binding studies demonstrated that the neutralizing antibody response to YFV-17D is primarily mediated by antibodies that recognize FL proximal epitopes within DII of the YFV E protein. A small set of DIII-targeting antibodies having potent neutralizing activity was also identified. Additionally, binding assays revealed that YFV-17D vaccination appears to induce a subset of antibodies that display broad flavivirus binding activity, the majority of which target the highly conserved FL and show little to no cross-neutralizing activity. Neutralization studies showed a proportion of antibodies display highly potent neutralizing activity. Altogether, the panel of antibodies described herein provides promising therapeutic candidates and a framework for the rational design of YFV vaccines.

Such antibodies may be useful when administered prophylactically (prior to exposure to the virus and infection with the virus) to lessen the severity, or duration of a primary infection with YFV, or ameliorate at least one symptom associated with the infection. The antibodies may be used alone or in conjunction with a second agent useful for treating an YFV infection. In certain embodiments, the antibodies may be given therapeutically (after exposure to and infection with the virus) either alone, or in conjunction with a second agent to lessen the severity or duration of the primary infection, or to ameliorate at least one symptom associated with the infection. In certain embodiments, the antibodies may be used prophylactically as stand-alone therapy to protect patients who are at risk for acquiring an infection with YFV, such as those described above. Any of these patient populations may benefit from treatment with the antibodies of the disclosure, when given alone or in conjunction with a second agent, including for example, an anti-viral therapy, or other anti-viral vaccines.

In certain embodiments are provided isolated antibodies or antigen-binding fragments thereof that specifically bind to YFV, wherein at least one of a CDRH1, a CDRH2, a CDRH3, a CDRL1, a CDRL2, and a CDRL3 amino acid sequence of such antibodies or the antigen-binding fragments thereof is at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to at least one of the CDRH1, CDRH2, CDRH3, CDRL1, CDRL2, and/or CDRL3 amino acid sequences as disclosed in Table 3 of an antibody selected from Antibody Number 1 through Antibody Number 152 as disclosed in Table 3.

The antibody or the antigen-binding fragment thereof may also have one or more of the following characteristics: a) the antibodies or antigen-binding fragments thereof display a clean or low polyreactivity profile; b) the antibodies or antigen-binding fragments thereof display an in vitro neutralization potency (IC₅₀) of between about 0.5 microgram/milliliter (μg/ml) to about 5 μg/ml; between about 0.05 μg/ml to about 0.5 μg/ml; or less than about 0.05 mg/ml; c) the antibodies or antigen-binding fragments thereof bind YFV-17D particles; or d) the antibody or antigen-binding fragment thereof binds to an envelope protein of YFV. In certain embodiments, the isolated antibodies or antigen-binding fragments thereof comprise at least two; at least three; or 4 of characteristics a) through d) above.

In certain other embodiments, the isolated antibodies or antigen-binding fragments thereof comprise: a) the CDRH1 amino acid sequence of any one of the antibodies designated Antibody Number 1 through Antibody Number 152 as disclosed in Table 3; b) the CDRH2 amino acid sequence of any one of the antibodies designated Antibody Number 1 through Antibody Number 152 as disclosed in Table 3; c) the CDRH3 amino acid sequence of any one of the antibodies designated Antibody Number 1 through Antibody Number 152 as disclosed in Table 3; d) the CDRL1 amino acid sequence of any one of the antibodies designated Antibody Number 1 through Antibody Number 152 as disclosed in Table 3; e) the CDRL2 amino acid sequence of any one of the antibodies designated Antibody Number 1 through Antibody Number 152 as disclosed in Table 3; f) the CDRL3 amino acid sequence of any one of the antibodies designated Antibody Number 1 through Antibody Number 152 as disclosed in Table 3; and/or g) any combination of two or more of a), b), c), d), e), and f).

In certain other embodiments, the isolated antibodies or antigen-binding fragments thereof are selected from the group consisting of antibodies that are at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to any one of the antibodies designated as Antibody Number 1 through Antibody Number 152 as disclosed in Table 3.

In certain other embodiments, the isolated antibodies or antigen-binding fragments thereof comprise: a) a heavy chain (HC) amino acid sequence of any one of the antibodies designated Antibody Number 1 through Antibody Number 152 as disclosed in Table 3; and/or b) a light chain (LC) amino acid sequence of any one of the antibodies designated Antibody Number 1 through Antibody Number 152 as disclosed in Table 3.

The disclosure also contemplates nucleic acids encoding the described anti-YFV antibodies and expression vectors comprising said nucleic acids, as well as host cells that express such antibodies via the nucleic acids and/or expression vectors.

In one embodiment is provided isolated nucleic acid sequences encoding antibodies or antigen-binding fragments thereof disclosed herein.

In other embodiments are provided expression vectors comprising isolated nucleic acid sequences encoding antibodies or antigen-binding fragments disclosed herein.

In other embodiments are provided host cells transfected, transformed, or transduced with nucleic acid sequences encoding antibodies or antigen-binding fragments disclosed herein or expression vectors comprising isolated nucleic acid sequences encoding antibodies or antigen-binding fragments disclosed herein.

In other embodiments are provided pharmaceutical compositions comprising one or more of the isolated antibodies or antigen-binding fragments thereof disclosed herein; and a pharmaceutically acceptable carrier and/or excipient.

In other embodiments are provided pharmaceutical compositions comprising one or more nucleic acid sequences encoding antibodies or antigen-binding fragments disclosed herein, or one or more expression vectors comprising nucleic acid sequences encoding antibodies or antigen-binding fragments disclosed herein; and a pharmaceutically acceptable carrier and/or excipient.

In other embodiments are provided expression vectors comprising nucleic acid sequences encoding antibodies or antigen-binding fragments disclosed herein; or a host cell comprising nucleic acid sequences encoding antibodies or antigen-binding fragments disclosed herein.

The disclosure further contemplates methods of prevention and/or treatment using the described anti-YFV antibodies (or nucleic acids encoding or expression vectors comprising such nucleic acids).

In one embodiment is provided methods of treating or preventing a Yellow Fever Virus (YFV) infection, or at least one symptom associated with YFV infection, comprising administering to a patient in need thereof or suspected of being in need thereof: a) one or more antibodies or antigen-binding fragments thereof according to other embodiments disclosed herein; b) one or more nucleic acid sequences encoding antibodies or antigen-binding fragments disclosed herein; an expression vector comprising nucleic acid sequences encoding antibodies or antigen-binding fragments disclosed herein; or a host cell comprising an expression vector comprising nucleic acid sequences encoding antibodies or antigen-binding fragments disclosed herein; or c) a pharmaceutical composition according to other embodiments disclosed herein; such that the YFV infection is treated or prevented, or the at least one symptom associated with YFV infection is treated, alleviated, or reduced in severity.

In other embodiments the methods further comprise administering to the patient a second therapeutic agent.

In embodiments the second therapeutic agent is selected from: an antiviral agent; a vaccine specific for YFV; a vaccine specific for a flavivirus; an siRNA specific for a YFV antigen; and a second antibody specific for a YFV antigen.

In certain embodiments are provided pharmaceutical compositions for use in preventing a YFV infection in a patient in need thereof or suspected of being in need thereof, or for treating a patient suffering from an YFV infection, or for ameliorating at least one symptom or complication associated with the infection, wherein the infection is either prevented, or at least one symptom or complication associated with the infection is prevented, ameliorated, or lessened in severity and/or duration as a result of such use. In certain embodiments are provided pharmaceutical compositions for use in preventing a YFV infection in a patient in need thereof or suspected of being in need thereof. In certain embodiments are provided pharmaceutical compositions for use in treating a patient suffering from an YFV infection. In certain embodiments are provided pharmaceutical compositions for use in ameliorating at least one symptom or complication associated with the infection. In certain embodiments the infection is prevented. In certain embodiments at least one symptom or complication associated with the infection is prevented, ameliorated, or lessened in severity and/or duration as a result of such use.

In certain embodiments are provided pharmaceutical compositions for use in treating or preventing a YFV infection, or at least one symptom associated with said YFV infection, in a patient in need thereof or suspected of being in need thereof, wherein the infection is either prevented, or at least one symptom or complication associated with the infection is prevented, ameliorated, or lessened in severity and/or duration as a result of such use.

In certain other embodiments are provided uses of the pharmaceutical compositions in the manufacture of a medicament for preventing a YFV infection in a patient in need thereof, or for treating a patient suffering from a YFV infection, or for ameliorating at least one symptom or complication associated with the infection, wherein the infection is either prevented, or at least one symptom or complication associated with the infection is prevented, ameliorated, or lessened in severity and/or duration.

In certain other embodiments are provided uses of the pharmaceutical compositions in the manufacture of a medicament for preventing a YFV infection, or at least one symptom associated with said YFV infection, in a patient in need thereof or suspected of being in need thereof, wherein the infection is either prevented, or at least one symptom or complication associated with the infection is prevented, ameliorated, or lessened in severity and/or duration as a result of such use.

In certain other embodiments, an antibody that binds to the YFV E-Protein is provided. This antibody can bind to at least one of an epitope within FL of Domain II of the YFV E protein, proximal to the FL of Domain II of the YFV E protein, and to a protein in Domain III of YFV. This antibody can also have one or more of the following characteristics: a) the antibodies or antigen-binding fragments thereof display a clean or low polyreactivity profile; b) the antibodies or antigen-binding fragments thereof display an in vitro neutralization potency (IC₅0) of between about 0.5 microgram/milliliter (μg/ml) to about 5 μg/ml; between about 0.05 μg/ml to about 0.5 μg/ml; or less than about 0.05 mg/ml; c) the antibodies or antigen-binding fragments thereof bind YFV-17D particles; and d) the antibody or antigen-binding fragment thereof binds to an envelope protein of YFV.

### BRIEF DESCRIPTION OF THE FIGURES

FIG. 1A and FIG. 1B illustrate donor serum analysis following YVF-14D vaccination. FIG. 1A: Serum neutralizing activity against YFV-17D at day −5 (pre-vaccination), 10, 14, 28, 90, 180, 270, and 360 post-vaccination. Averages ±SD (n=6) from two independent experiments are shown. FIG. 1B: Neutralization IC₅₀s of serum samples at each time point post-vaccination, expressed as reciprocal serum dilution.

FIG. 2A through FIG. 2D show characterization of the YFV-17D vaccination-induced plasmablast responses at days 10 and 14. FIG. 2A: Frequency of plasmablasts among CD19⁺CD20^{-/lo} B cells in peripheral blood at days 0, 10, and 14 post-vaccination. Plasmablasts are defined herein as CD19⁺CD3⁻CD8⁻CD14⁻CD16⁻CD20^{-/lo} CD38^{hi}CD27^{hi} cells. FIG. 2B: Percentage of PB-derived mAbs that showed ELISA binding reactivity to whole YFV-17D particles at 100 nM. FIG. 2C: Neutralizing activity of PB-derived mAbs against YFV-17D at 100 nM and 10 nM concentrations. Green dots indicate the number of nucleotide substitutions in V_{H}+V_{L}. FIG. 2D: Proportion of YFV-17D reactive PB-derived mAbs with the indicated neutralization potencies (IC₅₀s).

FIG. 3 illustrates the binding activity of germline-reverted plasmablast monoclonal antibodies. Binding traces and affinities of three somatically mutated PB-derived mAbs (ADI-46184, ADI-46185, and ADI-42168) and their corresponding UCAs, as determined by Biacore. UCA, unmutated common ancestor.

FIG. 4 shows neutralization screening of PB-derived mAbs. Representative YFV-17D neutralization titration curves for PB mAbs screened by micro-titer neutralization assay. Averages ±SD (n=6) from two independent experiments are shown.

FIGS. 5A and 5B show the presence of swIg⁺ B cells that display reactivity to YFV-17D. FIG. 5A: YFV E reactivity of swIg⁺ B cells at each sampling time point. Fluorescence activated cell sorting (FACS) plots shown are gated on CD19⁺CD20⁺IgD⁻IgM⁻ B cells. YFV E was labeled with two different colors to reduce background binding. FIG. 5B: Percentage of swIg+B cells at each sampling time point that display YFV E reactivity.

FIG. 6A through 6E illustrate that YFV E-specific antibodies show preferential usage of the VH3-72 germline gene. FIG. 6A: VH germline gene usage of YFV E-specific mAbs isolated from each sampling time point. VH germline gene frequencies of unselected human MBC repertoires (“Unselected”) are also included for comparison. Sequencing data for unselected human MBCs was obtained from multiple high-throughput sequencing studies. FIG. 6B: VL germline gene usage of mAbs utilizing the VH3-72 germline gene. MAbs from all sampling time points were pooled for this analysis. The numbers in the center of the pies denote the total number of VH3-72 mAbs. FIG. 6C: Length distribution of CDR H3 in YFV E-specific mAbs utilizing the VH3-72 germline gene, mAbs utilizing all other VH germline genes, or unselected Abs from MBCs. FIG. 6D: SHM loads (expressed as number of nucleotide substitutions in VH) of YFV E-specific mAbs utilizing the VH3-72 germline gene or all other VH germline genes. FIG. 6E: Apparent binding affinities of mAbs utilizing the VH3-72 germline gene or all other VH germline genes to the YFV E protein, as determined by BLI. Black bars indicate medians. Avid KD^{App}s are plotted for the mAbs isolated from day 14 MBCs because only a small subset of these mAb showed detectable binding to YFV E in a monovalent orientation. Statistical comparisons were made using the Mann-Whitney test (*** P<0.001, ** P<0.01, * P<0.05).

FIG. 7A through 7D illustrate antibodies targeting epitopes within or proximal to the FL dominate the memory B cell response to YFV-17D vaccination. FIG. 7A: Proportion of mAbs in each of the major competition groups at each sampling time point. FIG. 7B: VH3-72 utilizing mAbs are shaded according to the competition group; natively paired light chain germline genes are indicated. FIG. 7C: Proportion of mAbs that compete with 4G2 and use the VH3-72 germline gene. FIG. 7D: Apparent affinities of 4G2-competing mAbs that either use the VH3-72 germline gene or all other germline genes. Statistical comparisons were made using the Mann-Whitney test (** P<0.01).

FIG. 8A through 8D illustrate a majority of highly potent neutralizing antibodies recognize FL-proximal epitopes. FIG. 8A: Proportion of mAbs with neutralization IC₅0s (less than 1, 1-10, greater than 10-100, and greater than 100 nM) against YFV-17D in each epitope bin. n.n—non-binder. FIG. 8B: Neutralization IC₅₀s of individual mAbs against YFV-17D across the indicated epitope bins. Black bars indicate medians. FIG. 8C: Proportion of highly potent neutralizing antibodies (IC₅₀<1 nM) targeting the indicated antigenic sites on YFV E. The number in the center of the pie indicates the number of highly potent neutralizing antibodies. FIG. 8D: VH and VL germline gene usage of 5A-only or 5A/ADI-45107 competitor neutralizing antibodies. MAbs from both donors were combined for all analyses shown.

FIG. 9A through 9C shows a subset of monoclonal antibodies show broad flavivirus cross-reactivity. FIG. 9A: Proportion of mAbs that react with one or more of the flavivirus E proteins tested (YFV, DENV-1, DENV-2, ZIKV, and WNV). Recombinant E protein binding was measured in an avid orientation by BLI. Numbers in the center of the pies indicate the number of mAbs analyzed. FIG. 9B: Proportion of cross-reactive mAbs that recognize the indicated antigenic sites. Cross-reactive mAbs from both donors were combined for this analysis. FIG. 9C: Heatmap showing the cross-reactivity profiles of 50 mAbs that showed binding to at least one flavivirus E protein aside from YFV E. Apparent affinities (KD^{App}s) were determined in avid orientation using BLI. A heat map showing virus neutralizing activity against YFV-17D and ZIKV is shown below the binding heat map. Competition group assignments for the individual mAbs are indicated at the top of the heatmap. N.B., non-binding; n.n., non-neutralizing; neut., neutralization.

### DETAILED DESCRIPTION OF THE DISCLOSURE

An in-depth understanding of the human antibody response to YFV infection will aid the development and evaluation of YFV vaccine and therapeutic and/or prophylactic antibodies for the treatment and/or prevention of YFV infection. A high-throughput antibody isolation platform was used to dissect the human memory B cell response to YFV in two vaccinated adult donors and highly potent and selective YFV-neutralizing antibodies were isolated and characterized.

High-throughput epitope mapping studies revealed that epitopes within or proximal to the FL on DII of the YFV E protein are immunodominant. While many of the mAbs that bound to FL-specific epitopes were non-neutralizing, most of the mAbs that targeted FL-proximal epitopes overlapping the 5A epitope showed neutralizing activity. Furthermore, the vast majority of potent nAbs recognized this antigenic site suggesting that the nAb response induced by YFV-17D vaccination is primarily mediated by this class of Abs. A subset of these mAbs displayed exceptionally potent neutralizing activity, with IC₅₀s that were about 10 times lower than previously described YFV mAbs. Given the recent YFV outbreaks in Brazil and the Democratic Republic of Congo, coupled with YFV-17D vaccine supply shortages and the lack of effective treatments for YFV disease, these mAbs represent promising candidates for prophylaxis and/or therapy

Accordingly, disclosed herein are highly selective and potent anti-YFV antibodies, as well as possible vaccine candidates, for the treatment and/or prophylaxis of YFV infection. Additionally, the reagents disclosed here provide a useful set of tools for the evaluation of clinical trials, which will be critical for selecting the optimal YFV vaccination or antibody-based therapeutic strategy from those currently under investigation.

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

“Yellow Fever Virus”, also referred to as “YFV”, is an RNA virus typically spread by the bite of infected Aedes or Haemagogus species mosquito bites.

The term “YFV-17D” refers to the attenuated YFV vaccine strain developed by passaging a wild-type Asibi strain in chicken and mouse tissue. There are three 17D substrains in current production: 17DD manufactured in Brazil, 17D-213 manufactured in Russia, and 17D-204 manufactured in China, France, Senegal, and the USA. While the mechanism of attenuation is poorly understood, it is hypothesized that the limited genetic diversity of the 17D vaccine virus attributes to vaccine attenuation and safety. There is evidence that replication of 17D is not as error-prone as wild-type RNA viruses. See Pugachev et al., J Virol. 78(2):1032-8 (2004).

The term “envelope protein” or “E protein” refers to the structural YFV protein that is a primary immunogen that plays a central role in receptor binding and membrane fusion. The structure of the E protein ectodomain (the soluble N-terminal portion consisting of 395 residues) includes three distinct structural domains, referred to as domains I, II, and III. (Volk et al., Virology 2009, 394(1): 12-18). Domain II contains a S—S bridge stabilized loop at its distal end that functions as a highly conserved fusion loop (FL). When a virus enters a target host cell, the FL of Domain II is exposed and inserts into the host cellular membrane. (Zhang et al., Viruses 2017, 9(11): 338). In some embodiments, the antibodies and antigen-binding fragments thereof bind to the FL of Domain II YFV E protein. In other embodiments, the antibodies and antigen-binding fragments thereof bind to Domain III of the YFV E protein.

The development of an effective YFV therapeutic has presented a number of unique challenges. The in-depth analysis of the human antibody response to the YFV vaccine performed here provides insights for the development of such a therapeutic treatment. The antibody repertoire analysis disclosed herein reveals that the majority of neutralizing YFV-specific antibodies target FL-proximal epitopes overlapping the 5A epitope, whereas a small number of potent neutralizing antibodies targeted the DIII domain—a region of the E protein that, until now, was not the epitope for any effective anti-YFV antibodies.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. The term “antibody” (or “Ab”), as used herein, is intended to refer to immunoglobulin molecules comprised of four polypeptide chains, two heavy (H) chains and two light (L) chains interconnected by disulfide bonds (i.e., “full antibody molecules”), as well as multimers thereof (e.g. IgM) or antigen-binding fragments thereof.

The terms “antigen-binding portion”, “antigen-binding fragment”, and the like, as used herein, include any naturally occurring, enzymatically obtainable, synthetic, or genetically engineered polypeptide or glycoprotein that specifically binds an antigen to form a complex. In certain embodiments, the terms “antigen-binding portion” or “antibody fragment”, as used herein, refer to one or more fragments of an antibody that retains the ability to bind to YFV.

An antibody fragment may include a Fab fragment, a F(ab′)₂ fragment, a Fv fragment, a dAb fragment, a fragment containing a CDR, or an isolated CDR. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}³; (iv) V_{H}-C_{H}1-C_{H}2; (V) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present disclosure using routine techniques available in the art.

Each heavy chain is comprised of a heavy chain variable region (“HCVR” or “V_{H}”) and a heavy chain constant region (comprised of domains C_{H}1, C_{H}2, and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining region (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen binding fragment thereof) may be identical to the human germline sequences, or may be naturally or artificially modified. An amino acid consensus sequence may be defined based on a side-by-side analysis of two or more CDRs. Accordingly, the CDRs in a heavy chain are designated “CDRH1”, “CDRH2”, and “CDRH3”, respectively, and the CDRs in a light chain are designated “CDRL1”, “CDRL2”, and “CDRL3”.

In some embodiments, the antibody or antigen-binding fragment thereof contains a CDRL3 binding domain comprising a consensus motif having the sequence QQX₁X₂X₃X₄X₅X₆T. X₁ is Y, F, or A, X₂ is N, H, or Y, X₃ is R, S, T, or D, X₄ is D, F, Y, W, or P, X₅ is P or S, X₆ is Y, F, K, or W. The following clones include this consensus motif: ADI-50211; ADI-48899; ADI-45136; ADI-45078; ADI-49162; ADI-49141; ADI-42844; ADI-48910; ADI-45074; ADI-49041; ADI-50220; ADI-42172; ADI-42178; ADI-50218; and ADI-49194.

In some embodiments, the present disclosure provides an antibody comprising a YFV binding domain, CDRL3, wherein the CDRL3 binding domain comprises a consensus motif, the consensus motif comprising the sequence QX₁X₂X₃X₄TX₅X₆T, wherein X₁ is Q or H, X₂ is A or S, X₃ is S or Y, X₄ is T or S, X₅ is R or P, and X₆ is Y, L, W, or R. The following clones include this consensus motif: ADI-42201; ADI-45164; ADI-46729; ADI-42223; ADI-46718; ADI-45076; ADI-48968; ADI-45156; ADI-50536; and ADI-50537.

In some embodiments, the present disclosure provides an antibody comprising a YFV binding domain, CDRL3, wherein the CDRL3 binding domain comprises a consensus motif, the consensus motif comprising the sequence GTWDX₁SX₂X₃SAGX₄V, wherein X₁ is S or T, X₂ is S or no amino acid, X₃ is L or P, and X₄ is K, G, or R. The following clones include this consensus motif: ADI-45083; ADI-42225; ADI-42210; ADI-42198; ADI-42809; ADI-42830; ADI-42818; ADI-42151; and ADI-50533.

In some embodiments, the present disclosure provides an antibody comprising a YFV binding domain, CDRH3, wherein the CDRH3 binding domain comprises a consensus motif, the consensus motif comprising the sequence AX₁X₂YDSX₃X₄YYX₅X₆X₇X₈, wherein X₁ is K or R, X₂ is Y, F, T, A, G, Y, or H, X₃ is S, N, or R, X₄ is A or G, X₅ is W or Y, X₆ is F, L, I, A, or E, X₇ is D, E, or H, and X₅ is Y, H, or S. The following clones include this consensus motif: ADI-45085; ADI-50211; ADI-45078; ADI-49162; ADI-45136; ADI-42172; ADI-49194; ADI-50203; ADI-42178; ADI-48908; ADI-42844; ADI-48910; and ADI-49168.

In some embodiments, the present disclosure provides an antibody comprising a YFV binding domain, CDRL2, wherein the CDRL2 binding domain comprises a consensus motif, the consensus motif comprising the sequence X₁X₂X₃X₄RPS, wherein X₁ is D or E, X₂ is N, V, or D, X₃ is K, N, D, or S, and X₄ is K, E, or R. The following clones include this consensus motif: ADI-49039; ADI-42229; ADI-45097; ADI-45083; ADI-42225; ADI-49139; ADI-48969; ADI-48900; ADI-42786; ADI-42210; ADI-42198; ADI-49154; ADI-49188; ADI-42188; ADI-42809; ADI-46596; ADI-42830; ADI-46591; ADI-48955; ADI-42818; ADI-46586; ADI-42151; ADI-45140; ADI-46722; ADI-45128; ADI-45127; ADI-46739; ADI-46724; ADI-50539; ADI-42114; ADI-50533; and ADI-49205.

In some embodiments, present disclosure provides an antibody comprising a YFV binding domain, CDRL2, wherein the CDRL2 binding domain comprises a consensus motif, the consensus motif comprising the sequence X₁X₂X₃X₄LX₅X₆, wherein X₁ is A, G, or R, X₂ is A or T, X₃ is S or T, X₄ is T, G, S, or I, X₅ is Q or R, and X₆ is S or R. The following clones include this consensus motif: ADI-49133; ADI-49033; ADI-48895; ADI-42201; ADI-42230; ADI-48916; ADI-42211; ADI-5164; ADI-42191; ADI-49145; ADI-46729; ADI-42189; ADI-46718; ADI-45076; ADI-48968; ADI-50203; ADI-42227; ADI-48894; ADI-50218; ADI-45156; ADI-50536; ADI-50537; ADI-46737; ADI-45123; and ADI-50200.

In some embodiments, present disclosure provides an antibody comprising a YFV binding domain, CDRL2, wherein the CDRL2 binding domain comprises a consensus motif, the consensus motif comprising the sequence X₁X₂SX₃RAX₄, wherein X₁ is G, D, R, or A, X₂ is A or S, X₃ is S, T, or N, and X₄ is T or A. The following clones include this consensus motif: ADI-49147; ADI-50201; ADI-45113; ADI-50219; ADI-48897; ADI-42194; ADI-42847; ADI-48908; ADI-42231; ADI-42233; ADI-45148; ADI-42187; ADI-42787; ADI-49141; ADI-42213; ADI-42192; ADI-49590; ADI-48462; ADI-42200; ADI-42181; ADI-49037; ADI-49137; and ADI-42817.

In some embodiments, present disclosure provides an antibody comprising a YFV binding domain, CDRL2, wherein the CDRL2 binding domain comprises a consensus motif, the consensus motif comprising the sequence X₁VX₂X₃RPS, wherein X₁ is D, E, or R, X₂ is S, T, N, or A, and X₃ is N, K, or Q. The following clones include this consensus motif: ADI-42228; ADI-42190; ADI-49183; ADI-49189; ADI-50205; ADI-50531; ADI-49138; ADI-45154; ADI-49161; ADI-49561; ADI-42219; ADI-48435; ADI-45161; ADI-42193; ADI-42149; ADI-42216; ADI-42810; ADI-48890; ADI-42206; ADI-48950; and ADI-42124.

In some embodiments, the present disclosure provides an antibody comprising a YFV binding domain, CDRL2, wherein the CDRL2 binding domain comprises a consensus motif, the consensus motif comprising the sequence X₁ASX₂LEX₃, wherein X₁ is R, Q, or K, X₂ is T, S, G, R, or I, and X₃ is T or S. The following clones include this consensus motif: ADI-42831; ADI-42821; ADI-45085; ADI-50211; ADI-48899; ADI-49168; ADI-45136; ADI-45078; ADI-42844; ADI-48910; ADI-49041; ADI-42172; ADI-42178; ADI-49032; and ADI-49194.

In some embodiments, the present disclosure provides an antibody comprising a YFV binding domain, CDRH2, wherein the CDRH2 binding domain comprises a consensus motif, the consensus motif comprising the sequence X₁X₂X₃HX₄X₅X₆X₇X₈YX₉PX₁₀X₁₁X₁₂S, wherein X₁ is D, E, or S, X₂ is I or V, X₃ is F or Y, X₄ is X or T, X₅ is G or E, X₆ is S, G, or T, X₇ is T or A, X₅ is N, S, H, K, or T, X₉ is N or S, X₁₀ is S or F, X₁₁ is L or V, and X₁₂ of K or E. The following clones include this consensus motif: ADI-45083; ADI-42225; ADI-49139; ADI-48900; ADI-42232; ADI-42786; ADI-42210; ADI-42198; ADI-49154; ADI-42188; ADI-42809; ADI-42818; ADI-42151; ADI-46722; ADI-46742; ADI-49141; ADI-46739; ADI-46724; ADI-50539; ADI-48951; ADI-50538; and ADI-50533.

In some embodiments, the present disclosure provides an antibody comprising a YFV binding domain, CDRH2, wherein the CDRH2 binding domain comprises a consensus motif, the consensus motif comprising the sequence X₁X₂X₃X₄DX₅X₆X₇KX₈X₉ADSX₁₀X₁₁G, wherein X₁ is V or L, X₂ is I or M, X₃ is S, W, or L, X₄ is F or Y, X₅ is E or G, X₆ is S or T, X₇ is K, N, or Y, X₅ is F, W, or Y, X₉ is Y or F, X₁₀ is V or L, and X₁₁ is K or R. The following clones include this consensus motif: ADI-45097; ADI-42144; ADI-49138; ADI-45154; ADI-49561; ADI-42189; ADI-42844; ADI-45161; ADI-48462; ADI-42172; ADI-42178; ADI-42217; ADI-46737; ADI-49205; ADI-45151; and ADI-46728.

In some embodiments, the present disclosure provides an antibody comprising a YFV binding domain, CDRL1, wherein the CDRL1 binding domain comprises a consensus motif, the consensus motif comprising the sequence RX₁ SX₂X₃X₄X₅X₆X₇X₈X₉, wherein X₁ is A or T, X₂ is Q or R, X₃ is S or T, X₄ is I or V, X₅ is S or T, X₆ is S, N, T, F, D, or G, X₇ is N, Y, W, F, or K, X₅ is L or V, and X₉ is A or N. The following clones include this consensus motif: ADI-49147; ADI-50201; ADI-45113; ADI-42201; ADI-42194; ADI-42847; ADI-45085; ADI-48908; ADI-50211; ADI-42231; ADI-45164; ADI-48899; ADI-46729; ADI-49168; ADI-49040; ADI-45136; ADI-45078; ADI-46718; ADI-49141; ADI-42844; ADI-42192; ADI-48910; ADI-42200; ADI-50203; ADI-42181; ADI-49041; ADI-50220; ADI-42172; ADI-42178; ADI-49032; ADI-49137; ADI-42817; ADI-45156; ADI-50536; ADI-50537; and ADI-49194.

In some embodiments, the present disclosure provides an antibody comprising a YFV binding domain, CDRL1, wherein the CDRL1 binding domain comprises a consensus motif, the consensus motif comprising the sequence SGSX₁SNX₂GX₃X₄X₅VX₆, wherein X₁ is N or S, X₂ is I or F, X₃ is S or N, X₄ is N, Y, S, or D, X₅ is Y, F, or D, and X₆ is S or A. The following clones include this consensus motif: ADI-49039; ADI-42229; ADI-45097; ADI-45083; ADI-42225; ADI-48900; ADI-42786; ADI-42210; ADI-42198; ADI-49154; ADI-42188; ADI-42809; ADI-46596; ADI-42830; ADI-46591; ADI-48955; ADI-42818; ADI-46586; ADI-42151; ADI-45140; ADI-46722; ADI-45128; ADI-46739; ADI-46724; ADI-50539; ADI-42114; and ADI-50533.

In some embodiments, the present disclosure provides an antibody comprising a YFV binding domain, CDRL1, wherein the CDRL1 binding domain comprises a consensus motif, the consensus motif comprising the sequence X₁GTX₂X₃DX₄GX₅X₆X₇X₈VS, wherein X₁ is A or T, X₂ is S, G, or R, X₃ is S or T, X₄ is V, F, or I, X₅ is G or A, X₆ is Y, D, or F, X₇ K or N, and X₅ is Y or F. The following clones include this consensus motif: ADI-48969; ADI-42228; ADI-42190; ADI-49183; ADI-49189; ADI-50205; ADI-50531; ADI-49138; ADI-45154; ADI-49161; ADI-49561; ADI-42219; ADI-48435; ADI-45161; ADI-45127; ADI-42149; ADI-42216; ADI-42810; ADI-48890; ADI-42206; ADI-48950; ADI-42124; and ADI-49205.

In some embodiments, the present disclosure provides an antibody comprising a YFV binding domain, CDRH1, wherein the CDRH1 binding domain comprises a consensus motif, the consensus motif comprising the sequence: X₁X₂FX₃X₄X₅X₆X₇X₈, wherein X₁ is F, Y, or L, X₂ is T, A, S, or N, X₃ is S, or T, X₄ is S, T, or R, X₅ is Y or L, X₆ is G, A, T, W, S, or D, X₇ is M, I, or L, and X₈ is H, S, N, or T. The following clones include this consensus motif: ADI-45090; ADI-49044; ADI-45113; ADI-42144; ADI-50026; ADI-45075; ADI-42230; ADI-42154; ADI-45085; ADI-42211; ADI-50211; ADI-42231; ADI-42233; ADI-49168; ADI-42187; ADI-49561; ADI-42219; ADI-50535; ADI-45136; ADI-42189; ADI-48435; ADI-46718; ADI-42844; ADI-45161; ADI-48910; ADI-48462; ADI-42200; ADI-50203; ADI-42149; ADI-42172; ADI-42178; ADI-50197; ADI-42810; ADI-50218; ADI-45156; ADI-50536; ADI-50537; ADI-46737; ADI-42114; ADI-49194; ADI-42124; and ADI-46728.

In some embodiments, the present disclosure provides an antibody comprising a YFV binding domain, CDRH1, wherein the CDRH1 binding domain comprises a consensus motif, the consensus motif comprising the sequence: X₁SIX₂X₃X₄X₅X₆WX₇, wherein X₁ is G or I, X₂ is S or T, X₃ is S, T, G, or no amino acid, X₄ is D, S, T, or G, X₅ is Y, N, or D, X₆ is Wo r Y, and X₇ is S or T. The following clones include this consensus motif: ADI-45083; ADI-42225; ADI-48900; ADI-42786; ADI-42210; ADI-49188; ADI-42188; ADI-42818; ADI-42151; ADI-48913; ADI-46722; ADI-49141; ADI-46741; ADI-46739; ADI-50539; ADI-50538; and ADI-50533.

In some embodiments, the present disclosure provides an antibody comprising a YFV binding domain, CDRH1, wherein the CDRH1 binding domain comprises a consensus motif, the consensus motif comprising the sequence: FX₁FSDX₂YMX₃, wherein X₁ is I or T, X₂ is H or Y, and X₃ is A or D. The following clones include this consensus motif: ADI-42191; ADI-49040; ADI-42223; ADI-42193; ADI-48968; ADI-42212; ADI-45126; ADI-42141; ADI-49140; ADI-48894; ADI-42226; ADI-49137; ADI-48890; ADI-42206; and ADI-49030.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

The fully human monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes fully monoclonal antibodies comprising variants of any of the CDR amino acid sequences disclosed herein having one or more conservative substitutions. For example, the present disclosure includes antibodies having CDR amino acid sequences with, e.g., 10 or fewer, 8 or fewer, 6 or fewer, 4 or fewer, etc. conservative amino acid substitutions relative to any of the CDR amino acid sequences disclosed herein. In some embodiments, the anti-YFV antibodies and antigen-binding fragments disclosed are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3.

In some embodiments, the anti-YFV antibodies and antigen-binding fragments disclosed are recombinant antibodies. The term “recombinant” generally refers to any protein, polypeptide, or cell expressing a gene of interest that is produced by genetic engineering methods. The term “recombinant” as used with respect to a protein or polypeptide, means a polypeptide produced by expression of a recombinant polynucleotide. The proteins used in the immunogenic compositions of the disclosure may be isolated from a natural source or produced by genetic engineering methods.

The antibodies of the disclosure may, in some embodiments, be recombinant human antibodies. The term “recombinant human antibody”, as used herein, is intended to include all antibodies, including human or humanized antibodies, that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

In some embodiments, the anti-YFV antibodies and antigen-binding fragments thereof are isolated antibodies. An “isolated antibody”, as used herein, refers to an antibody that is substantially free of other antibodies having different antigenic specificities (e.g., an isolated antibody that specifically binds YFV, or a fragment thereof, is substantially free of Abs that specifically bind antigens other than YFV).In some embodiments, the anti-YFV antibodies and antigen-binding fragments specifically bind to the YFV E protein, e.g., the FL of DII domain or DIII. The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, biolayer interferometry measurements using, e.g., a ForteBio Octet HTX instrument (Pall Life Sciences), which bind specifically to YFV. Moreover, multi-specific antibodies that bind to YFV protein and one or more additional antigens, or a bi-specific that binds to two different regions of YFV are nonetheless considered antibodies that “specifically bind”, as used herein. In certain embodiments, the antibodies disclosed herein display equilibrium dissociation constants (and hence specificities) of about 1×10⁻⁶ M; about 1×10⁻⁷ M; about 1×10⁻⁸ M; about 1×10⁻⁹ M; about 1×10⁻¹⁰ M; between about 1×10⁻⁶ M and about 1×10⁻⁷ M; between about 1×10⁻⁷M and about 1×10⁻⁸ M; between about 1×10⁻⁸ M and about 1×10⁻⁹ M; between about 1×10⁻⁹ M and about 1×10⁻¹⁰ M; or between about 1×10⁻⁹ M and about 1×10⁻¹⁰ M.

In some embodiments, the anti-YFV antibodies and antigen-binding fragments are high affinity binders. The term “high affinity” refers to those mAbs having a binding affinity to YFV, expressed as K_{D}, of at least 10⁻⁹ M; more preferably 10⁻¹⁰ M, more preferably 10⁻¹¹ M, more preferably 10⁻¹² M as measured by surface plasmon resonance, e.g., BIACORE™, biolayer interferometry measurements using, e.g., a ForteBio Octet HTX instrument (Pall Life Sciences), or solution-affinity ELISA.

By the term “slow off rate”, “Koff” or “kd” is meant an antibody that dissociates from YFV, with a rate constant of 1×10⁻³ s-t or less, preferably 1×10⁻⁴ s-t or less, as determined by surface plasmon resonance, e.g., BIACORE™ or a ForteBio Octet HTX instrument (Pall Life Sciences).

The specific embodiments, antibody or antibody fragments of the disclosure may be conjugated to a therapeutic moiety (“immunoconjugate”), such as an antibiotic, a second anti-YFV antibody, a vaccine, or a toxoid, or any other therapeutic moiety useful for treating a YFV infection.

Also contemplated are antibodies and antigen-binding fragments substantially identical to the antibodies provided herein. The term “substantial identity”, or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. Accordingly, nucleic acid sequences that display a certain percentage “identity” share that percentage identity, and/or are that percentage “identical” to one another. A nucleic acid molecule having substantial identity to a reference nucleic acid molecule may, in certain instances, encode a polypeptide having the same or substantially similar amino acid sequence as the polypeptide encoded by the reference nucleic acid molecule.

In some embodiments, the antibody or antibody binding fragment thereof comprises at least one of a CDRH1, a CDRH2, a CDRH3, a CDRL1, a CDRL2, and a CDRL3 amino acid sequence of such antibodies or the antigen-binding fragments thereof are at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to at least one the CDRH1, a CDRH2, a CDRH3, a CDRL1, a CDRL2, and/or a CDRL3 amino acid sequences as disclosed in Table 3 of an antibody selected from Antibody Number 1 through Antibody Number 152 as disclosed in Table 3.

In certain embodiments, the antibodies and antigen-binding fragments thereof comprise the CDRH3 amino acid sequence of any one of the antibodies designated Antibody Number 1 through Antibody Number 152 as disclosed in Table 3.

In certain embodiments, the antibodies and antigen-binding fragments thereof comprise the CDRH2 amino acid sequence of any one of the antibodies designated Antibody Number 1 through Antibody Number 152 as disclosed in Table 3.

In certain embodiments, the antibodies and antigen-binding fragments thereof comprise the CDRH1 amino acid sequence of any one of the antibodies designated Antibody Number 1 through Antibody Number 152 as disclosed in Table 3.

In certain embodiments, the antibodies and antigen-binding fragments thereof comprise the CDRL3 amino acid sequence of any one of the antibodies designated Antibody Number 1 through Antibody Number 152 as disclosed in Table 3.

In certain embodiments, the antibodies and antigen-binding fragments thereof comprise the CDRL2 amino acid sequence of any one of the antibodies designated Antibody Number 1 through Antibody Number 152 as disclosed in Table 3.

In certain embodiments, the antibodies and antigen-binding fragments thereof comprise the CDRL1 amino acid sequence of any one of the antibodies designated Antibody Number 1 through Antibody Number 152 as disclosed in Table 3.

In some embodiments, an anti-YFV antibody and antigen-binding fragment thereof is at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to any one of the antibodies designated as Antibody Number 1 through Antibody Number 152 as disclosed in Table 3.

In certain embodiments, the antibodies and antigen-binding fragments thereof comprise a heavy chain (HC) amino acid sequence of any one of the antibodies designated Antibody Number 1 through Antibody Number 152 as disclosed in Table 3.

In certain embodiments, the inventive antibodies and antigen-binding fragments thereof comprise a light chain (LC) amino acid sequence of any one of the antibodies designated Antibody Number 1 through Antibody Number 152 as disclosed in Table 3. In certain embodiments, the inventive antibodies and antigen-binding fragments thereof comprise a heavy chain (HC) amino acid sequence and a light chain (LC) amino acid sequence of any one of the antibodies designated Antibody Number 1 through Antibody Number 152 as disclosed in Table 3.

In certain embodiments, the antibodies and antigen-binding fragments thereof are each selected from the group consisting of the antibodies designated as Antibody Number 1 through Antibody Number 152 as disclosed in Table 3.

Also provided are nucleic acids encoding the antibodies described herein. In certain embodiments, isolated nucleic acid sequences are provided that encode antibodies that specifically bind to YFV and antigen-binding fragments thereof, wherein at least one of the CDRH1, CDRH2, CDRH3, CDRL1, CDRL2, and/or CDRL3 amino acid sequences of the antibody or the antigen-binding fragment thereof is at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to at least one the CDRH1, CDRH2, CDRH3, CDRL1, CDRL2, and/or CDRL3 amino acid sequences as disclosed in Table 3 of an antibody selected from Antibody Number 1 through Antibody Number 152 as disclosed in Table 3.

In certain embodiments, isolated nucleic acid sequences are provided that encode the antibodies and antigen-binding fragments thereof, wherein such nucleic acid sequences comprise sequences that encode the CDRH3 amino acid sequence of any one of the antibodies designated Antibody Number 1 through Antibody Number 152 as disclosed in Table 3.

In certain embodiments, isolated nucleic acid sequences are provided that encode the antibodies and antigen-binding fragments thereof, wherein such nucleic acid sequences comprise sequences that encode the CDRH2 amino acid sequences of any one of the antibodies designated Antibody Number 1 through Antibody Number 152 as disclosed in Table 3.

In certain embodiments, isolated nucleic acid sequences are provided that encode the antibodies and antigen-binding fragments thereof, wherein such nucleic acid sequences comprise sequences that encode the CDRH1 amino acid sequences of any one of the antibodies designated Antibody Number 1 through Antibody Number 152 as disclosed in Table 3.

In certain embodiments, isolated nucleic acid sequences are provided that encode the antibodies and antigen-binding fragments thereof, wherein such nucleic acid sequences comprise sequences that encode the CDRL3 amino acid sequences of any one of the antibodies designated Antibody Number 1 through Antibody Number 152 as disclosed in Table 3.

In certain embodiments, isolated nucleic acid sequences are provided that encode the antibodies and antigen-binding fragments thereof, wherein such nucleic acid sequences comprise sequences that encode the CDRL2 amino acid sequences of any one of the antibodies designated Antibody Number 1 through Antibody Number 152 as disclosed in Table 3.

In certain embodiments, isolated nucleic acid sequences are provided that encode the antibodies and antigen-binding fragments thereof, wherein such nucleic acid sequences comprise sequences that encode the CDRL1 amino acid sequences of any one of the antibodies designated Antibody Number 1 through Antibody Number 152 as disclosed in Table 3.

In certain embodiments, isolated nucleic acid sequences are provided that encode the antibodies and antigen-binding fragments thereof, wherein such nucleic acid sequences comprise sequences that encode the heavy chain (HC) amino acid sequences of any one of the antibodies designated Antibody Number 1 through Antibody Number 152 as disclosed in Table 3

In certain embodiments, isolated nucleic acid sequences are provided that encode the antibodies and antigen-binding fragments thereof, wherein such nucleic acid sequences comprise sequences that encode the heavy chain (LC) amino acid sequences of any one of the antibodies designated Antibody Number 1 through Antibody Number 152 as disclosed in Table 3. As applied to polypeptides, the term “substantial identity” or “substantially identical” means that two peptide sequences, when optimally aligned, such as by the programs GAP or BESTFIT using default gap weights, share at least 90% sequence identity, even more preferably at least 95%, 98% or 99% sequence identity. Accordingly, amino acid sequences that display a certain percentage “identity” share that percentage identity, and/or are that percentage “identical” to one another. Accordingly, amino acid sequences that display a certain percentage “identity” share that percentage identity, and/or are that percentage “identical” to one another.

In certain embodiments, the disclosed antibody amino acid sequences are, e.g.,: at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to other sequences and/or share such percentage identities with one another (or with certain subsets of the herein-disclosed antibody sequences).

Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. (See, e.g., Pearson (1994) Methods Mol. Biol. 24: 307-331). Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. (See, e.g., Altschul et al. (1990) J. Mol. Biol. 215: 403 410 and (1997) Nucleic Acids Res. 25:3389 402).

In certain embodiments, the antibody or antibody fragment for use in the method of the disclosure may be mono-specific, bi-specific, or multi-specific. Multi-specific antibodies may be specific for different epitopes of one target polypeptide or may contain antigen-binding domains specific for epitopes of more than one target polypeptide.

As disclosed herein, anti-YFV antibodies may be obtained from human B cells using techniques available to the artisan, and, for example, as described in the EXAMPLES below. Methods for generating human antibodies in transgenic animals, such as mice, are also known in the art and may be employed in order to derive antibodies in accordance with the present disclosure. Any such known methods can be used in the context of the present disclosure to make human antibodies that specifically bind to YFV (see, for example, U.S. Pat. No. 6,596,541).

In certain embodiments, the antibodies of the instant disclosure possess affinities (K_{D}) ranging from about 1.0×10⁻⁷M to about 1.0×10⁻¹²M, when measured by binding to antigen either immobilized on solid phase or in solution phase. In certain embodiments, the antibodies of the disclosure possess affinities (K_{D}) ranging from about 1×10⁻⁷ M to about 6×10⁻¹⁰ M, when measured by binding to antigen either immobilized on solid phase or in solution phase. In certain embodiments, the antibodies of the disclosure possess affinities (K_{D}) ranging from about 1×10⁻⁷ M to about 9×10⁻¹⁰M, when measured by binding to antigen either immobilized on solid phase or in solution phase.

In addition to the specific anti-YFV antibodies and antibody fragments disclosed herein, the present disclosure also contemplates variants of those antibodies and antibody fragments that maintain bioequivalency. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present disclosure encompass sequences that comprise one or more additions, deletions, or substitutions of nucleotides when compared to the disclosed sequence, but that encode an antibody or antibody fragment that is essentially bioequivalent to an antibody or antibody fragment of the disclosure.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Biological and Biophysical Characteristics of the Antibodies

In certain embodiments, the inventive antibodies and antigen-binding fragments thereof specifically bind to YFV, wherein at least one of the CDRH1, CDRH2, CDRH3, CDRL1, CDRL2, and/or CDRL3 amino acid sequences is at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to the corresponding CDRH1, CDRH2, CDRH3, CDRL1, CDRL2, and/or CDRL3 amino acid sequence as disclosed in Table 3 of an antibody selected from Antibody Number 1 through Antibody Number 152 as disclosed in Table 3.

In some embodiments, the anti-YFV antibodies and antigen-binding fragments thereof are neutralizing antibodies, i.e., exhibit neutralizing potency. A “neutralizing antibody”, as used herein (or an “antibody that neutralizes YFV activity” or “an antibody with neutralizing activity”), refers to an antibody whose binding to an antigen, e.g., the YFV E protein as the case may be as disclosed herein, results in inhibition of at least one biological activity. For example, an antibody of the disclosure may aid in blocking the fusion of YFV to a host cell, or prevent syncytia formation, or prevent the primary disease caused by YFV. Alternatively, an antibody of the disclosure may demonstrate the ability to ameliorate at least one symptom of the YFV infection. This inhibition of the biological activity of YFV can be assessed by measuring one or more indicators of YFV biological activity by one or more of several standard in vitro assays (such as a neutralization assay, as described herein) or in vivo assays known in the art (for example, animal models to look at protection from challenge with YFV following administration of one or more of the antibodies described herein).

In certain embodiments, the antibodies and antigen-binding fragments thereof display an in vitro neutralization potency (IC₅₀) of between about 0.5 microgram/milliliter (μg/ml) to about 5 μg/ml; between about 0.05 μg/ml to about 0.5 μg/ml; or less than about 0.05 mg/ml.

The term “IC₅₀” refers to the “half maximal inhibitory concentration”, which value measures the effectiveness of compound (e.g. anti-YFV antibody) inhibition towards a biological or biochemical utility. This quantitative measure indicates the quantity required for a particular inhibitor to inhibit a given biological process by half. In certain embodiments, YFV neutralization potencies for anti-YFV neutralizing antibodies disclosed herein are expressed as neutralization IC₅₀ values. Of the antibodies described herein, generally the antibodies binding to DIII of the YFV E protein possess the highest neutralization potency.

In some embodiments, the antibodies and antigen-binding fragments thereof cross-react with DENV-2, DENV-4, WNV, or ZIKV E proteins, i.e., bind to YFV E protein and an E protein from one or more of the other flaviviruses. In certain embodiments, such antibodies and antigen-binding fragments thereof bind to DENV-2, DENV-4, WNV, YFV, and ZIKV E proteins with high apparent avid affinities (K_{D}^{Apps}<10 nM). In certain embodiments, the cross-reactive antibodies or antigen-binding fragments thereof have neutralizing activity against YFV-17D and another flavivirus. In certain embodiments, the cross-reactive antibodies and antigen-binding fragments thereof bind to the FL epitope. In certain embodiments, the cross-reactive antibodies and antigen-binding fragments thereof bind to DIII. In a certain embodiment, the cross-reactive antibody is ADI-48905.

### Epitope Binning and Related Technologies

As described above and as demonstrated in the EXAMPLES, Applicant has characterized the epitopic binning of the inventive antibodies and antigen-binding fragments thereof. In addition to the methods for conducting such characterization, various other techniques are available to the artisan that can be used to carry out such characterization or to otherwise ascertain whether an antibody “interacts with one or more amino acids” within a polypeptide or protein. Exemplary techniques include, for example, a routine cross-blocking assay such as that described Antibodies, Harlow and Lane (Cold Spring Harbor Press, Cold Spring Harb., NY) can be performed. Other methods include alanine scanning mutational analysis, peptide blot analysis (Reineke (2004) Methods Mol Biol 248:443-63), peptide cleavage analysis crystallographic studies and NMR analysis. In addition, methods such as epitope excision, epitope extraction and chemical modification of antigens can be employed (Tomer (2000) Protein Science 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues that correspond to the specific amino acids with which the antibody interacts. See, e.g., Ehring (1999) Analytical Biochemistry 267 (2):252-259; Engen and Smith (2001) Anal. Chem. 73:256A-265A.

As the artisan will understand, an epitope can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (US 2004/0101920). Each category may reflect a unique epitope either distinctly different from or partially overlapping with an epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

As the artisan understands, one can easily determine whether an antibody binds to the same epitope as, or competes for binding with, a reference anti-YFV antibody by using routine methods available in the art. For example, to determine if a test antibody binds to the same epitope as a reference YFV antibody of the disclosure, the reference antibody is allowed to bind to a YFV protein or peptide under saturating conditions. Next, the ability of a test antibody to bind to the YFV molecule is assessed. If the test antibody is able to bind to YFV following saturation binding with the reference anti-YFV antibody, it can be concluded that the test antibody binds to a different epitope than the reference anti-YFV antibody. On the other hand, if the test antibody is not able to bind to the YFV molecule following saturation binding with the reference anti-YFV antibody, then the test antibody may bind to the same epitope as the epitope bound by the reference anti-YFV antibody of the disclosure.

To determine if an antibody competes for binding with a reference anti-YFV antibody, the above-described binding methodology is performed in two orientations: In a first orientation, the reference antibody is allowed to bind to a YFV molecule under saturating conditions followed by assessment of binding of the test antibody to the YFV molecule. In a second orientation, the test antibody is allowed to bind to a YFV molecule under saturating conditions followed by assessment of binding of the reference antibody to the YFV molecule. If, in both orientations, only the first (saturating) antibody is capable of binding to the YFV molecule, then it is concluded that the test antibody and the reference antibody compete for binding to YFV. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. (1990) 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Immunoconjugates

The disclosure encompasses a human YFV monoclonal antibody conjugated to a therapeutic moiety (“immunoconjugate”), such as an agent that is capable of reducing the severity of primary infection with YFV, or to ameliorate at least one symptom associated with YFV infection, including fever, muscle pains, headache, vomiting, diarrhea, bleeding, or the severity thereof. Such an agent may be a second different antibody to YFV, or a vaccine. The type of therapeutic moiety that may be conjugated to the anti-YFV antibody will take into account the condition to be treated and the desired therapeutic effect to be achieved. Alternatively, if the desired therapeutic effect is to treat the sequelae or symptoms associated with YFV infection, or any other condition resulting from such infection, such as, but not limited to, disseminated intravascular coagulation, acute kidney failure, and acute respiratory distress syndrome, it may be advantageous to conjugate an agent appropriate to treat the sequelae or symptoms of the condition, or to alleviate any side effects of the antibodies of the disclosure. Examples of suitable agents for forming immunoconjugates are known in the art, see for example, WO 05/103081.

### Multi-Specific Antibodies

The antibodies of the present disclosure may be mono-specific, bi-specific, or multi-specific. Multi-specific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multi-specific antibody with a second binding specificity.

### Therapeutic Administration and Formulations

The disclosure provides therapeutic compositions comprising the inventive anti-YFV antibodies or antigen-binding fragments thereof. The administration of therapeutic compositions in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of each of the antibodies of the disclosure may vary depending upon the age and the size of a subject to be administered, target disease, conditions, route of administration, and the like. When the antibodies of the present disclosure are used for treating a YFV infection, or for treating one or more symptoms associated with a YFV infection, such as the fever, nausea, or muscle aches associated with a YFV infection in a patient, or for lessening the severity of the disease, it is advantageous to administer each of the antibodies of the present disclosure intravenously or subcutaneously. Normally, each of the antibodies would be administered at a single dose of about 0.01 to about 30 mg/kg body weight, more preferably about 0.1 to about 20 mg/kg body weight, or about 0.1 to about 15 mg/kg body weight, or about 0.02 to about 7 mg/kg body weight, about 0.03 to about 5 mg/kg body weight, or about 0.05 to about 3 mg/kg body weight, or about 1 mg/kg body weight, or about 3.0 mg/kg body weight, or about 10 mg/kg body weight, or about 20 mg/kg body weight. Multiple doses may be administered as necessary. Depending on the severity of the condition, the frequency and the duration of the treatment can be adjusted. In certain embodiments, the antibodies or antigen-binding fragments thereof of the disclosure can be administered as an initial dose of at least about 0.1 mg to about 800 mg, about 1 to about 600 mg, about 5 to about 300 mg, or about 10 to about 150 mg, to about 100 mg, or to about 50 mg. In certain embodiments, the initial dose may be followed by administration of a second or a plurality of subsequent doses of the antibodies or antigen-binding fragments thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings {e.g., oral mucosa, nasal mucosa, rectal and intestinal mucosa, etc.) and may be administered together with other biologically active agents. Administration can be systemic or local. It may be delivered as an aerosolized formulation (See US2011/0311515 and US2012/0128669). The delivery of agents useful for treating respiratory diseases by inhalation is becoming more widely accepted (See A. J. Bitonti and J. A. Dumont, (2006), Adv. Drug Deliv. Rev, 58:1 106-1 1 18). In addition to being effective at treating local pulmonary disease, such a delivery mechanism may also be useful for systemic delivery of antibodies (See Maillet et al. (2008), Pharmaceutical Research, Vol. 25, No. 6, 2008).

The pharmaceutical composition can be also delivered in a vesicle, in particular a liposome (see, for example, Langer (1990) Science 249:1527-1533).

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present disclosure include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.) and the HUMIRA™ Pen (Abbott Labs, Abbott Park, IL), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Administration Regimens

In some embodiments, a therapeutically effective amount of an anti-YFV antibody or antigen-binding fragment thereof is provided to a subject in feed thereof, e.g., infected with YFV or at risk for infection with YFV. By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

According to certain embodiments, multiple doses of an antibody to YFV may be administered to a subject over a defined time course. The methods according to this aspect of the disclosure comprise sequentially administering to a subject multiple doses of an antibody to YFV. As used herein, “sequentially administering” means that each dose of antibody to YFV is administered to the subject at a different point in time, e.g., on different days separated by a predetermined interval (e.g., hours, days, weeks or months). The present disclosure includes methods which comprise sequentially administering to the patient a single initial dose of an antibody to YFV, followed by one or more secondary doses of the antibody to YFV and optionally followed by one or more tertiary doses of the antibody to YFV.

The terms “initial dose,” “secondary doses,” and “tertiary doses,” refer to the temporal sequence of administration of the antibody to YFV. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of antibody to YFV, but generally may differ from one another in terms of frequency of administration. In certain embodiments, however, the amount of antibody to YFV contained in the initial, secondary and/or tertiary doses vary from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1½, 2, 2½, 3, 3½, 4, 4½, 5 5½, 6, 6½, 7, 7½8, 8½, 9, 9½, 10, 10½, 11, 11½, 12, 12½, 13, 13½, 14, 14½2, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of antibody to YFV which is administered to a patient prior to the administration of the very next dose in the sequence with no intervening doses.

The methods according to this aspect of the disclosure may comprise administering to a patient any number of secondary and/or tertiary doses of an antibody to YFV. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Accordingly, in certain embodiments are provided pharmaceutical compositions comprising: one or more of the inventive antibodies or antigen-binding fragments thereof disclosed herein and throughout and a pharmaceutically acceptable carrier and/or one or more excipients. In certain other embodiments are provided pharmaceutical compositions comprising: one or more nucleic acid sequences encoding one or more inventive antibodies or antigen-binding fragments thereof; or one or more the expression vectors harboring such nucleic acid sequences; and a pharmaceutically acceptable carrier and/or one or more excipients.

### Therapeutic Uses of the Antibodies

The anti-YFV antibodies disclosed herein may be used to treat a subject with YFV and/or prevent YFV infection.

As used herein, the terms “treat,” “treatment” and “treating” refer to the reduction or amelioration of the progression, severity, and/or duration of a YFV infection, or a symptom or condition related thereto (such as fever, chills, headache, low back pain, myalgia, loss of appetite, nausea, vomiting, fatigue, or a combination thereof) resulting from the administration of one or more therapies (including, but not limited to, the administration of one or more prophylactic or therapeutic agents). In certain embodiments, such terms refer to the reduction or inhibition of the replication of YFV, the inhibition or reduction in the spread of YFV to other subjects, the inhibition or reduction of infection of a cell with YFV, or the amelioration of one or more symptoms associated with a YFV infection.

As used herein, the terms “prevent,” “preventing,” and “prevention” refer to the prevention or inhibition of the development or onset of a YFV infection or condition related thereto in a subject, the prevention or inhibition of the progression of a YFV infection or a condition related thereto resulting from the administration of a therapy (e.g., a prophylactic or therapeutic agent), the prevention of a symptom of a YFV infection or condition related thereto, or the administration of a combination of therapies (e.g., a combination of prophylactic or therapeutic agents). As used herein, the terms “ameliorate” and “alleviate” refer to a reduction or diminishment in the severity a condition or any symptoms thereof.

Due to their binding to and interaction with YFV, it is believed that the inventive antibodies and antigen-binding fragments thereof are useful—without wishing to be bound to any theory—for preventing fusion of the virus with the host cell membrane, for preventing cell to cell virus spread, and for inhibition of syncytia formation. Alternatively, the antibodies of the present disclosure may be useful for ameliorating at least one symptom associated with the infection, such as fever, diarrhea, and bleeding, or for lessening the severity, duration, and/or frequency of the infection. The antibodies of the disclosure are also contemplated for prophylactic use in patients at risk for developing or acquiring a YFV infection. It is contemplated that the antibodies of the disclosure may be used alone, or in conjunction with a second agent, or third agent for treating YFV infection, or for alleviating at least one symptom or complication associated with the YFV infection, such as fever, nausea, or muscle aches associated with, or resulting from such an infection. The second or third agents may be delivered concurrently with the antibodies of the disclosure, or they may be administered separately, either before or after the antibodies of the disclosure. The second or third agent may be an anti-viral, an NSAID or other agents to reduce fever or pain, another second but different antibody that specifically binds YFV, an agent (e.g. an antibody) that binds to another YFV antigen, a vaccine against YFV, and an siRNA specific for a YFV antigen.

In yet a further embodiment of the disclosure the present antibodies are used for the preparation of a pharmaceutical composition for treating patients suffering from a YFV infection. In yet another embodiment of the disclosure the present antibodies are used for the preparation of a pharmaceutical composition for reducing the severity of a primary infection with YFV, or for reducing the duration of the infection, or for reducing at least one symptom associated with the YFV infection. In a further embodiment of the disclosure the present antibodies are used as adjunct therapy with any other agent useful for treating an YFV infection, including an antiviral, a toxoid, a vaccine, a second YFV antibody, or any other antibody specific for a YFV antigen, or any other palliative therapy known to those skilled in the art.

Accordingly, in certain embodiments are provided methods of treating or preventing a YFV infection, or at least one symptom associated with YFV infection, comprising administering to a patient in need thereof or suspected of being in need thereof one or more of the inventive antibodies or antigen-binding fragments thereof disclosed herein and throughout, such as, e.g., one or more of the anti-YFV antibodies disclosed in Table 3, such that the YFV infection is treated or prevented, or the at least one symptom associated with YFV infection is treated, alleviated, or reduced in severity.

In certain other embodiments are provided methods of treating or preventing a YFV infection, or at least one symptom associated with YFV infection, comprising administering to a patient in need thereof or suspected of being in need thereof a nucleic acid sequence encoding one or more of the inventive antibodies or antigen-binding fragments thereof, such nucleic acid sequence encoding an amino acid sequence disclosed in Table 3 and compliments thereof, such that the YFV infection is treated or prevented, or the at least one symptom associated with YFV infection is treated, alleviated, or reduced in severity.

In additional embodiments are provided methods of treating or preventing a YFV infection, or at least one symptom associated with YFV infection, comprising administering to a patient in need thereof or suspected of being in need thereof a host cell harboring a nucleic acid sequence or an expression vector comprising such a nucleic acid sequence, wherein such nucleic acid sequences encode an amino acid sequence selected from sequences disclosed in Table 3 and compliments thereof, such that the YFV infection is treated or prevented, or the at least one symptom associated with YFV infection is treated, alleviated, or reduced in severity.

In additional embodiments are provided methods of treating or preventing a YFV infection, or at least one symptom associated with YFV infection, comprising administering to a patient in need thereof or suspected of being in need thereof a pharmaceutical composition comprising either: one or more of the inventive antibodies or antigen-binding fragments thereof as disclosed in Table 3; one or more nucleic acid sequences or an expression vectors comprising such a nucleic acid sequence, wherein such nucleic acid sequences encode amino acid sequences selected from sequences disclosed in Table 3 and compliments thereof, one or more host cells harboring one or more nucleic acid sequences or expression vectors comprising such one or more nucleic acid sequences, wherein such nucleic acid sequences encode amino acid sequences selected from sequences disclosed in Table 3 and compliments thereof, and a pharmaceutically acceptable carrier and/or one or more excipients, such that the YFV infection is treated or prevented, or the at least one symptom associated with YFV infection is treated, alleviated, or reduced in severity.

In certain embodiments are provided methods of treating or preventing a YFV infection, or at least one symptom associated with said YFV infection, comprising administering to a patient in need thereof or suspected of being in need thereof one or more of the inventive antibodies or antigen-binding fragments thereof disclosed herein and throughout, such as, e.g., one or more of the anti-YFV antibodies disclosed in Table 3, such that the YFV infection is treated or prevented, or the at least one symptom associated with YFV infection is treated, alleviated, or reduced in severity.

In certain other embodiments are provided methods of treating or preventing a YFV infection, or at least one symptom associated with said YFV infection, comprising administering to a patient in need thereof or suspected of being in need thereof a nucleic acid sequence encoding one or more of the inventive antibodies or antigen-binding fragments thereof, such nucleic acid sequences encoding amino acid sequences disclosed in Table 3 and compliments thereof, such that the YFV infection is treated or prevented, or the at least one symptom associated with YFV infection is treated, alleviated, or reduced in severity.

In additional embodiments are provided methods of treating or preventing a YFV infection, or at least one symptom associated with said YFV infection, comprising administering to a patient in need thereof or suspected of being in need thereof a host cell harboring a nucleic acid sequence or an expression vector comprising such a nucleic acid sequence, wherein such nucleic acid sequences encode amino acid sequences selected from sequences disclosed in Table 3 and compliments thereof, such that the YFV infection is treated or prevented, or the at least one symptom associated with YFV infection is treated, alleviated, or reduced in severity.

In additional embodiments are provided methods of treating or preventing a YFV infection, or at least one symptom associated with said YFV infection, comprising administering to a patient in need thereof or suspected of being in need thereof a pharmaceutical composition comprising either: one or more of the inventive antibodies or antigen-binding fragments thereof as disclosed in Table 3; one or more nucleic acid sequences or an expression vectors comprising such a nucleic acid sequence, wherein such nucleic acid sequences encode amino acid sequences selected from sequences disclosed in Table 3 and compliments thereof; one or more host cells harboring one or more nucleic acid sequences or an expression vectors comprising such one or more nucleic acid sequences, wherein such nucleic acid sequences encode amino acid sequences selected from sequences disclosed in Table 3 and compliments thereof; and a pharmaceutically acceptable carrier and/or one or more excipients, such that the YFV infection is treated or prevented, or the at least one symptom associated with YFV infection is treated, alleviated, or reduced in severity.

### Combination Therapies

As noted above, according to certain embodiments, the disclosed methods comprise administering to the subject one or more additional therapeutic agents in combination with an antibody to YFV. As used herein, the expression “in combination with” means that the additional therapeutic agents are administered before, after, or concurrent with the pharmaceutical composition comprising the anti-YFV antibody. The term “in combination with” also includes sequential or concomitant administration of the anti-YFV antibody and a second therapeutic agent.

For example, when administered “before” the pharmaceutical composition comprising the anti-YFV antibody, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the anti-YFV antibody. When administered “after” the pharmaceutical composition comprising the anti-YFV antibody, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the anti-YFV antibodies. Administration “concurrent” or with the pharmaceutical composition comprising the anti-YFV antibody means that the additional therapeutic agent is administered to the subject in a separate dosage form within less than 5 minutes (before, after, or at the same time) of administration of the pharmaceutical composition comprising the anti-YFV antibody, or administered to the subject as a single combined dosage formulation comprising both the additional therapeutic agent and the anti-YFV antibody.

Combination therapies may include an anti-YFV antibody of the disclosure and any additional therapeutic agent that may be advantageously combined with an antibody of the disclosure, or with a biologically active fragment of an antibody of the disclosure.

For example, a second or third therapeutic agent may be employed to aid in reducing the viral load in the liver, such as an antiviral. The antibodies may also be used in conjunction with other therapies, as noted above, including a toxoid, a vaccine specific for YFV, a second antibody specific for YFV, or an antibody specific for another YFV antigen.

### Diagnostic Uses of the Antibodies

The inventive anti-YFV antibodies and antigen-binding fragments thereof may also be used to detect and/or measure YFV in a sample, e.g., for diagnostic purposes. It is envisioned that confirmation of an infection thought to be caused by YFV may be made by measuring the presence of the virus through use of any one or more of the antibodies of the disclosure. Exemplary diagnostic assays for YFV may comprise, e.g., contacting a sample, obtained from a patient, with an anti-YFV antibody of the disclosure, wherein the YFV antibody is labeled with a detectable label or reporter molecule or used as a capture ligand to selectively isolate the virus containing the protein from patient samples. Alternatively, an unlabeled YFV antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure YFV in a sample include enzyme-linked immunosorbent assay (ELISA), radioimmunoassay (RIA), and fluorescence-activated cell sorting (FACS).

Samples that can be used in YFV diagnostic assays according to the present disclosure include any tissue or fluid sample obtainable from a patient, which contains detectable quantities of YFV protein, or fragments thereof, under normal or pathological conditions. Generally, levels of YFV in a particular sample obtained from a healthy patient (e.g., a patient not afflicted with a disease or condition associated with the presence of YFV) will be measured to initially establish a baseline, or standard, level of YFV protein. This baseline level of YFV can then be compared against the levels of YFV measured in samples obtained from individuals suspected of having an YFV infection, or symptoms associated with such infection.

### EXAMPLES

The human antibody response to YFV was comprehensively profiled by isolating and characterizing 152 YFV-specific monoclonal antibodies from the memory B cells of two flavivirus-naive donors following immunization with YFV-17D, and these antibodies were then used to map the antigenic topology of YFV. The anti-YFV antibodies obtained were found to bind several antigenic sites, most commonly targeting an epitope within or proximal to the FL of Domain II of the YFV E protein and, thus, providing support for the development of YFV antibodies that target Domain II. However, a second less common class of antibodies with highly potent neutralizing activity were found to target DIII of the virus. Such DIII-directed antibodies may be particularly valuable in the context of therapeutic application of monoclonal antibodies or cocktails as this epitope is subdominant in the natural immune response. Taken together, these results have implications for the design and evaluation of YFV vaccine and antibody-based therapeutic candidates and offer new options for passive prophylaxis.

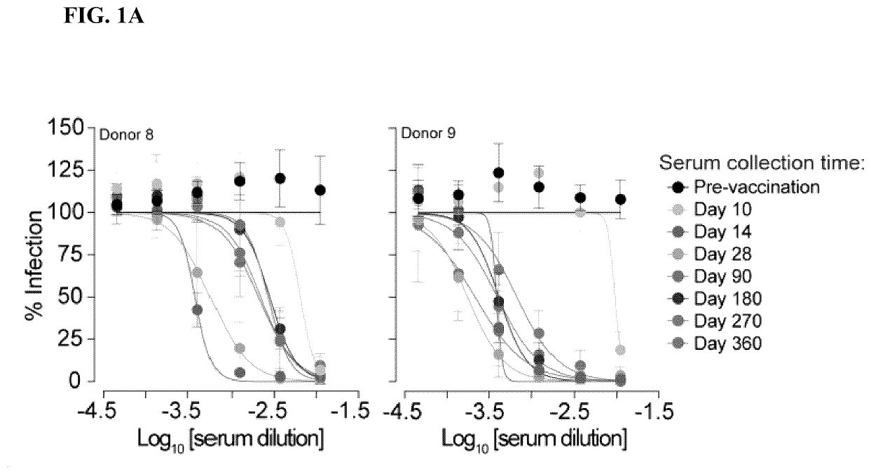

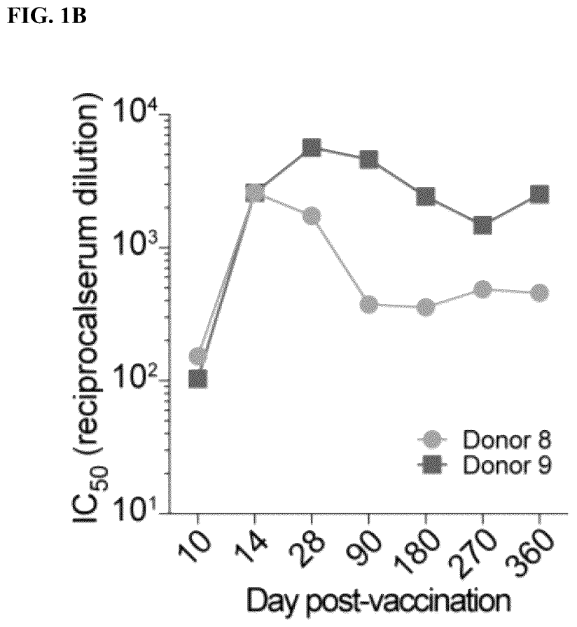

Study design: Two flavivirus-naïve healthy adult donors (“Donor 8” and “Donor 9”) were immunized with the YFV-17D vaccine (Stamaril; Sanofi) and blood samples were collected at 10, 14, 28, 90, 180, 270, and 360 days post-vaccination. Serum neutralizing activity against YFV-17D appeared in both donors by day 14 post-vaccination and persisted through the course of the study (FIG. 1A). Pre-vaccination sera from both donors lacked reactivity with YFV-17D and showed no detectable neutralizing activity against YFV-17D (data not shown) and also lacked reactivity with E and NS1 proteins from other commonly circulating flaviviruses, i.e., dengue virus serotypes 1-4 (DENV1-4), JEV, TBEV, West Nile virus (WNV) and Zika virus (ZIKV), confirming that both donors were likely flavivirus-naïve at the time of vaccination (data not shown).

### Molecular and Functional Characterization of YFV-17D Induced Plasmablast Response

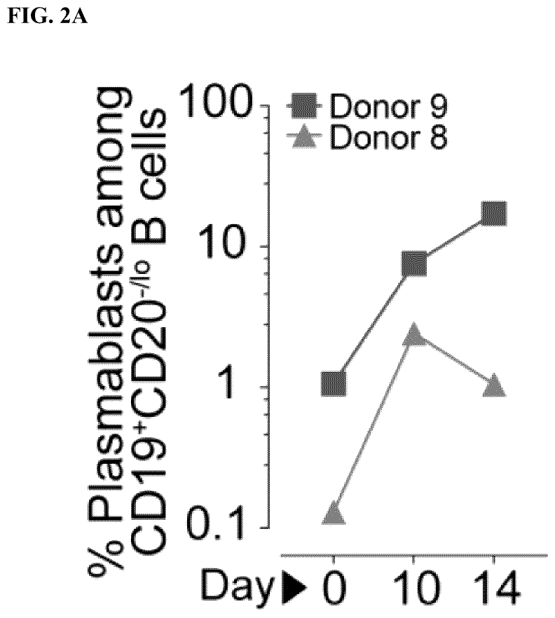

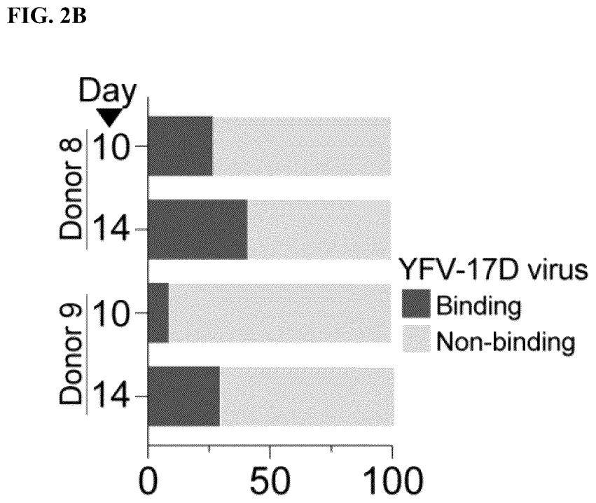

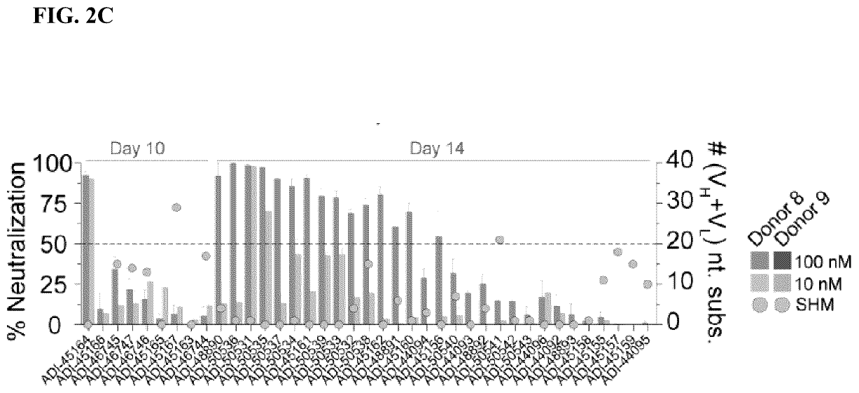

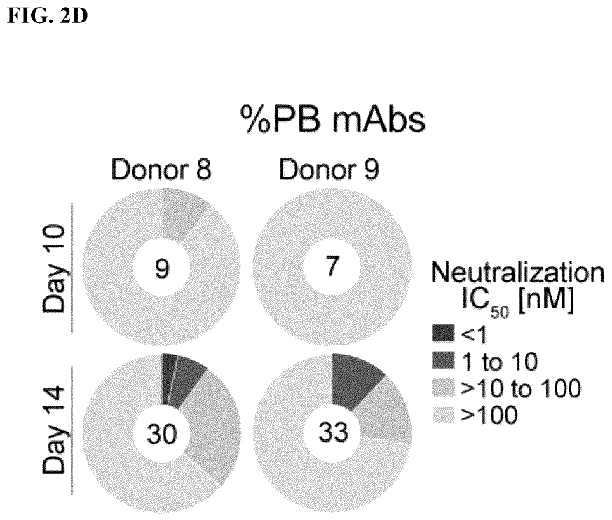

Plasmablast responses in both donors were monitored at 10 and 14 days post-vaccination. In both donors, expanded plasmablast populations were observed at both 10 and 14-day time points that were approximately 10-fold greater than pre-vaccination levels (FIG. 2A). Approximately 300 plasmablasts from each donor were sorted and amplified the corresponding VH and VL regions by single-cell PCR. 161 and 210 natively-paired antibodies were cloned from Donor 8 and Donor 9, respectively, and expressed as full-length IgGs in an engineered strain of Saccharomyces cerevisiae. Sequence analysis showed the plasmablast responses were highly diverse in both donors, with only about 15% of clones belonging to expanded clonal lineages (data not shown). A large fraction of plasmablast-derived antibodies from both donors contained high levels of somatic hypermutation (SHM), suggesting efficient recruitment of MBCs into the PB response (data not shown). The median level of SHM in the PB-derived mAbs was significantly higher on Day 10 than Day 14. Correspondingly, a larger proportion of mAbs cloned from day 14 PBs lacked SHM, suggesting an increased recruitment of cells from the naïve B cell compartment at this time point (data not shown).

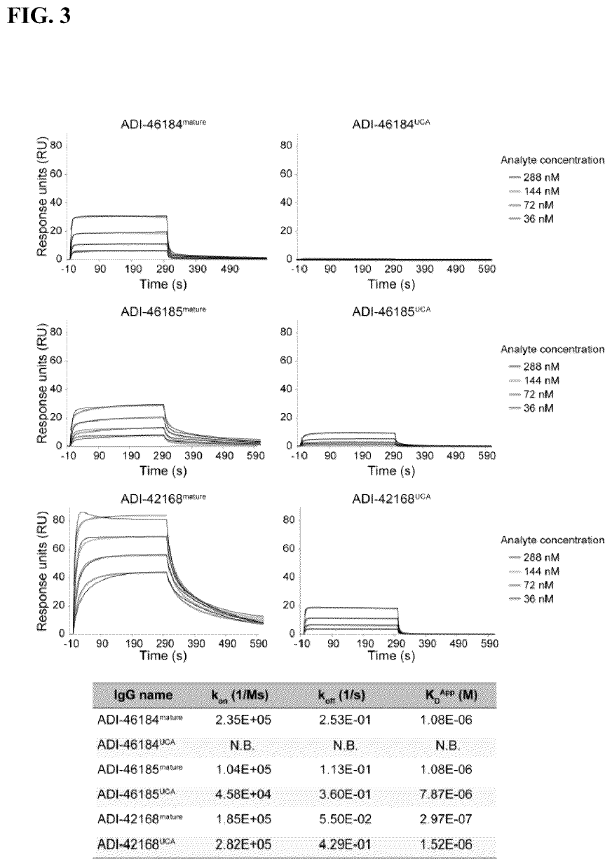

To analyze whether the somatic mutations in the PB-derived mAbs contribute to binding activity, inferred unmutated common ancestor (UCA) mAbs were generated from three somatically mutated PB clones and their binding affinities to a recombinant YFV E protein were measured. In all three cases, the UCA mAbs showed substantially reduced binding affinities compared to the mature mAbs, suggesting that somatic mutations in the PB mAbs are important for recognition of YFV E (FIG. 3).

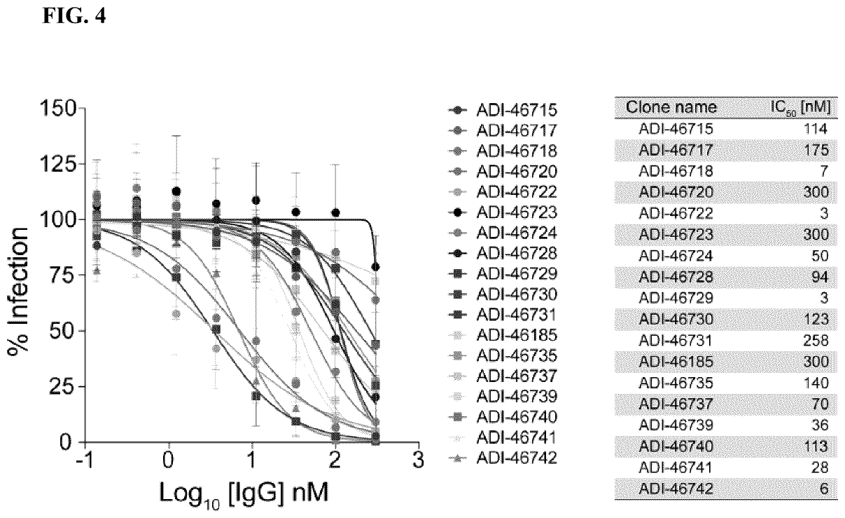

PB-derived mAbs were then tested for binding reactivity to YFV-17D particles using a sandwich ELISA assay (FIG. 2B). The frequency of YFV-17D binding mAbs isolated from day 10 and 14 PBs ranged from 8-41%. 45 and 46 YFV-17D binding mAbs were recovered from the expanded PB populations in Donor 8 and 9, respectively, and then analyzed the neutralizing activities of the mAbs in a micro-titer neutralization assay at 100 and 10 nM concentrations. Neutralizing activities ranged from complete neutralization at 10 nM to no detectable neutralization at 100 nM (FIG. 2C). A higher fraction of mAbs isolated from day 14 PBs displayed neutralizing activity compared to those isolated from day 10 PBs, which is consistent with the increased serum neutralizing activity on day 14 versus day 10 in both donors (data not shown). Neutralization titration experiments on the mAbs displaying at least 50% infection inhibition at 100 nM revealed that 9-12% of YFV-17D binding mAbs isolated from day 14 PBs displayed medium to high neutralizing activity (IC₅₀s≤10 nM) (FIG. 2D and FIG. 4). Sequence analysis showed that 12.5-33% of the PB-derived nAbs utilized VH4-4/VL1-51 germline gene pairing, suggesting recognition of a common antigenic site (data not shown).

About 50% and 22% of the neutralizing antibodies isolated from donor 8 and 9, respectively, lacked somatic mutations, indicating that YFV-17D neutralizing antibodies are present in the naïve B cell repertoire and suggesting that YFV-17D vaccination induces PB responses that originate from both naïve and MBCs, and only a minority of these B cells encode Abs that display neutralizing activity. See FIGS. 1A and 1B.

### Molecular and Functional Characterization of YFV-17D Induced MBC Response

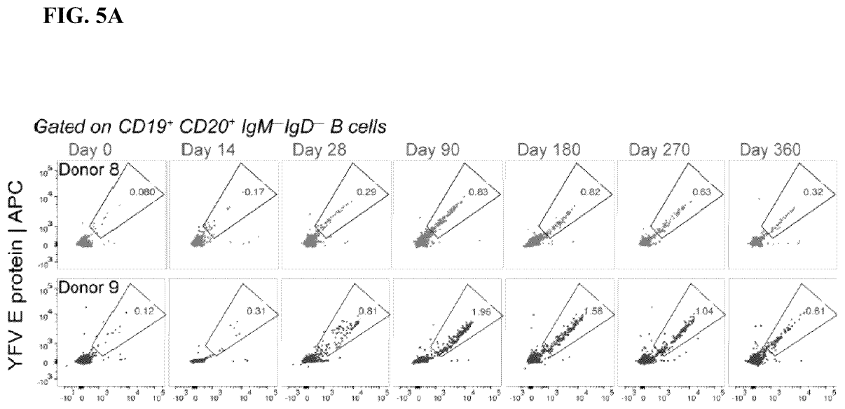

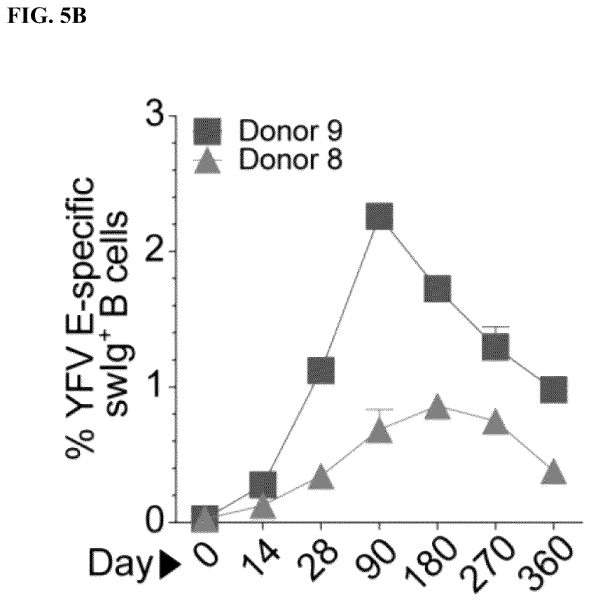

MBC responses in both donors were monitored by collecting PBMCs at days 14, 28, 90, 180, 270, and 360 post-vaccination and purified B cells were stained with a panel of previously described B cell surface markers (CD19, CD20, CD27, IgM, IgD, CD21, and CD71) and a fluorescently-labeled recombinant YFV E protein (FIG. 5A). YFV E-specific swIg⁺ MBCs emerged in both donors by Days 14-28, peaked between Days 90 and 180, and slowly declined between Days 180 and 360 (FIG. 5B).

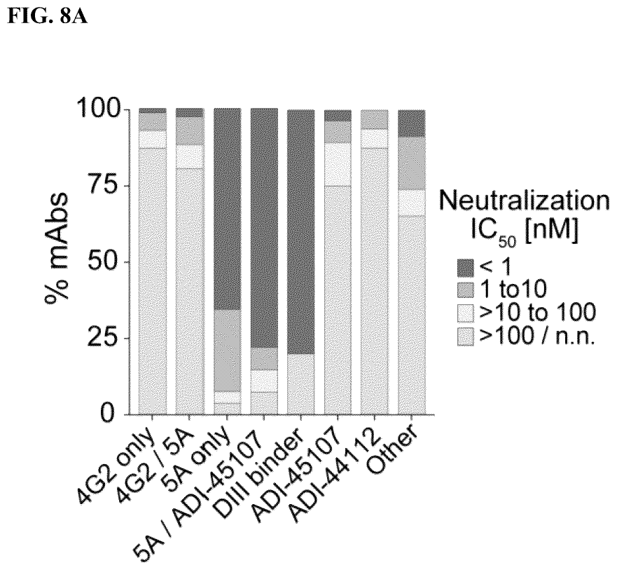

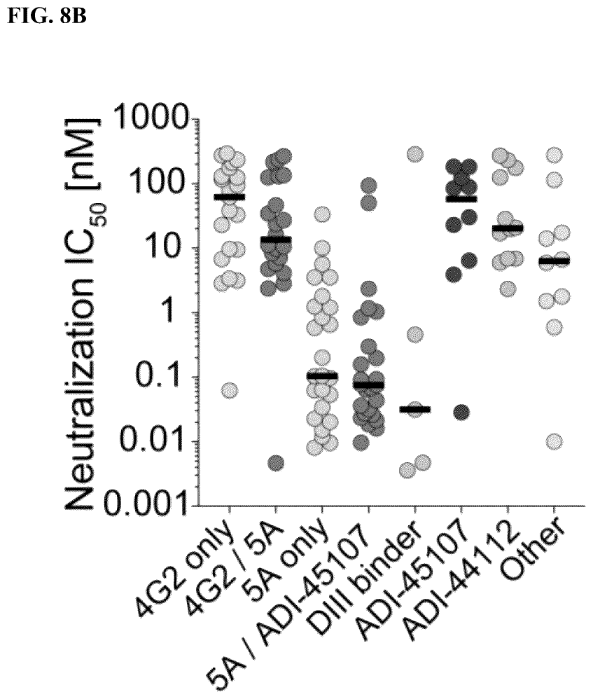

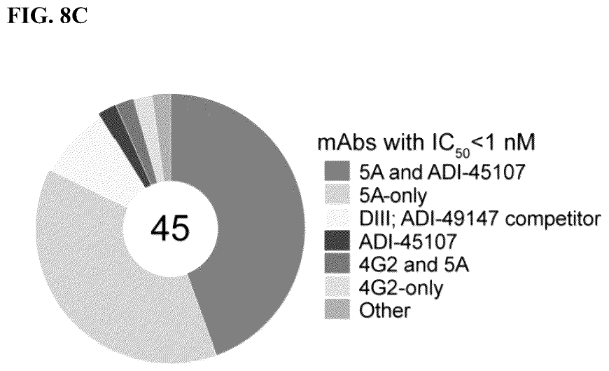

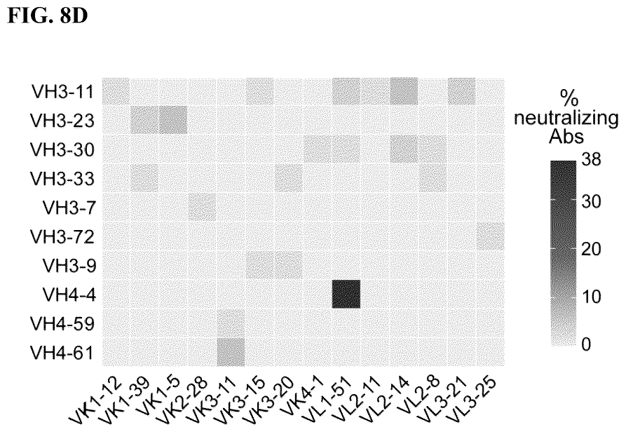

Between 100-400 YFV E-reactive B cells were sorted from both donors at each sampling time point. Naïve B cell-derived non-binding mAbs were captured via the sorting strategy employed but excluded from subsequent analyses. Analysis of the B cell surface markers expressed on the single-cell sorted, YFV E-reactive B cells revealed that the MBC response to YFV E was highly heterogenous at all time points (data not shown). At the earliest sampling time point (day 14), activated naïve B cells and IgM+CD27+ MBCs dominated the response in both donors, but these B cell populations waned rapidly over time. By day 90, less than 15% of the YFV E-specific response was comprised of IgM+CD27+ MBCs, and by day 360, only about 5% of YFV E-specific B cells belonged to this MBC population (FIG. 8B). In contrast, the swIg+ MBC population—which was comprised of both CD27+ and CD27− B cells—expanded between day 14 and day 90 and then remained stable throughout the course of the study. The MBC response observed following YFV-17D vaccination was also observed following natural infection with PUUV (data not shown).

SHM loads, apparent binding affinities (K_{D}^{Apps}), and neutralization potencies of the YFV E-specific mAbs were tracked at each sampling time point. In both donors, the median level of SHM was low at day 14—with over 50% of Abs lacking somatic mutations—and increased gradually over a 6-9-month time period, plateauing in both donors by 9 months post-vaccination, with a median of 9 and 7 nucleotide substitutions in VH for donor 8 and 9, respectively (data not shown). Binding studies with a recombinant YFV E protein showed that the K_{D}^{Apps} of the MBC-derived mAbs were very weak at early time points and progressively improved for 6-9 months following vaccination (data not shown). On days 14 and 28 post-vaccination, the majority of YFV E-specific mAbs displayed K_{D}^{Apps}>50 nM, whereas by day 180, about 50% of the YFV E-specific mAbs displayed K_{D}^{Apps}<5 nM. In parallel with the increase in affinity, the emergence of highly potent neutralizing antibodies (IC₅0<1 nM) were observed beginning at day 90 (data not shown). These neutralizing antibodies were derived from multiple MBC subsets, including atypical IgM+ and/or IgD+ MBCs (data not shown). Table 2 summarizes affinity and neutralization data for the isolated and characterized neutralizing mAbs.

Ongoing B cell activation was assessed by analyzing expression of CD71 and CD21 on YFV E-specific MBCs. CD71 was expressed on 75-85% YFV E-specific B cells at day 14 and remained elevated for about 6 months in both donors (data not shown). In both donors, YFV E-specific CD21^{lo} cells were present at high frequencies on days 14 and 28 post-vaccination, comprising about 40-80% of the YFV E-specific response, and then declined rapidly by day 90. While there was a high degree of overlap between the CD71⁺ and CD211^{lo} populations, with 50-80% of YFV E-specific activated B cells (defined as CD71⁺ and/or CD21^{lo}) displaying a CD71+CD21^{lo} phenotype at day 14, by day 28-90, the CD71+CD21^{lo} population waned to <50% of the activated B cell response in both donors and the majority of YFV E-specific activated B cells displayed either a CD71⁺CD21⁺ or CD71⁻CD21^{lo} phenotype and were heterogenous with regard to isotype and CD27 expression(data not shown).

### Isolation and Characterization of Anti-YFV Antibodies

Approximately 152 neutralizing monoclonal antibodies were isolated and characterized. Antibody variable heavy (VH) and variable light (VL) chain genes were rescued by single-cell PCR. Tiller et al. (2008) J Immunol Methods 329, 112-124. Cognate heavy and light chain pairs were subsequently cloned and expressed as full-length IgGs in an engineered strain of Saccharomyces cerevisiae for further characterization. Bornholdt et al., (2016) Science 351, 1078-1083.

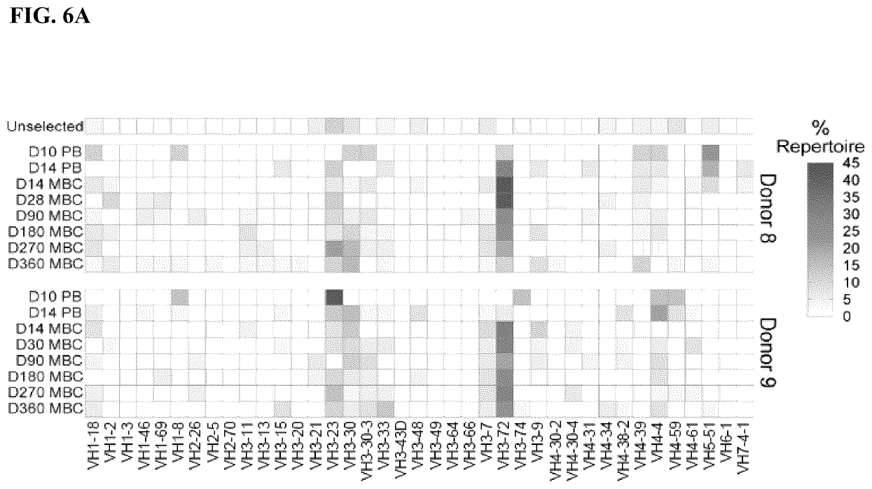

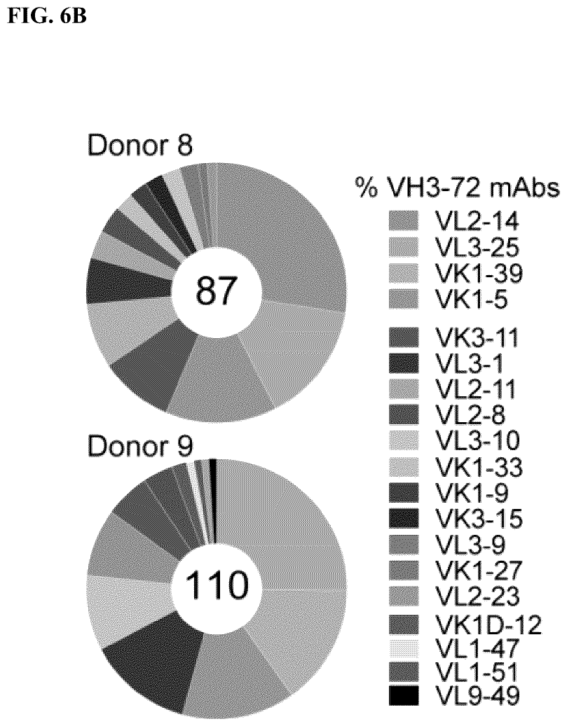

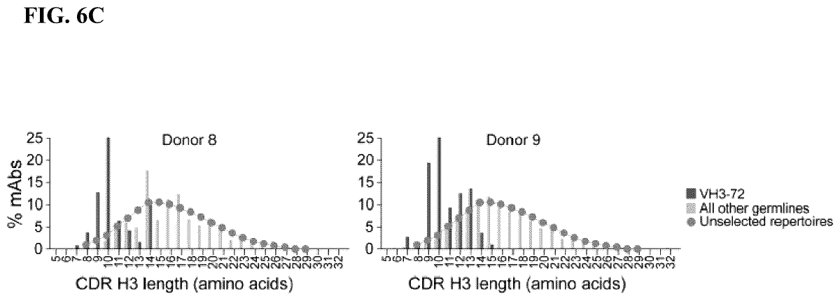

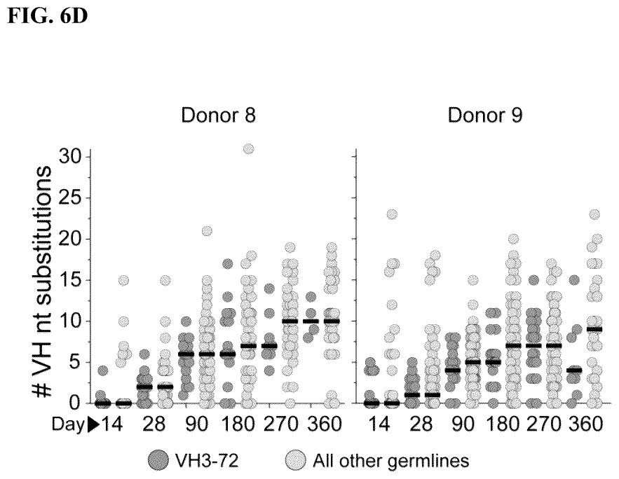

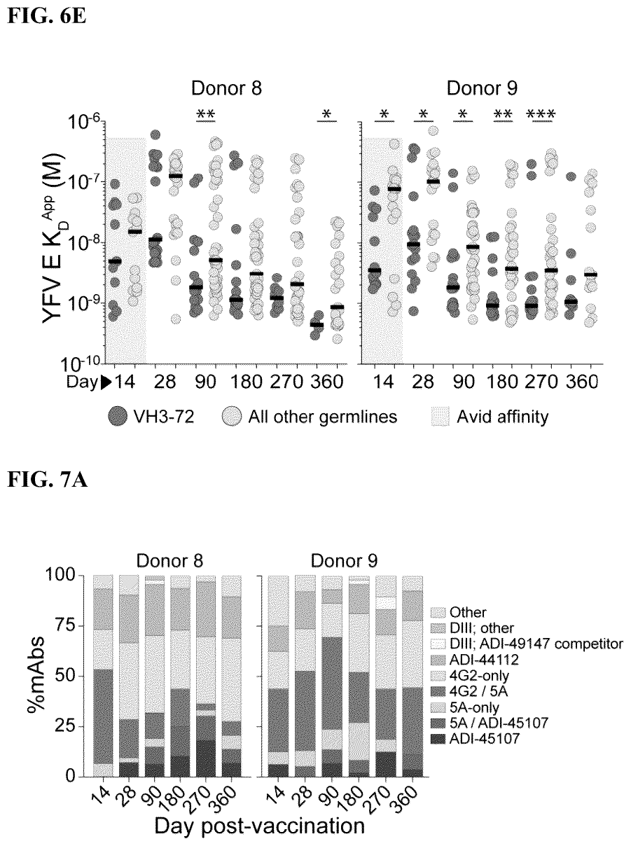

Germline gene usage of the isolated mAbs was analyzed. In both donors, mAbs utilizing the VH3-72 germline gene dominated the response at all time points (FIG. 6A). A large fraction of these mAbs also utilized one of five dominant light chain (LC) germline genes and displayed shorter-than-average heavy chain (HC) complementary determining region 3 (CDRH3) lengths, suggesting a shared mode of antigen recognition (FIG. 6B-C). The binding affinities of the mAbs utilizing VH3-72 were significantly higher than those observed for mAbs utilizing other VH germlines, despite containing similar levels of SHM (FIG. 6D-E). Table 1 summarizes germline usage and number of nucleotide substitions for isolated mAbs.

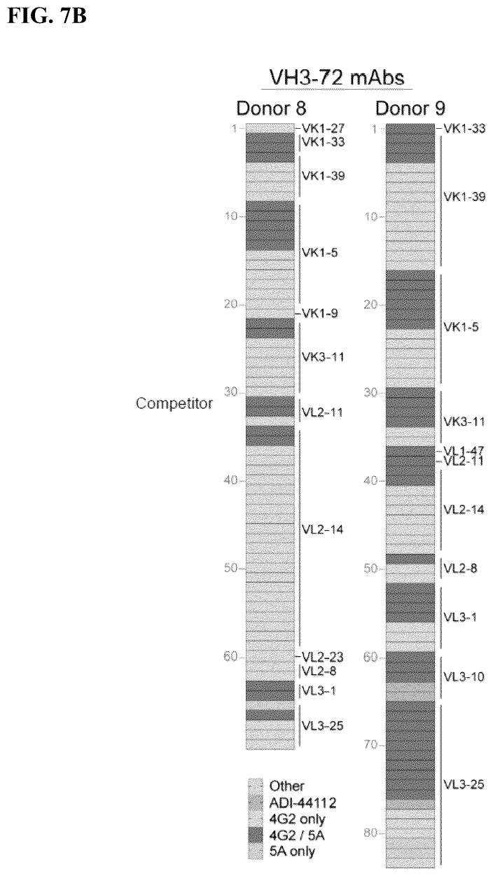

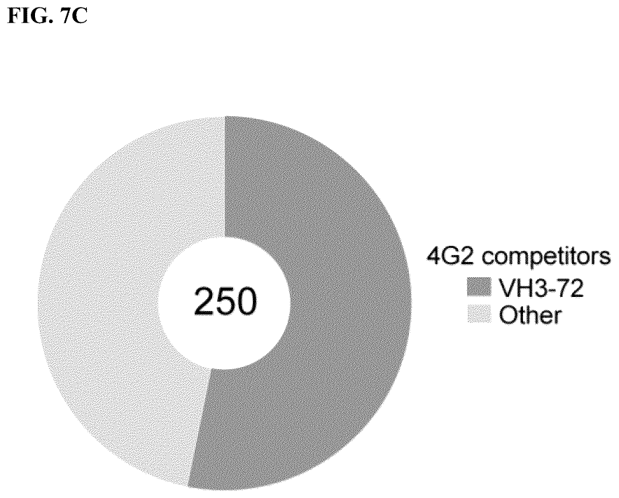

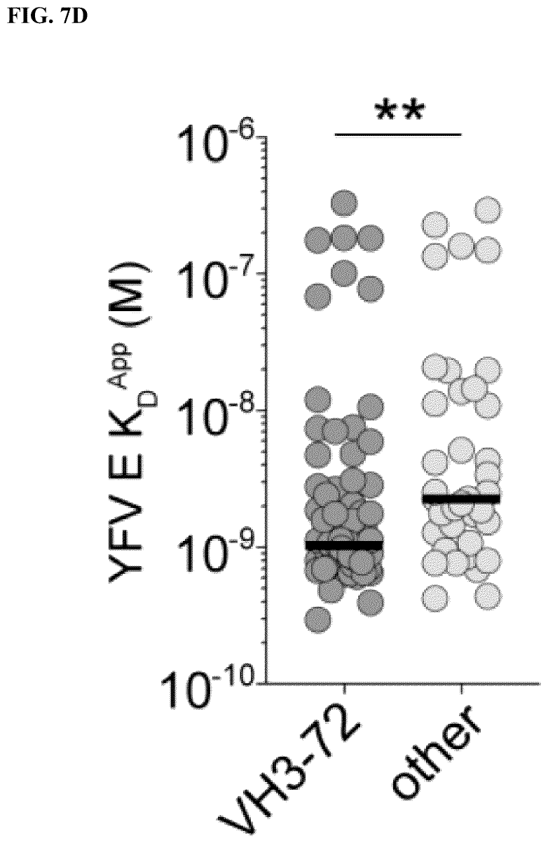

To explore the epitope coverage of the isolated mAbs, pairwise competition experiments were performed using the newly isolated mAbs and two well-characterized control mAbs, 4G2 and 5A, which recognize proximal but non-overlapping epitopes within DII of the YFV E monomer. 4G2 is a pan-flavivirus mAb that targets the FL, whereas 5A is a YFV E-specific mAb that binds to a FL-proximal epitope overlapping the proposed prM association region. Competition experiments were performed using high-throughput surface plasmon resonance (SPR) on a Carterra LSA instrument. Reactivity of the mAbs with a recombinant YFV-17D DIII protein by BLI was also evaluated. The majority of mAbs recognized one of eight distinct antigenic sites, which were defined based on reactivity with DIII and competition with 4G2, 5A, and three of the newly isolated mAbs (ADI-49147, ADI-44112, and ADI-45107) (FIG. 7A). A subset of mAbs competed with both 5A and ADI-45107, suggesting that these two antigenic sites are in close proximity. A small subset of mAbs (6 of 772) recognized epitopes within DIII. Five of the DIII-directed mAbs cross-competed, whereas the sixth, ADI-48945, may recognize a unique epitope. Over half of the mAbs from both donors competed with 4G2 and/or 5A, suggesting that the majority of the YFV E-specific response is mediated by Abs that target epitopes within or proximal to the FL on DII (FIG. 7A). Nearly all the mAbs that utilized the VH3-72 germline gene competed with 4G2 (FIG. 7B). Accordingly, analysis of the sequence features of the mAbs clustered by competition group revealed that over half of the mAbs that competed with 4G2 utilized the VH3-72 germline gene (FIG. 7C). The 4G2 competitor mAbs utilizing VH3-72 showed significantly higher affinities compared to those utilizing other VH germline genes (FIG. 7D). Although the proportion of mAbs targeting each antigenic site did not change dramatically over time, suppression of 4G2/5A competitor mAbs was observed at later timepoints in donor 8 (days 270 and 360). Furthermore, in both donors, mAbs that competed with both 5A and ADI-45107 did not emerge until day 28-90. Results suggest that the vast majority of the YFV E-specific response is directed against epitopes within or proximal to the FL on domain II, and there are only minor shifts in Ab immunodominance hierarchy during the maturation of the B cell response to YFV-17D.

### Highly Potent Neutralizing Antibodies Recognize FL Proximal Epitopes

The relationship between antigenic site and neutralization potency was investigated. Over 90% of the mAbs that competed either with 5A only or both 5A and ADI-45107 showed neutralizing activity (FIG. 8A). The majority (78%) of highly potent neutralizing antibodies (IC₅₀<1 nM) in the panel belonged to these two competition groups (FIG. 8B-8C). Table 2 provides bin data for these antibodies. Analysis of the sequence features of these 5A-only or 5A/ADI-45107 competitor neutralizing antibodies revealed that nearly 40% utilized VH4-4/VL1-51 germline gene pairing and did not show evidence of a convergent CDRH3 sequence, suggesting a common mode of germline-encoded antigen recognition (FIG. 8D). In line with prior studies, most of the DIII-directed mAbs also showed highly potent neutralizing activity. In contrast to the 5A competitors and DIII-directed mAbs, only a minority of the mAbs belonging to other competition groups showed neutralizing activity. For example, only 12% and 20% of mAbs that competed with 4G2 only or both 4G2 and 5A, respectively, displayed neutralization IC₅₀s<100 nM. The results demonstrate that the nAb response to YFV-17D is primarily mediated by Abs that recognize FL proximal epitopes within DII of the YFV E protein.

A Subset of mAbs Display Cross-Reactivity with E Proteins from Other Flaviviruses

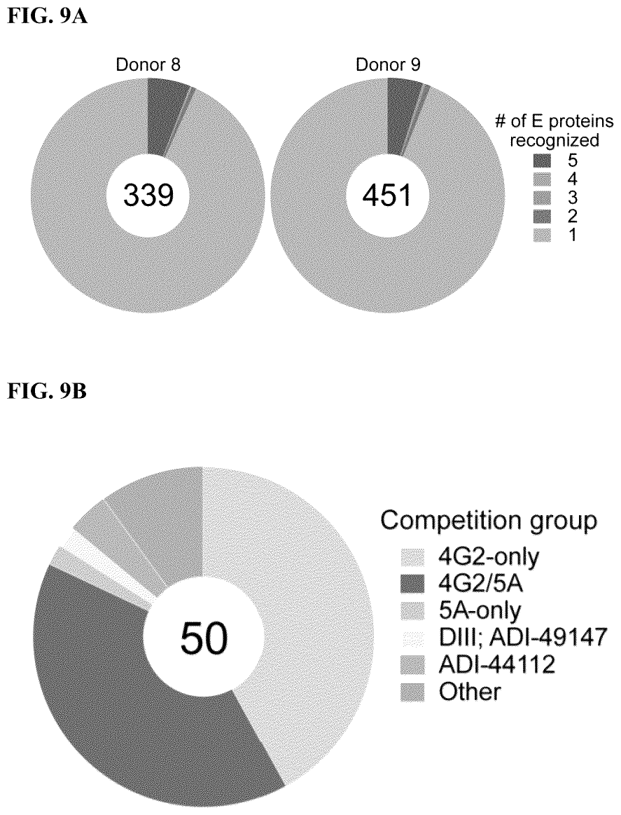

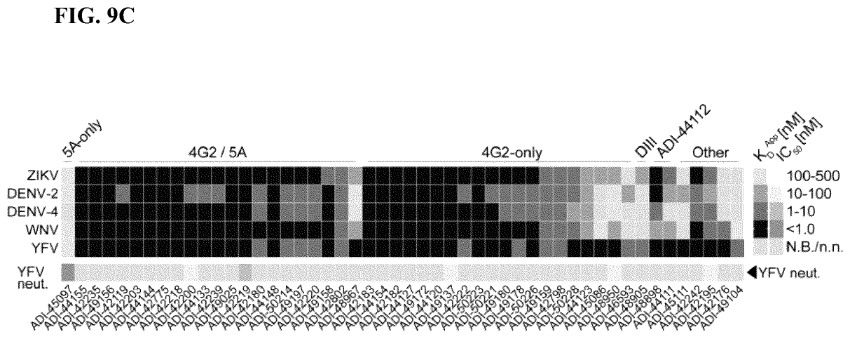

The isolated mAbs were evaluated for binding reactivity to recombinant DENV-2, DENV-4, WNV, or ZIKV E proteins. In both donors, about 6% of YFV E-reactive mAbs showed cross-reactivity to at least one heterologous flavivirus E protein (FIG. 9A). The majority of these cross-reactive mAbs targeted the highly conserved FL epitope and bound to all five flavivirus E proteins with high apparent avid affinities (K_{D}^{Apps}<10 nM) (FIG. 9B-9C). Correspondingly, the small subset of mAbs that bound to epitopes outside of the FL generally displayed more limited cross-reactivity profiles and lower K_{D}^{Apps} (FIG. 9C). Only 6 out of 50 cross-reactive mAbs showed neutralizing activity against YFV-17D, and only a single mAb, the DIII binder ADI-48905, showed detectable albeit weak neutralizing activity against ZIKV (IC₅₀˜100 nM). None of the mAbs had measurable neutralization activity against the West Nile virus or Japanese encephalitis virus reporter viral particles. YFV-17D vaccination thus appears to induce a subset of Abs that display broad flavivirus binding activity, the majority of which target the highly conserved FL and show little to no cross-neutralizing activity.

Table 1 below provides the germline usage and amino acid sequence information of 152 anti-YFV antibodies as described herein. The sequences provided in Table 1 include the CDRH3 sequence (SEQ ID NOs. 1, 3, 5, 7, 9, 11, 13, 15, 17, 19, 21, 23, 27, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, and 302) and the CDRL3 sequence (SEQ ID NOs. 2, 4, 6, 8, 10, 12, 14, 16, 18, 20, 22, 24, 26, 29, 31 33 35 37 39 41 43 45 47 49 51 53 55 57 59 61 63 65 67 69 71 73 75 77 79 81 83 85 87 89 91 93 95 97 99 101 103 105 107 109 111 113 115 117 119 121 123 125 127 129 131 133 135 137 139 141 143 145 147 149 151 153 155 157 159 161 163 165 167 169 171 173 175 177 179 181 183 185 187 189 191 193 195 197 199 201 203 205 207 209 211 213 215 217 219 221 223 225 227 229 231 233 235 237 239 241 243 245 247 249 251 253 255 257 259 261 263 265 267 269 271 273 275 277 279 281 283 285 287 289 291 293 295 297 299 301, and 303) for each listed antibody.

Table 2 below provides affinity and neutralization data for the 152 anti-YFV antibodies set forth in Table 1.

Table 3 below provides partial amino acid sequences for the CDRs of the heavy and light chains of each of the 152 anti-YFV antibodies set forth in Table 1. CDRs are indicated in bold/underlined. Each CDR amino acid sequence is also listed separately in the sequence listing (CDRH1 and CDRH2 correspond to SEQ ID NOs.: 607-840; CDRL1 and CDRL2 correspond to SEQ ID NOs.: 841-1005).

TABLE 1
Germline usage and sequence information of anti-YFV antibodies

| | | VH | LC | | | Number of | Number of |
|---|---|---|---|---|---|---|---|
| | | germline | germline | | | nucleotide | nucleotide |
| Antibody | | gene | gene | | | substitutions | substitutions |
| Number | Name | usage | usage | CDRH3 sequence | CDRL3 sequence | in VH | in VL |
| 1 | ADI- | VH4-38-2 | VL1-51 | ARNAPENYYGSGRESFDI | GTWDSSLSAWV | 7 | 5 |
| | 49039 | | | (SEQ ID NO: 1) | (SEQ ID NO: 2) | | |
| 2 | ADI- | VH3-23 | VK3-15 | AKDHGGKYGWWYFDL | QQYDNWPLT | 8 | 4 |
| | 49147 | | | (SEQ ID NO: 3) | (SEQ ID NO: 4) | | |
| 3 | ADI- | VH4-38-2 | VL1-51 | ARNAPENYYGSGRESFDI | GTWDSSLSAWV | 5 | 5 |
| | 42229 | | | (SEQ ID NO: 5) | (SEQ ID NO: 6) | | |
| 4 | ADI- | VH3-33 | VL1-44 | ARDLEVGAEYLYYHYGM | AAWDDSLNGW | 11 | 10 |
| | 45090 | | | DV (SEQ ID NO: 7) | V (SEQ ID NO: 8) | | |
| 5 | ADI- | VH3-30 | VL1-51 | AKDSSTSWYQVVYHIDY | ETWDSSLNAVV | 7 | 6 |
| | 45097 | | | (SEQ ID NO: 9) | (SEQ ID NO: 10) | | |
| 6 | ADI- | VH3-23 | VK1-39 | AKDLAVSTPRYWFDS | QQSYSIPRIT | 10 | 9 |
| | 49133 | | | (SEQ ID NO: 11) | (SEQ ID NO: 12) | | |
| 7 | ADI- | VH3-23 | VK1-39 | AKDMAVSVHRGWFDD | QQSYSPPMYT | 14 | 9 |
| | 49033 | | | (SEQ ID NO: 13) | (SEQ ID NO: 14) | | |
| 8 | ADI- | VH3-33 | VL1-44 | ARDLEVGAEYIYYYYGM | AAWDDSRNGW | 10 | 9 |
| | 49044 | | | DV (SEQ ID NO: 15) | V (SEQ ID | | |
| 9 | ADI- | VH4-4 | VL1-51 | ARSHWRSPQSVTFDL | GTWDTSSLSAG | 16 | 5 |
| | 45083 | | | (SEQ ID NO: 17) | RV (SEQ ID | | |
| 10 | ADI- | VH4-4 | VL1-51 | ARIAAGYSTSWYYFDY | GTWDTSLSAGR | 5 | 3 |
| | 42225 | | | (SEQ ID NO: 19) | V (SEQ ID | | |
| 11 | ADI- | VH4-4 | VL1-51 | AKDMWAGTTTNWFGP | GTWDTSLGVV | 9 | 5 |
| | 49139 | | | (SEQ ID NO: 21) | (SEQ ID NO: 22) | | |
| 12 | ADI- | VH3-11 | VL2-11 | AREFSSRPFDL (SEQ ID | CSYAGTYTSNY | 10 | 6 |
| | 48969 | | | NO: 23) | V (SEQ ID | | |
| 13 | ADI- | VH4-4 | VL1-51 | ARVNPPQYSSGWYSVY | GTWDNSLGAV | 7 | 3 |
| | 48900 | | | (SEQ ID NO: 25) | V (SEQ ID | | |
| 14 | ADI- | VH4-4 | N/A | ARVAWTSSSSCYYDY | N/A | 5 | 0 |
| | 42232 | | | (SEQ ID NO: 27) | | | |
| 15 | ADI- | VH4-4 | VL1-51 | ARDGEGHYYRSGDNWFD | GTWDSSLSAVV | 6 | 4 |
| | 42786 | | | R (SEQ ID NO: 28) | (SEQ ID NO: 29) | | |
| 16 | ADI- | VH4-4 | VL1-51 | ARAELSAWYYFDH (SEQ | GTWDTSLSAGR | 6 | 0 |
| | 42210 | | | ID NO: 30) | V (SEQ ID | | |
| 17 | ADI- | VH3-11 | VK3-15 | ARVSPLDDGYGYTYYGM | QQYNNWPPRT | 10 | 2 |
| | 50201 | | | DV (SEQ ID NO: 32) | (SEQ ID NO: 33) | | |
| 18 | ADI- | VH3-11 | VK1-12 | ARDWAELTTITNYFYP | QQAKSFPPT | 8 | 1 |
| | 48895 | | | (SEQ ID NO: 34) | (SEQ ID NO: 35) | | |
| 19 | ADI- | VH3-9 | VL2-14 | AKAENRIGYCSAGSCYLT | NSYTSSSTLV | 4 | 4 |
| | 42228 | | | YFDY (SEQ ID NO: 36) | (SEQ ID NO: 37) | | |
| 20 | ADI- | VH3-23 | VK3-15 | AKDPKYSSGWWAFDY | QQYDDWPL | 2 | 1 |
| | 45113 | | | (SEQ ID NO: 38) | (SEQ ID NO: 39) | | |
| 21 | ADI- | VH4-4 | VL1-51 | ARVEWAYSSSWWLDY | GTWDTSLSAGG | 4 | 3 |
| | 42198 | | | (SEQ ID NO: 40) | V (SEQ ID | | |
| 22 | ADI- | VH3-11 | VL2-14 | AKHTGDKPLVWAPSVYG | SSYTRRSTLV | 9 | 7 |
| | 42190 | | | LDV (SEQ ID NO: 42) | (SEQ ID NO: 43) | | |
| 23 | ADI- | VH4-4 | VL1-51 | ARVSVSTSAWYADY (SEQ | GTWDTSLSTV | 8 | 1 |
| | 49154 | | | ID NO: 44) | (SEQ ID NO: 45) | | |
| 24 | ADI- | VH3-11 | VL2-14 | ARELSSRIDY (SEQ ID | SSYPGTSALVI | 16 | 5 |
| | 49183 | | | NO: 46) | (SEQ ID NO: 47) | | |
| 25 | ADI- | VH3-33 | VK1-39 | ARAQDGQQLVNYYGMD | QQSYSTPYT | 8 | 4 |
| | 42201 | | | V (SEQ ID NO: 48) | (SEQ ID NO: 49) | | |
| 26 | ADI- | VH3-30 | VL1-40 | ARGGDYGDYESNNPAEYF | QSYDSSLSGHV | 1 | 0 |
| | 42144 | | | QH (SEQ ID NO: 50) | V (SEQ ID | | |
| 27 | ADI- | VH4-59 | VK3-11 | AGHREDPYGAYGAS (SEQ | QQRTNWPFT | 15 | 4 |
| | 50219 | | | ID NO: 52) | (SEQ ID NO: 53) | | |
| 28 | ADI- | VH4-61 | VK3-11 | ASRKEVRGTEDYFDY | HQRTNWPWT | 12 | 2 |
| | 48897 | | | (SEQ ID NO: 54) | (SEQ ID NO: 55) | | |
| 29 | ADI- | VH4-61 | VK3-11 | AKVEEDGYTNVVRDY | LQRTNWPFT | 6 | 4 |
| | 42194 | | | (SEQ ID NO: 56) | (SEQ ID NO: 57) | | |
| 30 | ADI- | VH3-11 | VL2-14 | AREGTRGRMD (SEQ ID | SSYTSGTTLGV | 9 | 4 |
| | 49189 | | | NO: 58) | (SEQ ID NO: 59) | | |
| 31 | ADI- | VH4-4 | VL1-51 | ARDSWSGPTRNWFDP | GTWDSSLGGVI | 14 | 8 |
| | 49188 | | | (SEQ ID NO: 60) | (SEQ ID NO: 61) | | |
| 32 | ADI- | VH4-4 | VL1-51 | ARVVWEYSNAWCVDF | ETWDSSLGVVV | 3 | 0 |
| | 42188 | | | (SEQ ID NO: 62) | (SEQ ID NO: 63) | | |
| 33 | ADI- | VH3-30 | VK1-33 | ARNTYYDRSGLIAY (SEQ | QQYDNLSRLT | 7 | 3 |
| | 50026 | | | ID NO: 64) | (SEQ ID NO: 65) | | |
| 34 | ADI- | VH4-4 | VL1-51 | ARGPLKSYWYFDL (SEQ | GTWDTSLSAGR | 7 | 0 |
| | 42809 | | | ID NO: 66) | V (SEQ ID | | |
| | | NO: 67) | | | | | |
| 35 | ADI- | VH4-4 | VL1-51 | ARYCSGATCYGSNGMDV | GTWDFRLSAL | 8 | 5 |
| | 46596 | | | (SEQ ID NO: 68) | (SEQ ID NO: 69) | | |
| 36 | ADI- | VH3-30 | VL2-14 | AKDQCGGDCTADY (SEQ | SSYTSSGTPVV | 6 | 3 |
| | 50205 | | | ID NO: 70) | (SEQ ID NO: 71) | | |
| 37 | ADI- | VH4-4 | VL1-51 | ASTLWGGPLSVASDY | GTWDSSPSAGR | 8 | 4 |
| | 42830 | | | (SEQ ID NO: 72) | V (SEQ ID | | |
| | | | | | NO: 73) | | |
| 38 | ADI- | VH3-30 | VK4-1 | ARDYYASGDGYFDY (SEQ | QQYYSTPRT | 17 | 8 |
| | 49186 | | | ID NO: 74) | (SEQ ID NO: 75) | | |
| 39 | ADI- | VH4-4 | VL1-51 | VRYCSSTSCYGLNGMDV | GTWDTRLSAL | 11 | 3 |
| | 46591 | | | (SEQ ID NO: 76) | (SEQ ID NO: 77) | | |
| 40 | ADI- | VH3-11 | VL1-51 | ARDGSLVNAIDY (SEQ ID | GTWDTSLSAAW | 8 | 3 |
| | 48955 | | | NO: 78) | V (SEQ ID | | |
| | | | | | NO: 79) | | |
| 41 | ADI- | VH4-4 | VL1-51 | ARVRWSGSTSWDLDY | GTWDTSPSAGG | 9 | 2 |
| | 42818 | | | (SEQ ID NO: 80) | V (SEQ ID | | |
| | | | | | NO: 81) | | |
| 42 | ADI- | VH2-5 | VL2-14 | AHSPRRITMVRGVIITWGD | SSYTSSSTLAV | 0 | 1 |
| | 50531 | | | GMDV (SEQ ID NO: 82) | (SEQ ID NO: 83) | | |
| 43 | ADI- | VH3-11 | VL1-51 | ARDGSMVNAIDY (SEQ ID | GTWDSSLSAAW | 6 | 2 |
| | 46586 | | | NO: 84) | V (SEQ ID | | |
| | | | | | NO: 85) | | |
| 44 | ADI- | VH3-33 | VL2-14 | ARDAYASGDGGIDY (SEQ | SSYRSSGTPYV | 6 | 3 |
| | 49138 | | | ID NO: 86) | (SEQ ID NO: 87) | | |
| 45 | ADI- | VH3-23 | VK1-33 | AKDLRGVGGWYYFDY | QQYDNLPLT | 2 | 2 |
| | 45075 | | | (SEQ ID NO: 88) | (SEQ ID NO: 89) | | |
| 46 | ADI- | VH3-23 | VK1-5 | AKDQGVTTDWPSDY (SEQ | QHYETYSVR | 20 | 13 |
| | 42831 | | | ID NO: 90) | (SEQ ID NO: 91) | | |
| 47 | ADI- | VH3-30-3 | VK1-27 | PRDGLPGANQYFFYYGM | QKYNSAPLT | 2 | 4 |
| | 42230 | | | DV (SEQ ID NO: 92) | (SEQ ID NO: 93) | | |
| 48 | ADI- | VH4-61 | VK3-11 | VRVEEYVNNEEVRDY | LQRTNWPFT | 11 | 1 |
| | 42847 | | | (SEQ ID NO: 94) | (SEQ ID NO: 95) | | |
| 49 | ADI- | VH3-23 | VK1-5 | ARDQGFTTDWPCDY (SEQ | QHYNSFSVK | 15 | 10 |
| | 42821 | | | ID NO: 96) | (SEQ ID NO: 97) | | |
| 50 | ADI- | VH3-11 | VL3-21 | ARDSNFNSNLDY (SEQ ID | QVWDSSSDHPW | 3 | 2 |
| | 42849 | | | NO: 98) | V (SEQ ID | | |
| | | | | | NO: 99) | | |
| 51 | ADI- | VH4-4 | VL1-51 | ARGPLKTYWYFDL (SEQ | GTWDTSLSAGR | 1 | 0 |
| | 42151 | | | ID NO: 100) | V (SEQ ID | | |
| | | | | | NO: 101) | | |
| 52 | ADI- | VH3-11 | VL3-21 | ARDSNYFYGLDV (SEQ ID | QVWDTSIDHHW | 3 | 7 |
| | 46001 | | | NO: 102) | V (SEQ ID | | |
| | | | | | NO: 103) | | |
| 53 | ADI- | VH3-30 | VL2-8 | AKDICSGDCGGGDY (SEQ | SSYAGSNNWVV | 3 | 1 |
| | 45154 | | | ID NO: 104) | (SEQ ID NO: 105) | | |
| 54 | ADI- | VH1-18 | VL2-14 | AREDDDYYSMDV (SEQ | SSYTTTSLVI | 15 | 6 |
| | 49161 | | | ID NO: 106) | (SEQ ID NO: 107) | | |
| 55 | ADI- | VH3-7 | VK2-28 | ARDISCISTSCYGGYYYYG | MQALQTPPRT | 1 | 0 |
| | 42154 | | | MDV (SEQ ID NO: 108) | (SEQ ID NO: 109) | | |
| 56 | ADI- | VH3-33 | VK1-17 | ARDYYASGDGSIDY (SEQ | LQHNSYPLT | 8 | 2 |
| | 48916 | | | ID NO: 110) | (SEQ ID NO: 111) | | |
| 57 | ADI- | VH3-23 | VK1-5 | AKYYDSSGYYYFDY (SEQ | KQYNRNPYT | 4 | 4 |
| | 45085 | | | ID NO: 112) | (SEQ ID NO: 113) | | |
| 58 | ADI- | VH3-30 | VK1-27 | AKGSVSVAGAEDY (SEQ | QKYNSAPQT | | |
| | 42211 | | | ID NO: 114) | (SEQ ID NO: 115) | | |
| 59 | ADI- | VH3-9 | VK3-15 | AKGYDSSGYYWADY | QQYNNWPPLT | 10 | 6 |
| | 48908 | | | (SEQ ID NO: 116) | (SEQ ID NO: 117) | | |
| 60 | ADI- | VH4-4 | VK4-1 | ARERGGYFTEPFDI (SEQ | QQYYRTPWT | 9 | 3 |
| | 48913 | | | ID NO: 118) | (SEQ ID NO: 119) | | |
| 61 | ADI- | VH3-48 | VL1-51 | AATIFGVVSFDY (SEQ ID | GTWDSALGAA | 7 | 1 |
| | 45140 | | | NO: 120) | V (SEQ ID | | |
| | | | | | NO: 121) | | |
| 62 | ADI- | VH3-23 | VK1-5 | AKYYDSSGYYYLDY (SEQ | QQYNRDPYT | 9 | 4 |
| | 50211 | | | ID NO: 122) | (SEQ ID NO: 123) | | |
| 63 | ADI- | VH3-72 | VL3-25 | CRESGEGFDP (SEQ ID | QSADRSGSVI | 5 | 11 |
| | 42199 | | | NO: 124) | (SEQ ID NO: 125) | | |
| 64 | ADI- | VH1-18 | VK3-11 | ARDQSHGTFGGVIDSTTLF | QQRSNWPS | 6 | 0 |
| | 42231 | | | YYYGMDV (SEQ ID | (SEQ ID NO: 127) | | |
| | | | | NO: 126) | | | |
| 65 | ADI- | VH4-39 | VK1-39 | ARGYCSSTSCFYYYYGMD | QQSYSTPLT | 0 | 0 |
| | 45164 | | | V (SEQ ID NO: 128) | (SEQ ID NO: 129) | | |
| 66 | ADI- | VH3-21 | VK3-20 | ARDHYFDSSGDYLSYYYN | QQYGSSPRA | 8 | 6 |
| | 42233 | | | GMDV (SEQ ID NO: 130) | (SEQ ID NO: 131) | | |
| 67 | ADI- | VH3-72 | VK1-39 | ARVYGGPDDY (SEQ ID | QQSSITPPT (SEQ | 3 | 2 |
| | 42191 | | | NO: 132) | ID NO: 133) | | |
| 68 | ADI- | VH3-23 | VK1-5 | AKDGVTTINGWFHFEY | QQYNSFPFT | 9 | 2 |
| | 48899 | | | (SEQ ID NO: 134) | (SEQ ID NO: 135) | | |
| 69 | ADI- | VH3-72 | VK1-39 | TRITGDRYWYLDL (SEQ | QQTYSASGS | 11 | 13 |
| | 49145 | | | ID NO: 136) | (SEQ ID NO: 137) | | |
| 70 | ADI- | VH4-61 | VK1-39 | ARGWFGYSNYGLYYYYG | QQSYSTPWT | 1 | 0 |
| | 46729 | | | MDV (SEQ ID NO: 138) | (SEQ ID NO: 139) | | |
| 71 | ADI- | VH4-4 | VL1-51 | ARDFWSGSNWFDP (SEQ | GTWDNSLGVV | 1 | 0 |
| | 46722 | | | ID NO: 140) | (SEQ ID NO: 141) | | |
| 72 | ADI- | VH3-9 | VK3-20 | AKDIGDSYGSGSYYLPYG | QQYGSSPG | 0 | 2 |
| | 45148 | | | AYYGMDV (SEQ ID | (SEQ ID NO: 143) | | |
| | | | | NO: 142) | | | |
| 73 | ADI- | VH3-23 | VK1-5 | AKHYDSSGYYYEDY (SEQ | HQYKDFPWT | 11 | 6 |
| | 49168 | | | ID NO: 144) | (SEQ ID NO: 145) | | |
| 74 | ADI- | VH3-72 | VK1-5 | ARVRDGEYDY (SEQ ID | QQYNSYSP | 9 | 4 |
| | 49040 | | | NO: 146) | (SEQ ID NO: 147) | | |
| 75 | ADI- | VH3-21 | VK3-20 | ARDNSEVEDYGDYVLYH | QQYGSSPF (SEQ | 4 | 3 |
| | 42187 | | | YYGMDV (SEQ ID | ID NO: 149) | | |
| | | | | NO: 148) | | | |
| 76 | ADI- | VH3-30 | VL2-14 | AKDQCGGDCTADY (SEQ | SSYTSSSTPVV | 2 | 3 |
| | 49561 | | | ID NO: 150) | (SEQ ID NO: 151) | | |
| 77 | ADI- | VH3-30-3 | VL2-11 | ARGYTGYDGFDY (SEQ ID | CSYATNYGVV | 8 | 2 |
| | 42219 | | | NO: 152) | (SEQ ID NO: 153) | | |
| 78 | ADI- | VH1-18 | VL6-57 | ARRPYYYGSRRPAGHMD | QSYDSSNVV | 0 | 0 |
| | 50535 | | | V (SEQ ID NO: 154) | (SEQ ID NO: 155) | | |
| 79 | ADI- | VH4-30-4 | VL1-51 | GRDSDKNYFDY (SEQ ID | GAWDSSLSAHV | 8 | 2 |
| | 45128 | | | NO: 156) | V (SEQ ID | | |
| | | | | | NO: 157) | | |
| 80 | ADI- | VH3-33 | VK1-5 | AKTYDSNAYYYLDY (SEQ | QQYNRYPYT | 7 | 7 |
| | 45136 | | | ID NO: 158) | (SEQ ID NO: 159) | | |
| 81 | ADI- | VH3-30 | VK1-17 | ASLWFIVMTMSKNPETDY | LQHHSYPWT | 6 | 2 |
| | 42189 | | | (SEQ ID NO: 160) | (SEQ ID NO: 161) | | |
| 82 | ADI- | VH3-23 | VK1-5 | AKYYDSSGYYYFDH (SEQ | QQYNRDPYT | 15 | 11 |
| | 45078 | | | ID NO: 162) | (SEQ ID NO: 163) | | |
| 83 | ADI- | VH3-23 | VK1-5 | AKFYDSSGYYYFDY (SEQ | QQYNTYPYT | 17 | 13 |
| | 49162 | | | ID NO: 164) | (SEQ ID NO: 165) | | |
| 84 | ADI- | VH3-72 | VK1-39 | VRLYGDYVAYFDY (SEQ | QQSYSTPWT | 5 | 7 |
| | 42223 | | | ID NO: 166) | (SEQ ID NO: 167) | | |
| 85 | ADI- | VH1-18 | VL2-14 | ARRGTTVTRFGVIQYYYG | SSYTSSSTLV | 0 | 1 |
| | 48435 | | | MDV (SEQ ID NO: 168) | (SEQ ID NO: 169) | | |
| 86 | ADI- | VH4-59 | VL3-21 | ARETANNWFDP (SEQ ID | QVWDNSSDRRV | 16 | 8 |
| | 46742 | | | NO: 170) | (SEQ ID NO: 171) | | |
| 87 | ADI- | VH3-30-3 | VK3-15 | ARASMNIPRPPVHDY (SEQ | QQYNTWWT | 9 | 3 |
| | 4278 | | | ID NO: 172) | (SEQ ID NO: 173) | | |
| 88 | ADI- | VH3-23 | VK1-39 | AKDRSQGDYGDYVADY | QQSYSTPLT | 0 | 0 |
| | 46718 | | | (SEQ ID NO: 174) | (SEQ ID NO: 175) | | |
| 89 | ADI- | VH4-4 | VK3-15 | ARVQTSHSELWFGEFGAD | QQYNTWPKT | 3 | 1 |
| | 49141 | | | (SEQ ID NO: 176) | (SEQ ID NO: 177) | | |
| 90 | ADI- | VH3-23 | VK3-20 | AKDGGYSTDWYFDL (SEQ | QQYGSSRRT | 7 | 2 |
| | 42213 | | | ID NO: 178) | (SEQ ID NO: 179) | | |
| 91 | ADI- | VH3-30 | VK1-5 | AKGYDSNGYYYIDY (SEQ | QQYNRYPYT | 5 | 1 |
| | 42844 | | | ID NO: 180) | (SEQ ID NO: 181) | | |
| 92 | ADI- | VH3-33 | VL2-14 | ARDVGYQLLQVYGMDV | SSYTSSSTLDVV | 0 | 0 |
| | 45161 | | | (SEQ ID NO: 182) | (SEQ ID NO: 183) | | |
| 93 | ADI- | VH4-31 | VK3-15 | ARAEYDTSGYYQQRLPEY | QQYNSWPPIT | 5 | 1 |
| | 42192 | | | FQH (SEQ ID NO: 184) | (SEQ ID NO: 185) | | |
| 94 | ADI- | VH3-23 | VK1-5 | AKYYDSSGYYYFHS (SEQ | QQYNRYPYT | 13 | 7 |
| | 48910 | | | ID NO: 186) | (SEQ ID NO: 187) | | |
| 95 | ADI- | VH3-72 | VL1-47 | AREHGDYGLDY (SEQ ID | ATWDVSLSNDV | 8 | 4 |
| | 42193 | | | NO: 188) | L (SEQ ID | | |
| | | | | | NO: 189) | | |
| 96 | ADI- | VH1-2 | VK3-20 | YVDYYYDSSGYYSPFDY | QQYGSSPPIT | 1 | 2 |
| | 49590 | | | (SEQ ID NO: 190) | (SEQ ID NO: 191) | | |
| 97 | ADI- | VH3-72 | VK1-39 | ARVDGEEVALIY (SEQ ID | QQSSTTRWT | 8 | 11 |
| | 45076 | | | NO: 192) | (SEQ ID NO: 193) | | |
| 98 | ADI- | VH3-72 | VK1-39 | VRVWGGEAARYDY (SEQ | QHASTTPWT | 13 | 12 |
| | 48968 | | | ID NO: 194) | (SEQ ID NO: 195) | | |
| 99 | ADI- | VH3-72 | VL3-1 | SRHMGFGLDL (SEQ ID | QAWDTTTAGG | 3 | 6 |
| | 42212 | | | NO: 196) | V (SEQ ID | | |
| | | | | | NO: 197) | | |
| 100 | ADI- | VH3-33 | VK3-20 | ARDYYGSGDGYFDY (SEQ | QQYGSSPRA | 0 | 0 |
| | 48462 | | | ID NO: 198) | (SEQ ID NO: 199) | | |
| 101 | ADI- | VH2-26 | VL2-8 | ARIPVEYGTPRGSFDT | SSYGGNNDLV | 11 | 8 |
| | 45127 | | | (SEQ ID NO: 200) | (SEQ ID NO: 201) | | |
| 102 | ADI- | VH3-30-3 | VK3-11 | AGGSPDY (SEQ ID | QQRSNWPYT | 9 | 4 |
| | 42200 | | | NO: 202) | (SEQ ID NO: 203) | | |
| 103 | ADI- | VH3-30 | VK1-5 | ARAYDSRGYYYIEH (SEQ | QQYKTYWT | 14 | 8 |
| | 50203 | | | ID NO: 204) | (SEQ ID NO: 205) | | |
| 104 | ADI- | VH1-18 | VL2-14 | AREIDSNYVFDY (SEQ ID | SSYTSSGTNI | 2 | 0 |
| | 42149 | | | NO: 206) | (SEQ ID NO: 207) | | |
| 105 | ADI- | VH3-7 | VK3-15 | ARKLSYSSGWYYFDY | QQYNNWPPLT | 2 | 3 |
| | 42181 | | | (SEQ ID NO: 208) | (SEQ ID NO: 209) | | |
| 106 | ADI- | VH3-72 | VL3-10 | VTTTVILFDY (SEQ ID | YSTDSSGLLGV | 9 | 8 |
| | 45126 | | | NO: 210) | (SEQ ID NO: 211) | | |
| 107 | ADI- | VH4-34 | VK4-1 | ARGRLAWGLRGQKSPNFF | QQFHSPPWT | 7 | 5 |
| | 45074 | | | AY (SEQ ID NO: 212) | (SEQ ID NO: 213) | | |
| 108 | ADI- | VH3-15 | VK1-5 | ATAGIFGVVIMKGFDH | QQYNDYPWT | 9 | 9 |
| | 49041 | | | (SEQ ID NO: 214) | (SEQ ID NO: 215) | | |
| 109 | ADI- | VH1-69 | VK1-17 | ARETYYYGSGSVPVHD | LQHNTYPWT | 9 | 1 |
| | 42227 | | | (SEQ ID NO: 216) | (SEQ ID NO: 217) | | |
| 110 | ADI- | VH3-30 | VK1-5 | ARGYDSSGYWGFGDN | QQYYSYPYT | 16 | 6 |
| | 50220 | | | (SEQ ID NO: 218) | (SEQ ID NO: 219) | | |
| 111 | ADI- | VH3-72 | VL3-25 | ARVEGGAWGAFDI (SEQ | QSADRSGTVV | 1 | 1 |
| | 42141 | | | ID NO: 220) | (SEQ ID NO: 221) | | |
| 112 | ADI- | VH2-26 | VL2-8 | ARLWFTEYPGAFDI (SEQ | SSYAGSNALV | 5 | 4 |
| | 42216 | | | ID NO: 222) | (SEQ ID NO: 223) | | |
| 113 | ADI- | VH4-39 | VL6-57 | ARHSSGSYYLAGYYFDY | QSYDSSNWV | 0 | 1 |
| | 50534 | | | (SEQ ID NO: 224) | (SEQ ID NO: 225) | | |
| 114 | ADI- | VH3-72 | VL3-25 | ARLTDSGYDD (SEQ ID | HSPDSHVV | 6 | 3 |
| | 49140 | | | NO: 226) | (SEQ ID NO: 227) | | |
| 115 | ADI- | VH4-59 | VL3-21 | ARETCSGGSCYYRVGSAF | QVWDSSSDHEV | 0 | 1 |
| | 46741 | | | DI (SEQ ID NO: 228) | (SEQ ID NO: 229) | | |
| 116 | ADI- | VH3-9 | VK1-33 | VKDYCSGGRCYSFDY | QQWGT (SEQ ID | 6 | 4 |
| | 42195 | | | (SEQ ID NO: 230) | NO: 231) | | |
| 117 | ADI- | VH3-30 | VK1-5 | AKAYDSSAYYYLDY (SEQ | QQYNRYPYT | 3 | 4 |
| | 42172 | | | ID NO: 232) | (SEQ ID NO: 233) | | |
| 118 | ADI- | VH3-30 | VK1-5 | AKAYDSRGYYYLDY (SEQ | QQYNRYSYT | 3 | 6 |
| | 42178 | | | ID NO: 234) | (SEQ ID NO: 235) | | |
| 119 | ADI- | VH3-23 | VK1-5 | AKDLTHRLGSIFGKLTFDA | QQYNNFWT | 23 | 4 |
| | 49032 | | | FDI (SEQ ID NO: 236) | (SEQ ID NO: 237) | | |
| 120 | ADI- | VH3-30 | VL1-40 | AKDLTPYFYDSGAFDH | HSYDSNMSGSV | 17 | 7 |
| | 50197 | | | (SEQ ID NO: 238) | (SEQ ID NO: 239) | | |
| 121 | ADI- | VH3-72 | VK1-27 | ARVFGGPTDY (SEQ ID | QKYYSAPLIT | 7 | 2 |
| | 48894 | | | NO: 240) | (SEQ ID NO: 241) | | |
| 122 | ADI- | VH3-72 | VL3-25 | ARVVNGLDV (SEQ ID | QSADSSVADSS | 7 | 1 |
| | 42226 | | | NO: 242) | VV (SEQ ID | | |
| | | | | | NO: 243) | | |
| 123 | ADI- | VH3-30-3 | VK3-11 | ARGQPDY (SEQ ID | QQRSNWPYT | 7 | 4 |
| | 49037 | | | NO: 244) | (SEQ ID NO: 245) | | |
| 124 | ADI- | VH4-4 | VL1-51 | AGKKWELLGFRFDP (SEQ | GTWDNSLGMV | 9 | 4 |
| | 46739 | | | ID NO: 246) | V (SEQ ID | | |
| | | | | | NO: 247) | | |
| 125 | ADI- | VH1-3 | VL2-14 | ARQWLGHFDY (SEQ ID | SSYTSSSTYV | 1 | 0 |
| | 42810 | | | NO: 248) | (SEQ ID NO: 249) | | |
| 126 | ADI- | VH3-72 | VK3-11 | ARVFSYYLDY (SEQ ID | QQPGNWPPAFT | 11 | 3 |
| | 49137 | | | NO: 250) | (SEQ ID NO: 251) | | |
| 127 | ADI- | VH2-5 | VK3-15 | AHRHIAARLYRDDDVFDV | QQYNNWIT | 2 | 2 |
| | 42817 | | | (SEQ ID NO: 252) | (SEQ ID NO: 253) | | |
| 128 | ADI- | VH1-8 | VK1D-12 | ARGLNTVTNSDY (SEQ ID | QQANSFPWT | 0 | 0 |
| | 50218 | | | NO: 254) | (SEQ ID NO: 255) | | |
| 129 | ADI- | VH1-2 | VK2-28 | ASGLSPDFSVLDV (SEQ ID | MQALQTPYT | 0 | 1 |
| | 42126 | | | NO: 256) | (SEQ ID NO: 257) | | |
| 130 | ADI- | VH6-1 | VL1-44 | AREGAGYYDSSGYYPLSY | AAWDDNLIGVV | 3 | 4 |
| | 42186 | | | DAFDI (SEQ ID NO: 258) | (SEQ ID NO: 259) | | |
| 131 | ADI- | VH3-72 | VL2-8 | ARVRGSYWDY (SEQ ID | SSFAGSNNLYV | 6 | 0 |
| | 48890 | | | NO: 260) | (SEQ ID NO: 261) | | |
| 132 | ADI- | VH3-72 | VL2-14 | GRDRGWLDI (SEQ ID | SSYTRSSTRV | 2 | 3 |
| | 42206 | | | NO: 262) | (SEQ ID NO: 263) | | |
| 133 | ADI- | VH4-4 | VL1-51 | ARVIRDLRDYYDGSGYGP | ETWDSRLSVV | 16 | 4 |
| | 46724 | | | DAFDI (SEQ ID NO: 264) | (SEQ ID NO: 265) | | |
| 134 | ADI- | VH4-4 | VL1-51 | ARARWEDGNYYYGMDV | GTWDSSLSAVV | 0 | 0 |
| | 50539 | | | (SEQ ID NO: 266) | (SEQ ID NO: 267) | | |
| 135 | ADI- | VH3-23 | VK1-39 | AKDQSSGWPNYYYGMDV | QQSYSTPWT | 0 | 0 |
| | 45156 | | | (SEQ ID NO: 268) | (SEQ ID NO: 269) | | |
| 136 | ADI- | VH7-4-1 | VK1-39 | VRGYCSSTSCYGGLYWFD | QQSYSTPRT | 0 | 1 |
| | 50536 | | | P (SEQ ID NO: 270) | (SEQ ID NO: 271) | | |
| 137 | ADI- | VH3-30-3 | VL1-40 | ARHSGGYSSKDKPTEYFQ | QSYDSSLSGVV | 6 | 2 |
| | 42217 | | | H (SEQ ID NO: 272) | (SEQ ID NO: 273) | | |
| 138 | ADI- | VH4-4 | VK4-1 | ARDVGVAAVITGSVR | QQFYTTPST | 6 | 4 |
| | 48951 | | | (SEQ ID NO: 274) | (SEQ ID NO: 275) | | |
| 139 | ADI- | VH7-4-1 | VK1-39 | ARGYCSSTSCYGGLYWFD | QQSYSTPRT | 0 | 0 |
| | 50537 | | | P (SEQ ID NO: 276) | (SEQ ID NO: 277) | | |
| 140 | ADI- | VH3-30 | VK1-17 | ARDGAGDYIWGSYRHKG | LQHNSYPLT | 0 | 0 |
| | 46737 | | | LHYYYGMDV (SEQ ID | (SEQ ID NO: 279) | | |
| | | | | NO: 278) | | | |
| 141 | ADI- | VH4-4 | VL6-57 | AKDPRTFYGVVMLLDDP | QSYDSTTVV | 9 | 7 |
| | 50538 | | | (SEQ ID NO: 280) | (SEQ ID NO: 281) | | |
| 142 | ADI- | VH3-30 | VL2-8 | ARGFGELPGFDI (SEQ ID | SSYAGSNNFVV | 15 | 4 |
| | 48950 | | | NO: 282) | (SEQ ID NO: 283) | | |
| 143 | ADI- | VH3-21 | VL1-51 | ARDSWGPFDY (SEQ ID | GTWDSSLSAKV | 0 | 0 |
| | 42114 | | | NO: 284) | (SEQ ID NO: 285) | | |
| 144 | ADI- | VH3-33 | VK1-5 | AKTYDSRAYYYLDY (SEQ | QQYNRYPYT | 8 | 7 |
| | 49194 | | | ID NO: 286) | (SEQ ID NO: 287) | | |
| 145 | ADI- | VH3-23 | VL2-11 | AKDLFYDFWTGITIDY | CSYAGSYTFVL | 4 | 0 |
| | 42124 | | | (SEQ ID NO: 288) | (SEQ ID NO: 289) | | |
| 146 | ADI- | VH3-7 | VK1-39 | ARDGGTVSDGLDV (SEQ | QQTFSIWT (SEQ | 8 | 7 |
| | 45123 | | | ID NO: 290) | ID NO: 291) | | |
| 147 | ADI- | VH4-4 | VL1-51 | ARVVWYSSSSHLFDY | GTWDSSLSAGK | 0 | 0 |
| | 50533 | | | (SEQ ID NO: 292) | V (SEQ ID | | |
| | | | | | NO: 293) | | |
| 148 | ADI- | VH3-33 | VL2-8 | ARIKSDAFDL (SEQ ID | FSYAGSNNYV | 10 | 6 |
| | 49205 | | | NO: 294) | (SEQ ID NO: 295) | | |
| 149 | ADI- | VH3-30 | VK2-24 | AKFPLRDGGSGEGFDY | MQASQFPLT | 17 | 3 |
| | 45151 | | | (SEQ ID NO: 296) | (SEQ ID NO: 297) | | |
| 150 | ADI- | VH3-30-3 | VK1-33 | ARNTYYDRRRTFDY (SEQ | QQYDNLPPVT | 0 | 0 |
| | 46728 | | | ID NO: 298) | (SEQ ID NO: 299) | | |
| 151 | ADI- | VH3-72 | VL3-1 | AGVGITGTTGIDY (SEQ ID | QAWDSSTDVV | 0 | 0 |
| | 49030 | | | NO: 300) | (SEQ ID NO: 301) | | |
| 152 | ADI- | VH3-9 | VK1-27 | AKGAAAGPFPYFYYAMD | QKYQSAPPT | 14 | 5 |
| | 50200 | | | V (SEQ ID NO: 302) | (SEQ ID NO: 303) | | |

TABLE 2
Affinity and Neutralization data for anti-YFV antibodies

| | | | Neut | Neut (10 nM) | | |
|---|---|---|---|---|---|---|
| | | | (100 nM) | Avg. | Epitope | |
| Antibody | | Monovalent | Avg. % | % | Binning | B Cell |
| Number | Name | Binding (KD) | Neutralization | Neutralization | Data | Classification |
| 1 | ADI-49039 | 4.15E−07 | 99.07 | 99.41 | 4G2 and 5A | Atypical IgM memory |
| | | | | | | (IgM+ IgD− CD27− SHM+) |
| 2 | ADI-49147 | 3.73E−09 | 97.78 | 99.65 | DIII; ADI- | IgD memory (IgM− IgD+ SHM+) |
| | | | | | 49147 | |
| | | | | | competitor | |
| 3 | ADI-42229 | 7.87E−09 | 99.64 | 99.76 | 4G2 and 5A | IgM-only (IgM+ IgD− CD27+) |
| 4 | ADI-45090 | 2.11E−09 | 99.56 | 98.83 | DIII; ADI- | swIg+ CD27+ |
| | | | | | 49147 | |
| | | | | | competitor | |
| 5 | ADI-45097 | 1.62E−09 | 99.32 | 99.38 | Blocks 5A | swIg+ CD27− |
| | | | | | only | |
| 6 | ADI-49133 | 6.91E−09 | 99.90 | 98.57 | Blocks 5A | IgM+ IgD+ CD27+ |
| | | | | | only | |
| 7 | ADI-49033 | 1.80E−08 | 99.86 | 99.95 | 5A and | Atypical IgM memory |
| | | | | | ADI-45107 | (IgM+ IgD+ CD27− SHM+) |
| 8 | ADI-49044 | 1.88E−09 | 99.93 | 99.68 | Other | swIg+ CD27− |
| 9 | ADI-45083 | 3.45E−09 | 99.69 | 99.59 | Blocks 5A | swIg+ CD27− |
| | | | | | only | |
| 10 | ADI-42225 | 3.15E−08 | 99.69 | 99.76 | Blocks 5A | swIg+ CD27+ |
| | | | | | only | |
| 11 | ADI-49139 | 3.45E−09 | 99.53 | 100.00 | 5A and | swIg+ CD27+ |
| | | | | | ADI-45107 | |
| 12 | ADI-48969 | 5.86E−09 | 99.88 | 98.12 | 5A and | IgG+ CD27− |
| | | | | | ADI-45107 | |
| 13 | ADI-48900 | 2.62E−08 | 99.70 | 99.94 | Blocks 5A | Atypical IgM memory |
| | | | | | only | (IgM+ IgD+ CD27− SHM+) |
| 14 | ADI-42232 | 1.46E−08 | 99.77 | 99.85 | 5A and | swIg+ CD27+ |
| | | | | | ADI-45107 | |
| 15 | ADI-42786 | 1.27E−08 | 92.79 | 89.16 | Blocks 5A | swIg+ CD27+ |
| | | | | | only | |
| 16 | ADI-42210 | 1.87E−08 | 99.64 | 99.76 | 5A and | swIg+ CD27+ |
| | | | | | ADI-45107 | |
| 17 | ADI-50201 | 1.31E−08 | 99.28 | 97.94 | 5A and | Atypical IgM memory |
| | | | | | ADI-45107 | (IgM+ IgD+ CD27− SHM+) |
| 18 | ADI-48895 | 4.49E−09 | 99.71 | 99.78 | 5A and | IgG+ CD27+ |
| | | | | | ADI-45107 | |
| 19 | ADI-42228 | 8.64E−09 | 97.69 | 69.43 | ADI-45107 | swIg+ CD27− |
| 20 | ADI-45113 | 2.53E−07 | 98.92 | 98.77 | DIII; ADI- | IgD memory (IgM− IgD+ SHM+) |
| | | | | | 49147 | |
| | | | | | competitor | |
| 21 | ADI-42198 | 3.32E−08 | 99.71 | 99.77 | 5A and | Atypical IgM memory |
| | | | | | ADI-45107 | (IgM+ IgD+ CD27− SHM+) |
| 22 | ADI-42190 | 1.31E−08 | 99.80 | 99.18 | Blocks 5A | swIg+ CD27+ |
| | | | | | only | |
| 23 | ADI-49154 | 1.41E−07 | 98.78 | 98.26 | 5A and | Atypical IgM memory |
| | | | | | ADI-45107 | (IgM+ IgD− CD27− SHM+) |
| 24 | ADI-49183 | 2.21E−09 | 99.90 | 99.86 | 5A and | IgG+ CD27+ |
| | | | | | ADI-45107 | |
| 25 | ADI-42201 | 1.13E−07 | 99.33 | 98.70 | Blocks 5A | IgM+ IgD+ CD27+ |
| | | | | | only | |
| 26 | ADI-42144 | 1.09E−08 | 5.24 | 16.76 | blocks 4G2 | swIg+ CD27− |
| | | | | | only | |
| 27 | ADI-50219 | 8.59E−10 | 99.54 | 99.48 | Blocks 5A | IgG+ CD27+ |
| | | | | | only | |
| 28 | ADI-48897 | 3.22E−09 | 96.74 | 98.45 | 5A and | IgG+ CD27− |
| | | | | | ADI-45107 | |
| 29 | ADI-42194 | 5.44E−09 | 99.59 | 99.58 | Blocks 5A | swIg+ CD27+ |
| | | | | | only | |
| 30 | ADI-49189 | 1.27E−08 | 99.84 | 98.09 | 5A and | Atypical IgM memory |
| | | | | | ADI-45107 | (IgM+ IgD+ CD27− SHM+) |
| 31 | ADI-49188 | 9.54E−09 | 99.96 | 99.47 | 5A and | IgG+ CD27+ |
| | | | | | ADI-45107 | |
| 32 | ADI-42188 | 3.71E−08 | 99.80 | 99.77 | 5A and | IgM-only (IgM+ IgD− CD27+) |
| | | | | | ADI-45107 | |
| 33 | ADI-50026 | >1.0E−07 | 99.93 | 99.91 | Other | swIg+ CD27+ |
| 34 | ADI-42809 | 3.78E−09 | 94.44 | 95.50 | 5A and | swIg+ CD27+ |
| | | | | | ADI-45107 | |
| 35 | ADI-46596 | 5.11E−09 | 96.03 | 99.34 | 5A and | Atypical IgM memory |
| | | | | | ADI-45107 | (IgM+ IgD+ CD27− SHM+) |
| 36 | ADI-50205 | 1.88E−08 | 99.70 | 99.64 | Blocks 5A | IgM+ IgD+ CD27+ |
| | | | | | only | |
| 37 | ADI-42830 | 3.48E−09 | 95.10 | 93.86 | Blocks 5A | swIg+ CD27− |
| | | | | | only | |
| 38 | ADI-49186 | 1.23E−08 | 99.46 | 99.81 | Blocks 5A | IgG+ CD27+ |
| | | | | | only | |
| 39 | ADI-46591 | 9.60E−09 | 98.23 | 97.22 | 5A and | Atypical IgM memory |
| | | | | | ADI-45107 | (IgM+ IgD+ CD27− SHM+) |
| 40 | ADI-48955 | 2.05E−09 | 99.87 | 96.91 | 5A and | IgG+ CD27+ |
| | | | | | ADI-45107 | |
| 41 | ADI-42818 | 2.41E−09 | 95.52 | 94.94 | Blocks 5A | swIg+ CD27+ |
| | | | | | only | |
| 42 | ADI-50531 | >1.0E−07 | 98.70 | 98.22 | Other | n.d. |
| 43 | ADI-46586 | 2.16E−09 | 97.63 | 97.56 | 5A and | IgG+ CD27+ |
| | | | | | ADI-45107 | |
| 44 | ADI-49138 | 1.75E−08 | 99.75 | 94.66 | Other | IgD memory (IgM− IgD+ SHM+) |
| 45 | ADI-45075 | 2.61E−08 | 99.36 | 98.41 | DIII; ADI- | Atypical IgM memory |
| | | | | | 49147 | (IgM+ IgD+ CD27− SHM+) |
| | | | | | competitor | |
| 46 | ADI-42831 | 1.96E−08 | 90.78 | 93.35 | Blocks 5A | IgD memory (IgM− IgD+ SHM+) |
| | | | | | only | |
| 47 | ADI-42230 | 1.99E−09 | 94.25 | 32.22 | Other | swIg+ CD27− |
| 48 | ADI-42847 | 3.21E−09 | 83.08 | 91.27 | Blocks 5A | swIg+ CD27+ |
| | | | | | only | |
| 49 | ADI-42821 | 1.21E−08 | 93.76 | 90.48 | Blocks 5A | swIg+ CD27+ |
| | | | | | only | |
| 50 | ADI-42849 | 1.98E−09 | 95.03 | 94.68 | 5A and | swIg+ CD27− |
| | | | | | ADI-45107 | |
| 51 | ADI-42151 | 2.97E−07 | 94.29 | 71.97 | 5A and | IgM+ IgD+ CD27+ |
| | | | | | ADI-45107 | |
| 52 | ADI-46001 | 6.17E−09 | 98.61 | 99.26 | 5A and | IgG+ CD27− |
| | | | | | ADI-45107 | |
| 53 | ADI-45154 | >1.0E−07 | 97.85 | 96.42 | Blocks 5A | IgM-only (IgM+ IgD− CD27+) |
| | | | | | only | |
| 54 | ADI-49161 | 1.81E−09 | 92.08 | 91.30 | Other | swIg+ CD27− |
| 55 | ADI-42154 | 1.64E−07 | 98.99 | 96.53 | Blocks 5A | Atypical IgM memory |
| | | | | | only | (IgM+ IgD+ CD27− SHM+) |
| 56 | ADI-48916 | 3.93E−07 | 99.79 | 88.13 | Other | Atypical IgM memory |
| | | | | | | (IgM+ IgD+ CD27− SHM+) |
| 57 | ADI-45085 | 1.52E−07 | 99.32 | 96.95 | Other | swIg+ CD27+ |
| 58 | ADI-42211 | 1.64E−09 | 99.73 | 92.83 | Other | IgM+ IgD+ CD27+ |
| 59 | ADI-48908 | 1.76E−09 | 99.80 | 74.90 | Blocks 5A | IgG+ CD27+ |
| | | | | | only | |
| 60 | ADI-48913 | 1.86E−07 | 98.86 | 81.30 | Other | Atypical IgM memory |
| | | | | | | (IgM+ IgD+ CD27− SHM+) |
| 61 | ADI-45140 | 4.60E−09 | 82.51 | 41.20 | ADI-44112 | swIg+ CD27+ |
| 62 | ADI-50211 | 2.08E−08 | 99.80 | 90.42 | 5A and | IgG+ CD27+ |
| | | | | | ADI-45107 | |
| 63 | ADI-42199 | 7.00E−10 | 93.81 | 45.88 | 4G2 and 5A | swIg+ CD27+ |
| 64 | ADI-42231 | 4.21E−08 | 86.32 | 42.38 | Other | swIg+ CD27+ |
| 65 | ADI-45164 | >1.0E−07 | 92.56 | 90.63 | Other | n.d. |
| 66 | ADI-42233 | 2.98E−09 | 88.94 | 33.48 | 4G2 and 5A | swIg+ CD27+ |
| 67 | ADI-42191 | 2.13E−09 | 99.56 | 80.41 | blocks 4G2 | swIg+ CD27+ |
| | | | | | only | |
| 68 | ADI-48899 | 4.34E−07 | 97.45 | 45.70 | Other | IgM+ IgD+ CD27+ |
| 69 | ADI-49145 | 1.05E−09 | 94.23 | 86.66 | blocks 4G2 | swIg+ CD27− |
| | | | | | only | |
| 70 | ADI-46729 | >1.0E−07 | 97.92 | 90.19 | blocks 4G2 | n.d. |
| | | | | | only | |
| 71 | ADI-46722 | >1.0E−07 | 94.16 | 89.67 | Blocks 5A | n.d. |
| | | | | | only | |
| 72 | ADI-45148 | >1.0E−07 | 94.92 | 23.00 | Blocks 5A | IgM-only (IgM+ IgD− CD27+) |
| | | | | | only | |
| 73 | ADI-49168 | 2.41E−08 | 99.78 | 94.55 | ADI-45107 | IgG+ CD27+ |
| 74 | ADI-49040 | 9.99E−10 | 97.00 | 47.80 | 4G2 and 5A | swIg+ CD27− |
| 75 | ADI-42187 | 4.01E−09 | 97.44 | 63.78 | 4G2 and 5A | swIg+ CD27+ |
| 76 | ADI-49561 | 2.42445E−07 | 98.78 | 76.85 | Blocks 5A | IgM+ IgD+ CD27+ |
| | | | | | only | |
| 77 | ADI-42219 | 4.05E−09 | 99.59 | 89.06 | 4G2 and 5A | swIg+ CD27+ |
| 78 | ADI-50535 | >1.0E−07 | 97.31 | 70.22 | Other | n.d. |
| 79 | ADI-45128 | 3.54E−09 | 71.21 | 32.72 | Other | Atypical IgM memory |
| | | | | | | (IgM+ IgD+ CD27− SHM+) |
| 80 | ADI-45136 | 1.68E−08 | 99.50 | 15.36 | Other | swIg+ CD27+ |
| 81 | ADI-42189 | 2.40E−09 | 96.78 | 71.86 | ADI-44112 | swIg+ CD27+ |
| 82 | ADI-45078 | 5.88E−09 | 98.28 | 46.04 | ADI-45107 | swIg+ CD27+ |
| 83 | ADI-49162 | 2.19E−09 | 99.64 | 41.35 | Other | swIg+ CD27+ |
| 84 | ADI-42223 | 1.04E−09 | 60.40 | 27.92 | blocks 4G2 | swIg+ CD27+ |
| | | | | | only | |
| 85 | ADI-48435 | >1.0E−07 | 98.28 | 82.33 | Other | IgM+ IgD+ CD27+ |
| 86 | ADI-46742 | >1.0E−07 | 97.87 | 70.85 | ADI-44112 | n.d. |
| 87 | ADI-42787 | 4.50E−10 | 0 | 0 | ADI-44112 | swIg+ CD27− |
| 88 | ADI-46718 | >1.0E−07 | 91.16 | 48.14 | Other | n.d. |
| 89 | ADI-49141 | 1.87E−07 | 99.87 | 80.61 | Other | swIg+ CD27− |
| 90 | ADI-42213 | 1.52E−08 | 99.00 | 47.91 | 4G2 and 5A | swIg+ CD27+ |
| 91 | ADI-42844 | 1.37E−07 | 95.65 | 79.34 | Other | swIg+ CD27+ |
| 92 | ADI-45161 | >1.0E−07 | 90.85 | 20.97 | Other | n.d. |
| 93 | ADI-42192 | 2.04E−07 | 89.44 | 38.91 | Other | IgM+ IgD+ CD27+ |
| 94 | ADI-48910 | 2.42E−09 | 99.95 | 59.48 | Other | IgG+ CD27+ |
| 95 | ADI-42193 | 9.22E−10 | 95.83 | 55.55 | 4G2 and 5A | swIg+ CD27+ |
| 96 | ADI-49590 | 1.09E−07 | 97.28 | 39.45 | Other | IgD memory (IgD+ IgM− CD27− SHM+) |
| 97 | ADI-45076 | 8.82E−10 | 40.69 | 13.70 | blocks 4G2 | swIg+ CD27+ |
| | | | | | only | |
| 98 | ADI-48968 | 9.63E−10 | 97.97 | 31.34 | blocks 4G2 | IgG+ CD27+ |
| | | | | | only | |
| 99 | ADI-42212 | 8.84E−10 | 99.71 | 89.90 | 4G2 and 5A | swIg+ CD27+ |
| 100 | ADI-48462 | >1.0E−07 | 97.94 | 48.44 | Blocks 5A | activated naïve |
| | | | | | only | (IgM+ IgD+ CD71+ /CD21loSHM−) |
| 101 | ADI-45127 | 6.69E−09 | 75.27 | 21.17 | 4G2 and 5A | swIg+ CD27− |
| 102 | ADI-42200 | 5.63E−09 | 86.80 | 30.37 | 4G2 and 5A | swIg+ CD27− |
| 103 | ADI-50203 | 2.24E−08 | 99.89 | 11.77 | Other | IgG+ CD27+ |
| 104 | ADI-42149 | 1.77E−07 | 88.08 | 65.95 | Other | swIg+ CD27− |
| 105 | ADI-42181 | 3.15E−08 | 82.16 | 7.59 | 4G2 and 5A | swIg+ CD27+ |
| 106 | ADI-45126 | 1.68E−09 | 86.51 | 35.29 | ADI-44112 | IgM-only (IgM+ IgD− CD27+) |
| 107 | ADI-45074 | 6.99E−10 | 49.44 | 15.50 | Other | swIg+ CD27+ |
| 108 | ADI-49041 | 9.97E−10 | 89.17 | 23.10 | ADI-44112 | swIg+ CD27− |
| 109 | ADI-42227 | 6.58E−10 | 96.46 | 49.47 | ADI-44112 | swIg+ CD27+ |
| 110 | ADI-50220 | 3.64E−09 | 99.84 | 49.33 | ADI-45107 | IgG+ CD27− |
| 111 | ADI-42141 | 1.75E−09 | 0 | 32.14 | blocks 4G2 | swIg+ CD27− |
| | | | | | only | |
| 112 | ADI-42216 | 1.14E−08 | 94.45 | 46.85 | 4G2 and 5A | swIg+ CD27− |
| 113 | ADI-50534 | >1.0E−07 | 85.81 | 43.79 | Other | n.d. |
| 114 | ADI-49140 | 1.04E−09 | 98.41 | 2.59 | 4G2 and 5A | swIg+ CD27− |
| 115 | ADI-46741 | >1.0E−07 | 94.68 | 23.14 | ADI-44112 | n.d. |
| 116 | ADI-42195 | 1.86E−09 | 92.25 | 31.33 | Other | swIg+ CD27− |
| 117 | ADI-42172 | 1.29E−07 | 97.07 | 29.12 | Other | swIg+ CD27+ |
| 118 | ADI-42178 | 3.44E−08 | 99.40 | 41.04 | Other | swIg+ CD27− |
| 119 | ADI-49032 | 4.86E−10 | 96.71 | 29.73 | ADI-45107 | swIg+ CD27+ |
| 120 | ADI-50197 | >1.0E−07 | 70.54 | 12.24 | Other | IgG+ CD27− |
| 121 | ADI-48894 | 9.56E−08 | 97.88 | 59.39 | blocks 4G2 | Atypical IgM memory |
| | | | | | only | (IgM+ IgD+ CD27− SHM+) |
| 122 | ADI-42226 | 8.43E−10 | 94.21 | 33.76 | Blocks 5A | swIg+ CD27− |
| | | | | | only | |
| 123 | ADI-49037 | 3.53E−09 | 79.59 | 7.14 | 4G2 and 5A | swIg+ CD27+ |
| 124 | ADI-46739 | >1.0E−07 | 95.66 | 55.09 | Other | n.d. |
| 125 | ADI-42810 | >1.0E−07 | 0 | 0 | Other | Atypical IgM memory |
| | | | | | | (IgM+ IgD+ CD27− SHM+) |
| 126 | ADI-49137 | 8.54E−10 | 46.64 | 50.93 | blocks 4G2 | IgD memory (IgM− IgD+ SHM+) |
| | | | | | only | |
| 127 | ADI-42817 | 4.15E−09 | 72.58 | 34.36 | Other | swIg+ CD27+ |
| 128 | ADI-50218 | >1.0E−07 | 66.77 | 14.49 | Other | Naïve (IgM+ IgD+ CD71− CD21+ SHM−) |
| 129 | ADI-42126 | 3.31E−07 | 20.23 | 1.07 | Other | swIg+ CD27+ |
| 130 | ADI-42186 | 1.21E−09 | 84.73 | 43.51 | Other | swIg+ CD27− |
| 131 | ADI-48890 | >1.0E−07 | 84.12 | 0 | Other | n.d. |
| 132 | ADI-42206 | 1.73E−09 | 71.04 | 23.09 | 4G2 and 5A | swIg+ CD27+ |
| 133 | ADI-46724 | >1.0E−07 | 93.22 | 62.53 | 5A and | n.d. |
| | | | | | ADI-45107 | |
| 134 | ADI-50539 | >1.0E−07 | 79.84 | 42.88 | Other | n.d. |
| 135 | ADI-45156 | >1.0E−07 | 54.57 | 2.90 | Other | n.d. |
| 136 | ADI-50536 | >1.0E−07 | 99.76 | 13.96 | Other | n.d. |
| 137 | ADI-42217 | 4.00E−09 | 50.24 | 3.33 | blocks 4G2 | swIg+ CD27+ |
| | | | | | only | |
| 138 | ADI-48951 | 2.67E−09 | 78.84 | 41.95 | Other | Atypical IgM memory |
| | | | | | | (IgM+ IgD+ CD27− SHM+) |
| 139 | ADI-50537 | >1.0E−07 | 90.56 | 13.47 | Other | n.d. |
| 140 | ADI-46737 | >1.0E−07 | 66.49 | 49.68 | Other | n.d. |
| 141 | ADI-50538 | >1.0E−07 | 74.32 | 19.58 | Other | n.d. |
| 142 | ADI-48950 | 1.53E−09 | 69.81 | 14.45 | blocks 4G2 | IgD memory (IgD+ IgM− CD27− SHM+) |
| | | | | | only | |
| 143 | ADI-42114 | >1.0E−07 | 70.93 | 23.01 | ADI-45107 | n.d. |
| 144 | ADI-49194 | 2.34E−07 | 80.83 | 46.50 | Other | IgG+ CD27+ |
| 145 | ADI-42124 | 5.88E−09 | 63.14 | 62.65 | Other | IgM-only (IgM+ IgD− CD27+) |
| 146 | ADI-45123 | 2.10E−09 | 59.03 | 27.03 | ADI-45107 | swIg+ CD27+ |
| 147 | ADI-50533 | >1.0E−07 | 78.78 | 43.72 | Other | n.d. |
| 148 | ADI-49205 | 1.29E−08 | 90.74 | 44.01 | 5A and | IgG+ CD27− |
| | | | | | ADI-45107 | |
| 149 | ADI-45151 | >1.0E−07 | 0 | 0 | blocks 4G2 | swIg+ CD27+ |
| | | | | | only | |
| 150 | ADI-46728 | >1.0E−07 | 89.49 | 48.99 | Other | n.d. |
| 151 | ADI-49030 | 1.25E−07 | 63.26 | 4.97 | Other | Naïve |
| 152 | ADI-50200 | >1.0E−07 | 97.30 | 0 | 4G2 and 5A | IgM+ IgD+ CD27+ |

* NN—non-neutralizing;
n.d.—not determined;
Other—did not block any of the listed competition assay controls

TABLE 3
Informal Sequence Listing

| Antibody Number | SEQ ID NO: | Sequence | Clone # (ADI) | Descriptors |
|---|---|---|---|---|
| 1 | 304 | QVQLQESGPGLVKPSETLSLTCAVSGYSISSG | ADI-49039 | Heavy chain |
| | | FYWGWIRQPPGKGLEWIGSMYQSGITYYNP | | variable region |
| | | SLKSRVTISVDTSKSQFSLKLTSVTAADTAM | | (“HC”) amino acid |
| | | YYCARNAPENYYGSGRESFDIWGQGTMVT | | sequence |
| | | VSS | | |
| 2 | 305 | QVQLQESGGDLVQPGGSLRLSCAASGFTFSN | ADI-49147 | Heavy chain |
| | | YAMNWVRQAPGKGLEWVSAINRGGDSTY | | variable region |
| | | YADSVKGRFTISRDNSKNTLYLQMNSLRAE | | (“HC”) amino acid |
| | | DTAVYYCAKDHGGKYGWWYFDLWGRGT | | sequence |
| | | LVTVSS | | |
| 3 | 306 | QVQLQESGPGLVKPSETLSLTCAVSGYSISSG | ADI-42229 | Heavy chain |
| | | FYWGWIRQPPGKGLEWIGSMYHSGITYYNP | | variable region |
| | | SLKSRVTISVDTSKNQFSLKLTSVTAADTAM | | (“HC”) amino acid |
| | | YYCARNAPENYYGSGRESFDIWGQGTTVT | | sequence |
| | | VSS | | |
| 4 | 307 | EVQLVESGGGLVQPGRPLRLSCAASGFAFSS | ADI-45090 | Heavy chain |
| | | YGMHWVRQAPGKGLEWVALIRFDGTIKY | | variable region |
| | | YADSVKGRFTISRDNAKNTLYLQMSSLRAE | | (“HC”) amino acid |
| | | DTAVYYCARDLEVGAEYLYYHYGMDVWG | | sequence |
| | | QGTTVTVSS | | |
| 5 | 308 | EVQLVESGGGVVQPGRSLRLSCAASGFTFNS | ADI-45097 | Heavy chain |
| | | HGMHWVRQAPGKGLEWVAVISYDGTKKY | | variable region |
| | | FADSVKGRFTISRDNSKNTLYLQMSSLRADD | | (“HC”) amino acid |
| | | TAVYYCAKDSSTSWYQVVYHIDYWGQGTL | | sequence |
| | | VTVSS | | |
| 6 | 309 | EVQLLESGGGLVQPGGSLRLSCAASGFTFRN | ADI-49133 | Heavy chain |
| | | YAMNWVRQTPGKGLEWVSGISGGGDSTNY | | variable region |
| | | ADSVKGRFTISRDNSRNTLYLQLNSLRAEDT | | (“HC”) amino acid |
| | | AVYYCAKDLAVSTPRYWFDSWGQGTLVTV | | sequence |
| | | SS | | |
| 7 | 310 | EVQLVESGGGLVQPGGSLRLSCAASGLIFRN | ADI-49033 | Heavy chain |
| | | YAMSWVRQAPGKGLEWVSSFSGSGGSAYY | | variable region |
| | | ADSVKGRFTISRDNSKSTVYLQMNRLRVED | | (“HC”) amino acid |
| | | TAVYYCAKDMAVSVHRGWFDDWGQGTLV | | sequence |
| | | TVSS | | |
| 8 | 311 | QVQLVESGGGVVQPGRSLRLSCAASGFAFSS | ADI-49044 | Heavy chain |
| | | YGMHWVRQAPGKGLEWVAGMRFDGTKIY | | variable region |
| | | YADSVKGRFTISRDNSKNTLYLQMNSLRAE | | (“HC”) amino acid |
| | | DTAVYFCARDLEVGAEYIYYYYGMDVWG | | sequence |
| | | QGTTVTVSS | | |
| 9 | 312 | QVQLVESGPGLVKPSGTLSLTCAVSGGSISS | ADI-45083 | Heavy chain |
| | | DYWWSWVRQPPGKGLEYIGEIYHTGSTNY | | variable region |
| | | NPSLKSRVTVSLDRSKNVFSLTLRSVTAADT | | (“HC”) amino acid |
| | | AVYYCARSHWRSPQSVTFDLWGQGTTVTV | | sequence |
| | | SS | | |
| 10 | 313 | QVQLQESGPGLVKPSGTLSLTCAVSGGSITSS | ADI-42225 | Heavy chain |
| | | NWWSWVRQPPGKGLEWIGDIYHSGSTSYN | | variable region |
| | | PSLKSRVTISVDKSKNHFSLKLTSVTAADTA | | (“HC”) amino acid |
| | | VYYCARIAAGYSTSWYYFDYWGQGTLVTV | | sequence |
| | | SS | | |
| 11 | 314 | EVQLVETGSGLVRPSGTLSLTCAVSGDSISSN | ADI-49139 | Heavy chain |
| | | NWWSWVRQPPGKGLEWIGEIYHSGSTSYN | | variable region |
| | | PSLKSRVTISIDKSNNHFSLKLTSVTAADTAV | | (“HC”) amino acid |
| | | YYCAKDMWAGTTTNWFGPWGQGTLVTVS | | sequence |
| | | S | | |
| 12 | 315 | QVTLKESGGALVKPAGSLTLSCAASGFTFG | ADI-48969 | Heavy chain |
| | | DYYMSWIRQAPGKGLEWISYISSSGSSIYYT | | variable region |
| | | DSVRGRFTISRDNARNSLYLQMNSLRVEDT | | (“HC”) amino acid |
| | | AVYYCAREFSSRPFDLWGQGTLVTVSS | | sequence |
| 13 | 316 | EVQLQESGPGLVKPSGTLSLTCAVSGGSISSS | ADI-48900 | Heavy chain |
| | | DWWSWVRQPPGKGLEWIGEIYHSGSTSYN | | variable region |
| | | PSVKSRVSISVDKSKNQFSLQLSSVTAADTAI | | (“HC”) amino acid |
| | | YYCARVNPPQYSSGWYSVYWGQGTLVTVS | | sequence |
| | | S | | |
| 14 | 317 | QVQLQQSGPGLVKPSGTLSLTCAVSGDSISSS | ADI-42232 | Heavy chain |
| | | HWWCWVRQPPGKGLEWIGEIYHSGSTSYN | | variable region |
| | | PSLKSRVTISVDKSKNQFSLKLSSVTAADTA | | (“HC”) amino acid |
| | | VYFCARVAWTSSSSCYYDYWGQGTLVTVS | | sequence |
| | | S | | |
| 15 | 318 | EVQLVESGPGLVKPSGTLSLTCAVSGGSISSS | ADI-42786 | Heavy chain |
| | | YWWSWVRQSPGKGLEWIGEVYHSGSTHY | | variable region |
| | | NPSLKSRVTISVDKSKNQFSLKLTSVTAADT | | (“HC”) amino acid |
| | | AVYYCARDGEGHYYRSGDNWFDRWGQGT | | sequence |
| | | LVTVSS | | |
| 16 | 319 | EVQLLESGPGLVQPSGTLSLTCTASGGSISSS | ADI-42210 | Heavy chain |
| | | NWWSWVRQPPGKGLEWIGDIYHTGSTSYN | | variable region |
| | | PSLKSRVTISVDKSKNQFSLKLSSVTAADTA | | (“HC”) amino acid |
| | | VYYCARAELSAWYYFDHWGQGTLVTVSS | | sequence |
| 17 | 320 | QVQLVESGGGLVKPGGSLRLSCAASGFIFSD | ADI-50201 | Heavy chain |
| | | YYMNWIRQAPGKGLDWVSTISGSGKSIYYA | | variable region |
| | | DSVKGRFTISRDNAKNSLYLQMNSLSAEDT | | (“HC”) amino acid |
| | | AVYYCARVSPLDDGYGYTYYGMDVWGQG | | sequence |
| | | TTVTVSS | | |
| 18 | 321 | EVQLLESGGGLVKPGGSLRLSCAASGFTFSD | ADI-48895 | Heavy chain |
| | | YYMSWIRQAPGKGLEWVSYITSSGNTKYY | | variable region |
| | | ADSVKGRFTISRDNAKNSLYLQISSLRAEDT | | (“HC”) amino acid |
| | | AVYYCARDWAELTTITNYFYPWGQGTTVT | | sequence |
| | | VSS | | |
| 19 | 322 | EVQLLESGGGLVQPGRSLRLSCAASGFTFDD | ADI-42228 | Heavy chain |
| | | YAMHWVRQPPGKGLEWVSGISWNGGGIG | | variable region |
| | | YADSVKGRFTISRDNAKNSLYLQMNSLRAD | | (“HC”) amino acid |
| | | DTALYYCAKAENRIGYCSAGSCYLTYFDY | | sequence |
| | | WGQGTLVTVSS | | |
| 20 | 323 | QVQLVQSGGGLVQPGGSLRLSCAASGFTFSS | ADI-45113 | Heavy chain |
| | | YAMSWVRQAPGKGLEWVSAISGSGGSTYY | | variable region |
| | | ADSVKGRFTISRDNSKNTLHLQMSSLRAEDT | | (“HC”) amino acid |
| | | AVYYCAKDPKYSSGWWAFDYWGQGTLVT | | sequence |
| | | VSS | | |
| 21 | 324 | EVQLVESGPGLVKPSGTLSLTCAVSGGSISSN | ADI-42198 | Heavy chain |
| | | KWWSWVRQPPGKGLEWIGEIYHSGSTSYN | | variable region |
| | | PSLKSRVSISVDKSKNQFSLKLSSVTAADTA | | (“HC”) amino acid |
| | | VYYCARVEWAYSSSWWLDYWGQGTLVTV | | sequence |
| | | SS | | |
| 22 | 325 | QVQLVESGGGLVKPGGSLRLSCAASGFTFSD | ADI-42190 | Heavy chain |
| | | DYMSWIRQAPGKGLEWVSYISGSGRAMYY | | variable region |
| | | ADSVQGRFTVSRDNAKNSLFLQMNNLRAED | | (“HC”) amino acid |
| | | TAVYYCAKHTGDKPLVWAPSVYGLDVWG | | sequence |
| | | QGTTVTVSS | | |
| 23 | 326 | QVQLQESGPGLVKPSGTLSLTCAVSGSSITSS | ADI-49154 | Heavy chain |
| | | HWWSWVRQPPGKGLAWIGDIYHSGGTTY | | variable region |
| | | NPSLKSRVTISVDKSKNQFSLKLSSVTAADT | | (“HC”) amino acid |
| | | AVYYCARVSVSTSAWYADYWGQGTLVTVS | | sequence |
| | | S | | |
| 24 | 327 | QVQLVESGGGLVKPGGSLRLSCVASGFTFN | ADI-49183 | Heavy chain |
| | | NYYMRWMRQAPGKGLEWVSQISSSGSIKD | | variable region |
| | | YADSVKGRFTVSRDNAKNSLYLQLNSLRAD | | (“HC”) amino acid |
| | | DTAVYFCARELSSRIDYWGQGTLVTVSS | | sequence |
| 25 | 328 | EVQLVESGGGVVQPGRSLRLSCVASGFTLRS | ADI-42201 | Heavy chain |
| | | YGMHWVRQVPGKGLEWVAVSWYDGSNK | | variable region |
| | | HYADSVKGRFSISRDNSKNTLYLQMNSLRA | | (“HC”) amino acid |
| | | EDTAVYYCARAQDGQQLVNYYGMDVWG | | sequence |
| | | QGTTVTVSS | | |
| 26 | 329 | QVQLVESGGGVVQPGRSLRLSCAASGFTFSS | ADI-42144 | Heavy chain |
| | | YTMHWVRQAPGKGLEWVAVISYDGSNKY | | variable region |
| | | YADSVKGRFTISRDNSKNTLYLQMNSLRAE | | (“HC”) amino acid |
| | | DTAVYYCARGGDYGDYESNNPAEYFQHW | | sequence |
| | | GQGTLVTVSS | | |
| 27 | 330 | QVQLQESGPGLVKPSETLSLTCTVSGDSISVS | ADI-50219 | Heavy chain |
| | | YWSWIRQFPGKGLEWIGYIYNSGNANYNPS | | variable region |
| | | LESRVTISIDTSKNRFSLRLSSVTAADTAVYY | | (“HC”) amino acid |
| | | CAGHREDPYGAYGASWGQGTLVTVSS | | sequence |
| 28 | 331 | EVQLLESGPGLVKPSETLSLTCTVSGGSLSSD | ADI-48897 | Heavy chain |
| | | SHFWGWIRQPPGKGLEWIGYIYYSGNANYN | | variable region |
| | | PSLQSRVTISLDKSKNQFSLRLTSVTAADTA | | (“HC”) amino acid |
| | | VYYCASRKEVRGTEDYFDYWGQGTLVTVS | | sequence |
| | | S | | |
| 29 | 332 | EVQLQESGPGLVKPSETLSLTCTVSGGSVSS | ADI-42194 | Heavy chain |
| | | GSYYWSWIRQPPGKGLEWIGYIYDSGNTNY | | variable region |
| | | NPSLKSRVTISVDTSKRQFSLRLTSVTAADT | | (“HC”) amino acid |
| | | AVYYCAKVEEDGYTNVVRDYWGQGTLVT | | sequence |
| | | VSS | | |
| 30 | 333 | EVQLVESGGGLVKPGGSLRLSCAASGFTFSD | ADI-49189 | Heavy chain |
| | | YYMSWIRQAPGKGLECIACISSSGSMIYYAD | | variable region |
| | | SVKGRFTISRDNAKNSLYLQLNSLRVEDTAV | | (“HC”) amino acid |
| | | YYCAREGTRGRMDWGQGTLVTVSS | | sequence |
| 31 | 334 | EVQLLESGPGLVRPSGTLSLTCAVSGGSISTT | ADI-49188 | Heavy chain |
| | | DWWSWVRQPPGKGLEWIGEINQSGSTSYSP | | variable region |
| | | SFKSRVSISVDKSKRQFSLKLTSVTAADTAV | | (“HC”) amino acid |
| | | YYCARDSWSGPTRNWFDPWGRGTLVTVSS | | sequence |
| 32 | 335 | EVQLLESGPGLVKPSGTLSLTCAVSGGSISSG | ADI-42188 | Heavy chain |
| | | NWWSWVRQPPGKGLEWIGEIYHSGSANYN | | variable region |
| | | PSLKSRVTISVDKSKNQFSLKLTSVTAADTA | | (“HC”) amino acid |
| | | VYYCARVVWEYSNAWCVDFWGQGTTVTV | | sequence |
| | | SS | | |
| 33 | 336 | EVQLLESGGGVVQPGRSLRLSCAASGFTFTT | ADI-50026 | Heavy chain |
| | | YAMHWVRQAPGKGLEWVAAVSYDGNNKY | | variable region |
| | | YADSVKGRFTISRDNSRNTLYLQMNSLRAE | | (“HC”) amino acid |
| | | DTAVYFCARNTYYDRSGLIAYWGQGALVT | | sequence |
| | | VSS | | |
| 34 | 337 | QVQLVESGPGLVKPSGTLSLTCAVSGDSISST | ADI-42809 | Heavy chain |
| | | NWWSWVRQPPGKGLEYIGEIFHSGSTNYNP | | variable region |
| | | FLKSRVTISVDKSKNHFSLKLSSVTAADTAV | | (“HC”) amino acid |
| | | YYCARGPLKSYWYFDLWGRGTLVTVSS | | sequence |
| 35 | 338 | QVQLQESGPGLVKPSGTLSLTCAVSGGSISS | ADI-46596 | Heavy chain |
| | | NNWWSWVRQPPGKGLEWIGDTYHSGSPSY | | variable region |
| | | NPSLKSRVTISVDKSKNEFSLKLSSVTAADT | | (“HC”) amino acid |
| | | AVYFCARYCSGATCYGSNGMDVWGQGTT | | sequence |
| | | VTVSS | | |
| 36 | 339 | QVQLQESGGGVVQPGRSLRLSCAASGFTFS | ADI-50205 | Heavy chain |
| | | NFGMHWVRQAPGKGLEWVAIISYDRSNKD | | variable region |
| | | YADSVKGRFTISRDNSKNTLYLQMNSLRAE | | (“HC”) amino acid |
| | | DTAVYYCAKDQCGGDCTADYWGQGTLVT | | sequence |
| | | VSS | | |
| 37 | 340 | EVQLLESGPGLVRPSGTLSLTCAVSGASISSN | ADI-42830 | Heavy chain |
| | | HWWTWVRQPPGKGLEWIGEIYHSGSPTYN | | variable region |
| | | PSLKSRVTISVDKSKNQFSLKLNSVTAADTA | | (“HC”) amino acid |
| | | VYYCASTLWGGPLSVASDYWGQGTLVTVS | | sequence |
| | | S | | |
| 38 | 341 | QVQLVESGGGVVQPGRSLRLSCAASGFTFS | ADI-49186 | Heavy chain |
| | | NSGMHWVRQAPGQGLEWVALISYTGETK | | variable region |
| | | YYSDSLKARFTISRDNSKNTLYLQMSSLSNE | | (“HC”) amino acid |
| | | DTAVYYCARDYYASGDGYFDYWGQGTLV | | sequence |
| | | TVSS | | |
| 39 | 342 | QVQLQQWGPELVKPSGTLSLTCTVSGGSISSI | ADI-46591 | Heavy chain |
| | | SWWSWVRQSPGKGLEWIGEINHSGSTVYN | | variable region |
| | | PSLKSRVTISVDKSKKQFSLKLRSVTAADTA | | (“HC”) amino acid |
| | | VYYCVRYCSSTSCYGLNGMDVWGQGTTV | | sequence |
| | | TVSS | | |
| 40 | 343 | QVQLVQSGGGLVNPGGSLRLSCAASGFTFT | ADI-48955 | Heavy chain |
| | | DYYMSWIRQAPGKGLEWVSYISSSGNTRYY | | variable region |
| | | ADSVKGRFTISRDNAKNSLSLQMNSLRPEDT | | (“HC”) amino acid |
| | | AIYYCARDGSLVNAIDYWGQGTLVTVSS | | sequence |
| 41 | 344 | EVQLVESGPGLVKPSGTLSLTCAVSGGSITG | ADI-42818 | Heavy chain |
| | | SNWWSWVRQPPGKGLEWIGEIYHTGSTSY | | variable region |
| | | NPSLKSRVTISVDNSKNHFSLRLTSVTAADT | | (“HC”) amino acid |
| | | AVYYCARVRWSGSTSWDLDYWGQGTLVT | | sequence |
| | | VSS | | |
| 42 | 345 | EVTLKESGPTLVKPTQTLTLTCTFSGFSLSTS | ADI-50531 | Heavy chain |
| | | GVGVGWIRQPPGKALEWLALIYWDDDKRY | | variable region |
| | | SPSLKSRLTITKDTSKNQVVLTMTNMDPVDT | | (“HC”) amino acid |
| | | ATYYCAHSPRRITMVRGVIITWGDGMDV | | sequence |
| | | WGQGTTVTVSS | | |
| 43 | 346 | EVQLVESGGGLVKPGGSLRLSCAASGFTFTD | ADI-46586 | Heavy chain |
| | | YYMSWIRQAPGKGLEWVSYITSSGNTKYY | | variable region |
| | | ADSVKGRFTISRDNAKNSLFLQMNSLRAEDT | | (“HC”) amino acid |
| | | AVYFCARDGSMVNAIDYWGQGTLVTVSS | | sequence |
| 44 | 347 | QVQLVESGGGVVQPGRSLRLSCAASGFTFS | ADI-49138 | Heavy chain |
| | | NSGMHWVRQAPGKGLEWVSVIWYDESNK | | variable region |
| | | YYADSVKGRFTISRDNSKNTVYLQMNTLRA | | (“HC”) amino acid |
| | | EDTAVYYCARDAYASGDGGIDYWGQGALV | | sequence |
| | | TVSS | | |
| 45 | 348 | EVQLLESGGGLVQPGGSLRLSCAASGFTFSS | ADI-45075 | Heavy chain |
| | | YAMSWVRQAPGKGLEWVSVISDSGGSTYY | | variable region |
| | | ADSVKGRFTISRDNSKNTLYLQMNSLRAED | | (“HC”) amino acid |
| | | TAVYYCAKDLRGVGGWYYFDYWGQGTLV | | sequence |
| | | TVSS | | |
| 46 | 349 | EVQLVESGGGLVQPGGSLRLSCAASGFTFIN | ADI-42831 | Heavy chain |
| | | YAMTWVRQAPGKGLEWVSAISGNGDGTY | | variable region |
| | | YADSVKGRFTLSRDNAKNTIYLHMSALRDE | | (“HC”) amino acid |
| | | DTALYYCAKDQGVTTDWPSDYWGQGTLV | | sequence |
| | | TVSS | | |
| 47 | 350 | QVQLVESGGGVVQPGRSLRLSCAASGFTFSS | ADI-42230 | Heavy chain |
| | | YAMHWVRQAPGKGLEWVAVISHDGSNKY | | variable region |
| | | YADSVKGRFTISRDNSKNTLYLQINSLRAED | | (“HC”) amino acid |
| | | TAVYYCPRDGLPGANQYFFYYGMDVWGQ | sequence | |
| | | GTTVTVSS | | |
| 48 | 351 | EVQLLESGPRLVKPSETLSLTCTVSGGSVRG | ADI-42847 | Heavy chain |
| | | GSHYWSWIRQPPGKGLEWIGYVYDSGSTNY | | variable region |
| | | NPSLKSRVSISVDMSKKQFSLKLRSVTAADT | | (“HC”) amino acid |
| | | AVYHCVRVEEYVNNEEVRDYWGQGTMVT | | sequence |
| | | VSS | | |
| 49 | 352 | EVQLLESGGGLVPPGGSLRLSCAASGFTFSN | ADI-42821 | Heavy chain |
| | | YAMSWVRQAPGKGLEWVSAISGSGDSTYY | | variable region |
| | | ADSVKGRFTLSRDTSKKMVYLHMSNLRDD | | (“HC”) amino acid |
| | | DTAVYYCARDQGFTTDWPCDYWGQGTLV | | sequence |
| | | TVSS | | |
| 50 | 353 | QVQLVESGGGLVKPGGSLRLSCAASGFTFSD | ADI-42849 | Heavy chain |
| | | YYMSWIRQAPGKGLEWVSYITSSGNTMYY | | variable region |
| | | ADSVKGRFTISRDNAKNSLYLQMNSLRAED | | (“HC”) amino acid |
| | | TAVYYCARDSNFNSNLDYWGQGTLVTVSS | | sequence |
| 51 | 354 | QVQLQESGPGLVKPSGTLSLTCAVSGGSISSS | ADI-42151 | Heavy chain |
| | | NWWSWVRQPPGKGLEWIGEIYHSGSTTYN | | variable region |
| | | PSLKSRVTISVDKSKNQFSLKLSSVTAADTA | | (“HC”) amino acid |
| | | VYYCARGPLKTYWYFDLWGRGTLVTVSS | | sequence |
| 52 | 355 | EVQLVESGGGLVKPGGSLRLSCAASGFTFSD | ADI-46001 | Heavy chain |
| | | YYMSWIRQAPGKGLEWVSYISSSGNTIYYA | | variable region |
| | | DSVKGRFTISRDNAKNSLYLQLNSLRAGDTA | | (“HC”) amino acid |
| | | VYYCARDSNYFYGLDVWGQGTTVTVSS | | sequence |
| 53 | 356 | EVQLVESGGGVVQPGRSLRLSCAASGFTFSN | ADI-45154 | Heavy chain |
| | | YGMHWVRQAPGKGLEWVAVISYDGSNKY | | variable region |
| | | YADSVKGRFTISRDDSKNTLYLQVNSLRAED | | (“HC”) amino acid |
| | | TAVYYCAKDICSGDCGGGDYWGQGTLVTV | | sequence |
| | | SS | | |
| 54 | 357 | QVQLVQSGAEVKKPGASVKVSCKASGYTFN | ADI-49161 | Heavy chain |
| | | TYAMTWVRQAPGQGLEWMGWISTYNGNT | | variable region |
| | | VFGQKFQGRVTLSTDTSTSTAYMELRSLTS | | (“HC”) amino acid |
| | | DDTAVYYCAREDDDYYSMDVWGQGTTVT | | sequence |
| | | VSS | | |
| 55 | 358 | EVQLVQSGGGLVQPGGSLRLSCAASGFTFST | ADI-42154 | Heavy chain |
| | | YWMSWVRQAPGKGLEWVANIKQDGSEKY | | variable region |
| | | YVDSVKGRFTISRDNAKNSLYLQMNSLRAE | | (“HC”) amino acid |
| | | DTAVYYCARDISCISTSCYGGYYYYGMDV | | sequence |
| | | WGQGTTVTVSS | | |
| 56 | 359 | EVQLVESGGGVVQPGRSLRLSCAASGFTFSN | ADI-48916 | Heavy chain |
| | | SGMHWVRQAPGKGLEWVAVIWYDSRNQN | | variable region |
| | | YADSVKGRFTISRDNSKNTLFLQMNSLRAED | | (“HC”) amino acid |
| | | TAVYYCARDYYASGDGSIDYWGQGTLVTV | | sequence |
| | | SS | | |
| 57 | 360 | EVQLVESGGGLVQPGGSLRLSCAASGFTFSS | ADI-45085 | Heavy chain |
| | | YAMSWVRQAPGKGLEWVSTFSGRGGSTYY | | variable region |
| | | ADFVKGRFTISRDNSKNTLYLQMNSLRAED | | (“HC”) amino acid |
| | | TAVYYCAKYYDSSGYYYFDYWGQGTLVTV | | sequence |
| | | SS | | |
| 58 | 361 | QVQLVESGGGVVQPGRSLRLSCGGSGFTFSS | ADI-42211 | Heavy chain |
| | | YGMHWVRQAPGKGLEWVAVISYDGSKKY | | variable region |
| | | SADSVKGRFTISRDNSKNTLYLQMNSLRAED | | (“HC”) amino acid |
| | | TAVYYCAKGSVSVAGAEDYWGQGTLVTVS | | sequence |
| | | S | | |
| 59 | 362 | EVQLLESGGGLVQPGRSLRLSCAVSGFTFAE | ADI-48908 | Heavy chain |
| | | YAMHWVRQAPGKGLEWVSSISWNSGRIGY | | variable region |
| | | VDSVRGRFTISRDNAKNSLYLQMNSLRVED | | (“HC”) amino acid |
| | | TAFYYCAKGYDSSGYYWADYWGQGTLVT | | sequence |
| | | VSS | | |
| 60 | 363 | EVQLLESGPGLVKPSETLSLTCTVSGGSISSY | ADI-48913 | Heavy chain |
| | | YWSWIRQPAGKGLELIGRIYTSGSGNYNPSL | | variable region |
| | | KRRVTMSVDTSKNQISLRLNSVTAADTAVY | | (“HC”) amino acid |
| | | YCARERGGYFTEPFDIWGQGTMVTVSS | | sequence |
| 61 | 364 | EVQLLESGGGLVHPGGSLRLSCAASGFTFSD | ADI-45140 | Heavy chain |
| | | YEMNWVRQAPGKGLEWVSHISSSGNIIYYA | | variable region |
| | | DSVKGRFTISRDNAKDSLYLQMNSLRAEDT | | (“HC”) amino acid |
| | | AVYYCAATIFGVVSFDYWGQGTLVTVSS | | sequence |
| 62 | 365 | EVQLVESGGGLVQPGGSLRLSCAASGFTFSA | ADI-50211 | Heavy chain |
| | | YAMSWVRQAPGRGLEWVSAISGSDRRIYY | | variable region |
| | | ADSVKGRFSISRDNSKNTLYLQMSSLRAEDT | | (“HC”) amino acid |
| | | AVYYCAKYYDSSGYYYLDYWGQGTLVTVS | | sequence |
| | | S | | |
| 63 | 366 | EVQLVESGGGLVQPGGSLRLSCAASGFTFSD | ADI-42199 | Heavy chain |
| | | HYMAWVRQAPGKGLEWVGRIRNKPNSYT | | variable region |
| | | TEYAASVKGRFTISRHDSENSLYLQMNSLKT | | (“HC”) amino acid |
| | | EDTAVYYCCRESGEGFDPWGQGTLVTVSS | | sequence |
| 64 | 367 | QVQLVQSGAEVKKPGASVKVSCKASGYSFT | ADI-42231 | Heavy chain |
| | | TYGISWVRQAPGQGLEWMGWISGYSGDTN | | variable region |
| | | YAQKVQGRVTMTTDTSTSTAYMELRSLRSD | | (“HC”) amino acid |
| | | DTAVYYCARDQSHGTFGGVIDSTTLFYYY | | sequence |
| | | GMDVWGQGTTVTVSS | | |
| 65 | 368 | EVQLQESGPGLVKPSETLSLTCTVSGGSISSS | ADI-45164 | Heavy chain |
| | | SYYWGWIRQPPGKGLEWIGSIYYSGSTYYN | | variable region |
| | | PSLKSRVTISVDTSKNQFSLKLSSVTAADTA | | (“HC”) amino acid |
| | | VYYCARGYCSSTSCFYYYYGMDVWGQGT | | sequence |
| | | TVTVSS | | |
| 66 | 369 | EVQLVESGGGLVKPGGSLRLSCVASGFTFSR | ADI-42233 | Heavy chain |
| | | YSMNWVRQAPGKGLEWVSSISHSGRYIYY | | variable region |
| | | ADSEKGRFTISRDNAKNSLYLQMNSLRAED | | (“HC”) amino acid |
| | | TAVYYCARDHYFDSSGDYLSYYYNGMDV | | sequence |
| | | WGQGTTVTVSS | | |
| 67 | 370 | EVQLVESGGGLVQPGGSLRLSCAASGFTFSD | ADI-42191 | Heavy chain |
| | | HYMDWVRQAPGKGLEWVGRTRNKPNSHT | | variable region |
| | | TEYAASVKGRFTISRDDSKNSLYLQMNSLQ | | (“HC”) amino acid |
| | | TEDTAVYYCARVYGGPDDYWGQGTLVTVS | | sequence |
| | | S | | |
| 68 | 371 | EVQLVESGGGLVQPGGSLRLSCAASGFIYTN | ADI-48899 | Heavy chain |
| | | YAMYWVRQAPGKGLEWVSAISGSGGITYY | | variable region |
| | | ADSVKGRFTISRDNSKNTLYLQMNSLRAED | | (“HC”) amino acid |
| | | KAVYYCAKDGVTTINGWFHFEYWGQGTL | | sequence |
| | | VTVSS | | |
| 69 | 372 | EVQLLESGGGLVQPGGSLRLSCAASGFIFSD | ADI-49145 | Heavy chain |
| | | YYMDWVRQTPGKGPEWVGRITNRPNSYTT | | variable region |
| | | EYAASVKGRFTISRDDSTNSLFLHMNSLKTE | | (“HC”) amino acid |
| | | DTAVYYCTRITGDRYWYLDLWGRGTLVTV | | sequence |
| | | SS | | |
| 70 | 373 | EVQLVESGPGLVKPSQTLSLTCTVSGGSISSG | ADI-46729 | Heavy chain |
| | | SYYWSWIRQPAGKGLEWIGRIYTSGSTNYN | | variable region |
| | | PSLKSRVTMSVDTSKNQFSLKLSSVTAADTA | | (“HC”) amino acid |
| | | VYYCARGWFGYSNYGLYYYYGMDVWGQ | | sequence |
| | | GTTVTVSS | | |
| 71 | 374 | EVQLVESGPGLVKPSGTLSLTCAVSGGSISSS | ADI-46722 | Heavy chain |
| | | NWWSWVRQPPGKGLEWIGEIYHSESTNYN | | variable region |
| | | PSLKSRVTISVDKSKNQFSLKLSSVTAADTA | | (“HC”) amino acid |
| | | VYYCARDFWSGSNWFDPWGQGTLVTVSS | | sequence |
| 72 | 375 | EVQLLESGGGLVQPGRSLRLSCAASGFTFDD | ADI-45148 | Heavy chain |
| | | YAMHWVRQAPGKGLEWVSGISWNSGSIGY | | variable region |
| | | ADSVKGRFTISRDNAKNSLYLQMNSLRAED | | (“HC”) amino acid |
| | | TALYYCAKDIGDSYGSGSYYLPYGAYYGM | | sequence |
| | | DVWGQGTTVTVSS | | |
| 73 | 376 | EVQLLESGGGLVQPGGSLRLSCAASGFTFSS | ADI-49168 | Heavy chain |
| | | YAMSWVRQATGRGLEWVSSIRSSGGRTEY | | variable region |
| | | ADSVKGRFTISRDNSKNTLYLQMDSLRAED | | (“HC”) amino acid |
| | | TALYYCAKHYDSSGYYYEDYWGQGTLVTV | | sequence |
| | | SS | | |
| 74 | 377 | EVQLVESGGALVHPGGSLGLSCAASGFTFSD | ADI-49040 | Heavy chain |
| | | HYMDWVRQAPGKGLEWVGRIRNKPNSYA | | variable region |
| | | TQYAASVKGRFTISRDDSKKSLYLQMNSLN | | (“HC”) amino acid |
| | | TEDTAVYYCARVRDGEYDYWGQGTLVTVS | | sequence |
| | | S | | |
| 75 | 378 | EVQLLESGGGLVKPGGSLRLSCAASGFTFSS | ADI-42187 | Heavy chain |
| | | YSMNWVRQAPGKGLEWVSSISSRSSFMYY | | variable region |
| | | ADSVKGRFTISRDNAKNSLYLQMNSLRVED | | (“HC”) amino acid |
| | | TAVYYCARDNSEVEDYGDYVLYHYYGMD | | sequence |
| | | VWGQGTTVTVSS | | |
| 76 | 379 | EVQLLESGGGVVQPGRSLRLSCVASGFTFSS | ADI-49561 | Heavy chain |
| | | YGMHWVRQAPGKGLEWVALISYDGSNKY | | variable region |
| | | YADSVKGRFTISRDNSKNTLYLQMNSLRAE | | (“HC”) amino acid |
| | | DTAVYYCAKDQCGGDCTADYWGQGTLVT | | sequence |
| | | VSS | | |
| 77 | 380 | QVQLVESGGGVVQPGRSLRLSCAASGFTFSS | ADI-42219 | Heavy chain |
| | | LAMHWVRQAPGKGLEWVATISYDVSNKY | | variable region |
| | | YADSVKGRFTISRDNSKNTLFLQMNSLRPED | | (“HC”) amino acid |
| | | TAVYYCARGYTGYDGFDYWGQGTLVTVSS | | sequence |
| 78 | 381 | QVQLVQSGAEVKKPGASVKVSCKASGYTFT | ADI-50535 | Heavy chain |
| | | SYGISWVRQAPGQGLEWMGWISAYNGNTN | | variable region |
| | | YAQKLQGRVTMTTDTSTSTAYMELRSLRSD | | (“HC”) amino acid |
| | | DTAVYYCARRPYYYGSRRPAGHMDVWGQ | | sequence |
| | | GTTVTVSS | | |
| 79 | 382 | QVQLQESGPGLVRPSQTLSLTCTVSGGAISS | ADI-45128 | Heavy chain |
| | | GDYYWSWVRQPPGKGLEWIGYIHYSGTTY | | variable region |
| | | NNPSLKSRVTIAVDTSKNQFSLKLSSVTAAD | | (“HC”) amino acid |
| | | TAVYFCGRDSDKNYFDYWGQGTLVTVSS | | sequence |
| 80 | 383 | EVQLVESGGGVVRPGRSLRLSCAASGFTFSS | ADI-45136 | Heavy chain |
| | | YGMHWVRQAPGKGLEWVAVIRFDGSNTV | | variable region |
| | | YADSVKGRFTISRDNSKNTLYLQMNSLRAE | | (“HC”) amino acid |
| | | DTAVYYCAKTYDSNAYYYLDYWGQGTLVT | | sequence |
| | | VSS | | |
| 81 | 384 | EVQLVESGGGVVQPGWSLRLSCAVSGFTFS | ADI-42189 | Heavy chain |
| | | SYAMHWVRQAPGKGLEWVAVISYDGSYK | | variable region |
| | | WYADSVKGRFTISRDNSKNTVYLQMNSLRA | | (“HC”) amino acid |
| | | EDTAVYYCASLWFIVMTMSKNPETDYWG | | sequence |
| | | QGTLVTVSS | | |
| 82 | 385 | EVQLVESGGGLIQPGGSLRLSCAASGFSFSSH | ADI-45078 | Heavy chain |
| | | AMTWVRQAPGKGLQWVSSIRGSDRTTNYA | | variable region |
| | | DSVKGRFTVSRDNSKNTLYLQMNSLRAEDT | | (“HC”) amino acid |
| | | AIYYCAKYYDSSGYYYFDHWGQGTLVTVS | | sequence |
| | | S | | |
| 83 | 386 | EVQLVESGGTFLQPGGSLRLSCVASGFTFGT | ADI-49162 | Heavy chain |
| | | HAMSWVRQAPGKGLEWVSTFSGSGGRTY | | variable region |
| | | YADSVKGRFTISRDNSKSTLYLEMSALRAED | | (“HC”) amino acid |
| | | TAVYYCAKFYDSSGYYYFDYWGQGTLVTV | | sequence |
| | | SS | | |
| 84 | 387 | EVQLVESGGGLVQPGGSLRLSCAASGFTFSD | ADI-42223 | Heavy chain |
| | | YYMDWVRQAPGKGLEWVGGIRNKPNSYT | | variable region |
| | | TEYAASVKGRFTISRDDSKNSLFLQMNSLKT | | (“HC”) amino acid |
| | | EDTAVYYCVRLYGDYVAYFDYWGQGTLVT | | sequence |
| | | VSS | | |
| 85 | 388 | QVQLVQSGAEVKKPGASVKVSCKASGYTFT | ADI-48435 | Heavy chain |
| | | SYGISWVRQAPGQGLEWMGWISAYNGNTN | | variable region |
| | | YAQKLQGRVTMTTDTSTSTAYMELRSLRSD | | (“HC”) amino acid |
| | | DTAVYYCARRGTTVTRFGVIQYYYGMDV | | sequence |
| | | WGQGTTVTVSS | | |
| 86 | 389 | QVQLQESGPGLVKPSETLSLTCTVSGASIRSY | ADI-46742 | Heavy chain |
| | | LWSWIRQPPGKELEWLGSIYHSGSTKYNPS | | variable region |
| | | LKSRVTISADTSKNQFSLKLNSVTAADTAVF | | (“HC”) amino acid |
| | | YCARETANNWFDPWGQGTLVTVSS | | sequence |
| 87 | 390 | EVQLVESGGGVVQSGRSLRLSCAASGFTFSG | ADI-42787 | Heavy chain |
| | | NAMHWVRQAPGKGLEWVAVILYDGSNQY | | variable region |
| | | YADSVKGRFTISRDNSKNTLYLQMNSLRPA | | (“HC”) amino acid |
| | | DTAVYYCARASMMPRPPVHDYWGQGTLV | | sequence |
| | | TVSS | | |
| 88 | 391 | EVQLLESGGGLVQPGGSLRLSCAASGFTFSS | ADI-46718 | Heavy chain |
| | | YAMSWVRQAPGKGLEWVSAISGSGGSTYY | | variable region |
| | | ADSVKGRFTISRDNSKNTLYLQMNSLRAED | | (“HC”) amino acid |
| | | TAVYYCAKDRSQGDYGDYVADYWSQGTL | | sequence |
| | | VTVSS | | |
| 89 | 392 | EVQLQESGPGLVKPSGTLSLTCAVSGGSISSS | ADI-49141 | Heavy chain |
| | | NWWTWVRQPPGKGLEWIGEIYHSGSTNYN | | variable region |
| | | PSLESRVTMSVDKSKNQFSLKLSSVTAADTA | | (“HC”) amino acid |
| | | VYYCARVQTSHSELWFGEFGADWGQGTL | | sequence |
| | | VTVSS | | |
| 90 | 393 | EVQLLESGGGLVQPGGSLRLSCAASGFTFTY | ADI-42213 | Heavy chain |
| | | YAMSWVRQAPGKGLEWVSGISGSGDSTYN | | variable region |
| | | ADSVKGRVTISRDNSKNTLYLQMNSLRAED | | (“HC”) amino acid |
| | | TAVYYCAKDGGYSTDWYFDLWGRGTLVT | | sequence |
| | | VSS | | |
| 91 | 394 | QVQLVESGGGVVQPGRSLRLSCTASGFTFSS | ADI-42844 | Heavy chain |
| | | YGMHWVRQAPGKGPEWVAVISYDGSKKY | | variable region |
| | | FADSVKGRFTISRDNSKNTLYLQMNSLRAE | | (“HC”) amino acid |
| | | DTAVYSCAKGYDSNGYYYIDYWGQGTPVT | | sequence |
| | | VSS | | |
| 92 | 395 | QVQLQESGGGVVQPGRSLRLSCAASGFTFSS | ADI-45161 | Heavy chain |
| | | YGMHWVRQAPGKGLEWVAVIWYDGSNKY | | variable region |
| | | YADSVKGRFTISRDNSKNTLYLQMNSLRAE | | (“HC”) amino acid |
| | | DTAVYYCARDVGYQLLQVYGMDVWGQG | | sequence |
| | | TTVTVSS | | |
| 93 | 396 | EVQLLESGPGLVKPSQTLSLTCSVSGGSISSG | ADI-42192 | Heavy chain |
| | | GYYWTWIRQPPGKGLEWIGYIYYTGSTYYN | | variable region |
| | | PSLKSRVTISVDTSKNQFSLKLSSVTAADTA | | (“HC”) amino acid |
| | | VYFCARAEYDTSGYYQQRLPEYFQHWGQ | | sequence |
| | | GTLVTVSS | | |
| 94 | 397 | EVQLVQSGGGLVQRGGSLRLSCAASGFTFSS | ADI-48910 | Heavy chain |
| | | YAMTWVRQAPGKGLEWVSDMNHSGDRTN | | variable region |
| | | YADSVRGRFTISRDNSKNTLYLQMNSLRAE | | (“HC”) amino acid |
| | | DTAVYYCAKYYDSSGYYYFHSWGQGTLVT | | sequence |
| | | VSS | | |
| 95 | 398 | EVQLLESGGGLVQPGGSLRLSCAASGFIFSD | ADI-42193 | Heavy chain |
| | | HYMAWVRQAPGKGLEWVGRSRNRPNSYT | | variable region |
| | | TEYAASAKGRFTISRDDSKTSLYLQMNSLKT | | (“HC”) amino acid |
| | | EDTAVYYCAREHGDYGLDYWGQGTLVTVS | | sequence |
| | | S | | |
| 96 | 399 | QVQLVQSGAEVKKPGASVKVSCKASGYTFT | ADI-49590 | Heavy chain |
| | | GYYMHWVRQAPGQGLEWMGRINPNSGGT | | variable region |
| | | NYAQKFQGRVTMTRDTSISTAYMELSRLRS | | (“HC”) amino acid |
| | | DDTAVYYCYVDYYYDSSGYYSPFDYWGQG | | sequence |
| | | TLVTVSS | | |
| 97 | 400 | EVQLVESGGGFVQPGGSLRLSCAASGFIFSD | ADI-45076 | Heavy chain |
| | | YYMDWVRQAPGKGLEWVGRIRNKPNSYT | | variable region |
| | | TEYAASVKGRFSISRDDLKNSLYLQMNSLK | | (“HC”) amino acid |
| | | TEDTAEYYCARVDGEEVALIYWGQGALVT | | sequence |
| | | VSS | | |
| 98 | 401 | EVQLLESGGGLGQPGGSLRLSCVASKFTFSD | ADI-48968 | Heavy chain |
| | | HYMDWVRQAPGKGLEWVGRIRNKPNGYT | | variable region |
| | | TEYAASVKGRFIISRDDSKNSLYLQMKSLKI | | (“HC”) amino acid |
| | | EDTAIYYCVRVWGGEAARYDYWGQGALV | | sequence |
| | | TVSS | | |
| 99 | 402 | EVQLVESGGGLVQPGGSLRLSCAASGFTFSD | ADI-42212 | Heavy chain |
| | | HYMDWVRQAPGKGLEWVGRSRNKPNSYIT | | variable region |
| | | EYAASVKGRFTISRDDSKNSLYLQMNSLKTE | | (“HC”) amino acid |
| | | DTAVYYCSRHMGFGLDLWGQGTLVTVSS | | sequence |
| 100 | 403 | QVQLVQSGGGVVQPGRSLRLSCAASGFTFSS | ADI-48462 | Heavy chain |
| | | YGMHWVRQAPGKGLEWVAVIWYDGSNKY | | variable region |
| | | YADSVKGRFTISRDNSKNTLYLQMNSLRAE | | (“HC”) amino acid |
| | | DTAVYYCARDYYGSGDGYFDYWGQGTTV | | sequence |
| | | TVSS | | |
| 101 | 404 | EVQLVESGPVLVKPTETLRLTCTVSGFSLSN | ADI-45127 | Heavy chain |
| | | TKLGVSWIRQPPGKALEWLAHIFSNAEKSS | | variable region |
| | | SKSLKSRLSISQDTSKSLVVLTMTNMDPVDT | | (“HC”) amino acid |
| | | ATYFCARIPVEYGTPRGSFDTWGQGTTVTV | | sequence |
| | | SS | | |
| 102 | 405 | EVQLVESGGGVVQPGRSLRLSCAASGLTFST | ADI-42200 | Heavy chain |
| | | YTLHWVRQAPGKGLEWVAVISSDGGNKYY | | variable region |
| | | ADSVKGRFTISRDSSKNTLYLQMNSLRTEDT | | (“HC”) amino acid |
| | | AVYYCAGGSPDYWGQGALVTVSS | | sequence |
| 103 | 406 | EVQLVESGGGVVQPGRSLRLSCVPSGFTFSS | ADI-50203 | Heavy chain |
| | | YAMHWVRQAPGKGLEWVAMMSYDGGDK | | variable region |
| | | NYADSVKGRFTISRDNSKNTLYLQMRSLRA | | (“HC”) amino acid |
| | | EDTAIYYCARAYDSRGYYYIEHWGQGTLVT | | sequence |
| | | VSS | | |
| 104 | 407 | QVQLVQSGAEVRKPGASVKVSCKASGYTFT | ADI-42149 | Heavy chain |
| | | SYGISWVRQAPGQGLEWMGWISTYNGNTN | | variable region |
| | | YAQKLQGRVTMTTDTSTSTAYMELRSLRSD | | (“HC”) amino acid |
| | | DTAVYYCAREIDSNYVFDYWGQGTLVTVSS | | sequence |
| 105 | 408 | EVQLVESGGGLVQPGGSLRLSCAASGFTFSN | ADI-42181 | Heavy chain |
| | | YWMNWVRQAPGKGLEWVANIKQDGSEKY | | variable region |
| | | YVDSVKGRFTISRDNAKNSLYLQMNSLRAE | | (“HC”) amino acid |
| | | DTAVYYCARKLSYSSGWYYFDYWGQGTL | | sequence |
| | | VTVSS | | |
| 106 | 409 | EVQLVESGGGLVQPGGSLRLSCAASGFTFSD | ADI-45126 | Heavy chain |
| | | HYMDWVRQAPGKGLEWVGRSTNKPNSYT | | variable region |
| | | TTYAASVRGRFTISRDESKNSLYLQMNSLKS | | (“HC”) amino acid |
| | | DDTAVYYCVTTTVILFDYWGQGTLVTVSS | | sequence |
| 107 | 410 | QVQLQQWGAGLLKPSETLSLTCAVYGGSFS | ADI-45074 | Heavy chain |
| | | GYYWSWIRQPPGKGLEWIGEINHRGSTDYN | | variable region |
| | | PSLKSRVTMSVDTSKNQFSLRLSSVTAADTA | | (“HC”) amino acid |
| | | LYYCARGRLAWGLRGQKSPNFFAYWGQG | | sequence |
| | | ATVTVSS | | |
| 108 | 411 | EVQLVESGGGLVKPGGSLRLSCAASGFTFSH | ADI-49041 | Heavy chain |
| | | AWMTWVRQAPGKGLEWVGRIKSETDGGT | | variable region |
| | | ANYAAPVKGRFTISRDDSKNTVYLQMVSLK | | (“HC”) amino acid |
| | | TEDTAVYYCATAGIFGVVIMKGFDHWGQG | | sequence |
| | | TTVTVSS | | |
| 109 | 412 | EVQLLESGAEVKEPGSSVKVSCKPSGGTFSS | ADI-42227 | Heavy chain |
| | | YVISWVRQAPGQGLEWMGGIIPIFGTPNYA | | variable region |
| | | QKFQGRVTITADDSTSTAHMELSSLTSDDTA | | (“HC”) amino acid |
| | | VYYCARETYYYGSGSVPVHDWGQGTLVTV | | sequence |
| | | SS | | |
| 110 | 413 | EVQLVESGGGVVQPGRSLRLSCAASGFIFSS | ADI-50220 | Heavy chain |
| | | NSMHWVRQAPGKGLKWVAIISNDGRNKFY | | variable region |
| | | ADAVKGRFTVSRDNSKNTLYLQMNSLRPED | | (“HC”) amino acid |
| | | TAVYYCARGYDSSGYWGFGDNWGQGTLV | | sequence |
| | | TVSS | | |
| 111 | 414 | QVQLVQSGGGLVQPGGSLRLSCAASGFTFS | ADI-42141 | Heavy chain |
| | | DHYMDWVRQAPGKGLEWVGRTRNKANSY | | variable region |
| | | TTKYAASVKGRFTISRDDSKNSLYLQMNSL | | (“HC”) amino acid |
| | | KTEDTAVYYCARVEGGAWGAFDIWGQGT | | sequence |
| | | TVTVSS | | |
| 112 | 415 | QVTLKESGPVLVKPTETLTLTCTVSGFSLSN | ADI-42216 | Heavy chain |
| | | TKMGVTWIRQPPGKALEWLAHIFSNDEKS | | variable region |
| | | CNTSLKSRLTISKDTSKSQVVLTMTNMDPV | | (“HC”) amino acid |
| | | DTATYYCARLWFTEYPGAFDIWGQGTMVT | | sequence |
| | | VSS | | |
| 113 | 416 | EVQLQESGPGLVKPSETLSLTCTVSGGSISSS | ADI-50534 | Heavy chain |
| | | SYYWGWIRQPPGKGLEWIGSIYYSGSTYYN | | variable region |
| | | PSLKSRVTISVDTSKNQFSLKLSSVTAADTA | | (“HC”) amino acid |
| | | VYYCARHSSGSYYLAGYYFDYWGQGTLVT | | sequence |
| | | VSS | | |
| 114 | 417 | EVQLVESGGGLVQPGGSLRLSCAASGFTFSD | ADI-49140 | Heavy chain |
| | | HYMDWVRQAPGRGLEWVGRSRNKVNSYT | | variable region |
| | | TDYAASVKGRFTISRDDSKNSLFLRMNSLKT | | (“HC”) amino acid |
| | | EDTAVYYCARLTDSGYDDWGLGTLVTVSS | | sequence |
| 115 | 418 | EVQLVESGPGLVKPSETLSLTCTVSGGSISSY | ADI-46741 | Heavy chain |
| | | YWSWIRQPPGKGLEWIGYIYYSGSTNYNPS | | variable region |
| | | LKSRVTISVDTSKNQFSLKLSSVTAADTAVY | | (“HC”) amino acid |
| | | YCARETCSGGSCYYRVGSAFDIWGQGTTV | | sequence |
| | | TVSS | | |
| 116 | 419 | EVQLLESGGGMVQPGRSLRLSCAASGFTFD | ADI-42195 | Heavy chain |
| | | DYDMHWVRQGPGKGLEWVSGISWNSGGR | | variable region |
| | | GYADSVKGRFTISRDNAKNSLYLQMNSLRV | | (“HC”) amino acid |
| | | EDTALYYCVKDYCSGGRCYSFDYWGQGTL | | sequence |
| | | VTVSS | | |
| 117 | 420 | QVQLVESGGGVVQPGRSLRLSCAASGFTFSS | ADI-42172 | Heavy chain |
| | | YGMHWVRQAPGKGLEWVAVMSYDGSNK | | variable region |
| | | YYADSLKGRFTISRDNSKNTLYLQMNSLRA | | (“HC”) amino acid |
| | | EDTAVYFCAKAYDSSAYYYLDYWGQGTLV | | sequence |
| | | TVSS | | |
| 118 | 421 | EVQLVESGGGVIQPGRSLRLSCAASGFNFSS | ADI-42178 | Heavy chain |
| | | YGMHWVRQAPGKGLEWVAVISYDGSNKY | | variable region |
| | | YADSVKGRFTISRDNSKNTLYLQMNSLRAE | | (“HC”) amino acid |
| | | DTAVHYCAKAYDSRGYYYLDYWGQGTLV | | sequence |
| | | TVSS | | |
| 119 | 422 | EVQLVQSGGGLVQPGGSLRLSCVGSGLTLSS | ADI-49032 | Heavy chain |
| | | SAMSWVRQAPGKGLECVSGITGSGSDSSYA | | variable region |
| | | ASVKGRFTISRDNSKNTVYLQMNSLRAEDT | | (“HC”) amino acid |
| | | AVYYCAKDLTHRLGSIFGKLTFDAFDIWG | | sequence |
| | | PGTMVTVSS | | |
| 120 | 423 | EVQLVESGGGVVQPGRSLRLSCAASGFTFSS | ADI-50197 | Heavy chain |
| | | YGMHWVRQAPGKGPEWVAVISEDGNKDH | | variable region |
| | | YVDSVKGRFSIYRDNSKSTVFLRMTSLRAED | | (“HC”) amino acid |
| | | TAVYYCAKDLTPYFYDSGAFDHWGQGTLV | | sequence |
| | | TVSS | | |
| 121 | 424 | EVQLVESGGGLVQPGGSLRLSCAVSGFTFSD | ADI-48894 | Heavy chain |
| | | HYMDWVRQAPGKGLEWVGRSRNKVNSYI | | variable region |
| | | TEYAASVKGRFSISRDDSKNSLYLQMNSLKI | | (“HC”) amino acid |
| | | EDTAVYYCARVFGGPTDYWGQGTLVTVSS | | sequence |
| 122 | 425 | EVQLLESGGGLVQPGGSLRLSCAASGFIFSD | ADI-42226 | Heavy chain |
| | | HYMDWVRQAPGKGLEWVGRIRNKPNSYT | | variable region |
| | | TDYAAYVKGRFSISRDDSKNSLFLQMNSLK | | (“HC”) amino acid |
| | | TEDTAVYYCARVVNGLDVWGQGTTVTVSS | | sequence |
| 123 | 426 | EVQLVESGGGVVQPGRSLRLSCAASGFTLSS | ADI-49037 | Heavy chain |
| | | YVMHWVRQAPGKGLEWVAVISSDGTNKY | | variable region |
| | | YADSVKGRFTISRDSSKNTLYLQMNSLRPED | | (“HC”) amino acid |
| | | SAVYYCARGQPDYWGQGTLVTVSS | | sequence |
| 124 | 427 | EVQLVESGPGLVKPSGTLSLTCAVSGGSISSD | ADI-46739 | Heavy chain |
| | | NWWSWVRQAPGKGLEWIGEIYHTGSTSYN | | variable region |
| | | PSLKSRVTISLDKSKNHFSLKLNSLTAADTA | | (“HC”) amino acid |
| | | VYYCAGKKWELLGFRFDPWGQGTLVTVSS | | sequence |
| 125 | 428 | QVQLVESGAEEKKPGASVKVSCKASGYTFT | ADI-42810 | Heavy chain |
| | | SYAMHWVRQAPGQRLEWMGWINAGNGNT | | variable region |
| | | KYSQKFQGRVTITRDTSASTAYMELSSLRSE | | (“HC”) amino acid |
| | | DTAVYYCARQWLGHFDYWGQGTLVTVSS | | sequence |
| 126 | 429 | EVQLVESGGGLVQPGGSLRLSCAASGFIFSD | ADI-49137 | Heavy chain |
| | | HYMAWVRQAPGKGLEWVGHVGNKANTY | | variable region |
| | | TTEYAASVKGRFTISRDDSKKSLYLQMNRL | | (“HC”) amino acid |
| | | KSEDTAVYYCARVFSYYLDYWGQGTPVTV | | sequence |
| | | SS | | |
| 127 | 430 | QVTLKESGPTLVKPTQTLTLTCTFSGFSLSTS | ADI-42817 | Heavy chain |
| | | GVGVGWTRQPPGKALEWLALIYWDDDKR | | variable region |
| | | YSPSLKSRLTITKDTSKNQVVLTMTKMDPV | | (“HC”) amino acid |
| | | DTATYYCAHRHIAARLYRDDDVFDVWGQ | | sequence |
| | | GTMVTVSS | | |
| 128 | 431 | QVQLVQSGAEVKKPGASVKVSCKASGYTFT | ADI-50218 | Heavy chain |
| | | SYDINWVRQATGQGLEWMGWMNPNSGNT | | variable region |
| | | GYAQKFQGRVTMTRNTSISTAYMELSSLRS | | (“HC”) amino acid |
| | | EDTAVYYCARGLNTVTNSDYWGQGTLVTV | | sequence |
| | | SS | | |
| 129 | 432 | QVQLVQSGAEVKKPGASVKVSCKASGYTFT | ADI-42126 | Heavy chain |
| | | GYYMHWVRQAPGQGLEWMGWINPNSGGT | | variable region |
| | | NYAQKFQGWVTMTRDTSISTAYMELSRLRS | | (“HC”) amino acid |
| | | DDTAVYYCASGLSPDFSVLDVWGQGTTVT | | sequence |
| | | VSS | | |
| 130 | 433 | QVQLQQSGPGLVKPSQTLSLTCAISGDSVST | ADI-42186 | Heavy chain |
| | | NSAAWNWIRQSPSRGLEWLGRTYYRSKWY | | variable region |
| | | NDYALSVKSRITIKPDTSKNQFSLQLNSVTPE | | (“HC”) amino acid |
| | | DTAVYYCAREGAGYYDSSGYYPLSYDAFD | | sequence |
| | | IWGRGTMVTVSS | | |
| 131 | 434 | EVQLVESGGGLVQPGGSLRLSCAASGFTFSD | ADI-48890 | Heavy chain |
| | | HYMDWVRQAPGKGLEWVGRARNRANSYT | | variable region |
| | | TEYAASVKGRFAASRDDSKNSLYLQMNSLK | | (“HC”) amino acid |
| | | TEDTAVYYCARVRGSYWDYWGQGTLVTVS | | sequence |
| | | S | | |
| 132 | 435 | QVQLVQSGGGLVQPGGSLRLSCAASGFTFS | ADI-42206 | Heavy chain |
| | | DHYMDWVRQAPGKGLEWVGRIRNKVNSY | | variable region |
| | | TTEYAASVKGRFTISRDDSKNSLYLQMNSL | | (“HC”) amino acid |
| | | KTEDTAVYYCGRDRGWLDIWGQGTMVTV | | sequence |
| | | SS | | |
| 133 | 436 | QVQLQESGPGLVEPSGTLSLTCVVTGDSISSR | ADI-46724 | Heavy chain |
| | | SWWSWVRQPPGKGLEWIGEIYHSGTTTYSP | | variable region |
| | | SLKSRVIISLDKSENHFSLKMTSVTAADTAV | | (“HC”) amino acid |
| | | YYCARVIRDLRDYYDGSGYGPDAFDIWGQ | | sequence |
| | | GTTVTVSS | | |
| 134 | 437 | EVQLVESGPGLVKPSGTLSLTCAVSGGSISSS | ADI-50539 | Heavy chain |
| | | NWWSWVRQPPGKGLEWIGEIYHSGSTNYN | | variable region |
| | | PSLKSRVTISVDKSKNQFSLKLSSVTAADTA | | (“HC”) amino acid |
| | | VYYCARARWEDGNYYYGMDVWGQGTTV | | sequence |
| | | TVSS | | |
| 135 | 438 | EVQLVESGGGLVQPGGSLRLSCAASGFTFSS | ADI-45156 | Heavy chain |
| | | YAMSWVRQAPGKGLEWVSAISGSGGSTYY | | variable region |
| | | ADSVKGRFTISRDNSKNTLYLQMNSLRAED | | (“HC”) amino acid |
| | | TAVYYCAKDQSSGWPNYYYGMDVWGQGT | | sequence |
| | | TVTVSS | | |
| 136 | 439 | QVQLVESGSELKKPGASVKVSCKASGYTFT | ADI-50536 | Heavy chain |
| | | SYAMNWVRQAPGQGLEWMGWINTNTGNP | | variable region |
| | | TYAQGFTGRFVFSLDTSVSTAYLQISSLKAE | | (“HC”) amino acid |
| | | DTAVYYCVRGYCSSTSCYGGLYWFDPWG | | sequence |
| | | QGTLVTVSS | | |
| 137 | 440 | EVQLVESGGGVVQPGRSLRLSCADSGFTFSY | ADI-42217 | Heavy chain |
| | | SAIHWVRQAPGKGLEWVAVISYDGSNKYY | | variable region |
| | | ADSVKGRFTISRDNSKNTLYLQMNSLRPEDT | | (“HC”) amino acid |
| | | AVYYCARHSGGYSSKDKPTEYFQHWGQG | | sequence |
| | | TLVTVSS | | |
| 138 | 441 | EVQLLESGPGLVKPSGTLSLTCAVSGASISSN | ADI-48951 | Heavy chain |
| | | NWWSWVRQSPGKGLEWIGEIFHSGTTNYN | | variable region |
| | | PSLKSRVTISVDKSKNQFSLKLNSVTAADTA | | (“HC”) amino acid |
| | | VYYCARDVGVAAVITGSVRWGQGTLVTVS | | sequence |
| | | S | | |
| 139 | 442 | QVQLVQSGSELKKPGASVKVSCKASGYTFT | ADI-50537 | Heavy chain |
| | | SYAMNWVRQAPGQGLEWMGWINTNTGNP | | variable region |
| | | TYAQGFTGRFVFSLDTSVSTAYLQISSLKAE | | (“HC”) amino acid |
| | | DTAVYYCARGYCSSTSCYGGLYWFDPWG | | sequence |
| | | QGTLVTVSS | | |
| 140 | 443 | QVQLVQSGGGVVQPGRSLRLSCAASGFTFSS | ADI-46737 | Heavy chain |
| | | YAMHWVRQAPGKGLEWVAVISYDGSNKY | | variable region |
| | | YADSVKGRFTISRDNSKNTLYLQMNSLRAE | | (“HC”) amino acid |
| | | DTAVYYCARDGAGDYIWGSYRHKGLHYY | | sequence |
| | | YGMDVWGQGTTVTVSS | | |
| 141 | 444 | EVQLVESGPGLVMPSGTLSLTCTVSGISISSS | ADI-50538 | Heavy chain |
| | | NWWSWVRQSPGKGLEWIGEVYHSGSTKY | | variable region |
| | | NPSLKSRVTISVDKSRNQFSLKLNSVTAADT | | (“HC”) amino acid |
| | | AVYYCAKDPRTFYGVVMLLDDPWGQGTL | | sequence |
| | | VTVSS | | |
| 142 | 445 | EVQLVESGGGVVQPGRSLRLSCAVSGFTFST | ADI-48950 | Heavy chain |
| | | SPLHWVRQAPGKGLEWVAVSSFVATDKYY | | variable region |
| | | ADSVKGRFTVSRDNSKNTLYLQMNSLRPED | | (“HC”) amino acid |
| | | TAVYYCARGFGELPGFDIWGQGTMVTVSS | | sequence |
| 143 | 446 | EVQLLESGGGLVKPGGSLRLSCAASGFTFSS | ADI-42114 | Heavy chain |
| | | YSMNWVRQAPGKGLEWVSSISSSSSYIYYA | | variable region |
| | | DSVKGRFTISRDNAKNSLYLQMNSLRAEDT | | (“HC”) amino acid |
| | | AVYYCARDSWGPFDYWGQGTLVTVSS | | sequence |
| 144 | 447 | EVQLVESGGAVVQPGRSLRLSCAASGFTFSS | ADI-49194 | Heavy chain |
| | | YGMHWVRQAPGKGLESVAVIWYDGSNKN | | variable region |
| | | YADSVKGRFTISRDNSKNTLFLQMNSLRAED | | (“HC”) amino acid |
| | | SAMYYCAKTYDSRAYYYLDYWGQGTLVT | | sequence |
| | | VSS | | |
| 145 | 448 | EVQLLESGGGLVQPGGSLRLSCAASGFTFSS | ADI-42124 | Heavy chain |
| | | YAMSWVRQAPGKGLELVSAISSSGGSTYYA | | variable region |
| | | DSVKGRFTISRDNSKNTLYLQMNSLRAEDT | | (“HC”) amino acid |
| | | ALYYCAKDLFYDFWTGITIDYWGQGTLVT | | sequence |
| | | VSS | | |
| 146 | 449 | EVQLLESGGGLVQPGGSLRLSCAASGFIFSN | ADI-45123 | Heavy chain |
| | | YWMSWVRQAPGKGLEWVANIKPDGSEKY | | variable region |
| | | YVESVRGRFTISRDNAKNSLYLQMNSLRAE | | (“HC”) amino acid |
| | | DTAVFYCARDGGTVSDGLDVWGQGTTVTV | | sequence |
| | | SS | | |
| 147 | 450 | QVQLQESGPGLVKPSGTLSLTCAVSGGSISSS | ADI-50533 | Heavy chain |
| | | NWWSWVRQPPGKGLEWIGEIYHSGSTNYN | | variable region |
| | | PSLKSRVTISVDKSKNQFSLKLSSVTAADTA | | (“HC”) amino acid |
| | | VYYCARVVWYSSSSHLFDYWGQGTLVTVS | | sequence |
| | | S | | |
| 148 | 451 | EVQLVESGGGVVQTGRSLRLSCAASGFTFSI | ADI-49205 | Heavy chain |
| | | SGMHWVRQAPGKGLEWVALIWYDGTKKY | | variable region |
| | | YADSVKGRFTISRDDFKNTVYLQMNSLRAD | | (“HC”) amino acid |
| | | DTAVYYCARIKSDAFDLWGQGTTVTVSS | | sequence |
| 149 | 452 | EVQLLESGGGVVQPGKSLRLSCAASGFSFGD | ADI-45151 | Heavy chain |
| | | YGMHWVRQTPDKGLEWVAVILFDGSKKF | | variable region |
| | | YADSVRGRFTISRDNSKNNLYLQMSSLRPED | | (“HC”) amino acid |
| | | TAVYYCAKFPLRDGGSGEGFDYWGQGTLV | | sequence |
| | | TVSS | | |
| 150 | 453 | EVQLVESGGGVVQPGRSLRLSCAASGFTFSS | ADI-46728 | Heavy chain |
| | | YAMHWVRQAPGKGLEWVAVISYDGSNKY | | variable region |
| | | YADSVKGRFTISRDNSKNTLYLQMNSLRAE | | (“HC”) amino acid |
| | | DTAVYYCARNTYYDRRRTFDYWGQGTLVT | | sequence |
| | | VSS | | |
| 151 | 454 | QVQLVQSGGGLVQPGGSLRLSCAASGFTFS | ADI-49030 | Heavy chain |
| | | DHYMDWVRQAPGKGLEWVGRTRNKANSY | | variable region |
| | | TTEYAASVKGRFTISRDDSKNSLYLQMNSL | | (“HC”) amino acid |
| | | KTEDTAVYYCAGVGITGTTGIDYWGQGTL | | sequence |
| | | VTVSS | | |
| 152 | 455 | EVQLLESGGDLVQPGRSLRLSCAASGFNLID | ADI-50200 | Heavy chain |
| | | YAMHWVRQVPGKGLEWVSGISWNSRSIGY | | variable region |
| | | ADSVKGRFTISRDNAKNSLYLQMDSLKHED | | (“HC”) amino acid |
| | | TALFYCAKGAAAGPFPYFYYAMDVWGQG | | sequence |
| | | TTVTVSS | | |
| 1 | 456 | QSVLTQPPSVSAAPGQKVTISCSGSSSNIGNN | ADI-49039 | Light chain |
| | | YVSWYQQLPGAAPKWYDNKKRPSGIPDRF | | variable region |
| | | SGSASGTSATMGITGLQTGDEADYYCGTWD | | (“LC”) amino acid |
| | | SSLSAWVFGGGTKVTVL | | sequence |
| 2 | 457 | DIRVTQSPATLSVSPGERATLSCRASQSVSSN | ADI-49147 | Light chain |
| | | LAWYQQKPGQAPRLLIYDASNRATGWVRFS | | variable region |
| | | GSGSGTDFTLTISSLQSEDFAVYYCQQYDN | | (“LC”) amino acid |
| | | WPLTFGGGTKVEK | | sequence |
| 3 | 458 | QSVVTQPPSVSAAPGQKVTISCSGSSSNIGNN | ADI-42229 | Light chain |
| | | YVSWYQQFPRTAPKLLIYDNKKRPSGIPDRF | | variable region |
| | | SGSASGTSATLGITGLQTGDEADYYCGTWD | | (“LC”) amino acid |
| | | SSLSAWVFGGGTKVTVL | | sequence |
| 4 | 459 | QPVLTQPPSASGTPGQRVTIFCSGSRSNIGTY | ADI-45090 | Light chain |
| | | TINWYQKLPGTAPKLLIYSNNRGPSGVPDRF | | variable region |
| | | SGSQSGTSASLAISGLQPEDEADYYCAAWD | | (“LC”) amino acid |
| | | DSLNGWVFGGGTKVTVL | | sequence |
| 5 | 460 | QSVVTQPPSVSAAPGQKVTISCSGSSSNIGNN | ADI-45097 | Light chain |
| | | YVAWYQQLPGRAPKLLIHDNKKRPSGIPDR | | variable region |
| | | FSGSASGTSATLGITGLQTGDEADYYCETW | | (“LC”) amino acid |
| | | DSSLNAVVFGGGTKLTVL | | sequence |
| 6 | 461 | DIQMTQSPSSLSASVGDRVTITCRASQTISVD | ADI-49133 | Light chain |
| | | LNWYQHKPGKAPKLLIFAASTLQSGVPSRFS | | variable region |
| | | GSGSGTDFTLTIRSLQPEDFATYYCQQSYSIP | | (“LC”) amino acid |
| | | RITFGQGTRLEK | | sequence |
| 7 | 462 | EIVMTQSPSALSASVRDRVTITCRASQSIGSD | ADI-49033 | Light chain |
| | | LNWYQQRPGKAPMLLIYAATGLQSGVPSRF | | variable region |
| | | SGSGSGTDFTLTISNLQPEDFATYYCQQSYSP | | (“LC”) amino acid |
| | | PMYTFGQGTKVDIK | | sequence |
| 8 | 463 | QPVLTQPPSASGTPGQRVTISCSGSSSNIGTN | ADI-49044 | Light chain |
| | | TVSWYQQLPGTAPQLLVFSRTQRPSGVPDR | | variable region |
| | | FSGSKSGTSASLAISGLQSDDEADYYCAAWD | | (“LC”) amino acid |
| | | DSRNGWVFGGGTKLTVL | | sequence |
| 9 | 464 | QPVLTQPPSVSAAPGQKVTISCSGSNSNIGNY | ADI-45083 | Light chain |
| | | YVSWYQQFPGTAPKLLIYDNNKRPSGIPDRF | | variable region |
| | | SGSKSGTSATLAITGLQTGDEAHYYCGTWD | | (“LC”) amino acid |
| | | TSSLSAGRVFGGGTKLTVL | | sequence |
| 10 | 465 | QSALTQPPSVSAAPGQKVTISCSGSSSNIGNS | ADI-42225 | Light chain |
| | | YVSWYQQVPGTAPKLLIYDNNKRPSGIPDRF | | variable region |
| | | SGSKSGTSATLGITGLQAGDEADYYCGTWD | | (“LC”) amino acid |
| | | TSLSAGRVFGRGTKLTVL | | sequence |
| 11 | 466 | QSVLTQPPSVSAAPGQKVTISCSGSSSNIGYS | ADI-49139 | Light chain |
| | | HVSWYQQLPGTAPKVLIYDNDKRPSGIPDRF | | variable region |
| | | SGSKSGTSATLGITGLQTGDEADYYCGTWD | | (“LC”) amino acid |
| | | TSLGVVFGGGTKLTVL | | sequence |
| 12 | 467 | QSVLTQPRSVSGSPGQSVTISCTGTSSDVGA | ADI-48969 | Light chain |
| | | YNFVSWYQQYPGKAPKLMIYDVNKRPSGV | | variable region |
| | | PDRFSGSKSGNTASLTISGLQAEDEADYHCC | | (“LC”) amino acid |
| | | SYAGTYTSNYVFGSGTKVTVL | | sequence |
| 13 | 468 | QSVVTQPPSVSAAPGQKVTISCSGSSSNIGNN | ADI-48900 | Light chain |
| | | YVSWYQQLSETAPKLLIYDNNKRPSGIPNRF | | variable region |
| | | SGSKSGTSATLGITGLQTGDEADYYCGTWD | | (“LC”) amino acid |
| | | NSLGAVVFGGGTKVTVL | | sequence |
| 14 | N/A | N/A | ADI-42232 | Light chain |
| | | | | variable region |
| | | | | (“LC”) amino acid |
| | | | | sequence |
| 15 | 469 | QSVLTQPPSVSAAPGQKVTISCSGSSSNIGSN | ADI-42786 | Light chain |
| | | YVSWYQQFPGTAPKLLIYDNSKRPSGIPDRF | | variable region |
| | | SGSMSGTSATLGITGLQTGDEADYYCGTWD | | (“LC”) amino acid |
| | | SSLSAVVFGGGTKVTVL | | sequence |
| 16 | 470 | QSVLTQPPSVSAAPGQKVTISCSGSSSNIGNN | ADI-42210 | Light chain |
| | | YVSWYQQLPGTAPKWYDNNKRPSGIPDRF | | variable region |
| | | SGSKSGTSATLGITGLQTGDEADYYCGTWD | | (“LC”) amino acid |
| | | TSLSAGRVFGGGTKLTVL | | sequence |
| 17 | 471 | EIVLTQSPATLSVSPGERATLSCRASRSVSSN | ADI-50201 | Light chain |
| | | LAWYQQKPGQAPRLLIYGASTRATGIPARFT | | variable region |
| | | GSGSGTEFTLTISSLQSEDFAVYYCQQYNNW | | (“LC”) amino acid |
| | | PPRTFGQGTKVDIK | | sequence |
| 18 | 472 | DIQLTQSPSSVSASVGDRVTITCRASQGISSW | ADI-48895 | Light chain |
| | | LAWYQQKPGKAPKLLIFIAASSLQSGVPSRFS | | variable region |
| | | GSGSGTDFTLTISSLQPEDFATYYCQQAKSF | | (“LC”) amino acid |
| | | PPTFGQGTRLEIK | | sequence |
| 19 | 473 | QPVLTQPASVSGSPGQSITISCTGTSSDVGGY | ADI-42228 | Light chain |
| | | NYVSWYQQHPGKAPKLLIYDVSNRPSGVSN | | variable region |
| | | RFSGSKSANSASLTISGLQAEDEADYYCNSY | | (“LC”) amino acid |
| | | TSSSTLVFGGGTKLTVL | | sequence |
| 20 | 474 | EIVMTQSPATLSVSPGERATLSCRASQSVSSN | ADI-45113 | Light chain |
| | | LAWYQQKPGQAPRLLIYGASTRATGIPARFS | | variable region |
| | | GSGSGTEFTLTISSLQSEDFALYYCQQYDDW | | (“LC”) amino acid |
| | | PLFGQGTRLEIK | | sequence |
| 21 | 475 | QSVLTQPPSVSAAPGQKVTISCSGSSSNIGNN | ADI-42198 | Light chain |
| | | YVSWYQQLPGTAPKLLIYDNNKRPSGIPDRF | | variable region |
| | | SGSKSGTSATLGITGLQTGDEADYICGTWDT | | (“LC”) amino acid |
| | | SLSAGGVFGGGTKLTVL | | sequence |
| 22 | 476 | QSVLTQPASVSGSPGQSITISCTGTSSDIGAY | ADI-42190 | Light chain |
| | | NYVSWYQQHPGKAPKLMIYDVTNRPSGVS | | variable region |
| | | NRFSGSKSGSSASLTISGLQTEDEADYYCSSY | | (“LC”) amino acid |
| | | TRRSTLVFGGGTKLTVL | | sequence |
| 23 | 477 | QSVLTQPPSVSAAPGQKVTISCSGSSSNIGNN | ADI-49154 | Light chain |
| | | YVSWYQQLPGTAPKLLIYDNNKRPSGIPDRF | | variable region |
| | | SGSKSGTSATLGITGLQTGDEANYYCGTWD | | (“LC”) amino acid |
| | | TSLSTVFGGGTKLTVL | | sequence |
| 24 | 478 | QSALTQPASVSGSPGQSITISCTGTGSDVGG | ADI-49183 | Light chain |
| | | YNFVSWYQQHPGKAPKLMLYDVNNRPSGV | | variable region |
| | | SNRFSGSKSGNTASLTISGLQAEDEADYYCSS | | (“LC”) amino acid |
| | | YPGTSALVIFGGGTRLTVL | | sequence |
| 25 | 479 | DIQMTQSPSSLSASVGDRVTITCRASQSISSY | ADI-42201 | Light chain |
| | | LNWYQQKPGEAPNLLIFAASILQSGVPSRFS | | variable region |
| | | GSGSGTDFTLTISSLQPEDFATYYCQQSYSTP | | (“LC”) amino acid |
| | | YTFGQGTKVEIK | | sequence |
| 26 | 480 | QSVLTQPPSVSGAPGQRVTISCTGSSSNIGAG | ADI-42144 | Light chain |
| | | YDVHWYQQLPGTAPKLLIYGNSNRPSGVPD | | variable region |
| | | RFSGSKSGTSASLAITGLQAEDEADYYCQSY | | (“LC”) amino acid |
| | | DSSLSGHVVFGGGTKLTVL | | sequence |
| 27 | 481 | EIVLTQSPATLSSSPGERATLSCRASQSVNSY | ADI-50219 | Light chain |
| | | LVWYQQKPGQAPRLLIYDASNRATGIPARFT | | variable region |
| | | GSGSGTDFTLTISSLEPEDFAVYYCQQRTNW | | (“LC”) amino acid |
| | | PFTFGQGTKVDIK | | sequence |
| 28 | 482 | EIVLTQSPATLSLSPGERATLSCRASQSVNRY | ADI-48897 | Light chain |
| | | LAWYQQKPGQAPRLLIYDASNRATGIPARFS | | variable region |
| | | GSGSGTDFTLTISSLEPEDFAVYYCHQRTNW | | (“LC”) amino acid |
| | | PWTFGQGTKVEIK | | sequence |
| 29 | 483 | EIVMTQSPATLSLSPGERATLSCRASQSVSNY | ADI-42194 | Light chain |
| | | LAWYQQKPGQAPRLLISDASSRATGIPARFR | | variable region |
| | | GSGSGTDFTLTISSLEPEDFAVYYCLQRTNW | | (“LC”) amino acid |
| | | PFTFGPGTKVEIK | | sequence |
| 30 | 484 | QSVLTQPASVSGSPGQSITISCTGTSSDIGGY | ADI-49189 | Light chain |
| | | NYVSWYQQHPGKVPKLVIYDVSNRPSGVSN | | variable region |
| | | RFSGSKSGNTASLTISGLQAEDEADYYCSSY | | (“LC”) amino acid |
| | | TSGTTLGVFGTGTKLTVL | | sequence |
| 31 | 485 | QSVVTQPPSVSAAPGQKVTISCSGRSSNIGNS | ADI-49188 | Light chain |
| | | DVSWYQQFPGRAPKLLIYDNDERPSGIPDRF | | variable region |
| | | SGSKSGTSATLDITGLQTGDEADYYCGTWD | | (“LC”) amino acid |
| | | SSLGGVIFGGGTKVTVL | | sequence |
| 32 | 486 | QSVLTQPPSVSAAPGQKVTISCSGSSSNIGNN | ADI-42188 | Light chain |
| | | YVSWYQQLPGTAPKLLIYDNNKRPSGIPDRF | | variable region |
| | | SGSKSGTSATLGITGLQTGDEADYYCETWD | | (“LC”) amino acid |
| | | SSLGVVVFGGGTKLTVL | | sequence |
| 33 | 487 | DIQVTQSPSSLSASVGDRVTITCQASQDISNY | ADI-50026 | Light chain |
| | | LNWYQHKPGRAPKLLIYDASNLERGVPSRF | | variable region |
| | | SGSGSGTDFTFTISSLQPEDIATYYCQQYDNL | | (“LC”) amino acid |
| | | SRLTFGGGTKLEK | | sequence |
| 34 | 488 | QSVLTQPPSVSAAPGQKVTISCSGSSSNIGNN | ADI-42809 | Light chain |
| | | YVSWYQQLPGTAPKLLIYDNNKRPSGIPDRF | | variable region |
| | | SGSKSGTSATLGITGLQTGDEADYYCGTWD | | (“LC”) amino acid |
| | | TSLSAGRVFGGGTKLTVL | | sequence |
| 35 | 489 | QSVLTQPPSMSAAPGQKVTISCSGSSSNIGNN | ADI-46596 | Light chain |
| | | YVSWYRQLPGTAPKLLIYDNDKRPSGIPDRF | | variable region |
| | | SGSKSGTTATLGITGLQTGDEAVYYCGTWD | | (“LC”) amino acid |
| | | FRLSALFGGGTKLTVL | | sequence |
| 36 | 490 | QSVLIQPASVSGSPGQSITISCTGTSSDVGGD | ADI-50205 | Light chain |
| | | KYVSWYQQHPGKAPKLVIYEVSNRPSGVSN | | variable region |
| | | RFSGSKSGNTASLTISGLQAEDEADYYCSSY | | (“LC”) amino acid |
| | | TSSGTPVVCGGGTKVTVL | | sequence |
| 37 | 491 | QSVLTQPPSVSAAPGQKVTISCSGSSSNIGNY | ADI-42830 | Light chain |
| | | YVSWYQQVPGTAPKLLIYDNNKRPSGIPDRF | | variable region |
| | | SGSKSGTSATLGITGLHTGDEAEYYCGTWD | | (“LC”) amino acid |
| | | SSPSAGRVFGGGTKLTVL | | sequence |
| 38 | 492 | DIVLTQSPDSLAVSLGERATINCKSSQSVLFG | ADI-49186 | Light chain |
| | | SNQKSCLAWYQQKPGQSPKLLIFIWASTRES | | variable region |
| | | GVPDRFSGSGSGTDFTLTISSLQAEDVAVYY | | (“LC”) amino acid |
| | | CQQYYSTPRTFGQGTKVEIK | | sequence |
| 39 | 493 | QSVLTQPPSVSAAPGQKVTISCSGSSSNIGSN | ADI-46591 | Light chain |
| | | FVSWYQQLPGTAPKLLIYDNNKRPSGIPDRF | | variable region |
| | | SGSKSGTSATLAITGLQTGDEADYYCGTWD | | (“LC”) amino acid |
| | | TRLSALFGGGTKVTVL | | sequence |
| 40 | 494 | QSVLTQPPSVSAAPGQKVTISCSGSSSNFGN | ADI-48955 | Light chain |
| | | DYVSWYQQLPGTAPKWYDNDKRPSGIPDR | | variable region |
| | | FSGSKSGTSATLGITGLQTGDEADYYCGTW | | (“LC”) amino acid |
| | | DTSLSAAWVFGGGTKVTVL | | sequence |
| 41 | 495 | QSVVTQPPSVSAAPGQKVTISCSGSSSNIGNN | ADI-42818 | Light chain |
| | | YVSWYQQLPGTAPKLLIYDNNKRPSGIPDRF | | variable region |
| | | SGSTSGTSATLGITGLQTGDEAVYYCGTWD | | (“LC”) amino acid |
| | | TSPSAGGVFGGGTKVTVL | | sequence |
| 42 | 496 | QPVLTQPASVSGSPGQSITISCTGTSSDVGGY | ADI-50531 | Light chain |
| | | NYVSWYQQHPGKAPKLMIYDVSNRPSGVSN | | variable region |
| | | RFSGSKSGNTASLTISGLQAEDEADYYCSSY | | (“LC”) amino acid |
| | | TSSSTLAVFGGGTKLTVL | | sequence |
| 43 | 497 | QPVLTQPPSVSAAPGQKVTISCSGSSSNIGND | ADI-46586 | Light chain |
| | | YVSWYQQLPGTAPKLLIYDNNKRPSGIPDRF | | variable region |
| | | SGSKSGTSATLGITGLQTGDEGDYYCGTWD | | (“LC”) amino acid |
| | | SSLSAAWVFGGGTKVTVL | | sequence |
| 44 | 498 | QPVLTQSASVSGSPGQSITISCTGTSSDVGGY | ADI-49138 | Light chain |
| | | KYVSWYQQHPGKAPKLMIYEVSNRPSGVSI | | variable region |
| | | RFSGSKSGNTASLTISGLQAADEADYYCSSY | | (“LC”) amino acid |
| | | RSSGTPYVFGTGTKVTVL | | sequence |
| 45 | 499 | EIVLTQSPSSLSASVGDRVTITCQASQDISNF | ADI-45075 | Light chain |
| | | LNWYQQKPGKAPKLLIYDASSLETGVPSRFS | | variable region |
| | | GSGSGTDFTFTISSLQPEDIATYYCQQYDNLP | | (“LC”) amino acid |
| | | LTFGGGTKLEIK | | sequence |
| 46 | 500 | DIRLTQSPSTLSASVGDRVTVTCRASQNINT | ADI-42831 | Light chain |
| | | YLAWYQQIPGKAPRLLIYRASTLESGVPSRF | | variable region |
| | | SGSGSGTEFTLTINSLQPDDYATYYCQHYET | | (“LC”) amino acid |
| | | YSVRFGQGTKVEIK | | sequence |
| 47 | 501 | DIQVTQSPSSLSASVGDRVTITCRASQGISNY | ADI-42230 | Light chain |
| | | LAWYQQKPGKVPKLLIFAASTLRSGVPSRFR | | variable region |
| | | GSGSGTDFTLTISSLQPEDVATYYCQKYNSA | | (“LC”) amino acid |
| | | PLTFGGGTKVEIK | | sequence |
| 48 | 502 | DIVMTQTPATLSLSPGERATLSCRASQSVSSY | ADI-42847 | Light chain |
| | | LAWYQQKPGQAPRLLIYGASNRATGIPARFS | | variable region |
| | | GSGSGTDFTLTISSLEPEDFAVYYCLQRTNW | | (“LC”) amino acid |
| | | PFTFGPGTKVEIK | | sequence |
| 49 | 503 | DIVLTQSPSTLSASVGDRVTVTCRASQNINT | ADI-42821 | Light chain |
| | | YLAWYQQIPGKAPRLLIYRASSLESGVPSRF | | variable region |
| | | SGSGSGTEFTLTISSLQPDDFATYYCQHYNSF | | (“LC”) amino acid |
| | | SVKFGQGTKVEIK | | sequence |
| 50 | 504 | SYELTQPPSVSVAPGQTARITCGGHNVGSKS | ADI-42849 | Light chain |
| | | VHWYQQKPGQAPVLVVYDDSDRPSGIPERF | | variable region |
| | | SGSNSGNTATLTISRVEAGDEADYYCQVWD | | (“LC”) amino acid |
| | | SSSDHPWVFGGGTKVTVL | | sequence |
| 51 | 505 | QSVVTQPPSVSAAPGQKVTISCSGSSSNIGNN | ADI-42151 | Light chain |
| | | YVSWYQQLPGTAPKLLIYDNNKRPSGIPDRF | | variable region |
| | | SGSKSGTSATLGITGLQTGDEADYYCGTWD | | (“LC”) amino acid |
| | | TSLSAGRVFGGGTKLTVL | | sequence |
| 52 | 506 | QPVLTQPPSVSVAPGQTARITCGGNNIGSKS | ADI-46001 | Light chain |
| | | VHWYQQKPGQAPMLVIYSNSDRPSGIPERFS | | variable region |
| | | GSNSGITATLTISRVEAGDEADYHCQVWDTS | | (“LC”) amino acid |
| | | IDHHWVFGGGTKLTVL | | sequence |
| 53 | 507 | QSVLIQPPSASGSPGQSVTISCTGTSSDVGGY | ADI-45154 | Light chain |
| | | NYVSWYQQHPGKAPKLMIYEVSKRPSGVPD | | variable region |
| | | RFSGSKSGNTASLTVSGLQAEDGADYYCSSY | | (“LC”) amino acid |
| | | AGSNNWVVFGGGTKLTVL | | sequence |
| 54 | 508 | QPVLTQPASVSGSPGQSITISCTGTSTDVGGY | ADI-49161 | Light chain |
| | | NYVSWYQQYPGKAPKLIIYDVTNRPSGVSH | | variable region |
| | | RFSGSKSGNTASLTISGLQAEDEADYYCSSY | | (“LC”) amino acid |
| | | TTTSLVIFGGGTKLTVL | | sequence |
| 55 | 509 | DIVMTQSPLSLPVTPGEPASISCRSSQSLLHS | ADI-42154 | Light chain |
| | | NGYNYLDWYLQKPGQSPQLLIYLGSNRASG | | variable region |
| | | VPDRFSGSGSGTDFTLKISRVEAEDVGVYYC | | (“LC”) amino acid |
| | | MQALQTPPRTFGQGTRLEIK | | sequence |
| 56 | 510 | DIQVTQSPSSLSASVGGRVTITCRASQGIRND | ADI-48916 | Light chain |
| | | LGWYQRKPGKAPKRLIYAASSLQSGVPSRFS | | variable region |
| | | GSGSGTEFTLTISSLQPEDFATYYCLQHNSYP | | (“LC”) amino acid |
| | | LTFGGGTKVIAK | | sequence |
| 57 | 511 | DIQLTQSPSTLSASVGDRVTITCRASQSISTW | ADI-45085 | Light chain |
| | | LAWYQQKPGKAPKLLIYRASSLESGVPSRFS | | variable region |
| | | ASGSGTEFTLSISSLQPDDFATYYCKQYNRN | | (“LC”) amino acid |
| | | PYTFGQGTKVEIK | | sequence |
| 58 | 512 | DIQMTQSPSSLSASVGDRVTITCRASQGISSY | ADI-42211 | Light chain |
| | | LAWFQQKPGKVPKLLIYAASTLQSGVPSRFS | | variable region |
| | | GSGSGTDFTLTISSLQPEDVATYYCQKYNSA | | (“LC”) amino acid |
| | | PQTFGQGTKVDIK | | sequence |
| 59 | 513 | EIVMTQSPATLSVSPGERATLSCRASQSVSFN | ADI-48908 | Light chain |
| | | LAWYQQKPGQAPRLLISRASTRAAGVPARF | | variable region |
| | | SGSGSGTEFTLTISSLQSEDFAVYYCQQYNN | | (“LC”) amino acid |
| | | WPPLTFGGGTKLEIK | | sequence |
| 60 | 514 | DIQMTQSPDSLTVSLGERATINCKSSQSVLYS | ADI-48913 | Light chain |
| | | SNNKNSLAWYQQKPGQPPKLLIYWASTRES | | variable region |
| | | GVPDRFSGSGSGTDFTLTISSLQAADVAVYY | | (“LC”) amino acid |
| | | CQQYYRTPWTFGQGTKVEK | | sequence |
| 61 | 515 | QSVLTQPPSVSAAPGQKVTISCSGSSSNIGNN | ADI-45140 | Light chain |
| | | YVSWYQQLPGTAPKVLIYDNNKRPSGIPDRF | | variable region |
| | | SGSKSGTSATLGITGLQTGDEADYYCGTWD | | (“LC”) amino acid |
| | | SALGAAVFGGGTKLTVL | | sequence |
| 62 | 516 | DIQLTQSPSTLSASVGDRVTITCRASQSVSSW | ADI-50211 | Light chain |
| | | LAWYQQKPGKAPRLLIYRASSLESGVPSRFS | | variable region |
| | | GSGSGTEFTLTISSLQPDDFAAYYCQQYNRD | | (“LC”) amino acid |
| | | PYTFGQGTKVEIK | | sequence |
| 63 | 517 | SYELTQLPSVSVSPGQTARVTCSGDAL-- | ADI-42199 | Light chain |
| | | QYVYWYQQKPGQAPVVVIYKDTERPSGIPE | | variable region |
| | | RFSGSSSGTTVTLTITGVQAEDEADYYCQSA | | (“LC”) amino acid |
| | | DRSGSVIFGGGTKVTVL | | sequence |
| 64 | 518 | DIVMTQSPATLSLSPGERATLSCRASQSVSSY | ADI-42231 | Light chain |
| | | LAWYQQKPGQAPRLLIYDASNRATGIPARFS | | variable region |
| | | GSGSGTDFTLTISSLEPEDFAVYYCQQRSNW | | (“LC”) amino acid |
| | | PSFGQGTKLEIK | | sequence |
| 65 | 519 | DIRLTQSPSSLSASVGDRVTITCRASQSISSYL | ADI-45164 | Light chain |
| | | NWYQQKPGKAPKLLIYAASSLQSGVPSRFSG | | variable region |
| | | SGSGTDFTLTISSLQPEDFATYYCQQSYSTPL | | (“LC”) amino acid |
| | | TFGGGTKVEIK | | sequence |
| 66 | 520 | ETTLTQSPGTLSLSPGERATLSCRASRSVSGN | ADI-42233 | Light chain |
| | | YLAWYQQKPGQAPRLLIYAASSRATGIPDRF | | variable region |
| | | SGGGSGTHFTLTISRLEPEDFAVYYCQQYGS | | (“LC”) amino acid |
| | | SPRAFGQGTKVEIK | | sequence |
| 67 | 521 | EIVMTQSPSSLSASVGDRVTITCRASQSIRSY | ADI-42191 | Light chain |
| | | LNWYQQKPGKAPKLLIYAASSLQSGVPLRFS | | variable region |
| | | GSGSGTDFTLTISSLQPEDFATYYCQQSSITP | | (“LC”) amino acid |
| | | PTFGQGTKLEIK | | sequence |
| 68 | 522 | DIQMTQSPSTLSASVGDRVTITCRASQSISSW | ADI-48899 | Light chain |
| | | LAWYQQKPGKAPKLLIYQASSLESGVPSRFS | | variable region |
| | | GSESGTEFTLTISSLQPDDFATYYCQQYNSFP | | (“LC”) amino acid |
| | | FTFGPGTKVEIK | | sequence |
| 69 | 523 | DIVLTQSPSSLSASVGDRVTITCRASQSINNY | ADI-49145 | Light chain |
| | | LNWYQQKPGKAPNLLIFGASTLQSGVPSRFT | | variable region |
| | | GSGSGTVFTLTISSLQRDDFVIYYCQQTYSAS | | (“LC”) amino acid |
| | | GSFGQGTKVEIK | | sequence |
| 70 | 524 | DIQLTQSPSSLSASVGDRVTITCRASQSISSYL | ADI-46729 | Light chain |
| | | NWYQQKPGKAPKLLIYAASSLQSGVPSRFSG | | variable region |
| | | SGSGTDFTLTISSLQPEDFATYYCQQSYSTP | | (“LC”) amino acid |
| | | WTFGQGTKVEIK | | sequence |
| 71 | 525 | QSALIQPPSVSAAPGQKVTISCSGSSSNIGNN | ADI-46722 | Light chain |
| | | YVSWYQQLPGTAPKLLIYDNNKRPSGIPDRF | | variable region |
| | | SGSKSGTSATLGITGLQTGDEADYYCGTWD | | (“LC”) amino acid |
| | | NSLGVVFGGGTQLTVL | | sequence |
| 72 | 526 | EIVLTQSPGTLSLSPGERATLSCRASQSVSSS | ADI-45148 | Light chain |
| | | YLAWYQQKPGQAPRLLIYGASSRATGIPDRF | | variable region |
| | | SGSGSGTDFTLSINRLEPEDFAVYYCQQYGS | | (“LC”) amino acid |
| | | SPGFGQGTKVEIK | | sequence |
| 73 | 527 | DIVLTQSPSTLSASVGDRVTITCRASQSISDW | ADI-49168 | Light chain |
| | | LAWYQQKPGKAPGLLIYRASGLESGVPSRF | | variable region |
| | | SGSGSGTEFTLTISSLQPDDFATYYCHQYKD | | (“LC”) amino acid |
| | | FPWTFGQGTKVDIK | | sequence |
| 74 | 528 | DIQMTQSPSTLSASVGDRVTITCRASQSISTW | ADI-49040 | Light chain |
| | | LAWYQLKPGKAPKLLIYKASNLQSGVPSRF | | variable region |
| | | SGSGSGTEFTLTISSLQPDDFATYYCQQYNS | | (“LC”) amino acid |
| | | YSPWGQGTKLEIK | | sequence |
| 75 | 529 | EIVLTQSPGTLSLSPGERATLSCRASQSVSSR | ADI-42187 | Light chain |
| | | YLAWYRQKPGQAPRLLIYGASSRATGIPDRF | | variable region |
| | | SGSGSGTDFTLTISRLEPEDFAVYYCQQYGS | | (“LC”) amino acid |
| | | SPFFGGGTKLEIK | | sequence |
| 76 | 530 | QSVLTQPASVSGSPGQSITISCTGTSSDVGGD | ADI-49561 | Light chain |
| | | KYVSWYQQHPGKAPKPMIYEVSNRPSGVSN | | variable region |
| | | RFSGSKSGNTASLTISGLQAEDEADYYCSSY | | (“LC”) amino acid |
| | | TSSSTPVVFGGGTKLTVL | | sequence |
| 77 | 531 | QPVLTQPRSVSGSPGQSVTISCTGTSSDVGG | ADI-42219 | Light chain |
| | | YNYVSWYQQHPGKAPKLMISDVSKRPSGVP | | variable region |
| | | DRFSGSKSGNTASLTISGLQADDEADYYCCS | | (“LC”) amino acid |
| | | YATNYGVVFGGGTKVTVL | | sequence |
| 78 | 532 | NFMLTQPHSVSESPGKTVTISCTRSSGSIASN | ADI-50535 | Light chain |
| | | YVQWYQQRPGSSPTTVIYEDNQRPSGVPDR | | variable region |
| | | FSGSIDSSSNSASLTISGLKTEDEADYYCQSY | | (“LC”) amino acid |
| | | DSSNVVFGGGTKVTVL | | sequence |
| 79 | 533 | QSVLTQPPSVSAAPGQKVTISCSGSSSNIGNN | ADI-45128 | Light chain |
| | | DVSWYQQLPGRAPKWYDNNKRPSGIPDRF | | variable region |
| | | SGSKSGTSATLGITGLQTGDEADYYCGAWD | | (“LC”) amino acid |
| | | SSLSAHVVFGGGTKVTVL | | sequence |
| 80 | 534 | DIVMTQTPSTLSASVGDRVTVTCRASQSISD | ADI-45136 | Light chain |
| | | WLAWYQQKAGKAPKLLIYRASSLESGVPPR | | variable region |
| | | FSGSGSGTEFTLTISSLRPDDFATYYCQQYNR | | (“LC”) amino acid |
| | | YPYTFGQGTKVDIK | | sequence |
| 81 | 535 | DIQVTQSPSSLSASVGDRVTITCRASQGIRND | ADI-42189 | Light chain |
| | | LAWYQQRPGKAPKRLIYAASSLQSGVPSRFS | | variable region |
| | | GSGSGTEFTLTISSLQPEDFATYYCLQHHSYP | | (“LC”) amino acid |
| | | WTFGQGTKVEIK | | sequence |
| 82 | 536 | DIRMTQSPSTLSASIGDRVTITCRASQSISDW | ADI-45078 | Light chain |
| | | LAWYLQKPGKAPSLLIYRASSLETGVPSRFS | | variable region |
| | | GRGSGTEFTLTISSLQPDDFGTYYCQQYNRD | | (“LC”) amino acid |
| | | PYTFGQGTKVDIK | | sequence |
| 83 | 537 | DIQLTQSPSTLSASVGDRVTVTCRASQNVGG | ADI-49162 | Light chain |
| | | WLAWYQQKPGKAPKLLIFQASRLENGVPSR | | variable region |
| | | FSANASGTEFTLTIGSLQPDDFATYYCQQYN | | (“LC”) amino acid |
| | | TYPYTFGQGTKVDIK | | sequence |
| 84 | 538 | DIQLTQSPSSLSASVGDRVTITCRASQSISQY | ADI-42223 | Light chain |
| | | LNWYQQKPGKAPKLLISPASSFQSGVPSRFS | | variable region |
| | | GSGSGTDFTLTITSLQPEDFATYYCQQSYSTP | | (“LC”) amino acid |
| | | WTFGQGTKVDIK | | sequence |
| 85 | 539 | QSVLTQPASVSGSPGQSITISCTGTSSDVGGY | ADI-48435 | Light chain |
| | | NYVSWYQQHPGKAPKLMIYDVSNRPSGVSN | | variable region |
| | | RFSGSKSGNTASLTISGLQAEDEADYYCSSY | | (“LC”) amino acid |
| | | TSSSTLVFGGGTQLTVL | | sequence |
| 86 | 540 | SYELTQPPSVSVAPGQTARIICGGNYIGGKS | ADI-46742 | Light chain |
| | | VHWYQQKPGQAPVLVVYNDNDRPSGIPERF | | variable region |
| | | SGSNSGNTATLTISRVAAGDEADYYCQVWD | | (“LC”) amino acid |
| | | NSSDRRVFGGGTKLTVL | | sequence |
| 87 | 541 | DIRVTQSPATLSVSPGERATLSCRASQRVNS | ADI-42787 | Light chain |
| | | NLAWYQQKPGQAPRLLIYGASTRATGIPVR | | variable region |
| | | FSGSGSGTEFTLTISSLQSEDFAVYYCQQYNT | | (“LC”) amino acid |
| | | WWTFGQGTKVEIK | | sequence |
| 88 | 542 | DIQMTQSPSSLSASVGDRVTITCRASQSISSY | ADI-46718 | Light chain |
| | | LNWYQQKPGKAPKLLIYAASSLQSGVPSRFS | | variable region |
| | | GSGSGTDFTLTISSLQPEDFATYYCQQSYSTP | | (“LC”) amino acid |
| | | LTFGGGTKVDIK | | sequence |
| 89 | 543 | DIVMTQSPATLSVSPGERATLSCRASQSVSS | ADI-49141 | Light chain |
| | | NLAWYQQKPGQAPRLLIYGASTRATGIPAR | | variable region |
| | | FSGSGSGTEFTLTISSLQSEDFAVYSCQQYNT | | (“LC”) amino acid |
| | | WPKTFGQGTKVEIK | | sequence |
| 90 | 544 | EIVLTQSPGTLSLSPGERATLSCRASQSVSSSF | ADI-42213 | Light chain |
| | | LAWYQQKPGQAPRLLIYGASSRATGIPDRFS | | variable region |
| | | GSGSGTDFTLT1RRLEPEDFAVYYCQQYGSS | | (“LC”) amino acid |
| | | RRTFGQGTKVEK | | sequence |
| 91 | 545 | DIRVTQSPSTLSASVGDRVTITCRASQSISSW | ADI-42844 | Light chain |
| | | LAWYQQKPGKAPKLLIYRASSLESGVPSRFS | | variable region |
| | | GSGSGTEFTLTISSLQPDDFATYYCQQYNRY | | (“LC”) amino acid |
| | | PYTFGQGTKVEK | | sequence |
| 92 | 546 | QSALTQPASVSGSPGQSITISCTGTSSDVGGY | ADI-45161 | Light chain |
| | | NYVSWYQQHPGKAPKLMIYEVSNRPSGVSN | | variable region |
| | | RFSGSKSGNTASLTISGLQAEDEADYYCSSY | | (“LC”) amino acid |
| | | TSSSTLDVVFGGGTKLTVL | | sequence |
| 93 | 547 | EIVMTQSPATLSVSPGERATLSCRASQSVSSN | ADI-42192 | Light chain |
| | | LAWYQQKPGQAPRLLIYGASTRATSIPARFS | | variable region |
| | | GSGSGTEFTLTISSLQSEDFAVYYCQQYNSW | | (“LC”) amino acid |
| | | PPITFGQGTRLEK | | sequence |
| 94 | 548 | DIRLTQSPSTLSASVGDRVSITCRASQSISDW | ADI-48910 | Light chain |
| | | LAWYQQKPGKAPKLLIYRASGLETGVPSRF | | variable region |
| | | SGSGSGTEFTLTISSLQPDDFATYYCQQYNR | | (“LC”) amino acid |
| | | YPYTFGQGTKVDIK | | sequence |
| 95 | 549 | QPVLIQPPSASGTPGQRVTISCSGSSSNFGSN | ADI-42193 | Light chain |
| | | FVYWYQQLPGTAPKLLIYRVNQRPSGVPDR | | variable region |
| | | FSGSKSGTSASLAISGLRSEDEADYYCATWD | | (“LC”) amino acid |
| | | VSLSNDVLFGGGTKLTVL | | sequence |
| 96 | 550 | DIVLTQSPATLSLSPGERATLSCRASQSVSSS | ADI-49590 | Light chain |
| | | YLSWYQQKPGQAPRLLIYGASSRATGIPDRF | | variable region |
| | | SGSGSGTDFTLTISRLEPEDFAVYYCQQYGS | | (“LC”) amino acid |
| | | SPPITFGGGTKVEIK | | sequence |
| 97 | 551 | DIQMTQSPSSLSASVGDRVTITCRASQTITRY | ADI-45076 | Light chain |
| | | MNWYQQKPGEAPKLLIYATSSLQSGVPSRF | | variable region |
| | | SGSGSGTDFTLTITNLQPADFATYYCQQSST | | (“LC”) amino acid |
| | | TRWTFGQGTKVDIK | | sequence |
| 98 | 552 | DIRLTQSPSSLSASVGDRVTITCRASQDIRKF | ADI-48968 | Light chain |
| | | LNWYQQKLGKAPSLLIYGASSLQSGVPSRFS | | variable region |
| | | GSGSGTDFTLTISSLQPEDFAIYYCQHASTTP | | (“LC”) amino acid |
| | | WTFGQGTKVEIK | | sequence |
| 99 | 553 | SYELTQPPSVSVSPGQTATITCSGDKLGYTY | ADI-42212 | Light chain |
| | | TCWYQQKPGQSPVLVIYQDTKRPSQPERFS | | variable region |
| | | GSNSGNTATLTITGTQAMDEADYYCQAWD | | (“LC”) amino acid |
| | | TTTAGGVFGGGTKLTVL | | sequence |
| 100 | 554 | DIVLTQSPGTLSLSPGERATLSCRASQSVSSS | ADI-48462 | Light chain |
| | | YLAWYQQKPGQAPRLLIYGASSRATGIPDRF | | variable region |
| | | SGSGSGTDFTLTISRLEPEDFAVYYCQQYGS | | (“LC”) amino acid |
| | | SPRAFGPGTKVEIK | | sequence |
| 101 | 555 | QSVLTQPPSASGSPGQSVTISCAGTRSDVGG | ADI-45127 | Light chain |
| | | YNFVSWYQQHPGKAPKLLIYEVNKRPSGVP | | variable region |
| | | DRFSGSKSANTASLTVSGLQAEDEAEYFCSS | | (“LC”) amino acid |
| | | YGGNNDLVFGGGTKVTVL | | sequence |
| 102 | 556 | EIVMTQSPATLSLSPGERGTLSCRTSQSVSSF | ADI-42200 | Light chain |
| | | LAWYQQKPGQAPRLLMYDASNRATGIPARF | | variable region |
| | | SGSGSGTDFTLTISSLEPEDFAVYYCQQRSN | | (“LC”) amino acid |
| | | WPYTFGQGTKVDIK | | sequence |
| 103 | 557 | GIQLTQSPSTLSASVGDRVTITCRASQSVSD | ADI-50203 | Light chain |
| | | WLAWYQQKPGRAPNLLIYRASSLQSGVPSR | | variable region |
| | | FSGSGSGTEFTLTINSLQPDDFATYYCQQYK | | (“LC”) amino acid |
| | | TYWTFGQGTKVEIK | | sequence |
| 104 | 558 | QSVLTQPASVSGSPGQSITISCTGTSSDVGGY | ADI-42149 | Light chain |
| | | NYVSWYQQHPGKAPKLMIYEVSNRPSGVSN | | variable region |
| | | RFSGSKSGNTASLTISGLQAEDEADYYCSSY | | (“LC”) amino acid |
| | | TSSGTNIFGTGTKLTVL | | sequence |
| 105 | 559 | DIVMTQTPATLSVSPGERATLSCRASQSVSS | ADI-42181 | Light chain |
| | | NVAWYQQKPGQAPRLLIHGASTRATGIPAR | | variable region |
| | | FSGSGSGTEFTLTISSLQSEDFAVYYCQQYN | | (“LC”) amino acid |
| | | NWPPLTFGGGTKLEIK | | sequence |
| 106 | 560 | SYELTQPPSVSVSPGQTARITCSGDALPKKY | ADI-45126 | Light chain |
| | | VYWFQQKSGQAPVLVIYEDRRGPSGIPERFS | | variable region |
| | | GSTSGTMATLT1RGAQVEDEADYFCYSTDSS | | (“LC”) amino acid |
| | | GLLGVFGGGTKLTVL | | sequence |
| 107 | 561 | DIQMTQSPDSLAVSLGERATINCKSSQSVFY | ADI-45074 | Light chain |
| | | SSNSQNYLAWYQQKPGQPPKLLIYWASTRE | | variable region |
| | | SGVPDRFSGSGSATDFSLTISSLQAEDVAVYY | | (“LC”) amino acid |
| | | CQQFHSPPWTFGQGTKLEIK | | sequence |
| 108 | 562 | DIVMTQSPSTLSASVGDRVVITCRASQSISN | ADI-49041 | Light chain |
| | | WLAWYQQKSGKAPKLLIYKASRLESGVPST | | variable region |
| | | FSGSGSGTEFTLTISSLQADDFASYYCQQYN | | (“LC”) amino acid |
| | | DYPWTFGQGTKVEK | | sequence |
| 109 | 563 | EIVMTQSPSSLSASVGDRVTITCRASQGIRND | ADI-42227 | Light chain |
| | | LGWYQQKPGKAPKRLIYAASSLQSGVPSRF | | variable region |
| | | SGSGSGREFTLTISSLQPEDFATYYCLQHNT | | (“LC”) amino acid |
| | | YPWTFGQGTKVEK | | sequence |
| 110 | 564 | DIQVTQSPSTLSASVGDRVSITCRASQTISSW | ADI-50220 | Light chain |
| | | LAWYQQKPGKAPKLLMYKASNLQSGVPSR | | variable region |
| | | FTGSGSGTEFTLTISSLQPDDFATYYCQQYYS | | (“LC”) amino acid |
| | | YPYTFGPGTKVDIK | | sequence |
| 111 | 565 | SYVLTQPPSVSVSPGQTARITCSGDALPKQY | ADI-42141 | Light chain |
| | | GYWYQQKPGQAPVLVIYKDSERPSQPERFS | | variable region |
| | | GSSSGTTVTLTISGVQAEDEADYYCQSADRS | | (“LC”) amino acid |
| | | GTVVFGGGTKLTVL | | sequence |
| 112 | 566 | QAVVTQPPSASGSPGQSVTISCTGTSSDVGG | ADI-42216 | Light chain |
| | | YNYVSWYQQHPGKAPKLMVYEVTKRPSGV | | variable region |
| | | PDRFSGSKSGNAASLTVSGLQAEDEAEYYCS | | (“LC”) amino acid |
| | | SYAGSNALVFSGGTKLTVL | | sequence |
| 113 | 567 | NFMLTQPHSVSESPGKTVTISCTRSSGSIASN | ADI-50534 | Light chain |
| | | YVQWYQQRPGSAPTTVIYEDNQRPSGVPDR | | variable region |
| | | FSGSIDSSSNSASLTISGLKTEDEADYYCQSY | | (“LC”) amino acid |
| | | DSSNWVFGGGTKLTVL | | sequence |
| 114 | 568 | QPELTQPPSVSVSPGQTARITCSGDALSKQY | ADI-49140 | Light chain |
| | | AYWYQQKPGQAPVVVIYKDSERPSGIPERFS | | variable region |
| | | GSRSGTTVTLTISGVQAEDEADYYCHSPDSH | | (“LC”) amino acid |
| | | VVFGGGTKLTVL | | sequence |
| 115 | 569 | SYELIQLPSASVAPGKTARITCGGNNIGSKSV | ADI-46741 | Light chain |
| | | HWYQQKPGQAPVLVVYDDSDRPSGIPERFS | | variable region |
| | | GSNSGNTATLTISRVEAGDEADYYCQVWDS | | (“LC”) amino acid |
| | | SSDHEVFGGGTKLTVL | | sequence |
| 116 | 570 | DIQMTQSPSSLSASVGDRVTITCQASQDISNY | ADI-42195 | Light chain |
| | | LNWYQQKPGKAPKLLIYDVSKLKTGVPPRF | | variable region |
| | | SGSGSGTDFTFTISSLQPEDIATYYCQQWGT | | (“LC”) amino acid |
| | | FGQGTKVDIK | | sequence |
| 117 | 571 | EIVLTQSPSTLSASVGDRVTITCRASQSISDW | ADI-42172 | Light chain |
| | | LAWYQQKPGKAPNLLIYRASSLESGVPSRFS | | variable region |
| | | GSGSGTEFTLTISSLQPDDFATYYCQQYNRY | | (“LC”) amino acid |
| | | PYTFGQGTKVEIK | | sequence |
| 118 | 572 | DIQLTQSPSTLSASVGDRVTITCRASQSISDW | ADI-42178 | Light chain |
| | | LAWFQQKPGKAPKLLIYRASGLETGVPSRFS | | variable region |
| | | GSGSGTEFTLTISSLQPDDFATYYCQQYNRY | | (“LC”) amino acid |
| | | SYTFGQGTKVEIK | | sequence |
| 119 | 573 | DIRLTQSPSTLSASVGDRVTITCRASQSISGW | ADI-49032 | Light chain |
| | | LAWYQQKPGKAPKLLIYKASILESGVPSRFS | | variable region |
| | | GSQSGTEFTLTISSLQPDDFATYYCQQYNNF | | (“LC”) amino acid |
| | | WTFGQGTKLEIK | | sequence |
| 120 | 574 | QSVLTQPPSVSGAPGQRVTISCTGNSSNIGA | ADI-50197 | Light chain |
| | | GYEVHWYQQLPGTAPKLLIYGNNNRPSGVP | | variable region |
| | | DRFSGSKSGASGSLAVTGLRAEDEADYYCH | | (“LC”) amino acid |
| | | SYDSNMSGSVFGGGTKVTVL | | sequence |
| 121 | 575 | EIVLTQSPSSLSASVGDRVTITCRASQGISNY | ADI-48894 | Light chain |
| | | LAWYQQKPGKAPKLLIYAASTLQSGVPSRF | | variable region |
| | | SGSGSGTDHLTISSLQPEDVATYYCQKYYSA | | (“LC”) amino acid |
| | | PLITFGPGTKVEK | | sequence |
| 122 | 576 | SYELTQPPSVSVSPGQTARITCSGDALPKQY | ADI-42226 | Light chain |
| | | AYWYQQKPGQAPVLVIYKDTERPSGIPERFS | | variable region |
| | | GSSSGTTVTLTISGVQAEDEADYYCQSADSS | | (“LC”) amino acid |
| | | VADSSVVFGGGTKLTVL | | sequence |
| 123 | 577 | EIVLTQSPATLSLSPGERATLSCRASQSVSNY | ADI-49037 | Light chain |
| | | FAWYQQKPGQAPRLLIYGASNRATGVPARF | | variable region |
| | | SGSGSGTDFTLTISSLEPEDFAVYYCQQRSN | | (“LC”) amino acid |
| | | WPYTFGQGTKVEIK | | sequence |
| 124 | 578 | NFMLTQPPSVSAAPGQKVTISCSGSNSNIGN | ADI-46739 | Light chain |
| | | NFVSWYQQLPGTAPKLLIYDNNERPSGIPDR | | variable region |
| | | FSGSKSVTSATLGITGLQTGDEADYYCGTW | | (“LC”) amino acid |
| | | DNSLGMVVFGGGTKLTVL | | sequence |
| 125 | 579 | QSALTQPASVSGSPGQSITISCTGTSSDVGGY | ADI-42810 | Light chain |
| | | NYVSWYQQHPGKAPKLMIYEVSNRPSGVSN | | variable region |
| | | RFSGSKSGNTASLTISGLQAEDEADYYCSSY | | (“LC”) amino acid |
| | | TSSSTYVFGTGTKVTVL | | sequence |
| 126 | 580 | EIVLTQSPGTLALSPGERATLSCRASQSVSSY | ADI-49137 | Light chain |
| | | LAWYQQKPGQAPRLLIYDSSNRATGIPARFS | | variable region |
| | | GSGSGTDFTLTISSLEPEDFAVYYCQQPGNW | | (“LC”) amino acid |
| | | PPAFTFGGGTKLEK | | sequence |
| 127 | 581 | DIVMTQSPATLSVSPGERATLSCRASQSVTS | ADI-42817 | Light chain |
| | | KLAWYQQKPGQAPRLLIYGASTRATGIPAR | | variable region |
| | | FSGSGSGTEFTLTISSLQSEDFAVYYCQQYN | | (“LC”) amino acid |
| | | NWITFGQGTRLEIK | | sequence |
| 128 | 582 | DIQLTQSPSSVSASVGDRVTITCRASQGISSW | ADI-50218 | Light chain |
| | | LAWYQQKPGKAPKLLIYAASSLQSGVPSRFS | | variable region |
| | | GSGSGTDFTLTISSLQPEDFATYYCQQANSFP | | (“LC”) amino acid |
| | | WTFGQGTKVDIK | | sequence |
| 129 | 583 | DIVMTQSPLSLPVTPGEPASISCRSSQSLLHS | ADI-42126 | Light chain |
| | | NGYNSLDWYLQKPGQSPQLLIYLGSNRASG | | variable region |
| | | VPDRFSGSGSGTDFTLKISRVEAEDVGVYYC | | (“LC”) amino acid |
| | | MQALQTPYTFGQGTKLEK | | sequence |
| 130 | 584 | QPVLTQPPSASGTPGQRVTISCSGSSSNIGSN | ADI-42186 | Light chain |
| | | TVHWYQQLPGTAPKLLIYSNNQRPSGVPDR | | variable region |
| | | LSGSRSGTSASLAISGLQSEDEAEYYCAAWD | | (“LC”) amino acid |
| | | DNLIGVVFGGGTKLTVL | | sequence |
| 131 | 585 | QSALTQPPSASGSPGQSVTISCTGTSSDVGG | ADI-48890 | Light chain |
| | | YNYVSWYQQHPGKAPKLMIYEVSKRPSGVP | | variable region |
| | | DRFSGSKSGNTASLTVSGLQAEDEADYYCSS | | (“LC”) amino acid |
| | | FAGSNNLYVFGTGTKVTVL | | sequence |
| 132 | 586 | QSVLTQPASVSGSPGQSITISCTGTSSDVGGY | ADI-42206 | Light chain |
| | | NYVSWYQQHPGKAPKLMIYDVTNRPSGVS | | variable region |
| | | NRFSGSRSGNTASLTISGLQAEDEADYYCSS | | (“LC”) amino acid |
| | | YTRSSTRVFGGGTKLTVL | | sequence |
| 133 | 587 | QPVLTQPPSVSAAPGQKVTISCSGSSSNIGSN | ADI-46724 | Light chain |
| | | FVSWYQQFPGTAPKLLIYDDNKRPSGIPDRF | | variable region |
| | | SGSKSGTSATLGITGLQTGDEADYYCETWD | | (“LC”) amino acid |
| | | SRLSVVFGGGTKLTVL | | sequence |
| 134 | 588 | QPVLTQPPSVSAAPGQKVTISCSGSSSNIGNN | ADI-50539 | Light chain |
| | | YVSWYQQLPGTAPKLLIYDNNKRPSGIPDRF | | variable region |
| | | SGSKSGTSATLGITGLQTGDEADYYCGTWD | | (“LC”) amino acid |
| | | SSLSAVVFGGGTKVTVL | | sequence |
| 135 | 589 | DIQLTQSPSSLSASVGDRVTITCRASQSISSYL | ADI-45156 | Light chain |
| | | NWYQQKPGKAPKLLIYAASSLQSGVPSRFSG | | variable region |
| | | SGSGTDFTLTISSLQPEDFATYYCQQSYSTP | | (“LC”) amino acid |
| | | WTFGQGTKVDIK | | sequence |
| 136 | 590 | DIRVTQSPSSLSASVGDRVTITSRASQSISSYL | ADI-50536 | Light chain |
| | | NWYQQKPGKAPKLLIYAASSLQSGVPSRFSG | | variable region |
| | | SGSGTDFTLTISSLQPEDFATYYCQQSYSTPR | | (“LC”) amino acid |
| | | TFGGGTKVDIK | | sequence |
| 137 | 591 | QSVLTQPPSVSGAPGQRVTISCTGSSSDIGAG | ADI-42217 | Light chain |
| | | YDVHWYQQLPGTAPKLLIYGNTNRPSGVPD | | variable region |
| | | RFSGSKSGTSASLAITGLQAEDEADYYCQSY | | (“LC”) amino acid |
| | | DSSLSGVVFGGGTKLTVL | | sequence |
| 138 | 592 | DIVLTQSPDSLAVSLGERAAINCKSSQSVFFS | ADI-48951 | Light chain |
| | | SDNKNYLAWYQQKPGQPPKLLIYWASTRES | | variable region |
| | | GVPDRFSGSGSGTDFTLTISSLQAEDVAVYY | | (“LC”) amino acid |
| | | CQQFYTTPSTFGQGTKVEIK | | sequence |
| 139 | 593 | DIQLTQSPSSLSASVGDRVTITCRASQSISSYL | ADI-50537 | Light chain |
| | | NWYQQKPGKAPKLLIYAASSLQSGVPSRFSG | | variable region |
| | | SGSGTDFTLTISSLQPEDFATYYCQQSYSTPR | | (“LC”) amino acid |
| | | TFGGGTKVEIK | | sequence |
| 140 | 594 | DIQLTQSPSSLSASVGDRVTITCRASQGIRND | ADI-46737 | Light chain |
| | | LGWYQQKPGKAPKRLIYAASSLQSGVPSRF | | variable region |
| | | SGSGSGTEFTLTISSLQPEDFATYYCLQHNSY | | (“LC”) amino acid |
| | | PLTFGGGTKVEIK | | sequence |
| 141 | 595 | NFMLTQPHSVSESPGNTVTISCTRSSGSIAST | ADI-50538 | Light chain |
| | | YVQWYQQRPGSAPSTVIYEDNQRPPGVPAR | | variable region |
| | | FSGSIDSSSNSASLTISGLETEDEADYYCQSY | | (“LC”) amino acid |
| | | DSTTVVFGGGTKVTVL | | sequence |
| 142 | 596 | SYVLTQPPSASGSPGQSVTISCTGTSSDFGGY | ADI-48950 | Light chain |
| | | NYVSWYQQHPGKAPKLMVYEVAKRPSGVP | | variable region |
| | | DRFSGSKSGNTASLTVSGLQAEDEADYYCSS | | (“LC”) amino acid |
| | | YAGSNNFVVFGGGTKLTVL | | sequence |
| 143 | 597 | QSVLTQPPSVSAAPGQKVTISCSGSSSNIGNN | ADI-42114 | Light chain |
| | | YVSWYQQLPGTAPKLLIYDNNKRPSGIPDRF | | variable region |
| | | SGSKSGTSATLGITGLQTGDEADYYCGTWD | | (“LC”) amino acid |
| | | SSLSAKVFGGGTKLTVL | | sequence |
| 144 | 598 | ETTLTQSPSTLSTSVGDRVTITCRASQSISSW | ADI-49194 | Light chain |
| | | LAWYQQKPGKAPKLLIYRASSLETEVPSRFS | | variable region |
| | | GSGSGTDFTLTISRLQPDDFATYFCQQYNRY | | (“LC”) amino acid |
| | | PYTFGQGTKLEK | | sequence |
| 145 | 599 | QPVLTQPRSVSGSPGQSVTISCTGTSSDVGG | ADI-42124 | Light chain |
| | | YNYVSWYQQHPGKAPKLMIYDVSKRPSGV | | variable region |
| | | PDRFSGSKSGNTASLTISGLQAEDEADYYCC | | (“LC”) amino acid |
| | | SYAGSYTFVLFGGGTKLTVL | | sequence |
| 146 | 600 | DIRVTQSPSSLSASVGDRVTISCRASESISIYL | ADI-45123 | Light chain |
| | | NWYQQKPGKAPNLLIYAASSLQRGVPSRFS | | variable region |
| | | GSGSGTDFTLTITSLQAEDFATYYCQQTFSI | | (“LC”) amino acid |
| | | WTFGQGTKVEIK | | sequence |
| 147 | 601 | QPVLTQPPSVSAAPGQKVTISCSGSSSNIGNN | ADI-50533 | Light chain |
| | | YVSWYQQLPGTAPKLLIYDNNKRPSGIPDRF | | variable region |
| | | SGSKSGTSATLGITGLQTGDEADYYCGTWD | | (“LC”) amino acid |
| | | SSLSAGKVFGGGTKLTVL | | sequence |
| 148 | 602 | QSALTQPPSASGSPGQSVTISCTGTSSDVGGF | ADI-49205 | Light chain |
| | | NYVSWYQQHPGRAPKLVIYEVNRRPSGVPD | | variable region |
| | | RFSGSKSGYTASLTVSGLQAEDEADYYCFSY | | (“LC”) amino acid |
| | | AGSNNYVFGTGTKVTVL | | sequence |
| 149 | 603 | DIVMTQTPLSSPVTLGQPASISCRSSQSLVHS | ADI-45151 | Light chain |
| | | DGNTYLSWLQQRPGQPPRFLIYKISNRFSGV | | variable region |
| | | PDRFSGGGAGTDFTLKISRVEAEDVGVYYC | | (“LC”) amino acid |
| | | MQASQFPLTFGGGTKVEIK | | sequence |
| 150 | 604 | EIVMTQSPSSLSASVGDRVTITCQASQDISNY | ADI-46728 | Light chain |
| | | LNWYQQKPGKAPKLLIYDASNLETGVPSRF | | variable region |
| | | SGSGSGTDFTFTISSLQPEDIATYYCQQYDNL | | (“LC”) amino acid |
| | | PPVTFGQGTRLEIK | | sequence |
| 151 | 605 | QPVLTQPPSVSVSPGQTASITCSGDKLGDKY | ADI-49030 | Light chain |
| | | ACWYQQKPGQSPVLVIYQDSKRPSQPERFS | | variable region |
| | | GSNSGNTATLTISGTQAMDEADYYCQAWDS | | (“LC”) amino acid |
| | | STDVVFGGGTKVTVL | | sequence |
| 152 | 606 | DIQVTQSPSSLSASVGDRVTITCRASQGISNN | ADI-50200 | Light chain |
| | | LAWYQQKPGIFPKLLIYAASTLQSGVPSRFS | | variable region |
| | | GSGSGTDFILTISSLQPEDVATYYCQKYQSA | | (“LC”) amino acid |
| | | PPTFGGGTKLE1K | | sequence |

### Materials and Methods

### Study Design

Study subjects aged 30 and 31 years of age were vaccinated with the YFV-17D Stamaril vaccine. Heparinized blood (50-100 cc) was obtained from subjects before vaccination and on days 10, 14, 28, 90, 180, 270, and 360 following vaccination. Samples were processed in the Immune Monitoring and Flow Cytometry core laboratory at the Geisel School of Medicine at Dartmouth to obtain plasma and to isolate peripheral blood-derived B cells. Isolated cells and plasma were stored frozen in aliquots at −80° C.

Cells: Huh 7.5.1 cells (received from Dr. Jan Carette; originally from Dr. Frank Chisari) were passaged every 3 to 4 days using 0.05% Trypsin/EDTA solution (Gibco) and maintained in Dulbecco's Modified Eagle Medium (DMEM high glucose, Gibco) supplemented with 10% heat-inactivated fetal bovine serum (FBS, Atlanta Biologicals), 1% Penicillin/Streptomycin (P/S, Gibco), 1% Gluta-MAX (Gibco) and 25 mM HEPES (Gibco). Vero African grivet monkey kidney cells (obtained from ATCC) were passaged every 3 to 4 days using 0.05% Trypsin/EDTA solution (Gibco) and maintained in Dulbecco's Modified Eagle Medium (DMEM high glucose, Gibco) supplemented with 2% heat-inactivated fetal bovine serum (FBS, Atlanta Biologicals), 1% Penicillin/Streptomycin (P/S, Gibco), 1% Gluta-MAX (Gibco) and 25 mM HEPES (Gibco).

Yellow Fever virus 17D generation: YFV-17D was obtained from BEI Resources (cat #NR-115). 15 cm plates with Huh 7.5.1 in a confluency of 80% were infected with 90 μL of passage 2 stock of YFV-17D supernatant in 3 mL of infection media (DMEM low glucose (Gibco), 7% FBS, 1% Pen-Strep, 1% Gluta-MAX (Gibco), 25 mM HEPES (Gibco)) for 1 hour at 37 C and 5% CO2. After 3 days the supernatant was harvested and centrifuged twice at 4,000 rpm for 15 min at 4° C. to remove cell debris. The YFV-17D viral stock for neutralization assays was generated by ultracentrifugation of the pre-cleared supernatant at 28,000 rpm using a SW28 rotor (Beckman Coulter) in a Beckman Coulter Optima LE-80K ultracentrifuge for 4 hours through a 2 mL 30% (v/v) D-sucrose/PBS cushion. The pellet was allowed to resuspend overnight on ice in 300 ul PBS and afterwards aliquoted and frozen at −80 C.

Zika virus generation: The Zika virus strain MR 766 was obtained from ATCC (ATCC® VR-84™). For neutralization assay 15 cm plates with Vero cells in a confluency of 80% were infected with 90 μL of passage 1 stock of Zika supernatant in 3 mL of infection media (DMEM low glucose (Gibco), 2% FBS, 1% Pen-Strep, 1% Gluta-MAX (Gibco), 25 mM HEPES (Gibco)) for 1 hour at 37 C and 5% CO2. After 3 days the supernatant was harvested and centrifuged twice at 4,000 rpm for 15 min at 4° C. to remove cell debris.

### Antigens and Antibodies

Production of recombinant YFV antigens: The coding region for the entire prM and soluble E (sE) region of the YFV Asibi Strain (Uniprot ID: Q6DV88, residues 122-678 of the genome polyprotein) was cloned into pMT-puro, an insect expression vector encoding a C-terminal double strep tag. Expression construct design was based on previously published structures of flavivirus antigens61, 62, 63. The YFV prM/E construct was used to generate an inducible, stable Drosophila S2 line. Protein expression was induced with addition of copper sulfate and allowed to proceed for 5-7 days. Recombinant protein was affinity-purified from the culture supernatant with a StrepTrap HP column (GE Healthcare). An additional purification step was carried out using size-exclusion chromatography step using an S200Increase column (GE Healthcare). The final protein preparations were stored in phosphate-buffered saline pH 7.4 supplemented with an additional 150 mM NaCl. Small aliquots were stored at −70° C. until use. The additional flavivirus antigens used int his study—DENV-2 E, DENV-4 E, WNV E and ZIKV E were expressed and purified essentially as described for YFV sE.

Flavivirus NS1 protein antigens: The NS1 proteins from dengue virus (serotypes 1-4), JEV, TBEV, WNV, YFV were purchased from Native Antigen Company (Cat #FLAVX4-NS1-100 and DENVX4-NS1-100) and the ZIKV NS1 was purchased from Meridian Life Science (Cat #R01636). The positive control antibodies reactive to the above NS1 proteins were obtained from Native Antigen Company: anti-DENV NS1 (Cat #AbDENVNS1-DA034), anti-ZIKV NS1 antibody (Cat #AbZIKVNS1-B4-100). The anti-YFV NS1 protein antibody was purchased from Meridain Life Sciences (Cat #C01906M). The anti-WNV NS1 antibody (Cat #HM484-X0632) and anti-TBEV NS1 antibody (Cat #HM477-X1462) were purchased from East Coast Bio. Flavivirus cross-reactive serum was used to detect the JEV NS1 protein.

YFV-17D DIII protein: The DIII region (aa 293-397) of YFV-17D E protein (Uniprot ID: P03314) was produced in Drosophila S2 cells using a modified pT350 vector (Felix Rey, Institut Pasteur, France). Protein expression was induced by CdCl2 and the supernatant was harvested 5-7 days post-induction. Recombinant protein was purified using a Strep-Tactin column (IBA) and size-exclusion chromatography using a S200Increase column (GE Healthcare) and 10mMTris pH8/150 mM NaCl buffer.

### Single B-Cell Sorting

For plasmablast sorting, PBMCs were stained using anti-human CD38 (PE), CD27 (BV421), CD20 (PE-Cy7), CD3 (PerCP-Cy5.5), CD8 (PerCP-Cy5.5), CD14 (PerCP-Cy5.5) and CD16 (PerCP-Cy5.5). Plasmablasts were defined as CD19+CD3−CD20−/loCD27highCD38high cells. For MBC sorting, B cells were purified using a MACS B cell isolation kit (Miltenyi Biotec; cat #130-091-151) and subsequently stained using anti-human CD19 (PE-Cy7), CD20 (PE-Cy7), CD3 (PerCP-Cy5.5), CD8 (PerCP-Cy5.5), CD14 (PerCP-Cy5.5), CD16 (PerCP-Cy5.5), IgD (BV421), IgM (AF-488), CD27 (BV510), CD21 (BV605), CD71 (APC-Cy7 and a mixture of dual-labeled (APC and PE) YFV E tetramers (25 nM each). Tetramers were prepared fresh for each experiment, and B cells that showed reactivity to the YFV E tetramers were single cell sorted. Single cells were sorted using a BD FACS Aria II (BD Biosciences) into 96-well PCR plates (BioRAD) containing 20 uL/well of lysis buffer [5 uL of 5× first strand cDNA buffer (Invitrogen), 0.625 uL of NP-40 (New England Biolabs), 0.25 uL RNaseOUT (Invitrogen), 1.25 uL dithiothreitol (Invitrogen), and 12.6 uL dH2O]. Plates were immediately stored at −80° C. Flow cytometry data were analyzed using FlowJo software.

### Amplification and Cloning of Antibody Variable Genes

Antibody variable genes (IgH, IgK, and IgL) were amplified by reverse transcription PCR and nested PCRs using cocktails of IgG- and IgM-specific primers, as described previously (Tiller et al, J Immunol 2008). The primers used in the second round of PCR contained 40 base pairs of 5′ and 3′ homology to the digested expression vectors, which allowed for cloning by homologous recombination into S. cerevisiae. The lithium acetate method for chemical transformation was used to clone the PCR products into S. cerevisiae (Gietz and Schiestl, Nat Protoc 2007). 10 uL of unpurified heavy chain and light chain PCR product and 200 ng of the digested expression vectors were used per transformation reaction. Following transformation, individual yeast colonies were picked for sequencing and characterization.

### Expression and Purification of IgGs and Fab Fragments

IgGs were expressed in S. cerevisiae cultures grown in 24-well plates, as described previously (Bornholdt et al, Science 2016b). After 6 days, the cultures were harvested by centrifugation and IgGs were purified by protein A-affinity chromatography. The bound antibodies were eluted with 200 mM acetic acid/50 mM NaCl (pH 3.5) into ⅛th volume 2 M Hepes (pH 8.0), and buffer-exchanged into PBS (pH 7.0).

The two YFV E-reactive control mAbs, 5A and 4G2, were produced in the human IgG1 constant region. The publicly available variable region sequences of the two control antibodies, 4G2 and 5A, were synthesized as gBlock fragments (IDT) with homologous overhangs for recombinational cloning into S. cerevisiae. Subsequent production was carried out as described above.

Fab fragments were generated by digesting the IgGs with papain for 2 h at 30° C. The digestion was terminated by the addition of iodoacetamide, and the Fab and Fc mixtures were passed over Protein A agarose to remove Fc fragments and undigested IgG. The flowthrough of the Protein A resin was then passed over CaptureSelect™ IgG-CH1 affinity resin (ThermoFischer Scientific), and eluted with 200 mM acetic acid/50 mM NaCl pH 3.5 into ⅛th volume 2M Hepes pH 8.0. Fab fragments then were buffer-exchanged into PBS pH 7.0.

### Kinetics of Binding Measurements

Surface Plasmon Resonance Kinetic Measurements (SPR) of IgG binding: A Biacore 8K system, docked with a CAP sensor chip, sample compartment was set to 10° C., flow cell temperature to 25° C., and the data collection rate to 10 Hz. HBS-EP+(10 mM HEPES pH 7.3, 150 mM NaCl, 3 mM EDTA, 0.05% Surfactant P20) was used as the running buffer. In each cycle, biotin CAPture reagent (GE Healthcare) diluted 1:20 in running buffer was injected over flow cells 1 and 2 for 600 s, at 20 a flow rate of 5 uL/min, followed by a 900 s capture (1 uL/min) of biotinylated YFV E antigen (25 nM in HBS-EP+) over flow cell 2 to reach a minimum capture level of 400 RU. The antibodies (36-288 nM in HBS-EP+) were then injected over flow cells 1 and 2 for 300 s (30 uL/min), the dissociation monitored for 300 s (30 uL/min), and the surface regenerated at the oligonucleotide level with 6M Guanidine-HCl in 0.25 M NaOH for 120 s (10 uL/min). A minimum of two blank (HBS-EP+) injections also were run under identical conditions as described above and used to assess and subtract system artifacts. The data were aligned, double referenced, and fit to bivalent analyte binding model using Biacore 8K Evaluation Software, version 1.0.

Surface Plasmon Resonance Kinetic Measurements (SPR) of Fab binding: A Biacore 8K system, docked with a CAP sensor chip, sample compartment was set to 10° C., flow cell temperature to 25° C., and the data collection rate to 10 Hz. HBS-EP+(10 mM HEPES pH 7.3, 150 mM NaCl, 3 mM EDTA, 0.05% Surfactant P20) was used as the running buffer. In each cycle, biotin CAPture reagent (GE Healthcare) diluted 1:20 in running buffer was injected over flow cells 1 and 2 for 600 s, at a flow rate of 1 uL/min, followed by a 900 s capture (1 uL/min) of biotinylated YFV E protein (15 nM in HBS-EP+) over flow cell 2 to reach a minimum capture level of 275 RU. The Fabs (A5: 27-1 nM in HBS-EP+; 4G2: 4-0.125 nM in HBS-EP+) were then injected over flow cells 1 and 2 for 300 s (30 uL/min), the dissociation monitored for 1200 s (30 uL/min), and the surface regenerated at the oligonucleotide level with 6M Guanidine-HCl in 0.25 M NaOH for 185 s (10 uL/min). A minimum of two blank (HBS-EP+) injections also were run under identical conditions as described above and used to assess and subtract system artifacts. The data were aligned, double referenced, and fit to a 1:1 binding model using Biacore 8K Evaluation Software, version 1.0.

Bio-Layer Interferometry Kinetic Measurements (BLI): For monovalent apparent KD determination, IgG binding to recombinant YFV E antigen was measured by biolayer interferometry (BLI) using a ForteBio Octet HTX instrument (Molecular Devices). The IgGs were captured (1.5 nm) to anti-human IgG capture (AHC) biosensors Molecular Devices) and allowed to stand in PBSF (PBS with 0.1% w/v BSA) for a minimum of 30 min. After a short (60 s) baseline step in PBSF, the IgG-loaded biosensor tips were exposed (180 s, 1000 rpm of orbital shaking) to YFV E antigen (100 nM in PBSF) and then dipped (180 s, 1000 rpm of orbital shaking) into PBSF to measure any dissociation of the antigen from the biosensor tip surface. Data for which binding responses were >0.1 nm were aligned, inter-step corrected (to the association step) and fit to a 1:1 binding model using the ForteBio Data Analysis Software, version 11.1.

For bivalent apparent KD determination, IgG binding to recombinant biotinylated YFV E antigen was measured by biolayer interferometry (BLI) using a ForteBio Octet HTX instrument (Molecular Devices). Recombinant biotinylated YFV E was immobilized on streptavidin biosensors (Molecular Devices) and allowed to stand in PBSF (PBS with 0.1% w/v BSA) for a minimum of 30 min. After a short (60 s) baseline step in PBSF, the antigen-loaded biosensor tips were exposed (180 s, 1000 rpm of orbital shaking) to the IgGs (100 nM in PBSF) and then dipped (180 s, 1000 rpm of orbital shaking) into PBSF to measure any dissociation of the IgGs from the biosensor tip surface. Data for which binding responses were >0.1 nm were aligned, interstep corrected (to the association step) and fit to a 1:1 binding model using the ForteBio Data Analysis Software, version 11.1.

### High Throughput Antibody Epitope Assignment

Bio-Layer Interferometry (BLI) Epitope Binning: For epitope binning, control antibodies A5 and 4G2 (produced as human IgG1 chimeras) were captured on anti-human IgG capture biosensors (0.9 nm) (Molecular Devices) and the biosensors were then blocked by exposing them to adalimumab (0.5 mg/mL; 20 min, 350 rpm of orbital shaking). After a short (60 s) baseline step in PBSF, a cross-interaction check was performed between the sample IgGs and the loaded biosensors (180 s, 1000 rpm of orbital shaking). No cross-interaction was observed for this panel of IgGs. The loaded biosensors were then subjected to a second short (60 s) baseline step in PBSF, followed by an association step in 100 nM recombinant YFV E monomer (180 s, 1000 rpm of orbital shaking). Finally, the binning step was performed in 100 nM sample IgGs in PBS with 0.1% BSA (PBSF) (180 s, 1000 rpm of orbital shaking). Data were analyzed using the ForteBio Data Analysis Software, version 11.1. Sample IgGs with a binning response lower than 0.1 nm were determined to compete with the control antibody. Sample IgGs with a binning response greater than 0.1 nm were determined to be non-competitors to the control antibody.

### High-Throughput Epitope Binning Using Carterra LSA (SPR)

Binding kinetics and affinities. The kinetic rate and affinity constants for Yellow Fever antigen (supplied by Adimab as purified recombinant monomer, MW of 45 kDa) binding to a library of 770+ ADI mAbs (supplied as purified human IgG) were determined at a temperature of 25° C. in a “Capture Kinetics” assay format using Carterra's high throughput surface plasmon resonance (SPR) biosensor platform equipped with HC-30M chip type. To prepare the surfaces for this experiment, the chip was coated as a “lawn” with a capture reagent, namely goat anti-human-IgG Fc polyclonal cross-adsorbed to serum proteins from multiple other species (Southern Biotech, cat #2014-01) using standard amine coupling in a run buffer of 10 mM Hepes pH 7.4, 150 mM NaCl, 3 mM EDTA, 0.05% Tween20 (HBSET). Briefly, this involved priming the single flow cell (SFC) with HBSET run buffer, injecting a freshly prepared activation solution of 1:1:1 v/v/v 0.1 M N-hydroxysulfosuccinimide (Sulfo-NHS, Pierce)+0.4 M 1-ethyl-3-(3-dimethylaminopropyl)carbodiimide hydrochloride) (EDC, Pierce)+0.1 M MES pH 5.5 (Carterra) for 10 min, coupling 50 μg/ml goat anti-human-IgG Fc diluted into 10 mM sodium acetate pH 4.3 for 15 min, and quenching excess reactive esters with 1 M ethanolamine pH 8.5 for 7 min. This resulted in mean final coupled levels of 6256 RU+4% variance (as judged by the 384 reaction spots). The 96-channel printhead (96PH) was then primed in run buffer and used to capture the ADI mAbs as ligands, which were diluted to 2 μg/ml in run buffer and batch-printed 96 at a time onto discrete spots. Four serial docks of the 96PH were used to address all 4 print block locations, thus generating a 384-ligand array. The 96PH was returned to water for cleaning and the SFC was docked over the printed array and primed with the assay run buffer of HBSET+0.5 g/l BSA. Analyte samples of Yellow Fever Monomer antigen were prepared as an 8membered 4fold dilution series spanning nominal concentrations of 0.02-367 nM and injected in the SFC in ascending concentration after several buffer (blank) injections. Association and dissociation times were 5 min and 20 min respectively. Data were analyzed in Carterra's Kinetic Software as follows. The binding data on the reaction spots were double referenced by subtracting the responses from local reference spots (representing naked capture reagent) and then subtracting the responses from a buffer blank analyte66. Double-referenced data were fit globally to a simple Langmuir model allowing each spot its own association rate constant (ka), dissociation rate constant (kd), and Rmax value. The equilibrium dissociation (affinity) constant (KD) was computed from the ratio of the kinetic rate constants, KD=kd/ka).

Epitope binning experiments: Carterra's LSA was used to perform epitope binning assays in a classical sandwich assay format67 using 6 benchmark mAbs (ADI-49582, ADI-44112, ADI-45107, ADI-49147, 4G2, and 5A) as analyte to probe the epitope diversity of the 770+ ADI library as ligands. An HCX-30M (pre-activated) chip type was used and experiments were performed at 25° C. The SFC and 96PH were primed in run buffer of 25 mM Mes pH5.5+0.01% Tween20. The ADI mAbs were diluted to 2 μg/ml in 10 mM sodium acetate pH 4.5 (coupling buffer) and coupled via the 96PH using 7 min contact time at each print block location. After 4 serial docks of the 96PH to build up a 384-ligand array, the SFC was docked over the entire surface to quench excess reactive esters by injecting ethanolamine pH8.5 for 7 min. Final coupled levels of each mAb ranged from 1000-4000 RU per spot. The 96PH was returned to water for cleaning and the SFC was primed in an assay run buffer of HBSET+0.5 g/l BSA. Each binning cycle involved a co-inject style of sample delivery whereby the antigen (50 nM Yellow Fever Monomer) and antibody analyte (20 ug/ml mAb or buffer) samples were injected back-to-back, with minimal dissociation time between them over the 384-ligand array. Typical association times were 3 or 5 min and surfaces were regenerated with 75 mM phosphoric acid after each binning cycle. The binding data were analyzed in Carterra's Epitope Software.

### Micro-Titer Neutralization Assays

Monoclonal antibodies were serially diluted in DMEM high glucose medium (Gibco) containing 10% heat-inactivated FBS (Gibco), 1% Gluta-MAX Gibco), 1% P/S (Gibco) and 25 mM HEPES (Gibco) and incubated at room temperature with YFV-17D or ZIKV for 1 hour. YFV-17D or ZIKV was diluted to achieve 60% endpoint infection. The antibody-virus mixture was added in triplicates to 96-well plates (Costar 3595) containing 5×10\hat{ }3 Huh 7.5.1 cell monolayers seeded the day before. Cells were incubated for 2 days at 37° C. and 5% CO2. Cells were then fixed with 4% paraformaldehyde (Sigma) for 10 minutes and were washed afterwards with a Tris buffer (50 mM Tris, 150 mM NaCl (all Fisher Scientific), pH 7.6, three times. Fixed cells were incubated with a pan-flavivirus mouse mAb 4G2 (ATCC) at 2 μg/ml in Tris buffer containing 3% nonfat dry milk powder (BioRad), 0.5% Triton X-100 (MP Biomedicals), and 0.05% Tween 20 (Fisher Scientific) for one hour at room temperature (RT). Afterwards, cells were washed three times and incubated with the secondary antibody conjugated to Alexa Fluor 488 goat anti-mouse (Invitrogen) at 1:500 dilution for one hour at RT. Cells were washed again and nuclei were stained with Hoechst-33342 (Invitrogen) in a 1:2,000 dilution in PBS. Viral infectivity was measured by automated enumeration of Alexa Fluor 488-positive cells from captured images using Cytation-5 automated fluorescence microscope (BioTek) and analyzed using the Gen5 data analysis software (BioTek). The half maximal inhibitory concentration (IC50) of the mAbs was calculated using a nonlinear regression analysis with GraphPad Prism software. Viral neutralization data were subjected to nonlinear regression analysis to extract the half maximal inhibitory concentration (IC50) values (4-parameter, variable slope sigmoidal dose-response equation; GraphPad Prism).

Neutralization of donor plasma samples was carried out exactly as described above for purified IgGs. Serial dilutions of plasma were pre-incubated with YFV-17D infectious stock for 1 hour before adding to cell monolayers.

Purified total human IgG from non-immunized donors was used as negative control in purified IgG neutralization assays against YFV-17D and ZIKV (Cat #AB_2337042, Jackson Immuno Research).

### FRNT Assay

Virus-specific mAbs were screened as previously described. Briefly, all purified mAbs were serially diluted in 199 medium (Thermo Scientific) containing 5% heat-inactivated fetal bovine serum (FBS) (Gibco-Invitrogen) and incubated at 37° C. with YFV-17DD. After 1 hr incubation, the Ab-virus mixture was added in duplicate to 96-well plates containing 80% confluent monolayers of Vero E6 cells. Plates were incubated for 1.5 h at 37° C. Wells were then overlaid with 1% methylcellulose in supplemented OptiMEM GlutaMAX media (Invitrogen) with 5% heat-inactivated FBS (Gibco-Invitrogen) and 1% amphotericin B and incubated at 37° C., 5% CO2 for 72 hours. Cells were then fixed and permeabilized with Perm/Wash buffer (BD Biosciences) for 30 min. After permeabilization, cells were washed with phosphate-buffered saline (PBS) and incubated with 1:2000 dilution of anti-flavivirus antibody (MAB10216, EMD Millipore) in Perm/Wash buffer for 2 hours. After incubation, cells were washed with PBS and incubated with anti-mouse horseradish peroxidase (HRP)-conjugated secondary antibody (115035146, Jackson ImmunoResearch Laboratories) for 2 hrs. Plates were washed and developed with peroxidase substrate (KPL). The half maximal inhibitory concentration (IC50) of the mAbs was calculated using a nonlinear regression analysis with GraphPad Prism software.

### Serum and Purified IgG ELISAs

For NS1 and E binding ELISAs, 96-well plates (Corning; Cat #3690) were coated with 5 g/ml of NS1 or E protein diluted in PBS and incubated overnight at 4° C. Wells were washed and then blocked with 5% non-fat dried milk (NFDM) in PBS for 1 hour at 37° C. Wells were washed 3 times with PBS and serial dilutions of human plasm in 5% NFDM-PBS were added and incubated for 1 hour at 37° C. Plates were then washed 3 times with PBS and secondary cross-adsorbed anti-human IgG-HRP (Thermo Fisher Scientific; cat #31413) or anti-human-IgM (Sigma Aldrich; cat #AP114P) detection antibodies were added at 1:8000 dilution in 5% NFDM-PBS for 1 hour at 37° C. After washing 3 times with PBS detection reagent was added per manufacturer recommendations (Thermo Scientific; Cat #34029) and absorbance was measures at 450 nM wavelength using a Spectramax microplate Reader (Molecular Devices).

For virus binding ELISAs, 96-well ELISA plates were coated with 5 ug/ml of 4G2 (Millipore MAB10216) diluted in PBS and incubated for 2 hours at 37° C. After washing 3 times with PBS, whole YFV-17D viral particles diluted in PBS pH 7.4 and incubated overnight at 4° C. Plates were then washed 3 times with PBS and blocked with 5% NFDM-PBS for 1 hour at 37° C. After removal of the blocking solution, test antibodies diluted in 5% NFDM-PBS were allowed to bind for 1 hour at 37° C. Plates were then washed 3 times with PBS and secondary cross-adsorbed anti-human IgG-HRP (Thermo Fisher Scientific; cat #31413) or anti-human-IgM (Sigma Aldrich; cat #AP114P) detection antibodies were added at 1:8000 dilution in 5% NFDM-PBS for 1 hour at 37° C. After washing 3 times with PBS detection reagent was added per manufacturer recommendations (Thermo Scientific; Cat #34029) and absorbance was measures at 450 nM wavelength using a Spectramax microplate Reader (Molecular Devices).

Binding of purified IgGs to viral particles was performed as described above. IgGs were diluted in 5% NFDM-PBS and tested at 100 nM concentration for single point reactivity test of plasmablast- and MBC-derived day 14 antibodies.

All references, patents, and patent publications cited herein are hereby incorporated by reference in their entireties for all that is taught therein.

1.-54. (canceled)

55. A method of treating or preventing a Yellow Fever Virus (YFV) infection or at least one symptom arising therefrom, comprising administering to a patient in need thereof or suspected of being in need thereof an isolated antibody or antigen-binding fragment thereof,
wherein the antibody or antigen-binding fragment thereof specifically binds to a YFV protein, wherein the antibody or antigen-binding fragment thereof comprises:
(i) a heavy chain variable region comprising the amino acid sequence of SEQ ID NO:307 and a light chain variable region comprising the amino acid sequence of SEQ ID NO:459;
(ii) a heavy chain variable region comprising the amino acid sequence of SEQ ID NO:308 and a light chain variable region comprising the amino acid sequence of SEQ ID NO:460;
(iii) a heavy chain variable region comprising the amino acid sequence of SEQ ID NO:311 and a light chain variable region comprising the amino acid sequence of SEQ ID NO:463;
(iv) a heavy chain variable region comprising the amino acid sequence of SEQ ID NO:320 and a light chain variable region comprising the amino acid sequence of SEQ ID NO:471; or
(v) a heavy chain variable region comprising the amino acid sequence of SEQ ID NO:341 and a light chain variable region comprising the amino acid sequence of SEQ ID NO:492.

56. The method of claim 55, wherein the antibody or antigen-binding fragment thereof comprises a heavy chain variable region comprising the amino acid sequence of SEQ ID NO:307 and a light chain variable region comprising the amino acid sequence of SEQ ID NO:459.

57. The method of claim 55, wherein the antibody or antigen-binding fragment thereof comprises a heavy chain variable region comprising the amino acid sequence of SEQ ID NO:308 and a light chain variable region comprising the amino acid sequence of SEQ ID NO:460.

58. The method of claim 55, wherein the antibody or antigen-binding fragment thereof comprises a heavy chain variable region comprising the amino acid sequence of SEQ ID NO:311 and a light chain variable region comprising the amino acid sequence of SEQ ID NO:463.

59. The method of claim 55, wherein the antibody or antigen-binding fragment thereof comprises a heavy chain variable region comprising the amino acid sequence of SEQ ID NO:320 and a light chain variable region comprising the amino acid sequence of SEQ ID NO:471.

60. The method of claim 55, wherein the antibody or antigen-binding fragment thereof comprises a heavy chain variable region comprising the amino acid sequence of SEQ ID NO:341 and a light chain variable region comprising the amino acid sequence of SEQ ID NO:492.

61. The method of claim 55, wherein the at least one symptom associated with the YFV infection is treated, alleviated, or reduced in severity.

62. The method of claim 55, wherein the at least one symptom associated with the YFV infection is fever, chills, headache, low back pain, myalgia, loss of appetite, nausea, vomiting, or fatigue.

63. The method of claim 55, wherein the isolated antibody or antigen-binding fragment thereof:
(a) displays neutralization activity toward YFV in vitro;
(b) displays an in vitro neutralization potency (IC50) of between about 0.5 microgram/milliliter (μl/ml) to about 5 μl/ml; between about 0.05 μl/ml to about 0.5 μl/ml; or less than about 0.05 mg/ml; and/or
(c) binds to an envelope protein of YFV.

64. The method of claim 63, wherein the isolated antibody or antigen-binding fragment thereof comprises at least two of characteristics (a), (b), and/or (c).

65. The method of claim 55, wherein the isolated antibody or antigen-binding fragment thereof displays an equilibrium dissociation constant of between about 1×10⁶ M to about 1×10¹⁰ M.

66. A method of treating or preventing a Yellow Fever Virus (YFV) infection or at least one symptom arising therefrom, comprising administering to a patient in need thereof or suspected of being in need thereof an isolated nucleic acid encoding an antibody or antigen-binding fragment thereof,
wherein the antibody or antigen-binding fragment thereof specifically binds to a YFV protein, wherein the antibody or antigen-binding fragment thereof comprises:
(i) a heavy chain variable region comprising the amino acid sequence of SEQ ID NO:307 and a light chain variable region comprising the amino acid sequence of SEQ ID NO:459;
(ii) a heavy chain variable region comprising the amino acid sequence of SEQ ID NO:308 and a light chain variable region comprising the amino acid sequence of SEQ ID NO:460;
(iii) a heavy chain variable region comprising the amino acid sequence of SEQ ID NO:311 and a light chain variable region comprising the amino acid sequence of SEQ ID NO:463;
(iv) a heavy chain variable region comprising the amino acid sequence of SEQ ID NO:320 and a light chain variable region comprising the amino acid sequence of SEQ ID NO:471; or
(v) a heavy chain variable region comprising the amino acid sequence of SEQ ID NO:341 and a light chain variable region comprising the amino acid sequence of SEQ ID NO:492.

67. The method of claim 66, wherein the at least one symptom associated with the YFV infection is fever, chills, headache, low back pain, myalgia, loss of appetite, nausea, vomiting, or fatigue.

68. A method of treating or preventing a Yellow Fever Virus (YFV) infection or at least one symptom arising therefrom, comprising administering to a patient in need thereof or suspected of being in need thereof a pharmaceutical composition comprising a pharmaceutically acceptable excipient and an isolated antibody or antigen-binding fragment thereof;
wherein the antibody or antigen-binding fragment thereof specifically binds to a YFV protein, wherein the antibody or antigen-binding fragment thereof comprises:
(i) a heavy chain variable region comprising the amino acid sequence of SEQ ID NO:307 and a light chain variable region comprising the amino acid sequence of SEQ ID NO:459;
(ii) a heavy chain variable region comprising the amino acid sequence of SEQ ID NO:308 and a light chain variable region comprising the amino acid sequence of SEQ ID NO:460;
(iii) a heavy chain variable region comprising the amino acid sequence of SEQ ID NO:311 and a light chain variable region comprising the amino acid sequence of SEQ ID NO:463;
(iv) a heavy chain variable region comprising the amino acid sequence of SEQ ID NO:320 and a light chain variable region comprising the amino acid sequence of SEQ ID NO:471; or
(v) a heavy chain variable region comprising the amino acid sequence of SEQ ID NO:341 and a light chain variable region comprising the amino acid sequence of SEQ ID NO:492.

69. The method of claim 68, wherein the at least one symptom associated with the YFV infection is fever, chills, headache, low back pain, myalgia, loss of appetite, nausea, vomiting, or fatigue.

70. A method of treating or preventing a Yellow Fever Virus (YFV) infection or at least one symptom arising therefrom, comprising administering to a patient in need thereof or suspected of being in need thereof a pharmaceutical composition comprising a pharmaceutically acceptable excipient and an isolated nucleic acid encoding an antibody or antigen-binding fragment thereof;
wherein the antibody or antigen-binding fragment thereof specifically binds to a YFV protein, wherein the antibody or antigen-binding fragment thereof comprises:
(i) a heavy chain variable region comprising the amino acid sequence of SEQ ID NO:307 and a light chain variable region comprising the amino acid sequence of SEQ ID NO:459;
(ii) a heavy chain variable region comprising the amino acid sequence of SEQ ID NO:308 and a light chain variable region comprising the amino acid sequence of SEQ ID NO:460;
(iii) a heavy chain variable region comprising the amino acid sequence of SEQ ID NO:311 and a light chain variable region comprising the amino acid sequence of SEQ ID NO:463;
(iv) a heavy chain variable region comprising the amino acid sequence of SEQ ID NO:320 and a light chain variable region comprising the amino acid sequence of SEQ ID NO:471; or
(v) a heavy chain variable region comprising the amino acid sequence of SEQ ID NO:341 and a light chain variable region comprising the amino acid sequence of SEQ ID NO:492.

71. The method of claim 70, wherein the at least one symptom associated with the YFV infection is fever, chills, headache, low back pain, myalgia, loss of appetite, nausea, vomiting, or fatigue.


SEQUENCE LISTING (1014 sequences)

SEQ ID NO: 1 - protein, 18 aa, synthetic construct
  REGION 1..18: note = Synthetic polypeptide
ARNAPENYYGSGRESFDI

SEQ ID NO: 2 - protein, 11 aa, synthetic construct
  REGION 1..11: note = Synthetic polypeptide
GTWDSSLSAWV

SEQ ID NO: 3 - protein, 15 aa, synthetic construct
  REGION 1..15: note = Synthetic polypeptide
AKDHGGKYGWWYFDL

SEQ ID NO: 4 - protein, 9 aa, synthetic construct
  REGION 1..9: note = Synthetic polypeptide
QQYDNWPLT

SEQ ID NO: 5 - protein, 18 aa, synthetic construct
  REGION 1..18: note = Synthetic polypeptide
ARNAPENYYGSGRESFDI

SEQ ID NO: 6 - protein, 11 aa, synthetic construct
  REGION 1..11: note = Synthetic polypeptide
GTWDSSLSAWV

SEQ ID NO: 7 - protein, 19 aa, synthetic construct
  REGION 1..19: note = Synthetic polypeptide
ARDLEVGAEYLYYHYGMDV

SEQ ID NO: 8 - protein, 11 aa, synthetic construct
  REGION 1..11: note = Synthetic polypeptide
AAWDDSLNGWV

SEQ ID NO: 9 - protein, 17 aa, synthetic construct
  REGION 1..17: note = Synthetic polypeptide
AKDSSTSWYQVVYHIDY

SEQ ID NO: 10 - protein, 11 aa, synthetic construct
  REGION 1..11: note = Synthetic polypeptide
ETWDSSLNAVV

SEQ ID NO: 11 - protein, 15 aa, synthetic construct
  REGION 1..15: note = Synthetic polypeptide
AKDLAVSTPRYWFDS

SEQ ID NO: 12 - protein, 10 aa, synthetic construct
  REGION 1..10: note = Synthetic polypeptide
QQSYSIPRIT

SEQ ID NO: 13 - protein, 15 aa, synthetic construct
  REGION 1..15: note = Synthetic polypeptide
AKDMAVSVHRGWFDD

SEQ ID NO: 14 - protein, 10 aa, synthetic construct
  REGION 1..10: note = Synthetic polypeptide
QQSYSPPMYT

SEQ ID NO: 15 - protein, 19 aa, synthetic construct
  REGION 1..19: note = Synthetic polypeptide
ARDLEVGAEYIYYYYGMDV

SEQ ID NO: 16 - protein, 11 aa, synthetic construct
  REGION 1..11: note = Synthetic polypeptide
AAWDDSRNGWV

SEQ ID NO: 17 - protein, 15 aa, synthetic construct
  REGION 1..15: note = Synthetic polypeptide
ARSHWRSPQSVTFDL

SEQ ID NO: 18 - protein, 13 aa, synthetic construct
  REGION 1..13: note = Synthetic polypeptide
GTWDTSSLSAGRV

SEQ ID NO: 19 - protein, 16 aa, synthetic construct
  REGION 1..16: note = Synthetic polypeptide
ARIAAGYSTSWYYFDY

SEQ ID NO: 20 - protein, 12 aa, synthetic construct
  REGION 1..12: note = Synthetic polypeptide
GTWDTSLSAGRV

SEQ ID NO: 21 - protein, 15 aa, synthetic construct
  REGION 1..15: note = Synthetic polypeptide
AKDMWAGTTTNWFGP

SEQ ID NO: 22 - protein, 10 aa, synthetic construct
  REGION 1..10: note = Synthetic polypeptide
GTWDTSLGVV

SEQ ID NO: 23 - protein, 11 aa, synthetic construct
  REGION 1..11: note = Synthetic polypeptide
AREFSSRPFDL

SEQ ID NO: 24 - protein, 12 aa, synthetic construct
  REGION 1..12: note = Synthetic polypeptide
CSYAGTYTSNYV

SEQ ID NO: 25 - protein, 16 aa, synthetic construct
  REGION 1..16: note = Synthetic polypeptide
ARVNPPQYSSGWYSVY

SEQ ID NO: 26 - protein, 11 aa, synthetic construct
  REGION 1..11: note = Synthetic polypeptide
GTWDNSLGAVV

SEQ ID NO: 27 - protein, 15 aa, synthetic construct
  REGION 1..15: note = Synthetic polypeptide
ARVAWTSSSSCYYDY

SEQ ID NO: 28 - protein, 18 aa, synthetic construct
  REGION 1..18: note = Synthetic polypeptide
ARDGEGHYYRSGDNWFDR

SEQ ID NO: 29 - protein, 11 aa, synthetic construct
  REGION 1..11: note = Synthetic polypeptide
GTWDSSLSAVV

SEQ ID NO: 30 - protein, 13 aa, synthetic construct
  REGION 1..13: note = Synthetic polypeptide
ARAELSAWYYFDH

SEQ ID NO: 31 - protein, 12 aa, synthetic construct
  REGION 1..12: note = Synthetic polypeptide
GTWDTSLSAGRV

SEQ ID NO: 32 - protein, 19 aa, synthetic construct
  REGION 1..19: note = Synthetic polypeptide
ARVSPLDDGYGYTYYGMDV

SEQ ID NO: 33 - protein, 10 aa, synthetic construct
  REGION 1..10: note = Synthetic polypeptide
QQYNNWPPRT

SEQ ID NO: 34 - protein, 16 aa, synthetic construct
  REGION 1..16: note = Synthetic polypeptide
ARDWAELTTITNYFYP

SEQ ID NO: 35 - protein, 9 aa, synthetic construct
  REGION 1..9: note = Synthetic polypeptide
QQAKSFPPT

SEQ ID NO: 36 - protein, 22 aa, synthetic construct
  REGION 1..22: note = Synthetic polypeptide
AKAENRIGYCSAGSCYLTYFDY

SEQ ID NO: 37 - protein, 10 aa, synthetic construct
  REGION 1..10: note = Synthetic polypeptide
NSYTSSSTLV

SEQ ID NO: 38 - protein, 15 aa, synthetic construct
  REGION 1..15: note = Synthetic polypeptide
AKDPKYSSGWWAFDY

SEQ ID NO: 39 - protein, 8 aa, synthetic construct
  REGION 1..8: note = Synthetic polypeptide
QQYDDWPL

SEQ ID NO: 40 - protein, 15 aa, synthetic construct
  REGION 1..15: note = Synthetic polypeptide
ARVEWAYSSSWWLDY

SEQ ID NO: 41 - protein, 12 aa, synthetic construct
  REGION 1..12: note = Synthetic polypeptide
GTWDTSLSAGGV

SEQ ID NO: 42 - protein, 20 aa, synthetic construct
  REGION 1..20: note = Synthetic polypeptide
AKHTGDKPLVWAPSVYGLDV

SEQ ID NO: 43 - protein, 10 aa, synthetic construct
  REGION 1..10: note = Synthetic polypeptide
SSYTRRSTLV

SEQ ID NO: 44 - protein, 14 aa, synthetic construct
  REGION 1..14: note = Synthetic polypeptide
ARVSVSTSAWYADY

SEQ ID NO: 45 - protein, 10 aa, synthetic construct
  REGION 1..10: note = Synthetic polypeptide
GTWDTSLSTV

SEQ ID NO: 46 - protein, 10 aa, synthetic construct
  REGION 1..10: note = Synthetic polypeptide
ARELSSRIDY

SEQ ID NO: 47 - protein, 11 aa, synthetic construct
  REGION 1..11: note = Synthetic polypeptide
SSYPGTSALVI

SEQ ID NO: 48 - protein, 17 aa, synthetic construct
  REGION 1..17: note = Synthetic polypeptide
ARAQDGQQLVNYYGMDV

SEQ ID NO: 49 - protein, 9 aa, synthetic construct
  REGION 1..9: note = Synthetic polypeptide
QQSYSTPYT

SEQ ID NO: 50 - protein, 20 aa, synthetic construct
  REGION 1..20: note = Synthetic polypeptide
ARGGDYGDYESNNPAEYFQH

SEQ ID NO: 51 - protein, 12 aa, synthetic construct
  REGION 1..12: note = Synthetic polypeptide
QSYDSSLSGHVV

SEQ ID NO: 52 - protein, 14 aa, synthetic construct
  REGION 1..14: note = Synthetic polypeptide
AGHREDPYGAYGAS

SEQ ID NO: 53 - protein, 9 aa, synthetic construct
  REGION 1..9: note = Synthetic polypeptide
QQRTNWPFT

SEQ ID NO: 54 - protein, 15 aa, synthetic construct
  REGION 1..15: note = Synthetic polypeptide
ASRKEVRGTEDYFDY

SEQ ID NO: 55 - protein, 9 aa, synthetic construct
  REGION 1..9: note = Synthetic polypeptide
HQRTNWPWT

SEQ ID NO: 56 - protein, 15 aa, synthetic construct
  REGION 1..15: note = Synthetic polypeptide
AKVEEDGYTNVVRDY

SEQ ID NO: 57 - protein, 9 aa, synthetic construct
  REGION 1..9: note = Synthetic polypeptide
LQRTNWPFT

SEQ ID NO: 58 - protein, 10 aa, synthetic construct
  REGION 1..10: note = Synthetic polypeptide
AREGTRGRMD

SEQ ID NO: 59 - protein, 11 aa, synthetic construct
  REGION 1..11: note = Synthetic polypeptide
SSYTSGTTLGV

SEQ ID NO: 60 - protein, 15 aa, synthetic construct
  REGION 1..15: note = Synthetic polypeptide
ARDSWSGPTRNWFDP

SEQ ID NO: 61 - protein, 11 aa, synthetic construct
  REGION 1..11: note = Synthetic polypeptide
GTWDSSLGGVI

SEQ ID NO: 62 - protein, 15 aa, synthetic construct
  REGION 1..15: note = Synthetic polypeptide
ARVVWEYSNAWCVDF

SEQ ID NO: 63 - protein, 11 aa, synthetic construct
  REGION 1..11: note = Synthetic polypeptide
ETWDSSLGVVV

SEQ ID NO: 64 - protein, 14 aa, synthetic construct
  REGION 1..14: note = Synthetic polypeptide
ARNTYYDRSGLIAY

SEQ ID NO: 65 - protein, 10 aa, synthetic construct
  REGION 1..10: note = Synthetic polypeptide
QQYDNLSRLT

SEQ ID NO: 66 - protein, 13 aa, synthetic construct
  REGION 1..13: note = Synthetic polypeptide
ARGPLKSYWYFDL

SEQ ID NO: 67 - protein, 12 aa, synthetic construct
  REGION 1..12: note = Synthetic polypeptide
GTWDTSLSAGRV

SEQ ID NO: 68 - protein, 17 aa, synthetic construct
  REGION 1..17: note = Synthetic polypeptide
ARYCSGATCYGSNGMDV

SEQ ID NO: 69 - protein, 10 aa, synthetic construct
  REGION 1..10: note = Synthetic polypeptide
GTWDFRLSAL

SEQ ID NO: 70 - protein, 13 aa, synthetic construct
  REGION 1..13: note = Synthetic polypeptide
AKDQCGGDCTADY

SEQ ID NO: 71 - protein, 11 aa, synthetic construct
  REGION 1..11: note = Synthetic polypeptide
SSYTSSGTPVV

SEQ ID NO: 72 - protein, 15 aa, synthetic construct
  REGION 1..15: note = Synthetic polypeptide
ASTLWGGPLSVASDY

SEQ ID NO: 73 - protein, 12 aa, synthetic construct
  REGION 1..12: note = Synthetic polypeptide
GTWDSSPSAGRV

SEQ ID NO: 74 - protein, 14 aa, synthetic construct
  REGION 1..14: note = Synthetic polypeptide
ARDYYASGDGYFDY

SEQ ID NO: 75 - protein, 9 aa, synthetic construct
  REGION 1..9: note = Synthetic polypeptide
QQYYSTPRT

SEQ ID NO: 76 - protein, 17 aa, synthetic construct
  REGION 1..17: note = Synthetic polypeptide
VRYCSSTSCYGLNGMDV

SEQ ID NO: 77 - protein, 10 aa, synthetic construct
  REGION 1..10: note = Synthetic polypeptide
GTWDTRLSAL

SEQ ID NO: 78 - protein, 12 aa, synthetic construct
  REGION 1..12: note = Synthetic polypeptide
ARDGSLVNAIDY

SEQ ID NO: 79 - protein, 12 aa, synthetic construct
  REGION 1..12: note = Synthetic polypeptide
GTWDTSLSAAWV

SEQ ID NO: 80 - protein, 15 aa, synthetic construct
  REGION 1..15: note = Synthetic polypeptide
ARVRWSGSTSWDLDY

SEQ ID NO: 81 - protein, 12 aa, synthetic construct
  REGION 1..12: note = Synthetic polypeptide
GTWDTSPSAGGV

SEQ ID NO: 82 - protein, 23 aa, synthetic construct
  REGION 1..23: note = Synthetic polypeptide
AHSPRRITMVRGVIITWGDGMDV

SEQ ID NO: 83 - protein, 11 aa, synthetic construct
  REGION 1..11: note = Synthetic polypeptide
SSYTSSSTLAV

SEQ ID NO: 84 - protein, 12 aa, synthetic construct
  REGION 1..12: note = Synthetic polypeptide
ARDGSMVNAIDY

SEQ ID NO: 85 - protein, 12 aa, synthetic construct
  REGION 1..12: note = Synthetic polypeptide
GTWDSSLSAAWV

SEQ ID NO: 86 - protein, 14 aa, synthetic construct
  REGION 1..14: note = Synthetic polypeptide
ARDAYASGDGGIDY

SEQ ID NO: 87 - protein, 11 aa, synthetic construct
  REGION 1..11: note = Synthetic polypeptide
SSYRSSGTPYV

SEQ ID NO: 88 - protein, 15 aa, synthetic construct
  REGION 1..15: note = Synthetic polypeptide
AKDLRGVGGWYYFDY

SEQ ID NO: 89 - protein, 9 aa, synthetic construct
  REGION 1..9: note = Synthetic polypeptide
QQYDNLPLT

SEQ ID NO: 90 - protein, 14 aa, synthetic construct
  REGION 1..14: note = Synthetic polypeptide
AKDQGVTTDWPSDY

SEQ ID NO: 91 - protein, 9 aa, synthetic construct
  REGION 1..9: note = Synthetic polypeptide
QHYETYSVR

SEQ ID NO: 92 - protein, 19 aa, synthetic construct
  REGION 1..19: note = Synthetic polypeptide
PRDGLPGANQYFFYYGMDV

SEQ ID NO: 93 - protein, 9 aa, synthetic construct
  REGION 1..9: note = Synthetic polypeptide
QKYNSAPLT

SEQ ID NO: 94 - protein, 15 aa, synthetic construct
  REGION 1..15: note = Synthetic polypeptide
VRVEEYVNNEEVRDY

SEQ ID NO: 95 - protein, 9 aa, synthetic construct
  REGION 1..9: note = Synthetic polypeptide
LQRTNWPFT

SEQ ID NO: 96 - protein, 14 aa, synthetic construct
  REGION 1..14: note = Synthetic polypeptide
ARDQGFTTDWPCDY

SEQ ID NO: 97 - protein, 9 aa, synthetic construct
  REGION 1..9: note = Synthetic polypeptide
QHYNSFSVK

SEQ ID NO: 98 - protein, 12 aa, synthetic construct
  REGION 1..12: note = Synthetic polypeptide
ARDSNFNSNLDY

SEQ ID NO: 99 - protein, 12 aa, synthetic construct
  REGION 1..12: note = Synthetic polypeptide
QVWDSSSDHPWV

SEQ ID NO: 100 - protein, 13 aa, synthetic construct
  REGION 1..13: note = Synthetic polypeptide
ARGPLKTYWYFDL

SEQ ID NO: 101 - protein, 12 aa, synthetic construct
  REGION 1..12: note = Synthetic polypeptide
GTWDTSLSAGRV

SEQ ID NO: 102 - protein, 12 aa, synthetic construct
  REGION 1..12: note = Synthetic polypeptide
ARDSNYFYGLDV

SEQ ID NO: 103 - protein, 12 aa, synthetic construct
  REGION 1..12: note = Synthetic polypeptide
QVWDTSIDHHWV

SEQ ID NO: 104 - protein, 14 aa, synthetic construct
  REGION 1..14: note = Synthetic polypeptide
AKDICSGDCGGGDY

SEQ ID NO: 105 - protein, 11 aa, synthetic construct
  REGION 1..11: note = Synthetic polypeptide
SSYAGSNNWVV

SEQ ID NO: 106 - protein, 12 aa, synthetic construct
  REGION 1..12: note = Synthetic polypeptide
AREDDDYYSMDV

SEQ ID NO: 107 - protein, 10 aa, synthetic construct
  REGION 1..10: note = Synthetic polypeptide
SSYTTTSLVI

SEQ ID NO: 108 - protein, 22 aa, synthetic construct
  REGION 1..22: note = Synthetic polypeptide
ARDISCISTSCYGGYYYYGMDV

SEQ ID NO: 109 - protein, 10 aa, synthetic construct
  REGION 1..10: note = Synthetic polypeptide
MQALQTPPRT

SEQ ID NO: 110 - protein, 14 aa, synthetic construct
  REGION 1..14: note = Synthetic polypeptide
ARDYYASGDGSIDY

SEQ ID NO: 111 - protein, 9 aa, synthetic construct
  REGION 1..9: note = Synthetic polypeptide
LQHNSYPLT

SEQ ID NO: 112 - protein, 14 aa, synthetic construct
  REGION 1..14: note = Synthetic polypeptide
AKYYDSSGYYYFDY

SEQ ID NO: 113 - protein, 9 aa, synthetic construct
  REGION 1..9: note = Synthetic polypeptide
KQYNRNPYT

SEQ ID NO: 114 - protein, 13 aa, synthetic construct
  REGION 1..13: note = Synthetic polypeptide
AKGSVSVAGAEDY

SEQ ID NO: 115 - protein, 9 aa, synthetic construct
  REGION 1..9: note = Synthetic polypeptide
QKYNSAPQT

SEQ ID NO: 116 - protein, 14 aa, synthetic construct
  REGION 1..14: note = Synthetic polypeptide
AKGYDSSGYYWADY

SEQ ID NO: 117 - protein, 10 aa, synthetic construct
  REGION 1..10: note = Synthetic polypeptide
QQYNNWPPLT

SEQ ID NO: 118 - protein, 14 aa, synthetic construct
  REGION 1..14: note = Synthetic polypeptide
ARERGGYFTEPFDI

SEQ ID NO: 119 - protein, 9 aa, synthetic construct
  REGION 1..9: note = Synthetic polypeptide
QQYYRTPWT

SEQ ID NO: 120 - protein, 12 aa, synthetic construct
  REGION 1..12: note = Synthetic polypeptide
AATIFGVVSFDY

SEQ ID NO: 121 - protein, 11 aa, synthetic construct
  REGION 1..11: note = Synthetic polypeptide
GTWDSALGAAV

SEQ ID NO: 122 - protein, 14 aa, synthetic construct
  REGION 1..14: note = Synthetic polypeptide
AKYYDSSGYYYLDY

SEQ ID NO: 123 - protein, 9 aa, synthetic construct
  REGION 1..9: note = Synthetic polypeptide
QQYNRDPYT

SEQ ID NO: 124 - protein, 10 aa, synthetic construct
  REGION 1..10: note = Synthetic polypeptide
CRESGEGFDP

SEQ ID NO: 125 - protein, 10 aa, synthetic construct
  REGION 1..10: note = Synthetic polypeptide
QSADRSGSVI

SEQ ID NO: 126 - protein, 26 aa, synthetic construct
  REGION 1..26: note = Synthetic polypeptide
ARDQSHGTFGGVIDSTTLFYYYGMDV

SEQ ID NO: 127 - protein, 8 aa, synthetic construct
  REGION 1..8: note = Synthetic polypeptide
QQRSNWPS

SEQ ID NO: 128 - protein, 19 aa, synthetic construct
  REGION 1..19: note = Synthetic polypeptide
ARGYCSSTSCFYYYYGMDV

SEQ ID NO: 129 - protein, 9 aa, synthetic construct
  REGION 1..9: note = Synthetic polypeptide
QQSYSTPLT

SEQ ID NO: 130 - protein, 22 aa, synthetic construct
  REGION 1..22: note = Synthetic polypeptide
ARDHYFDSSGDYLSYYYNGMDV

SEQ ID NO: 131 - protein, 9 aa, synthetic construct
  REGION 1..9: note = Synthetic polypeptide
QQYGSSPRA

SEQ ID NO: 132 - protein, 10 aa, synthetic construct
  REGION 1..10: note = Synthetic polypeptide
ARVYGGPDDY

SEQ ID NO: 133 - protein, 9 aa, synthetic construct
  REGION 1..9: note = Synthetic polypeptide
QQSSITPPT

SEQ ID NO: 134 - protein, 16 aa, synthetic construct
  REGION 1..16: note = Synthetic polypeptide
AKDGVTTINGWFHFEY

SEQ ID NO: 135 - protein, 9 aa, synthetic construct
  REGION 1..9: note = Synthetic polypeptide
QQYNSFPFT

SEQ ID NO: 136 - protein, 13 aa, synthetic construct
  REGION 1..13: note = Synthetic polypeptide
TRITGDRYWYLDL

SEQ ID NO: 137 - protein, 9 aa, synthetic construct
  REGION 1..9: note = Synthetic polypeptide
QQTYSASGS

SEQ ID NO: 138 - protein, 20 aa, synthetic construct
  REGION 1..20: note = Synthetic polypeptide
ARGWFGYSNYGLYYYYGMDV

SEQ ID NO: 139 - protein, 9 aa, synthetic construct
  REGION 1..9: note = Synthetic polypeptide
QQSYSTPWT

SEQ ID NO: 140 - protein, 13 aa, synthetic construct
  REGION 1..13: note = Synthetic polypeptide
ARDFWSGSNWFDP

SEQ ID NO: 141 - protein, 10 aa, synthetic construct
  REGION 1..10: note = Synthetic polypeptide
GTWDNSLGVV

SEQ ID NO: 142 - protein, 25 aa, synthetic construct
  REGION 1..25: note = Synthetic polypeptide
AKDIGDSYGSGSYYLPYGAYYGMDV

SEQ ID NO: 143 - protein, 8 aa, synthetic construct
  REGION 1..8: note = Synthetic polypeptide
QQYGSSPG

SEQ ID NO: 144 - protein, 14 aa, synthetic construct
  REGION 1..14: note = Synthetic polypeptide
AKHYDSSGYYYEDY

SEQ ID NO: 145 - protein, 9 aa, synthetic construct
  REGION 1..9: note = Synthetic polypeptide
HQYKDFPWT

SEQ ID NO: 146 - protein, 10 aa, synthetic construct
  REGION 1..10: note = Synthetic polypeptide
ARVRDGEYDY

SEQ ID NO: 147 - protein, 8 aa, synthetic construct
  REGION 1..8: note = Synthetic polypeptide
QQYNSYSP

SEQ ID NO: 148 - protein, 23 aa, synthetic construct
  REGION 1..23: note = Synthetic polypeptide
ARDNSEVEDYGDYVLYHYYGMDV

SEQ ID NO: 149 - protein, 8 aa, synthetic construct
  REGION 1..8: note = Synthetic polypeptide
QQYGSSPF

SEQ ID NO: 150 - protein, 13 aa, synthetic construct
  REGION 1..13: note = Synthetic polypeptide
AKDQCGGDCTADY

SEQ ID NO: 151 - protein, 11 aa, synthetic construct
  REGION 1..11: note = Synthetic polypeptide
SSYTSSSTPVV

SEQ ID NO: 152 - protein, 12 aa, synthetic construct
  REGION 1..12: note = Synthetic polypeptide
ARGYTGYDGFDY

SEQ ID NO: 153 - protein, 10 aa, synthetic construct
  REGION 1..10: note = Synthetic polypeptide
CSYATNYGVV

SEQ ID NO: 154 - protein, 18 aa, synthetic construct
  REGION 1..18: note = Synthetic polypeptide
ARRPYYYGSRRPAGHMDV

SEQ ID NO: 155 - protein, 9 aa, synthetic construct
  REGION 1..9: note = Synthetic polypeptide
QSYDSSNVV

SEQ ID NO: 156 - protein, 11 aa, synthetic construct
  REGION 1..11: note = Synthetic polypeptide
GRDSDKNYFDY

SEQ ID NO: 157 - protein, 12 aa, synthetic construct
  REGION 1..12: note = Synthetic polypeptide
GAWDSSLSAHVV

SEQ ID NO: 158 - protein, 14 aa, synthetic construct
  REGION 1..14: note = Synthetic polypeptide
AKTYDSNAYYYLDY

SEQ ID NO: 159 - protein, 9 aa, synthetic construct
  REGION 1..9: note = Synthetic polypeptide
QQYNRYPYT

SEQ ID NO: 160 - protein, 18 aa, synthetic construct
  REGION 1..18: note = Synthetic polypeptide
ASLWFIVMTMSKNPETDY

SEQ ID NO: 161 - protein, 9 aa, synthetic construct
  REGION 1..9: note = Synthetic polypeptide
LQHHSYPWT

SEQ ID NO: 162 - protein, 14 aa, synthetic construct
  REGION 1..14: note = Synthetic polypeptide
AKYYDSSGYYYFDH

SEQ ID NO: 163 - protein, 9 aa, synthetic construct
  REGION 1..9: note = Synthetic polypeptide
QQYNRDPYT

SEQ ID NO: 164 - protein, 14 aa, synthetic construct
  REGION 1..14: note = Synthetic polypeptide
AKFYDSSGYYYFDY

SEQ ID NO: 165 - protein, 9 aa, synthetic construct
  REGION 1..9: note = Synthetic polypeptide
QQYNTYPYT

SEQ ID NO: 166 - protein, 13 aa, synthetic construct
  REGION 1..13: note = Synthetic polypeptide
VRLYGDYVAYFDY

SEQ ID NO: 167 - protein, 9 aa, synthetic construct
  REGION 1..9: note = Synthetic polypeptide
QQSYSTPWT

SEQ ID NO: 168 - protein, 21 aa, synthetic construct
  REGION 1..21: note = Synthetic polypeptide
ARRGTTVTRFGVIQYYYGMDV

SEQ ID NO: 169 - protein, 10 aa, synthetic construct
  REGION 1..10: note = Synthetic polypeptide
SSYTSSSTLV

SEQ ID NO: 170 - protein, 11 aa, synthetic construct
  REGION 1..11: note = Synthetic polypeptide
ARETANNWFDP

SEQ ID NO: 171 - protein, 11 aa, synthetic construct
  REGION 1..11: note = Synthetic polypeptide
QVWDNSSDRRV

SEQ ID NO: 172 - protein, 14 aa, synthetic construct
  REGION 1..14: note = Synthetic polypeptide
ARASMMPRPPVHDY

SEQ ID NO: 173 - protein, 8 aa, synthetic construct
  REGION 1..8: note = Synthetic polypeptide
QQYNTWWT

SEQ ID NO: 174 - protein, 16 aa, synthetic construct
  REGION 1..16: note = Synthetic polypeptide
AKDRSQGDYGDYVADY

SEQ ID NO: 175 - protein, 9 aa, synthetic construct
  REGION 1..9: note = Synthetic polypeptide
QQSYSTPLT

SEQ ID NO: 176 - protein, 18 aa, synthetic construct
  REGION 1..18: note = Synthetic polypeptide
ARVQTSHSELWFGEFGAD

SEQ ID NO: 177 - protein, 9 aa, synthetic construct
  REGION 1..9: note = Synthetic polypeptide
QQYNTWPKT

SEQ ID NO: 178 - protein, 14 aa, synthetic construct
  REGION 1..14: note = Synthetic polypeptide
AKDGGYSTDWYFDL

SEQ ID NO: 179 - protein, 9 aa, synthetic construct
  REGION 1..9: note = Synthetic polypeptide
QQYGSSRRT

SEQ ID NO: 180 - protein, 14 aa, synthetic construct
  REGION 1..14: note = Synthetic polypeptide
AKGYDSNGYYYIDY

SEQ ID NO: 181 - protein, 9 aa, synthetic construct
  REGION 1..9: note = Synthetic polypeptide
QQYNRYPYT

SEQ ID NO: 182 - protein, 16 aa, synthetic construct
  REGION 1..16: note = Synthetic polypeptide
ARDVGYQLLQVYGMDV

SEQ ID NO: 183 - protein, 12 aa, synthetic construct
  REGION 1..12: note = Synthetic polypeptide
SSYTSSSTLDVV

SEQ ID NO: 184 - protein, 21 aa, synthetic construct
  REGION 1..21: note = Synthetic polypeptide
ARAEYDTSGYYQQRLPEYFQH

SEQ ID NO: 185 - protein, 10 aa, synthetic construct
  REGION 1..10: note = Synthetic polypeptide
QQYNSWPPIT

SEQ ID NO: 186 - protein, 14 aa, synthetic construct
  REGION 1..14: note = Synthetic polypeptide
AKYYDSSGYYYFHS

SEQ ID NO: 187 - protein, 9 aa, synthetic construct
  REGION 1..9: note = Synthetic polypeptide
QQYNRYPYT

SEQ ID NO: 188 - protein, 11 aa, synthetic construct
  REGION 1..11: note = Synthetic polypeptide
AREHGDYGLDY

SEQ ID NO: 189 - protein, 12 aa, synthetic construct
  REGION 1..12: note = Synthetic polypeptide
ATWDVSLSNDVL

SEQ ID NO: 190 - protein, 17 aa, synthetic construct
  REGION 1..17: note = Synthetic polypeptide
YVDYYYDSSGYYSPFDY

SEQ ID NO: 191 - protein, 10 aa, synthetic construct
  REGION 1..10: note = Synthetic polypeptide
QQYGSSPPIT

SEQ ID NO: 192 - protein, 12 aa, synthetic construct
  REGION 1..12: note = Synthetic polypeptide
ARVDGEEVALIY

SEQ ID NO: 193 - protein, 9 aa, synthetic construct
  REGION 1..9: note = Synthetic polypeptide
QQSSTTRWT

SEQ ID NO: 194 - protein, 13 aa, synthetic construct
  REGION 1..13: note = Synthetic polypeptide
VRVWGGEAARYDY

SEQ ID NO: 195 - protein, 9 aa, synthetic construct
  REGION 1..9: note = Synthetic polypeptide
QHASTTPWT

SEQ ID NO: 196 - protein, 10 aa, synthetic construct
  REGION 1..10: note = Synthetic polypeptide
SRHMGFGLDL

SEQ ID NO: 197 - protein, 11 aa, synthetic construct
  REGION 1..11: note = Synthetic polypeptide
QAWDTTTAGGV

SEQ ID NO: 198 - protein, 14 aa, synthetic construct
  REGION 1..14: note = Synthetic polypeptide
ARDYYGSGDGYFDY

SEQ ID NO: 199 - protein, 9 aa, synthetic construct
  REGION 1..9: note = Synthetic polypeptide
QQYGSSPRA

SEQ ID NO: 200 - protein, 16 aa, synthetic construct
  REGION 1..16: note = Synthetic polypeptide
ARIPVEYGTPRGSFDT

SEQ ID NO: 201 - protein, 10 aa, synthetic construct
  REGION 1..10: note = Synthetic polypeptide
SSYGGNNDLV

SEQ ID NO: 202 - protein, 7 aa, synthetic construct
  REGION 1..7: note = Synthetic polypeptide
AGGSPDY

SEQ ID NO: 203 - protein, 9 aa, synthetic construct
  REGION 1..9: note = Synthetic polypeptide
QQRSNWPYT

SEQ ID NO: 204 - protein, 14 aa, synthetic construct
  REGION 1..14: note = Synthetic polypeptide
ARAYDSRGYYYIEH

SEQ ID NO: 205 - protein, 8 aa, synthetic construct
  REGION 1..8: note = Synthetic polypeptide
QQYKTYWT

SEQ ID NO: 206 - protein, 12 aa, synthetic construct
  REGION 1..12: note = Synthetic polypeptide
AREIDSNYVFDY

SEQ ID NO: 207 - protein, 10 aa, synthetic construct
  REGION 1..10: note = Synthetic polypeptide
SSYTSSGTNI

SEQ ID NO: 208 - protein, 15 aa, synthetic construct
  REGION 1..15: note = Synthetic polypeptide
ARKLSYSSGWYYFDY

SEQ ID NO: 209 - protein, 10 aa, synthetic construct
  REGION 1..10: note = Synthetic polypeptide
QQYNNWPPLT

SEQ ID NO: 210 - protein, 10 aa, synthetic construct
  REGION 1..10: note = Synthetic polypeptide
VTTTVILFDY

SEQ ID NO: 211 - protein, 11 aa, synthetic construct
  REGION 1..11: note = Synthetic polypeptide
YSTDSSGLLGV

SEQ ID NO: 212 - protein, 20 aa, synthetic construct
  REGION 1..20: note = Synthetic polypeptide
ARGRLAWGLRGQKSPNFFAY

SEQ ID NO: 213 - protein, 9 aa, synthetic construct
  REGION 1..9: note = Synthetic polypeptide
QQFHSPPWT

SEQ ID NO: 214 - protein, 16 aa, synthetic construct
  REGION 1..16: note = Synthetic polypeptide
ATAGIFGVVIMKGFDH

SEQ ID NO: 215 - protein, 9 aa, synthetic construct
  REGION 1..9: note = Synthetic polypeptide
QQYNDYPWT

SEQ ID NO: 216 - protein, 16 aa, synthetic construct
  REGION 1..16: note = Synthetic polypeptide
ARETYYYGSGSVPVHD

SEQ ID NO: 217 - protein, 9 aa, synthetic construct
  REGION 1..9: note = Synthetic polypeptide
LQHNTYPWT

SEQ ID NO: 218 - protein, 15 aa, synthetic construct
  REGION 1..15: note = Synthetic polypeptide
ARGYDSSGYWGFGDN

SEQ ID NO: 219 - protein, 9 aa, synthetic construct
  REGION 1..9: note = Synthetic polypeptide
QQYYSYPYT

SEQ ID NO: 220 - protein, 13 aa, synthetic construct
  REGION 1..13: note = Synthetic polypeptide
ARVEGGAWGAFDI

SEQ ID NO: 221 - protein, 10 aa, synthetic construct
  REGION 1..10: note = Synthetic polypeptide
QSADRSGTVV

SEQ ID NO: 222 - protein, 14 aa, synthetic construct
  REGION 1..14: note = Synthetic polypeptide
ARLWFTEYPGAFDI

SEQ ID NO: 223 - protein, 10 aa, synthetic construct
  REGION 1..10: note = Synthetic polypeptide
SSYAGSNALV

SEQ ID NO: 224 - protein, 17 aa, synthetic construct
  REGION 1..17: note = Synthetic polypeptide
ARHSSGSYYLAGYYFDY

SEQ ID NO: 225 - protein, 9 aa, synthetic construct
  REGION 1..9: note = Synthetic polypeptide
QSYDSSNWV

SEQ ID NO: 226 - protein, 10 aa, synthetic construct
  REGION 1..10: note = Synthetic polypeptide
ARLTDSGYDD

SEQ ID NO: 227 - protein, 8 aa, synthetic construct
  REGION 1..8: note = Synthetic polypeptide
HSPDSHVV

SEQ ID NO: 228 - protein, 20 aa, synthetic construct
  REGION 1..20: note = Synthetic polypeptide
ARETCSGGSCYYRVGSAFDI

SEQ ID NO: 229 - protein, 11 aa, synthetic construct
  REGION 1..11: note = Synthetic polypeptide
QVWDSSSDHEV

SEQ ID NO: 230 - protein, 15 aa, synthetic construct
  REGION 1..15: note = Synthetic polypeptide
VKDYCSGGRCYSFDY

SEQ ID NO: 231 - protein, 5 aa, synthetic construct
  REGION 1..5: note = Synthetic polypeptide
QQWGT

SEQ ID NO: 232 - protein, 14 aa, synthetic construct
  REGION 1..14: note = Synthetic polypeptide
AKAYDSSAYYYLDY

SEQ ID NO: 233 - protein, 9 aa, synthetic construct
  REGION 1..9: note = Synthetic polypeptide
QQYNRYPYT

SEQ ID NO: 234 - protein, 14 aa, synthetic construct
  REGION 1..14: note = Synthetic polypeptide
AKAYDSRGYYYLDY

SEQ ID NO: 235 - protein, 9 aa, synthetic construct
  REGION 1..9: note = Synthetic polypeptide
QQYNRYSYT

SEQ ID NO: 236 - protein, 22 aa, synthetic construct
  REGION 1..22: note = Synthetic polypeptide
AKDLTHRLGSIFGKLTFDAFDI

SEQ ID NO: 237 - protein, 8 aa, synthetic construct
  REGION 1..8: note = Synthetic polypeptide
QQYNNFWT

SEQ ID NO: 238 - protein, 16 aa, synthetic construct
  REGION 1..16: note = Synthetic polypeptide
AKDLTPYFYDSGAFDH

SEQ ID NO: 239 - protein, 11 aa, synthetic construct
  REGION 1..11: note = Synthetic polypeptide
HSYDSNMSGSV

SEQ ID NO: 240 - protein, 10 aa, synthetic construct
  REGION 1..10: note = Synthetic polypeptide
ARVFGGPTDY

SEQ ID NO: 241 - protein, 10 aa, synthetic construct
  REGION 1..10: note = Synthetic polypeptide
QKYYSAPLIT

SEQ ID NO: 242 - protein, 9 aa, synthetic construct
  REGION 1..9: note = Synthetic polypeptide
ARVVNGLDV

SEQ ID NO: 243 - protein, 13 aa, synthetic construct
  REGION 1..13: note = Synthetic polypeptide
QSADSSVADSSVV

SEQ ID NO: 244 - protein, 7 aa, synthetic construct
  REGION 1..7: note = Synthetic polypeptide
ARGQPDY

SEQ ID NO: 245 - protein, 9 aa, synthetic construct
  REGION 1..9: note = Synthetic polypeptide
QQRSNWPYT

SEQ ID NO: 246 - protein, 14 aa, synthetic construct
  REGION 1..14: note = Synthetic polypeptide
AGKKWELLGFRFDP

SEQ ID NO: 247 - protein, 11 aa, synthetic construct
  REGION 1..11: note = Synthetic polypeptide
GTWDNSLGMVV

SEQ ID NO: 248 - protein, 10 aa, synthetic construct
  REGION 1..10: note = Synthetic polypeptide
ARQWLGHFDY

SEQ ID NO: 249 - protein, 10 aa, synthetic construct
  REGION 1..10: note = Synthetic polypeptide
SSYTSSSTYV

SEQ ID NO: 250 - protein, 10 aa, synthetic construct
  REGION 1..10: note = Synthetic polypeptide
ARVFSYYLDY

SEQ ID NO: 251 - protein, 11 aa, synthetic construct
  REGION 1..11: note = Synthetic polypeptide
QQPGNWPPAFT

SEQ ID NO: 252 - protein, 18 aa, synthetic construct
  REGION 1..18: note = Synthetic polypeptide
AHRHIAARLYRDDDVFDV

SEQ ID NO: 253 - protein, 8 aa, synthetic construct
  REGION 1..8: note = Synthetic polypeptide
QQYNNWIT

SEQ ID NO: 254 - protein, 12 aa, synthetic construct
  REGION 1..12: note = Synthetic polypeptide
ARGLNTVTNSDY

SEQ ID NO: 255 - protein, 9 aa, synthetic construct
  REGION 1..9: note = Synthetic polypeptide
QQANSFPWT

SEQ ID NO: 256 - protein, 13 aa, synthetic construct
  REGION 1..13: note = Synthetic polypeptide
ASGLSPDFSVLDV

SEQ ID NO: 257 - protein, 9 aa, synthetic construct
  REGION 1..9: note = Synthetic polypeptide
MQALQTPYT

SEQ ID NO: 258 - protein, 23 aa, synthetic construct
  REGION 1..23: note = Synthetic polypeptide
AREGAGYYDSSGYYPLSYDAFDI

SEQ ID NO: 259 - protein, 11 aa, synthetic construct
  REGION 1..11: note = Synthetic polypeptide
AAWDDNLIGVV

SEQ ID NO: 260 - protein, 10 aa, synthetic construct
  REGION 1..10: note = Synthetic polypeptide
ARVRGSYWDY

SEQ ID NO: 261 - protein, 11 aa, synthetic construct
  REGION 1..11: note = Synthetic polypeptide
SSFAGSNNLYV

SEQ ID NO: 262 - protein, 9 aa, synthetic construct
  REGION 1..9: note = Synthetic polypeptide
GRDRGWLDI

SEQ ID NO: 263 - protein, 10 aa, synthetic construct
  REGION 1..10: note = Synthetic polypeptide
SSYTRSSTRV

SEQ ID NO: 264 - protein, 23 aa, synthetic construct
  REGION 1..23: note = Synthetic polypeptide
ARVIRDLRDYYDGSGYGPDAFDI

SEQ ID NO: 265 - protein, 10 aa, synthetic construct
  REGION 1..10: note = Synthetic polypeptide
ETWDSRLSVV

SEQ ID NO: 266 - protein, 16 aa, synthetic construct
  REGION 1..16: note = Synthetic polypeptide
ARARWEDGNYYYGMDV

SEQ ID NO: 267 - protein, 11 aa, synthetic construct
  REGION 1..11: note = Synthetic polypeptide
GTWDSSLSAVV

SEQ ID NO: 268 - protein, 17 aa, synthetic construct
  REGION 1..17: note = Synthetic polypeptide
AKDQSSGWPNYYYGMDV

SEQ ID NO: 269 - protein, 9 aa, synthetic construct
  REGION 1..9: note = Synthetic polypeptide
QQSYSTPWT

SEQ ID NO: 270 - protein, 19 aa, synthetic construct
  REGION 1..19: note = Synthetic polypeptide
VRGYCSSTSCYGGLYWFDP

SEQ ID NO: 271 - protein, 9 aa, synthetic construct
  REGION 1..9: note = Synthetic polypeptide
QQSYSTPRT

SEQ ID NO: 272 - protein, 19 aa, synthetic construct
  REGION 1..19: note = Synthetic polypeptide
ARHSGGYSSKDKPTEYFQH

SEQ ID NO: 273 - protein, 11 aa, synthetic construct
  REGION 1..11: note = Synthetic polypeptide
QSYDSSLSGVV

SEQ ID NO: 274 - protein, 15 aa, synthetic construct
  REGION 1..15: note = Synthetic polypeptide
ARDVGVAAVITGSVR

SEQ ID NO: 275 - protein, 9 aa, synthetic construct
  REGION 1..9: note = Synthetic polypeptide
QQFYTTPST

SEQ ID NO: 276 - protein, 19 aa, synthetic construct
  REGION 1..19: note = Synthetic polypeptide
ARGYCSSTSCYGGLYWFDP

SEQ ID NO: 277 - protein, 9 aa, synthetic construct
  REGION 1..9: note = Synthetic polypeptide
QQSYSTPRT

SEQ ID NO: 278 - protein, 26 aa, synthetic construct
  REGION 1..26: note = Synthetic polypeptide
ARDGAGDYIWGSYRHKGLHYYYGMDV

SEQ ID NO: 279 - protein, 9 aa, synthetic construct
  REGION 1..9: note = Synthetic polypeptide
LQHNSYPLT

SEQ ID NO: 280 - protein, 17 aa, synthetic construct
  REGION 1..17: note = Synthetic polypeptide
AKDPRTFYGVVMLLDDP

SEQ ID NO: 281 - protein, 9 aa, synthetic construct
  REGION 1..9: note = Synthetic polypeptide
QSYDSTTVV

SEQ ID NO: 282 - protein, 12 aa, synthetic construct
  REGION 1..12: note = Synthetic polypeptide
ARGFGELPGFDI

SEQ ID NO: 283 - protein, 11 aa, synthetic construct
  REGION 1..11: note = Synthetic polypeptide
SSYAGSNNFVV

SEQ ID NO: 284 - protein, 10 aa, synthetic construct
  REGION 1..10: note = Synthetic polypeptide
ARDSWGPFDY

SEQ ID NO: 285 - protein, 11 aa, synthetic construct
  REGION 1..11: note = Synthetic polypeptide
GTWDSSLSAKV

SEQ ID NO: 286 - protein, 14 aa, synthetic construct
  REGION 1..14: note = Synthetic polypeptide
AKTYDSRAYYYLDY

SEQ ID NO: 287 - protein, 9 aa, synthetic construct
  REGION 1..9: note = Synthetic polypeptide
QQYNRYPYT

SEQ ID NO: 288 - protein, 16 aa, synthetic construct
  REGION 1..16: note = Synthetic polypeptide
AKDLFYDFWTGITIDY

SEQ ID NO: 289 - protein, 11 aa, synthetic construct
  REGION 1..11: note = Synthetic polypeptide
CSYAGSYTFVL

SEQ ID NO: 290 - protein, 13 aa, synthetic construct
  REGION 1..13: note = Synthetic polypeptide
ARDGGTVSDGLDV

SEQ ID NO: 291 - protein, 8 aa, synthetic construct
  REGION 1..8: note = Synthetic polypeptide
QQTFSIWT

SEQ ID NO: 292 - protein, 15 aa, synthetic construct
  REGION 1..15: note = Synthetic polypeptide
ARVVWYSSSSHLFDY

SEQ ID NO: 293 - protein, 12 aa, synthetic construct
  REGION 1..12: note = Synthetic polypeptide
GTWDSSLSAGKV

SEQ ID NO: 294 - protein, 10 aa, synthetic construct
  REGION 1..10: note = Synthetic polypeptide
ARIKSDAFDL

SEQ ID NO: 295 - protein, 10 aa, synthetic construct
  REGION 1..10: note = Synthetic polypeptide
FSYAGSNNYV

SEQ ID NO: 296 - protein, 16 aa, synthetic construct
  REGION 1..16: note = Synthetic polypeptide
AKFPLRDGGSGEGFDY

SEQ ID NO: 297 - protein, 9 aa, synthetic construct
  REGION 1..9: note = Synthetic polypeptide
MQASQFPLT

SEQ ID NO: 298 - protein, 14 aa, synthetic construct
  REGION 1..14: note = Synthetic polypeptide
ARNTYYDRRRTFDY

SEQ ID NO: 299 - protein, 10 aa, synthetic construct
  REGION 1..10: note = Synthetic polypeptide
QQYDNLPPVT

SEQ ID NO: 300 - protein, 13 aa, synthetic construct
  REGION 1..13: note = Synthetic polypeptide
AGVGITGTTGIDY

SEQ ID NO: 301 - protein, 10 aa, synthetic construct
  REGION 1..10: note = Synthetic polypeptide
QAWDSSTDVV

SEQ ID NO: 302 - protein, 18 aa, synthetic construct
  REGION 1..18: note = Synthetic polypeptide
AKGAAAGPFPYFYYAMDV

SEQ ID NO: 303 - protein, 9 aa, synthetic construct
  REGION 1..9: note = Synthetic polypeptide
QKYQSAPPT

SEQ ID NO: 304 - protein, 125 aa, synthetic construct
  REGION 1..125: note = Synthetic polypeptide
QVQLQESGPGLVKPSETLSLTCAVSGYSISSGFYWGWIRQPPGKGLEWIGSMYQSGITYYNPSLKSRVTISVDTSKSQFSLKLTSVTAADTAMYYCARNAPENYYGSGRESFDIWGQGTMVTVSS

SEQ ID NO: 305 - protein, 122 aa, synthetic construct
  REGION 1..122: note = Synthetic polypeptide
QVQLQESGGDLVQPGGSLRLSCAASGFTFSNYAMNWVRQAPGKGLEWVSAINRGGDSTYYADSVKGRFTISRDNSKNTLYLQMNSLRAEDTAVYYCAKDHGGKYGWWYFDLWGRGTLVTVSS

SEQ ID NO: 306 - protein, 125 aa, synthetic construct
  REGION 1..125: note = Synthetic polypeptide
QVQLQESGPGLVKPSETLSLTCAVSGYSISSGFYWGWIRQPPGKGLEWIGSMYHSGITYYNPSLKSRVTISVDTSKNQFSLKLTSVTAADTAMYYCARNAPENYYGSGRESFDIWGQGTTVTVSS

SEQ ID NO: 307 - protein, 126 aa, synthetic construct
  REGION 1..126: note = Synthetic polypeptide
EVQLVESGGGLVQPGRPLRLSCAASGFAFSSYGMHWVRQAPGKGLEWVALIRFDGTIKYYADSVKGRFTISRDNAKNTLYLQMSSLRAEDTAVYYCARDLEVGAEYLYYHYGMDVWGQGTTVTVSS

SEQ ID NO: 308 - protein, 124 aa, synthetic construct
  REGION 1..124: note = Synthetic polypeptide
EVQLVESGGGVVQPGRSLRLSCAASGFTFNSHGMHWVRQAPGKGLEWVAVISYDGTKKYFADSVKGRFTISRDNSKNTLYLQMSSLRADDTAVYYCAKDSSTSWYQVVYHIDYWGQGTLVTVSS

SEQ ID NO: 309 - protein, 122 aa, synthetic construct
  REGION 1..122: note = Synthetic polypeptide
EVQLLESGGGLVQPGGSLRLSCAASGFTFRNYAMNWVRQTPGKGLEWVSGISGGGDSTNYADSVKGRFTISRDNSRNTLYLQLNSLRAEDTAVYYCAKDLAVSTPRYWFDSWGQGTLVTVSS

SEQ ID NO: 310 - protein, 122 aa, synthetic construct
  REGION 1..122: note = Synthetic polypeptide
EVQLVESGGGLVQPGGSLRLSCAASGLIFRNYAMSWVRQAPGKGLEWVSSFSGSGGSAYYADSVKGRFTISRDNSKSTVYLQMNRLRVEDTAVYYCAKDMAVSVHRGWFDDWGQGTLVTVSS

SEQ ID NO: 311 - protein, 126 aa, synthetic construct
  REGION 1..126: note = Synthetic polypeptide
QVQLVESGGGVVQPGRSLRLSCAASGFAFSSYGMHWVRQAPGKGLEWVAGMRFDGTKIYYADSVKGRFTISRDNSKNTLYLQMNSLRAEDTAVYFCARDLEVGAEYIYYYYGMDVWGQGTTVTVSS

SEQ ID NO: 312 - protein, 122 aa, synthetic construct
  REGION 1..122: note = Synthetic polypeptide
QVQLVESGPGLVKPSGTLSLTCAVSGGSISSDYWWSWVRQPPGKGLEYIGEIYHTGSTNYNPSLKSRVTVSLDRSKNVFSLTLRSVTAADTAVYYCARSHWRSPQSVTFDLWGQGTTVTVSS

SEQ ID NO: 313 - protein, 123 aa, synthetic construct
  REGION 1..123: note = Synthetic polypeptide
QVQLQESGPGLVKPSGTLSLTCAVSGGSITSSNWWSWVRQPPGKGLEWIGDIYHSGSTSYNPSLKSRVTISVDKSKNHFSLKLTSVTAADTAVYYCARIAAGYSTSWYYFDYWGQGTLVTVSS

SEQ ID NO: 314 - protein, 122 aa, synthetic construct
  REGION 1..122: note = Synthetic polypeptide
EVQLVETGSGLVRPSGTLSLTCAVSGDSISSNNWWSWVRQPPGKGLEWIGEIYHSGSTSYNPSLKSRVTISIDKSNNHFSLKLTSVTAADTAVYYCAKDMWAGTTTNWFGPWGQGTLVTVSS

SEQ ID NO: 315 - protein, 118 aa, synthetic construct
  REGION 1..118: note = Synthetic polypeptide
QVTLKESGGALVKPAGSLTLSCAASGFTFGDYYMSWIRQAPGKGLEWISYISSSGSSIYYTDSVRGRFTISRDNARNSLYLQMNSLRVEDTAVYYCAREFSSRPFDLWGQGTLVTVSS

SEQ ID NO: 316 - protein, 123 aa, synthetic construct
  REGION 1..123: note = Synthetic polypeptide
EVQLQESGPGLVKPSGTLSLTCAVSGGSISSSDWWSWVRQPPGKGLEWIGEIYHSGSTSYNPSVKSRVSISVDKSKNQFSLQLSSVTAADTAIYYCARVNPPQYSSGWYSVYWGQGTLVTVSS

SEQ ID NO: 317 - protein, 122 aa, synthetic construct
  REGION 1..122: note = Synthetic polypeptide
QVQLQQSGPGLVKPSGTLSLTCAVSGDSISSSHWWCWVRQPPGKGLEWIGEIYHSGSTSYNPSLKSRVTISVDKSKNQFSLKLSSVTAADTAVYFCARVAWTSSSSCYYDYWGQGTLVTVSS

SEQ ID NO: 318 - protein, 125 aa, synthetic construct
  REGION 1..125: note = Synthetic polypeptide
EVQLVESGPGLVKPSGTLSLTCAVSGGSISSSYWWSWVRQSPGKGLEWIGEVYHSGSTHYNPSLKSRVTISVDKSKNQFSLKLTSVTAADTAVYYCARDGEGHYYRSGDNWFDRWGQGTLVTVSS

SEQ ID NO: 319 - protein, 120 aa, synthetic construct
  REGION 1..120: note = Synthetic polypeptide
EVQLLESGPGLVQPSGTLSLTCTASGGSISSSNWWSWVRQPPGKGLEWIGDIYHTGSTSYNPSLKSRVTISVDKSKNQFSLKLSSVTAADTAVYYCARAELSAWYYFDHWGQGTLVTVSS

SEQ ID NO: 320 - protein, 126 aa, synthetic construct
  REGION 1..126: note = Synthetic polypeptide
QVQLVESGGGLVKPGGSLRLSCAASGFIFSDYYMNWIRQAPGKGLDWVSTISGSGKSIYYADSVKGRFTISRDNAKNSLYLQMNSLSAEDTAVYYCARVSPLDDGYGYTYYGMDVWGQGTTVTVSS

SEQ ID NO: 321 - protein, 123 aa, synthetic construct
  REGION 1..123: note = Synthetic polypeptide
EVQLLESGGGLVKPGGSLRLSCAASGFTFSDYYMSWIRQAPGKGLEWVSYITSSGNTKYYADSVKGRFTISRDNAKNSLYLQISSLRAEDTAVYYCARDWAELTTITNYFYPWGQGTTVTVSS

SEQ ID NO: 322 - protein, 129 aa, synthetic construct
  REGION 1..129: note = Synthetic polypeptide
EVQLLESGGGLVQPGRSLRLSCAASGFTFDDYAMHWVRQPPGKGLEWVSGISWNGGGIGYADSVKGRFTISRDNAKNSLYLQMNSLRADDTALYYCAKAENRIGYCSAGSCYLTYFDYWGQGTLVTVSS

SEQ ID NO: 323 - protein, 122 aa, synthetic construct
  REGION 1..122: note = Synthetic polypeptide
QVQLVQSGGGLVQPGGSLRLSCAASGFTFSSYAMSWVRQAPGKGLEWVSAISGSGGSTYYADSVKGRFTISRDNSKNTLHLQMSSLRAEDTAVYYCAKDPKYSSGWWAFDYWGQGTLVTVSS

SEQ ID NO: 324 - protein, 122 aa, synthetic construct
  REGION 1..122: note = Synthetic polypeptide
EVQLVESGPGLVKPSGTLSLTCAVSGGSISSNKWWSWVRQPPGKGLEWIGEIYHSGSTSYNPSLKSRVSISVDKSKNQFSLKLSSVTAADTAVYYCARVEWAYSSSWWLDYWGQGTLVTVSS

SEQ ID NO: 325 - protein, 127 aa, synthetic construct
  REGION 1..127: note = Synthetic polypeptide
QVQLVESGGGLVKPGGSLRLSCAASGFTFSDDYMSWIRQAPGKGLEWVSYISGSGRAMYYADSVQGRFTVSRDNAKNSLFLQMNNLRAEDTAVYYCAKHTGDKPLVWAPSVYGLDVWGQGTTVTVSS

SEQ ID NO: 326 - protein, 121 aa, synthetic construct
  REGION 1..121: note = Synthetic polypeptide
QVQLQESGPGLVKPSGTLSLTCAVSGSSITSSHWWSWVRQPPGKGLAWIGDIYHSGGTTYNPSLKSRVTISVDKSKNQFSLKLSSVTAADTAVYYCARVSVSTSAWYADYWGQGTLVTVSS

SEQ ID NO: 327 - protein, 117 aa, synthetic construct
  REGION 1..117: note = Synthetic polypeptide
QVQLVESGGGLVKPGGSLRLSCVASGFTFNNYYMRWMRQAPGKGLEWVSQISSSGSIKDYADSVKGRFTVSRDNAKNSLYLQLNSLRADDTAVYFCARELSSRIDYWGQGTLVTVSS

SEQ ID NO: 328 - protein, 124 aa, synthetic construct
  REGION 1..124: note = Synthetic polypeptide
EVQLVESGGGVVQPGRSLRLSCVASGFTLRSYGMHWVRQVPGKGLEWVAVSWYDGSNKHYADSVKGRFSISRDNSKNTLYLQMNSLRAEDTAVYYCARAQDGQQLVNYYGMDVWGQGTTVTVSS

SEQ ID NO: 329 - protein, 127 aa, synthetic construct
  REGION 1..127: note = Synthetic polypeptide
QVQLVESGGGVVQPGRSLRLSCAASGFTFSSYTMHWVRQAPGKGLEWVAVISYDGSNKYYADSVKGRFTISRDNSKNTLYLQMNSLRAEDTAVYYCARGGDYGDYESNNPAEYFQHWGQGTLVTVSS

SEQ ID NO: 330 - protein, 120 aa, synthetic construct
  REGION 1..120: note = Synthetic polypeptide
QVQLQESGPGLVKPSETLSLTCTVSGDSISVSYWSWIRQFPGKGLEWIGYIYNSGNANYNPSLESRVTISIDTSKNRFSLRLSSVTAADTAVYYCAGHREDPYGAYGASWGQGTLVTVSS

SEQ ID NO: 331 - protein, 123 aa, synthetic construct
  REGION 1..123: note = Synthetic polypeptide
EVQLLESGPGLVKPSETLSLTCTVSGGSLSSDSHFWGWIRQPPGKGLEWIGYIYYSGNANYNPSLQSRVTISLDKSKNQFSLRLTSVTAADTAVYYCASRKEVRGTEDYFDYWGQGTLVTVSS

SEQ ID NO: 332 - protein, 123 aa, synthetic construct
  REGION 1..123: note = Synthetic polypeptide
EVQLQESGPGLVKPSETLSLTCTVSGGSVSSGSYYWSWIRQPPGKGLEWIGYIYDSGNTNYNPSLKSRVTISVDTSKRQFSLRLTSVTAADTAVYYCAKVEEDGYTNVVRDYWGQGTLVTVSS

SEQ ID NO: 333 - protein, 117 aa, synthetic construct
  REGION 1..117: note = Synthetic polypeptide
EVQLVESGGGLVKPGGSLRLSCAASGFTFSDYYMSWIRQAPGKGLECIACISSSGSMIYYADSVKGRFTISRDNAKNSLYLQLNSLRVEDTAVYYCAREGTRGRMDWGQGTLVTVSS

SEQ ID NO: 334 - protein, 122 aa, synthetic construct
  REGION 1..122: note = Synthetic polypeptide
EVQLLESGPGLVRPSGTLSLTCAVSGGSISTTDWWSWVRQPPGKGLEWIGEINQSGSTSYSPSFKSRVSISVDKSKRQFSLKLTSVTAADTAVYYCARDSWSGPTRNWFDPWGRGTLVTVSS

SEQ ID NO: 335 - protein, 122 aa, synthetic construct
  REGION 1..122: note = Synthetic polypeptide
EVQLLESGPGLVKPSGTLSLTCAVSGGSISSGNWWSWVRQPPGKGLEWIGEIYHSGSANYNPSLKSRVTISVDKSKNQFSLKLTSVTAADTAVYYCARVVWEYSNAWCVDFWGQGTTVTVSS

SEQ ID NO: 336 - protein, 121 aa, synthetic construct
  REGION 1..121: note = Synthetic polypeptide
EVQLLESGGGVVQPGRSLRLSCAASGFTFTTYAMHWVRQAPGKGLEWVAAVSYDGNNKYYADSVKGRFTISRDNSRNTLYLQMNSLRAEDTAVYFCARNTYYDRSGLIAYWGQGALVTVSS

SEQ ID NO: 337 - protein, 120 aa, synthetic construct
  REGION 1..120: note = Synthetic polypeptide
QVQLVESGPGLVKPSGTLSLTCAVSGDSISSTNWWSWVRQPPGKGLEYIGEIFHSGSTNYNPFLKSRVTISVDKSKNHFSLKLSSVTAADTAVYYCARGPLKSYWYFDLWGRGTLVTVSS

SEQ ID NO: 338 - protein, 124 aa, synthetic construct
  REGION 1..124: note = Synthetic polypeptide
QVQLQESGPGLVKPSGTLSLTCAVSGGSISSNNWWSWVRQPPGKGLEWIGDTYHSGSPSYNPSLKSRVTISVDKSKNEFSLKLSSVTAADTAVYFCARYCSGATCYGSNGMDVWGQGTTVTVSS

SEQ ID NO: 339 - protein, 120 aa, synthetic construct
  REGION 1..120: note = Synthetic polypeptide
QVQLQESGGGVVQPGRSLRLSCAASGFTFSNFGMHWVRQAPGKGLEWVAIISYDRSNKDYADSVKGRFTISRDNSKNTLYLQMNSLRAEDTAVYYCAKDQCGGDCTADYWGQGTLVTVSS

SEQ ID NO: 340 - protein, 122 aa, synthetic construct
  REGION 1..122: note = Synthetic polypeptide
EVQLLESGPGLVRPSGTLSLTCAVSGASISSNHWWTWVRQPPGKGLEWIGEIYHSGSPTYNPSLKSRVTISVDKSKNQFSLKLNSVTAADTAVYYCASTLWGGPLSVASDYWGQGTLVTVSS

SEQ ID NO: 341 - protein, 121 aa, synthetic construct
  REGION 1..121: note = Synthetic polypeptide
QVQLVESGGGVVQPGRSLRLSCAASGFTFSNSGMHWVRQAPGQGLEWVALISYTGETKYYSDSLKARFTISRDNSKNTLYLQMSSLSNEDTAVYYCARDYYASGDGYFDYWGQGTLVTVSS

SEQ ID NO: 342 - protein, 124 aa, synthetic construct
  REGION 1..124: note = Synthetic polypeptide
QVQLQQWGPELVKPSGTLSLTCTVSGGSISSISWWSWVRQSPGKGLEWIGEINHSGSTVYNPSLKSRVTISVDKSKKQFSLKLRSVTAADTAVYYCVRYCSSTSCYGLNGMDVWGQGTTVTVSS

SEQ ID NO: 343 - protein, 119 aa, synthetic construct
  REGION 1..119: note = Synthetic polypeptide
QVQLVQSGGGLVNPGGSLRLSCAASGFTFTDYYMSWIRQAPGKGLEWVSYISSSGNTRYYADSVKGRFTISRDNAKNSLSLQMNSLRPEDTAIYYCARDGSLVNAIDYWGQGTLVTVSS

SEQ ID NO: 344 - protein, 122 aa, synthetic construct
  REGION 1..122: note = Synthetic polypeptide
EVQLVESGPGLVKPSGTLSLTCAVSGGSITGSNWWSWVRQPPGKGLEWIGEIYHTGSTSYNPSLKSRVTISVDNSKNHFSLRLTSVTAADTAVYYCARVRWSGSTSWDLDYWGQGTLVTVSS

SEQ ID NO: 345 - protein, 131 aa, synthetic construct
  REGION 1..131: note = Synthetic polypeptide
EVTLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHSPRRITMVRGVIITWGDGMDVWGQGTTVTVSS

SEQ ID NO: 346 - protein, 119 aa, synthetic construct
  REGION 1..119: note = Synthetic polypeptide
EVQLVESGGGLVKPGGSLRLSCAASGFTFTDYYMSWIRQAPGKGLEWVSYITSSGNTKYYADSVKGRFTISRDNAKNSLFLQMNSLRAEDTAVYFCARDGSMVNAIDYWGQGTLVTVSS

SEQ ID NO: 347 - protein, 121 aa, synthetic construct
  REGION 1..121: note = Synthetic polypeptide
QVQLVESGGGVVQPGRSLRLSCAASGFTFSNSGMHWVRQAPGKGLEWVSVIWYDESNKYYADSVKGRFTISRDNSKNTVYLQMNTLRAEDTAVYYCARDAYASGDGGIDYWGQGALVTVSS

SEQ ID NO: 348 - protein, 122 aa, synthetic construct
  REGION 1..122: note = Synthetic polypeptide
EVQLLESGGGLVQPGGSLRLSCAASGFTFSSYAMSWVRQAPGKGLEWVSVISDSGGSTYYADSVKGRFTISRDNSKNTLYLQMNSLRAEDTAVYYCAKDLRGVGGWYYFDYWGQGTLVTVSS

SEQ ID NO: 349 - protein, 121 aa, synthetic construct
  REGION 1..121: note = Synthetic polypeptide
EVQLVESGGGLVQPGGSLRLSCAASGFTFINYAMTWVRQAPGKGLEWVSAISGNGDGTYYADSVKGRFTLSRDNAKNTIYLHMSALRDEDTALYYCAKDQGVTTDWPSDYWGQGTLVTVSS

SEQ ID NO: 350 - protein, 126 aa, synthetic construct
  REGION 1..126: note = Synthetic polypeptide
QVQLVESGGGVVQPGRSLRLSCAASGFTFSSYAMHWVRQAPGKGLEWVAVISHDGSNKYYADSVKGRFTISRDNSKNTLYLQINSLRAEDTAVYYCPRDGLPGANQYFFYYGMDVWGQGTTVTVSS

SEQ ID NO: 351 - protein, 123 aa, synthetic construct
  REGION 1..123: note = Synthetic polypeptide
EVQLLESGPRLVKPSETLSLTCTVSGGSVRGGSHYWSWIRQPPGKGLEWIGYVYDSGSTNYNPSLKSRVSISVDMSKKQFSLKLRSVTAADTAVYHCVRVEEYVNNEEVRDYWGQGTMVTVSS

SEQ ID NO: 352 - protein, 121 aa, synthetic construct
  REGION 1..121: note = Synthetic polypeptide
EVQLLESGGGLVPPGGSLRLSCAASGFTFSNYAMSWVRQAPGKGLEWVSAISGSGDSTYYADSVKGRFTLSRDTSKKMVYLHMSNLRDDDTAVYYCARDQGFTTDWPCDYWGQGTLVTVSS

SEQ ID NO: 353 - protein, 119 aa, synthetic construct
  REGION 1..119: note = Synthetic polypeptide
QVQLVESGGGLVKPGGSLRLSCAASGFTFSDYYMSWIRQAPGKGLEWVSYITSSGNTMYYADSVKGRFTISRDNAKNSLYLQMNSLRAEDTAVYYCARDSNFNSNLDYWGQGTLVTVSS

SEQ ID NO: 354 - protein, 120 aa, synthetic construct
  REGION 1..120: note = Synthetic polypeptide
QVQLQESGPGLVKPSGTLSLTCAVSGGSISSSNWWSWVRQPPGKGLEWIGEIYHSGSTTYNPSLKSRVTISVDKSKNQFSLKLSSVTAADTAVYYCARGPLKTYWYFDLWGRGTLVTVSS

SEQ ID NO: 355 - protein, 119 aa, synthetic construct
  REGION 1..119: note = Synthetic polypeptide
EVQLVESGGGLVKPGGSLRLSCAASGFTFSDYYMSWIRQAPGKGLEWVSYISSSGNTIYYADSVKGRFTISRDNAKNSLYLQLNSLRAGDTAVYYCARDSNYFYGLDVWGQGTTVTVSS

SEQ ID NO: 356 - protein, 121 aa, synthetic construct
  REGION 1..121: note = Synthetic polypeptide
EVQLVESGGGVVQPGRSLRLSCAASGFTFSNYGMHWVRQAPGKGLEWVAVISYDGSNKYYADSVKGRFTISRDDSKNTLYLQVNSLRAEDTAVYYCAKDICSGDCGGGDYWGQGTLVTVSS

SEQ ID NO: 357 - protein, 119 aa, synthetic construct
  REGION 1..119: note = Synthetic polypeptide
QVQLVQSGAEVKKPGASVKVSCKASGYTFNTYAMTWVRQAPGQGLEWMGWISTYNGNTVFGQKFQGRVTLSTDTSTSTAYMELRSLTSDDTAVYYCAREDDDYYSMDVWGQGTTVTVSS

SEQ ID NO: 358 - protein, 129 aa, synthetic construct
  REGION 1..129: note = Synthetic polypeptide
EVQLVQSGGGLVQPGGSLRLSCAASGFTFSTYWMSWVRQAPGKGLEWVANIKQDGSEKYYVDSVKGRFTISRDNAKNSLYLQMNSLRAEDTAVYYCARDISCISTSCYGGYYYYGMDVWGQGTTVTVSS

SEQ ID NO: 359 - protein, 121 aa, synthetic construct
  REGION 1..121: note = Synthetic polypeptide
EVQLVESGGGVVQPGRSLRLSCAASGFTFSNSGMHWVRQAPGKGLEWVAVIWYDSRNQNYADSVKGRFTISRDNSKNTLFLQMNSLRAEDTAVYYCARDYYASGDGSIDYWGQGTLVTVSS

SEQ ID NO: 360 - protein, 121 aa, synthetic construct
  REGION 1..121: note = Synthetic polypeptide
EVQLVESGGGLVQPGGSLRLSCAASGFTFSSYAMSWVRQAPGKGLEWVSTFSGRGGSTYYADFVKGRFTISRDNSKNTLYLQMNSLRAEDTAVYYCAKYYDSSGYYYFDYWGQGTLVTVSS

SEQ ID NO: 361 - protein, 120 aa, synthetic construct
  REGION 1..120: note = Synthetic polypeptide
QVQLVESGGGVVQPGRSLRLSCGGSGFTFSSYGMHWVRQAPGKGLEWVAVISYDGSKKYSADSVKGRFTISRDNSKNTLYLQMNSLRAEDTAVYYCAKGSVSVAGAEDYWGQGTLVTVSS

SEQ ID NO: 362 - protein, 121 aa, synthetic construct
  REGION 1..121: note = Synthetic polypeptide
EVQLLESGGGLVQPGRSLRLSCAVSGFTFAEYAMHWVRQAPGKGLEWVSSISWNSGRIGYVDSVRGRFTISRDNAKNSLYLQMNSLRVEDTAFYYCAKGYDSSGYYWADYWGQGTLVTVSS

SEQ ID NO: 363 - protein, 120 aa, synthetic construct
  REGION 1..120: note = Synthetic polypeptide
EVQLLESGPGLVKPSETLSLTCTVSGGSISSYYWSWIRQPAGKGLELIGRIYTSGSGNYNPSLKRRVTMSVDTSKNQISLRLNSVTAADTAVYYCARERGGYFTEPFDIWGQGTMVTVSS

SEQ ID NO: 364 - protein, 119 aa, synthetic construct
  REGION 1..119: note = Synthetic polypeptide
EVQLLESGGGLVHPGGSLRLSCAASGFTFSDYEMNWVRQAPGKGLEWVSHISSSGNIIYYADSVKGRFTISRDNAKDSLYLQMNSLRAEDTAVYYCAATIFGVVSFDYWGQGTLVTVSS

SEQ ID NO: 365 - protein, 121 aa, synthetic construct
  REGION 1..121: note = Synthetic polypeptide
EVQLVESGGGLVQPGGSLRLSCAASGFTFSAYAMSWVRQAPGRGLEWVSAISGSDRRIYYADSVKGRFSISRDNSKNTLYLQMSSLRAEDTAVYYCAKYYDSSGYYYLDYWGQGTLVTVSS

SEQ ID NO: 366 - protein, 119 aa, synthetic construct
  REGION 1..119: note = Synthetic polypeptide
EVQLVESGGGLVQPGGSLRLSCAASGFTFSDHYMAWVRQAPGKGLEWVGRIRNKPNSYTTEYAASVKGRFTISRHDSENSLYLQMNSLKTEDTAVYYCCRESGEGFDPWGQGTLVTVSS

SEQ ID NO: 367 - protein, 133 aa, synthetic construct
  REGION 1..133: note = Synthetic polypeptide
QVQLVQSGAEVKKPGASVKVSCKASGYSFTTYGISWVRQAPGQGLEWMGWISGYSGDTNYAQKVQGRVTMTTDTSTSTAYMELRSLRSDDTAVYYCARDQSHGTFGGVIDSTTLFYYYGMDVWGQGTTVTVSS

SEQ ID NO: 368 - protein, 127 aa, synthetic construct
  REGION 1..127: note = Synthetic polypeptide
EVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGYCSSTSCFYYYYGMDVWGQGTTVTVSS

SEQ ID NO: 369 - protein, 129 aa, synthetic construct
  REGION 1..129: note = Synthetic polypeptide
EVQLVESGGGLVKPGGSLRLSCVASGFTFSRYSMNWVRQAPGKGLEWVSSISHSGRYIYYADSEKGRFTISRDNAKNSLYLQMNSLRAEDTAVYYCARDHYFDSSGDYLSYYYNGMDVWGQGTTVTVSS

SEQ ID NO: 370 - protein, 119 aa, synthetic construct
  REGION 1..119: note = Synthetic polypeptide
EVQLVESGGGLVQPGGSLRLSCAASGFTFSDHYMDWVRQAPGKGLEWVGRTRNKPNSHTTEYAASVKGRFTISRDDSKNSLYLQMNSLQTEDTAVYYCARVYGGPDDYWGQGTLVTVSS

SEQ ID NO: 371 - protein, 123 aa, synthetic construct
  REGION 1..123: note = Synthetic polypeptide
EVQLVESGGGLVQPGGSLRLSCAASGFIYTNYAMYWVRQAPGKGLEWVSAISGSGGITYYADSVKGRFTISRDNSKNTLYLQMNSLRAEDKAVYYCAKDGVTTINGWFHFEYWGQGTLVTVSS

SEQ ID NO: 372 - protein, 122 aa, synthetic construct
  REGION 1..122: note = Synthetic polypeptide
EVQLLESGGGLVQPGGSLRLSCAASGFIFSDYYMDWVRQTPGKGPEWVGRITNRPNSYTTEYAASVKGRFTISRDDSTNSLFLHMNSLKTEDTAVYYCTRITGDRYWYLDLWGRGTLVTVSS

SEQ ID NO: 373 - protein, 128 aa, synthetic construct
  REGION 1..128: note = Synthetic polypeptide
EVQLVESGPGLVKPSQTLSLTCTVSGGSISSGSYYWSWIRQPAGKGLEWIGRIYTSGSTNYNPSLKSRVTMSVDTSKNQFSLKLSSVTAADTAVYYCARGWFGYSNYGLYYYYGMDVWGQGTTVTVSS

SEQ ID NO: 374 - protein, 120 aa, synthetic construct
  REGION 1..120: note = Synthetic polypeptide
EVQLVESGPGLVKPSGTLSLTCAVSGGSISSSNWWSWVRQPPGKGLEWIGEIYHSESTNYNPSLKSRVTISVDKSKNQFSLKLSSVTAADTAVYYCARDFWSGSNWFDPWGQGTLVTVSS

SEQ ID NO: 375 - protein, 132 aa, synthetic construct
  REGION 1..132: note = Synthetic polypeptide
EVQLLESGGGLVQPGRSLRLSCAASGFTFDDYAMHWVRQAPGKGLEWVSGISWNSGSIGYADSVKGRFTISRDNAKNSLYLQMNSLRAEDTALYYCAKDIGDSYGSGSYYLPYGAYYGMDVWGQGTTVTVSS

SEQ ID NO: 376 - protein, 121 aa, synthetic construct
  REGION 1..121: note = Synthetic polypeptide
EVQLLESGGGLVQPGGSLRLSCAASGFTFSSYAMSWVRQATGRGLEWVSSIRSSGGRTEYADSVKGRFTISRDNSKNTLYLQMDSLRAEDTALYYCAKHYDSSGYYYEDYWGQGTLVTVSS

SEQ ID NO: 377 - protein, 119 aa, synthetic construct
  REGION 1..119: note = Synthetic polypeptide
EVQLVESGGALVHPGGSLGLSCAASGFTFSDHYMDWVRQAPGKGLEWVGRIRNKPNSYATQYAASVKGRFTISRDDSKKSLYLQMNSLNTEDTAVYYCARVRDGEYDYWGQGTLVTVSS

SEQ ID NO: 378 - protein, 130 aa, synthetic construct
  REGION 1..130: note = Synthetic polypeptide
EVQLLESGGGLVKPGGSLRLSCAASGFTFSSYSMNWVRQAPGKGLEWVSSISSRSSFMYYADSVKGRFTISRDNAKNSLYLQMNSLRVEDTAVYYCARDNSEVEDYGDYVLYHYYGMDVWGQGTTVTVSS

SEQ ID NO: 379 - protein, 120 aa, synthetic construct
  REGION 1..120: note = Synthetic polypeptide
EVQLLESGGGVVQPGRSLRLSCVASGFTFSSYGMHWVRQAPGKGLEWVALISYDGSNKYYADSVKGRFTISRDNSKNTLYLQMNSLRAEDTAVYYCAKDQCGGDCTADYWGQGTLVTVSS

SEQ ID NO: 380 - protein, 119 aa, synthetic construct
  REGION 1..119: note = Synthetic polypeptide
QVQLVESGGGVVQPGRSLRLSCAASGFTFSSLAMHWVRQAPGKGLEWVATISYDVSNKYYADSVKGRFTISRDNSKNTLFLQMNSLRPEDTAVYYCARGYTGYDGFDYWGQGTLVTVSS

SEQ ID NO: 381 - protein, 125 aa, synthetic construct
  REGION 1..125: note = Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARRPYYYGSRRPAGHMDVWGQGTTVTVSS

SEQ ID NO: 382 - protein, 119 aa, synthetic construct
  REGION 1..119: note = Synthetic polypeptide
QVQLQESGPGLVRPSQTLSLTCTVSGGAISSGDYYWSWVRQPPGKGLEWIGYIHYSGTTYNNPSLKSRVTIAVDTSKNQFSLKLSSVTAADTAVYFCGRDSDKNYFDYWGQGTLVTVSS

SEQ ID NO: 383 - protein, 121 aa, synthetic construct
  REGION 1..121: note = Synthetic polypeptide
EVQLVESGGGVVRPGRSLRLSCAASGFTFSSYGMHWVRQAPGKGLEWVAVIRFDGSNTVYADSVKGRFTISRDNSKNTLYLQMNSLRAEDTAVYYCAKTYDSNAYYYLDYWGQGTLVTVSS

SEQ ID NO: 384 - protein, 125 aa, synthetic construct
  REGION 1..125: note = Synthetic polypeptide
EVQLVESGGGVVQPGWSLRLSCAVSGFTFSSYAMHWVRQAPGKGLEWVAVISYDGSYKWYADSVKGRFTISRDNSKNTVYLQMNSLRAEDTAVYYCASLWFIVMTMSKNPETDYWGQGTLVTVSS

SEQ ID NO: 385 - protein, 121 aa, synthetic construct
  REGION 1..121: note = Synthetic polypeptide
EVQLVESGGGLIQPGGSLRLSCAASGFSFSSHAMTWVRQAPGKGLQWVSSIRGSDRTTNYADSVKGRFTVSRDNSKNTLYLQMNSLRAEDTAIYYCAKYYDSSGYYYFDHWGQGTLVTVSS

SEQ ID NO: 386 - protein, 121 aa, synthetic construct
  REGION 1..121: note = Synthetic polypeptide
EVQLVESGGTFLQPGGSLRLSCVASGFTFGTHAMSWVRQAPGKGLEWVSTFSGSGGRTYYADSVKGRFTISRDNSKSTLYLEMSALRAEDTAVYYCAKFYDSSGYYYFDYWGQGTLVTVSS

SEQ ID NO: 387 - protein, 122 aa, synthetic construct
  REGION 1..122: note = Synthetic polypeptide
EVQLVESGGGLVQPGGSLRLSCAASGFTFSDYYMDWVRQAPGKGLEWVGGIRNKPNSYTTEYAASVKGRFTISRDDSKNSLFLQMNSLKTEDTAVYYCVRLYGDYVAYFDYWGQGTLVTVSS

SEQ ID NO: 388 - protein, 128 aa, synthetic construct
  REGION 1..128: note = Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARRGTTVTRFGVIQYYYGMDVWGQGTTVTVSS

SEQ ID NO: 389 - protein, 117 aa, synthetic construct
  REGION 1..117: note = Synthetic polypeptide
QVQLQESGPGLVKPSETLSLTCTVSGASIRSYLWSWIRQPPGKELEWLGSIYHSGSTKYNPSLKSRVTISADTSKNQFSLKLNSVTAADTAVFYCARETANNWFDPWGQGTLVTVSS

SEQ ID NO: 390 - protein, 121 aa, synthetic construct
  REGION 1..121: note = Synthetic polypeptide
EVQLVESGGGVVQSGRSLRLSCAASGFTFSGNAMHWVRQAPGKGLEWVAVILYDGSNQYYADSVKGRFTISRDNSKNTLYLQMNSLRPADTAVYYCARASMMPRPPVHDYWGQGTLVTVSS

SEQ ID NO: 391 - protein, 123 aa, synthetic construct
  REGION 1..123: note = Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDRSQGDYGDYVADYWSQGTLVTVSS

SEQ ID NO: 392 - protein, 125 aa, synthetic construct
  REGION 1..125: note = Synthetic polypeptide
EVQLQESGPGLVKPSGTLSLTCAVSGGSISSSNWWTWVRQPPGKGLEWIGEIYHSGSTNYNPSLESRVTMSVDKSKNQFSLKLSSVTAADTAVYYCARVQTSHSELWFGEFGADWGQGTLVTVSS

SEQ ID NO: 393 - protein, 121 aa, synthetic construct
  REGION 1..121: note = Synthetic polypeptide
EVQLLESGGGLVQPGGSLRLSCAASGFTFTYYAMSWVRQAPGKGLEWVSGISGSGDSTYNADSVKGRVTISRDNSKNTLYLQMNSLRAEDTAVYYCAKDGGYSTDWYFDLWGRGTLVTVSS

SEQ ID NO: 394 - protein, 121 aa, synthetic construct
  REGION 1..121: note = Synthetic polypeptide
QVQLVESGGGVVQPGRSLRLSCTASGFTFSSYGMHWVRQAPGKGPEWVAVISYDGSKKYFADSVKGRFTISRDNSKNTLYLQMNSLRAEDTAVYSCAKGYDSNGYYYIDYWGQGTPVTVSS

SEQ ID NO: 395 - protein, 123 aa, synthetic construct
  REGION 1..123: note = Synthetic polypeptide
QVQLQESGGGVVQPGRSLRLSCAASGFTFSSYGMHWVRQAPGKGLEWVAVIWYDGSNKYYADSVKGRFTISRDNSKNTLYLQMNSLRAEDTAVYYCARDVGYQLLQVYGMDVWGQGTTVTVSS

SEQ ID NO: 396 - protein, 129 aa, synthetic construct
  REGION 1..129: note = Synthetic polypeptide
EVQLLESGPGLVKPSQTLSLTCSVSGGSISSGGYYWTWIRQPPGKGLEWIGYIYYTGSTYYNPSLKSRVTISVDTSKNQFSLKLSSVTAADTAVYFCARAEYDTSGYYQQRLPEYFQHWGQGTLVTVSS

SEQ ID NO: 397 - protein, 121 aa, synthetic construct
  REGION 1..121: note = Synthetic polypeptide
EVQLVQSGGGLVQRGGSLRLSCAASGFTFSSYAMTWVRQAPGKGLEWVSDMNHSGDRTNYADSVRGRFTISRDNSKNTLYLQMNSLRAEDTAVYYCAKYYDSSGYYYFHSWGQGTLVTVSS

SEQ ID NO: 398 - protein, 120 aa, synthetic construct
  REGION 1..120: note = Synthetic polypeptide
EVQLLESGGGLVQPGGSLRLSCAASGFIFSDHYMAWVRQAPGKGLEWVGRSRNRPNSYTTEYAASAKGRFTISRDDSKTSLYLQMNSLKTEDTAVYYCAREHGDYGLDYWGQGTLVTVSS

SEQ ID NO: 399 - protein, 124 aa, synthetic construct
  REGION 1..124: note = Synthetic polypeptide
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCYVDYYYDSSGYYSPFDYWGQGTLVTVSS

SEQ ID NO: 400 - protein, 121 aa, synthetic construct
  REGION 1..121: note = Synthetic polypeptide
EVQLVESGGGFVQPGGSLRLSCAASGFIFSDYYMDWVRQAPGKGLEWVGRIRNKPNSYTTEYAASVKGRFSISRDDLKNSLYLQMNSLKTEDTAEYYCARVDGEEVALIYWGQGALVTVSS

SEQ ID NO: 401 - protein, 122 aa, synthetic construct
  REGION 1..122: note = Synthetic polypeptide
EVQLLESGGGLGQPGGSLRLSCVASKFTFSDHYMDWVRQAPGKGLEWVGRIRNKPNGYTTEYAASVKGRFIISRDDSKNSLYLQMKSLKIEDTAIYYCVRVWGGEAARYDYWGQGALVTVSS

SEQ ID NO: 402 - protein, 119 aa, synthetic construct
  REGION 1..119: note = Synthetic polypeptide
EVQLVESGGGLVQPGGSLRLSCAASGFTFSDHYMDWVRQAPGKGLEWVGRSRNKPNSYITEYAASVKGRFTISRDDSKNSLYLQMNSLKTEDTAVYYCSRHMGFGLDLWGQGTLVTVSS

SEQ ID NO: 403 - protein, 121 aa, synthetic construct
  REGION 1..121: note = Synthetic polypeptide
QVQLVQSGGGVVQPGRSLRLSCAASGFTFSSYGMHWVRQAPGKGLEWVAVIWYDGSNKYYADSVKGRFTISRDNSKNTLYLQMNSLRAEDTAVYYCARDYYGSGDGYFDYWGQGTTVTVSS

SEQ ID NO: 404 - protein, 124 aa, synthetic construct
  REGION 1..124: note = Synthetic polypeptide
EVQLVESGPVLVKPTETLRLTCTVSGFSLSNTKLGVSWIRQPPGKALEWLAHIFSNAEKSSSKSLKSRLSISQDTSKSLVVLTMTNMDPVDTATYFCARIPVEYGTPRGSFDTWGQGTTVTVSS

SEQ ID NO: 405 - protein, 114 aa, synthetic construct
  REGION 1..114: note = Synthetic polypeptide
EVQLVESGGGVVQPGRSLRLSCAASGLTFSTYTLHWVRQAPGKGLEWVAVISSDGGNKYYADSVKGRFTISRDSSKNTLYLQMNSLRTEDTAVYYCAGGSPDYWGQGALVTVSS

SEQ ID NO: 406 - protein, 121 aa, synthetic construct
  REGION 1..121: note = Synthetic polypeptide
EVQLVESGGGVVQPGRSLRLSCVPSGFTFSSYAMHWVRQAPGKGLEWVAMMSYDGGDKNYADSVKGRFTISRDNSKNTLYLQMRSLRAEDTAIYYCARAYDSRGYYYIEHWGQGTLVTVSS

SEQ ID NO: 407 - protein, 119 aa, synthetic construct
  REGION 1..119: note = Synthetic polypeptide
QVQLVQSGAEVRKPGASVKVSCKASGYTFTSYGISWVRQAPGQGLEWMGWISTYNGNTNYAQKLQGRVTMTTDTSTSTAYMELRSLRSDDTAVYYCAREIDSNYVFDYWGQGTLVTVSS

SEQ ID NO: 408 - protein, 122 aa, synthetic construct
  REGION 1..122: note = Synthetic polypeptide
EVQLVESGGGLVQPGGSLRLSCAASGFTFSNYWMNWVRQAPGKGLEWVANIKQDGSEKYYVDSVKGRFTISRDNAKNSLYLQMNSLRAEDTAVYYCARKLSYSSGWYYFDYWGQGTLVTVSS

SEQ ID NO: 409 - protein, 119 aa, synthetic construct
  REGION 1..119: note = Synthetic polypeptide
EVQLVESGGGLVQPGGSLRLSCAASGFTFSDHYMDWVRQAPGKGLEWVGRSTNKPNSYTTTYAASVRGRFTISRDESKNSLYLQMNSLKSDDTAVYYCVTTTVILFDYWGQGTLVTVSS

SEQ ID NO: 410 - protein, 126 aa, synthetic construct
  REGION 1..126: note = Synthetic polypeptide
QVQLQQWGAGLLKPSETLSLTCAVYGGSFSGYYWSWIRQPPGKGLEWIGEINHRGSTDYNPSLKSRVTMSVDTSKNQFSLRLSSVTAADTALYYCARGRLAWGLRGQKSPNFFAYWGQGATVTVSS

SEQ ID NO: 411 - protein, 125 aa, synthetic construct
  REGION 1..125: note = Synthetic polypeptide
EVQLVESGGGLVKPGGSLRLSCAASGFTFSHAWMTWVRQAPGKGLEWVGRIKSETDGGTANYAAPVKGRFTISRDDSKNTVYLQMVSLKTEDTAVYYCATAGIFGVVIMKGFDHWGQGTTVTVSS

SEQ ID NO: 412 - protein, 123 aa, synthetic construct
  REGION 1..123: note = Synthetic polypeptide
EVQLLESGAEVKEPGSSVKVSCKPSGGTFSSYVISWVRQAPGQGLEWMGGIIPIFGTPNYAQKFQGRVTITADDSTSTAHMELSSLTSDDTAVYYCARETYYYGSGSVPVHDWGQGTLVTVSS

SEQ ID NO: 413 - protein, 122 aa, synthetic construct
  REGION 1..122: note = Synthetic polypeptide
EVQLVESGGGVVQPGRSLRLSCAASGFIFSSNSMHWVRQAPGKGLKWVAIISNDGRNKFYADAVKGRFTVSRDNSKNTLYLQMNSLRPEDTAVYYCARGYDSSGYWGFGDNWGQGTLVTVSS

SEQ ID NO: 414 - protein, 122 aa, synthetic construct
  REGION 1..122: note = Synthetic polypeptide
QVQLVQSGGGLVQPGGSLRLSCAASGFTFSDHYMDWVRQAPGKGLEWVGRTRNKANSYTTKYAASVKGRFTISRDDSKNSLYLQMNSLKTEDTAVYYCARVEGGAWGAFDIWGQGTTVTVSS

SEQ ID NO: 415 - protein, 122 aa, synthetic construct
  REGION 1..122: note = Synthetic polypeptide
QVTLKESGPVLVKPTETLTLTCTVSGFSLSNTKMGVTWIRQPPGKALEWLAHIFSNDEKSCNTSLKSRLTISKDTSKSQVVLTMTNMDPVDTATYYCARLWFTEYPGAFDIWGQGTMVTVSS

SEQ ID NO: 416 - protein, 125 aa, synthetic construct
  REGION 1..125: note = Synthetic polypeptide
EVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARHSSGSYYLAGYYFDYWGQGTLVTVSS

SEQ ID NO: 417 - protein, 119 aa, synthetic construct
  REGION 1..119: note = Synthetic polypeptide
EVQLVESGGGLVQPGGSLRLSCAASGFTFSDHYMDWVRQAPGRGLEWVGRSRNKVNSYTTDYAASVKGRFTISRDDSKNSLFLRMNSLKTEDTAVYYCARLTDSGYDDWGLGTLVTVSS

SEQ ID NO: 418 - protein, 126 aa, synthetic construct
  REGION 1..126: note = Synthetic polypeptide
EVQLVESGPGLVKPSETLSLTCTVSGGSISSYYWSWIRQPPGKGLEWIGYIYYSGSTNYNPSLKSRVTISVDTSKNQFSLKLSSVTAADTAVYYCARETCSGGSCYYRVGSAFDIWGQGTTVTVSS

SEQ ID NO: 419 - protein, 122 aa, synthetic construct
  REGION 1..122: note = Synthetic polypeptide
EVQLLESGGGMVQPGRSLRLSCAASGFTFDDYDMHWVRQGPGKGLEWVSGISWNSGGRGYADSVKGRFTISRDNAKNSLYLQMNSLRVEDTALYYCVKDYCSGGRCYSFDYWGQGTLVTVSS

SEQ ID NO: 420 - protein, 121 aa, synthetic construct
  REGION 1..121: note = Synthetic polypeptide
QVQLVESGGGVVQPGRSLRLSCAASGFTFSSYGMHWVRQAPGKGLEWVAVMSYDGSNKYYADSLKGRFTISRDNSKNTLYLQMNSLRAEDTAVYFCAKAYDSSAYYYLDYWGQGTLVTVSS

SEQ ID NO: 421 - protein, 121 aa, synthetic construct
  REGION 1..121: note = Synthetic polypeptide
EVQLVESGGGVIQPGRSLRLSCAASGFNFSSYGMHWVRQAPGKGLEWVAVISYDGSNKYYADSVKGRFTISRDNSKNTLYLQMNSLRAEDTAVHYCAKAYDSRGYYYLDYWGQGTLVTVSS

SEQ ID NO: 422 - protein, 129 aa, synthetic construct
  REGION 1..129: note = Synthetic polypeptide
EVQLVQSGGGLVQPGGSLRLSCVGSGLTLSSSAMSWVRQAPGKGLECVSGITGSGSDSSYAASVKGRFTISRDNSKNTVYLQMNSLRAEDTAVYYCAKDLTHRLGSIFGKLTFDAFDIWGPGTMVTVSS

SEQ ID NO: 423 - protein, 123 aa, synthetic construct
  REGION 1..123: note = Synthetic polypeptide
EVQLVESGGGVVQPGRSLRLSCAASGFTFSSYGMHWVRQAPGKGPEWVAVISEDGNKDHYVDSVKGRFSIYRDNSKSTVFLRMTSLRAEDTAVYYCAKDLTPYFYDSGAFDHWGQGTLVTVSS

SEQ ID NO: 424 - protein, 119 aa, synthetic construct
  REGION 1..119: note = Synthetic polypeptide
EVQLVESGGGLVQPGGSLRLSCAVSGFTFSDHYMDWVRQAPGKGLEWVGRSRNKVNSYITEYAASVKGRFSISRDDSKNSLYLQMNSLKIEDTAVYYCARVFGGPTDYWGQGTLVTVSS

SEQ ID NO: 425 - protein, 118 aa, synthetic construct
  REGION 1..118: note = Synthetic polypeptide
EVQLLESGGGLVQPGGSLRLSCAASGFIFSDHYMDWVRQAPGKGLEWVGRIRNKPNSYTTDYAAYVKGRFSISRDDSKNSLFLQMNSLKTEDTAVYYCARVVNGLDVWGQGTTVTVSS

SEQ ID NO: 426 - protein, 114 aa, synthetic construct
  REGION 1..114: note = Synthetic polypeptide
EVQLVESGGGVVQPGRSLRLSCAASGFTLSSYVMHWVRQAPGKGLEWVAVISSDGTNKYYADSVKGRFTISRDSSKNTLYLQMNSLRPEDSAVYYCARGQPDYWGQGTLVTVSS

SEQ ID NO: 427 - protein, 121 aa, synthetic construct
  REGION 1..121: note = Synthetic polypeptide
EVQLVESGPGLVKPSGTLSLTCAVSGGSISSDNWWSWVRQAPGKGLEWIGEIYHTGSTSYNPSLKSRVTISLDKSKNHFSLKLNSLTAADTAVYYCAGKKWELLGFRFDPWGQGTLVTVSS

SEQ ID NO: 428 - protein, 117 aa, synthetic construct
  REGION 1..117: note = Synthetic polypeptide
QVQLVESGAEEKKPGASVKVSCKASGYTFTSYAMHWVRQAPGQRLEWMGWINAGNGNTKYSQKFQGRVTITRDTSASTAYMELSSLRSEDTAVYYCARQWLGHFDYWGQGTLVTVSS

SEQ ID NO: 429 - protein, 119 aa, synthetic construct
  REGION 1..119: note = Synthetic polypeptide
EVQLVESGGGLVQPGGSLRLSCAASGFIFSDHYMAWVRQAPGKGLEWVGHVGNKANTYTTEYAASVKGRFTISRDDSKKSLYLQMNRLKSEDTAVYYCARVFSYYLDYWGQGTPVTVSS

SEQ ID NO: 430 - protein, 126 aa, synthetic construct
  REGION 1..126: note = Synthetic polypeptide
QVTLKESGPTLVKPTQTLTLTCTFSGFSLSTSGVGVGWTRQPPGKALEWLALIYWDDDKRYSPSLKSRLTITKDTSKNQVVLTMTKMDPVDTATYYCAHRHIAARLYRDDDVFDVWGQGTMVTVSS

SEQ ID NO: 431 - protein, 119 aa, synthetic construct
  REGION 1..119: note = Synthetic polypeptide
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARGLNTVTNSDYWGQGTLVTVSS

SEQ ID NO: 432 - protein, 120 aa, synthetic construct
  REGION 1..120: note = Synthetic polypeptide
QVQLVQSGAEVKKPGASVKVSCKASGYTFTGYYMHWVRQAPGQGLEWMGWINPNSGGTNYAQKFQGWVTMTRDTSISTAYMELSRLRSDDTAVYYCASGLSPDFSVLDVWGQGTTVTVSS

SEQ ID NO: 433 - protein, 133 aa, synthetic construct
  REGION 1..133: note = Synthetic polypeptide
QVQLQQSGPGLVKPSQTLSLTCAISGDSVSTNSAAWNWIRQSPSRGLEWLGRTYYRSKWYNDYALSVKSRITIKPDTSKNQFSLQLNSVTPEDTAVYYCAREGAGYYDSSGYYPLSYDAFDIWGRGTMVTVSS

SEQ ID NO: 434 - protein, 119 aa, synthetic construct
  REGION 1..119: note = Synthetic polypeptide
EVQLVESGGGLVQPGGSLRLSCAASGFTFSDHYMDWVRQAPGKGLEWVGRARNRANSYTTEYAASVKGRFAASRDDSKNSLYLQMNSLKTEDTAVYYCARVRGSYWDYWGQGTLVTVSS

SEQ ID NO: 435 - protein, 118 aa, synthetic construct
  REGION 1..118: note = Synthetic polypeptide
QVQLVQSGGGLVQPGGSLRLSCAASGFTFSDHYMDWVRQAPGKGLEWVGRIRNKVNSYTTEYAASVKGRFTISRDDSKNSLYLQMNSLKTEDTAVYYCGRDRGWLDIWGQGTMVTVSS

SEQ ID NO: 436 - protein, 130 aa, synthetic construct
  REGION 1..130: note = Synthetic polypeptide
QVQLQESGPGLVEPSGTLSLTCVVTGDSISSRSWWSWVRQPPGKGLEWIGEIYHSGTTTYSPSLKSRVIISLDKSENHFSLKMTSVTAADTAVYYCARVIRDLRDYYDGSGYGPDAFDIWGQGTTVTVSS

SEQ ID NO: 437 - protein, 123 aa, synthetic construct
  REGION 1..123: note = Synthetic polypeptide
EVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCARARWEDGNYYYGMDVWGQGTTVTVSS

SEQ ID NO: 438 - protein, 124 aa, synthetic construct
  REGION 1..124: note = Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDQSSGWPNYYYGMDVWGQGTTVTVSS

SEQ ID NO: 439 - protein, 126 aa, synthetic construct
  REGION 1..126: note = Synthetic polypeptide
QVQLVESGSELKKPGASVKVSCKASGYTFTSYAMNWVRQAPGQGLEWMGWINTNTGNPTYAQGFTGRFVFSLDTSVSTAYLQISSLKAEDTAVYYCVRGYCSSTSCYGGLYWFDPWGQGTLVTVSS

SEQ ID NO: 440 - protein, 126 aa, synthetic construct
  REGION 1..126: note = Synthetic polypeptide
EVQLVESGGGVVQPGRSLRLSCADSGFTFSYSAIHWVRQAPGKGLEWVAVISYDGSNKYYADSVKGRFTISRDNSKNTLYLQMNSLRPEDTAVYYCARHSGGYSSKDKPTEYFQHWGQGTLVTVSS

SEQ ID NO: 441 - protein, 122 aa, synthetic construct
  REGION 1..122: note = Synthetic polypeptide
EVQLLESGPGLVKPSGTLSLTCAVSGASISSNNWWSWVRQSPGKGLEWIGEIFHSGTTNYNPSLKSRVTISVDKSKNQFSLKLNSVTAADTAVYYCARDVGVAAVITGSVRWGQGTLVTVSS

SEQ ID NO: 442 - protein, 126 aa, synthetic construct
  REGION 1..126: note = Synthetic polypeptide
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARGYCSSTSCYGGLYWFDPWGQGTLVTVSS

SEQ ID NO: 443 - protein, 133 aa, synthetic construct
  REGION 1..133: note = Synthetic polypeptide
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDGAGDYIWGSYRHKGLHYYYGMDVWGQGTTVTVSS

SEQ ID NO: 444 - protein, 124 aa, synthetic construct
  REGION 1..124: note = Synthetic polypeptide
EVQLVESGPGLVMPSGTLSLTCTVSGISISSSNWWSWVRQSPGKGLEWIGEVYHSGSTKYNPSLKSRVTISVDKSRNQFSLKLNSVTAADTAVYYCAKDPRTFYGVVMLLDDPWGQGTLVTVSS

SEQ ID NO: 445 - protein, 119 aa, synthetic construct
  REGION 1..119: note = Synthetic polypeptide
EVQLVESGGGVVQPGRSLRLSCAVSGFTFSTSPLHWVRQAPGKGLEWVAVSSFVATDKYYADSVKGRFTVSRDNSKNTLYLQMNSLRPEDTAVYYCARGFGELPGFDIWGQGTMVTVSS

SEQ ID NO: 446 - protein, 117 aa, synthetic construct
  REGION 1..117: note = Synthetic polypeptide
EVQLLESGGGLVKPGGSLRLSCAASGFTFSSYSMNWVRQAPGKGLEWVSSISSSSSYIYYADSVKGRFTISRDNAKNSLYLQMNSLRAEDTAVYYCARDSWGPFDYWGQGTLVTVSS

SEQ ID NO: 447 - protein, 121 aa, synthetic construct
  REGION 1..121: note = Synthetic polypeptide
EVQLVESGGAVVQPGRSLRLSCAASGFTFSSYGMHWVRQAPGKGLESVAVIWYDGSNKNYADSVKGRFTISRDNSKNTLFLQMNSLRAEDSAMYYCAKTYDSRAYYYLDYWGQGTLVTVSS

SEQ ID NO: 448 - protein, 123 aa, synthetic construct
  REGION 1..123: note = Synthetic polypeptide
EVQLLESGGGLVQPGGSLRLSCAASGFTFSSYAMSWVRQAPGKGLELVSAISSSGGSTYYADSVKGRFTISRDNSKNTLYLQMNSLRAEDTALYYCAKDLFYDFWTGITIDYWGQGTLVTVSS

SEQ ID NO: 449 - protein, 120 aa, synthetic construct
  REGION 1..120: note = Synthetic polypeptide
EVQLLESGGGLVQPGGSLRLSCAASGFIFSNYWMSWVRQAPGKGLEWVANIKPDGSEKYYVESVRGRFTISRDNAKNSLYLQMNSLRAEDTAVFYCARDGGTVSDGLDVWGQGTTVTVSS

SEQ ID NO: 450 - protein, 122 aa, synthetic construct
  REGION 1..122: note = Synthetic polypeptide
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARVVWYSSSSHLFDYWGQGTLVTVSS

SEQ ID NO: 451 - protein, 117 aa, synthetic construct
  REGION 1..117: note = Synthetic polypeptide
EVQLVESGGGVVQTGRSLRLSCAASGFTFSISGMHWVRQAPGKGLEWVALIWYDGTKKYYADSVKGRFTISRDDFKNTVYLQMNSLRADDTAVYYCARIKSDAFDLWGQGTTVTVSS

SEQ ID NO: 452 - protein, 123 aa, synthetic construct
  REGION 1..123: note = Synthetic polypeptide
EVQLLESGGGVVQPGKSLRLSCAASGFSFGDYGMHWVRQTPDKGLEWVAVILFDGSKKFYADSVRGRFTISRDNSKNNLYLQMSSLRPEDTAVYYCAKFPLRDGGSGEGFDYWGQGTLVTVSS

SEQ ID NO: 453 - protein, 121 aa, synthetic construct
  REGION 1..121: note = Synthetic polypeptide
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARNTYYDRRRTFDYWGQGTLVTVSS

SEQ ID NO: 454 - protein, 122 aa, synthetic construct
  REGION 1..122: note = Synthetic polypeptide
QVQLVQSGGGLVQPGGSLRLSCAASGFTFSDHYMDWVRQAPGKGLEWVGRTRNKANSYTTEYAASVKGRFTISRDDSKNSLYLQMNSLKTEDTAVYYCAGVGITGTTGIDYWGQGTLVTVSS

SEQ ID NO: 455 - protein, 125 aa, synthetic construct
  REGION 1..125: note = Synthetic polypeptide
EVQLLESGGDLVQPGRSLRLSCAASGFNLIDYAMHWVRQVPGKGLEWVSGISWNSRSIGYADSVKGRFTISRDNAKNSLYLQMDSLKHEDTALFYCAKGAAAGPFPYFYYAMDVWGQGTTVTVSS

SEQ ID NO: 456 - protein, 110 aa, synthetic construct
  REGION 1..110: note = Synthetic polypeptide
QSVLTQPPSVSAAPGQKVTISCSGSSSNIGNNYVSWYQQLPGAAPKLLIYDNKKRPSGIPDRFSGSASGTSATMGITGLQTGDEADYYCGTWDSSLSAWVFGGGTKVTVL

SEQ ID NO: 457 - protein, 107 aa, synthetic construct
  REGION 1..107: note = Synthetic polypeptide
DIRVTQSPATLSVSPGERATLSCRASQSVSSNLAWYQQKPGQAPRLLIYDASNRATGIPVRFSGSGSGTDFTLTISSLQSEDFAVYYCQQYDNWPLTFGGGTKVEIK

SEQ ID NO: 458 - protein, 110 aa, synthetic construct
  REGION 1..110: note = Synthetic polypeptide
QSVVTQPPSVSAAPGQKVTISCSGSSSNIGNNYVSWYQQFPRTAPKLLIYDNKKRPSGIPDRFSGSASGTSATLGITGLQTGDEADYYCGTWDSSLSAWVFGGGTKVTVL

SEQ ID NO: 459 - protein, 110 aa, synthetic construct
  REGION 1..110: note = Synthetic polypeptide
QPVLTQPPSASGTPGQRVTIFCSGSRSNIGTYTINWYQKLPGTAPKLLIYSNNRGPSGVPDRFSGSQSGTSASLAISGLQPEDEADYYCAAWDDSLNGWVFGGGTKVTVL

SEQ ID NO: 460 - protein, 110 aa, synthetic construct
  REGION 1..110: note = Synthetic polypeptide
QSVVTQPPSVSAAPGQKVTISCSGSSSNIGNNYVAWYQQLPGRAPKLLIHDNKKRPSGIPDRFSGSASGTSATLGITGLQTGDEADYYCETWDSSLNAVVFGGGTKLTVL

SEQ ID NO: 461 - protein, 108 aa, synthetic construct
  REGION 1..108: note = Synthetic polypeptide
DIQMTQSPSSLSASVGDRVTITCRASQTISVDLNWYQHKPGKAPKLLIFAASTLQSGVPSRFSGSGSGTDFTLTIRSLQPEDFATYYCQQSYSIPRITFGQGTRLEIK

SEQ ID NO: 462 - protein, 108 aa, synthetic construct
  REGION 1..108: note = Synthetic polypeptide
EIVMTQSPSALSASVRDRVTITCRASQSIGSDLNWYQQRPGKAPMLLIYAATGLQSGVPSRFSGSGSGTDFTLTISNLQPEDFATYYCQQSYSPPMYTFGQGTKVDIK

SEQ ID NO: 463 - protein, 110 aa, synthetic construct
  REGION 1..110: note = Synthetic polypeptide
QPVLTQPPSASGTPGQRVTISCSGSSSNIGTNTVSWYQQLPGTAPQLLVFSRTQRPSGVPDRFSGSKSGTSASLAISGLQSDDEADYYCAAWDDSRNGWVFGGGTKLTVL

SEQ ID NO: 464 - protein, 112 aa, synthetic construct
  REGION 1..112: note = Synthetic polypeptide
QPVLTQPPSVSAAPGQKVTISCSGSNSNIGNYYVSWYQQFPGTAPKLLIYDNNKRPSGIPDRFSGSKSGTSATLAITGLQTGDEAHYYCGTWDTSSLSAGRVFGGGTKLTVL

SEQ ID NO: 465 - protein, 111 aa, synthetic construct
  REGION 1..111: note = Synthetic polypeptide
QSALTQPPSVSAAPGQKVTISCSGSSSNIGNSYVSWYQQVPGTAPKLLIYDNNKRPSGIPDRFSGSKSGTSATLGITGLQAGDEADYYCGTWDTSLSAGRVFGRGTKLTVL

SEQ ID NO: 466 - protein, 109 aa, synthetic construct
  REGION 1..109: note = Synthetic polypeptide
QSVLTQPPSVSAAPGQKVTISCSGSSSNIGYSHVSWYQQLPGTAPKVLIYDNDKRPSGIPDRFSGSKSGTSATLGITGLQTGDEADYYCGTWDTSLGVVFGGGTKLTVL

SEQ ID NO: 467 - protein, 112 aa, synthetic construct
  REGION 1..112: note = Synthetic polypeptide
QSVLTQPRSVSGSPGQSVTISCTGTSSDVGAYNFVSWYQQYPGKAPKLMIYDVNKRPSGVPDRFSGSKSGNTASLTISGLQAEDEADYHCCSYAGTYTSNYVFGSGTKVTVL

SEQ ID NO: 468 - protein, 110 aa, synthetic construct
  REGION 1..110: note = Synthetic polypeptide
QSVVTQPPSVSAAPGQKVTISCSGSSSNIGNNYVSWYQQLSETAPKLLIYDNNKRPSGIPNRFSGSKSGTSATLGITGLQTGDEADYYCGTWDNSLGAVVFGGGTKVTVL

SEQ ID NO: 469 - protein, 110 aa, synthetic construct
  REGION 1..110: note = Synthetic polypeptide
QSVLTQPPSVSAAPGQKVTISCSGSSSNIGSNYVSWYQQFPGTAPKLLIYDNSKRPSGIPDRFSGSMSGTSATLGITGLQTGDEADYYCGTWDSSLSAVVFGGGTKVTVL

SEQ ID NO: 470 - protein, 111 aa, synthetic construct
  REGION 1..111: note = Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDTSLSAGRVFGGGTKLTVL

SEQ ID NO: 471 - protein, 108 aa, synthetic construct
  REGION 1..108: note = Synthetic polypeptide
EIVLTQSPATLSVSPGERATLSCRASRSVSSNLAWYQQKPGQAPRLLIYGASTRATGIPARFTGSGSGTEFTLTISSLQSEDFAVYYCQQYNNWPPRTFGQGTKVDIK

SEQ ID NO: 472 - protein, 107 aa, synthetic construct
  REGION 1..107: note = Synthetic polypeptide
DIQLTQSPSSVSASVGDRVTITCRASQGISSWLAWYQQKPGKAPKLLIHAASSLQSGVPSRFSGSGSGTDFTLTISSLQPEDFATYYCQQAKSFPPTFGQGTRLEIK

SEQ ID NO: 473 - protein, 110 aa, synthetic construct
  REGION 1..110: note = Synthetic polypeptide
QPVLTQPASVSGSPGQSITISCTGTSSDVGGYNYVSWYQQHPGKAPKLLIYDVSNRPSGVSNRFSGSKSANSASLTISGLQAEDEADYYCNSYTSSSTLVFGGGTKLTVL

SEQ ID NO: 474 - protein, 106 aa, synthetic construct
  REGION 1..106: note = Synthetic polypeptide
EIVMTQSPATLSVSPGERATLSCRASQSVSSNLAWYQQKPGQAPRLLIYGASTRATGIPARFSGSGSGTEFTLTISSLQSEDFALYYCQQYDDWPLFGQGTRLEIK

SEQ ID NO: 475 - protein, 111 aa, synthetic construct
  REGION 1..111: note = Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYICGTWDTSLSAGGVFGGGTKLTVL

SEQ ID NO: 476 - protein, 110 aa, synthetic construct
  REGION 1..110: note = Synthetic polypeptide
QSVLTQPASVSGSPGQSITISCTGTSSDIGAYNYVSWYQQHPGKAPKLMIYDVTNRPSGVSNRFSGSKSGSSASLTISGLQTEDEADYYCSSYTRRSTLVFGGGTKLTVL

SEQ ID NO: 477 - protein, 109 aa, synthetic construct
  REGION 1..109: note = Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEANYYCGTWDTSLSTVFGGGTKLTVL

SEQ ID NO: 478 - protein, 111 aa, synthetic construct
  REGION 1..111: note = Synthetic polypeptide
QSALTQPASVSGSPGQSITISCTGTGSDVGGYNFVSWYQQHPGKAPKLMLYDVNNRPSGVSNRFSGSKSGNTASLTISGLQAEDEADYYCSSYPGTSALVIFGGGTRLTVL

SEQ ID NO: 479 - protein, 107 aa, synthetic construct
  REGION 1..107: note = Synthetic polypeptide
DIQMTQSPSSLSASVGDRVTITCRASQSISSYLNWYQQKPGEAPNLLIFAASILQSGVPSRFSGSGSGTDFTLTISSLQPEDFATYYCQQSYSTPYTFGQGTKVEIK

SEQ ID NO: 480 - protein, 112 aa, synthetic construct
  REGION 1..112: note = Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGHVVFGGGTKLTVL

SEQ ID NO: 481 - protein, 107 aa, synthetic construct
  REGION 1..107: note = Synthetic polypeptide
EIVLTQSPATLSSSPGERATLSCRASQSVNSYLVWYQQKPGQAPRLLIYDASNRATGIPARFTGSGSGTDFTLTISSLEPEDFAVYYCQQRTNWPFTFGQGTKVDIK

SEQ ID NO: 482 - protein, 107 aa, synthetic construct
  REGION 1..107: note = Synthetic polypeptide
EIVLTQSPATLSLSPGERATLSCRASQSVNRYLAWYQQKPGQAPRLLIYDASNRATGIPARFSGSGSGTDFTLTISSLEPEDFAVYYCHQRTNWPWTFGQGTKVEIK

SEQ ID NO: 483 - protein, 107 aa, synthetic construct
  REGION 1..107: note = Synthetic polypeptide
EIVMTQSPATLSLSPGERATLSCRASQSVSNYLAWYQQKPGQAPRLLISDASSRATGIPARFRGSGSGTDFTLTISSLEPEDFAVYYCLQRTNWPFTFGPGTKVEIK

SEQ ID NO: 484 - protein, 111 aa, synthetic construct
  REGION 1..111: note = Synthetic polypeptide
QSVLTQPASVSGSPGQSITISCTGTSSDIGGYNYVSWYQQHPGKVPKLVIYDVSNRPSGVSNRFSGSKSGNTASLTISGLQAEDEADYYCSSYTSGTTLGVFGTGTKLTVL

SEQ ID NO: 485 - protein, 110 aa, synthetic construct
  REGION 1..110: note = Synthetic polypeptide
QSVVTQPPSVSAAPGQKVTISCSGRSSNIGNSDVSWYQQFPGRAPKLLIYDNDERPSGIPDRFSGSKSGTSATLDITGLQTGDEADYYCGTWDSSLGGVIFGGGTKVTVL

SEQ ID NO: 486 - protein, 110 aa, synthetic construct
  REGION 1..110: note = Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCETWDSSLGVVVFGGGTKLTVL

SEQ ID NO: 487 - protein, 108 aa, synthetic construct
  REGION 1..108: note = Synthetic polypeptide
DIQVTQSPSSLSASVGDRVTITCQASQDISNYLNWYQHKPGRAPKLLIYDASNLERGVPSRFSGSGSGTDFTFTISSLQPEDIATYYCQQYDNLSRLTFGGGTKLEIK

SEQ ID NO: 488 - protein, 111 aa, synthetic construct; identical to SEQ ID NO: 470
  REGION 1..111: note = Synthetic polypeptide

SEQ ID NO: 489 - protein, 109 aa, synthetic construct
  REGION 1..109: note = Synthetic polypeptide
QSVLTQPPSMSAAPGQKVTISCSGSSSNIGNNYVSWYRQLPGTAPKLLIYDNDKRPSGIPDRFSGSKSGTTATLGITGLQTGDEAVYYCGTWDFRLSALFGGGTKLTVL

SEQ ID NO: 490 - protein, 111 aa, synthetic construct
  REGION 1..111: note = Synthetic polypeptide
QSVLIQPASVSGSPGQSITISCTGTSSDVGGDKYVSWYQQHPGKAPKLVIYEVSNRPSGVSNRFSGSKSGNTASLTISGLQAEDEADYYCSSYTSSGTPVVCGGGTKVTVL

SEQ ID NO: 491 - protein, 111 aa, synthetic construct
  REGION 1..111: note = Synthetic polypeptide
QSVLTQPPSVSAAPGQKVTISCSGSSSNIGNYYVSWYQQVPGTAPKLLIYDNNKRPSGIPDRFSGSKSGTSATLGITGLHTGDEAEYYCGTWDSSPSAGRVFGGGTKLTVL

SEQ ID NO: 492 - protein, 113 aa, synthetic construct
  REGION 1..113: note = Synthetic polypeptide
DIVLTQSPDSLAVSLGERATINCKSSQSVLFGSNQKSCLAWYQQKPGQSPKLLIHWASTRESGVPDRFSGSGSGTDFTLTISSLQAEDVAVYYCQQYYSTPRTFGQGTKVEIK

SEQ ID NO: 493 - protein, 109 aa, synthetic construct
  REGION 1..109: note = Synthetic polypeptide
QSVLTQPPSVSAAPGQKVTISCSGSSSNIGSNFVSWYQQLPGTAPKLLIYDNNKRPSGIPDRFSGSKSGTSATLAITGLQTGDEADYYCGTWDTRLSALFGGGTKVTVL

SEQ ID NO: 494 - protein, 111 aa, synthetic construct
  REGION 1..111: note = Synthetic polypeptide
QSVLTQPPSVSAAPGQKVTISCSGSSSNFGNDYVSWYQQLPGTAPKLLIYDNDKRPSGIPDRFSGSKSGTSATLGITGLQTGDEADYYCGTWDTSLSAAWVFGGGTKVTVL

SEQ ID NO: 495 - protein, 111 aa, synthetic construct
  REGION 1..111: note = Synthetic polypeptide
QSVVTQPPSVSAAPGQKVTISCSGSSSNIGNNYVSWYQQLPGTAPKLLIYDNNKRPSGIPDRFSGSTSGTSATLGITGLQTGDEAVYYCGTWDTSPSAGGVFGGGTKVTVL

SEQ ID NO: 496 - protein, 111 aa, synthetic construct
  REGION 1..111: note = Synthetic polypeptide
QPVLTQPASVSGSPGQSITISCTGTSSDVGGYNYVSWYQQHPGKAPKLMIYDVSNRPSGVSNRFSGSKSGNTASLTISGLQAEDEADYYCSSYTSSSTLAVFGGGTKLTVL

SEQ ID NO: 497 - protein, 111 aa, synthetic construct
  REGION 1..111: note = Synthetic polypeptide
QPVLTQPPSVSAAPGQKVTISCSGSSSNIGNDYVSWYQQLPGTAPKLLIYDNNKRPSGIPDRFSGSKSGTSATLGITGLQTGDEGDYYCGTWDSSLSAAWVFGGGTKVTVL

SEQ ID NO: 498 - protein, 111 aa, synthetic construct
  REGION 1..111: note = Synthetic polypeptide
QPVLTQSASVSGSPGQSITISCTGTSSDVGGYKYVSWYQQHPGKAPKLMIYEVSNRPSGVSIRFSGSKSGNTASLTISGLQAADEADYYCSSYRSSGTPYVFGTGTKVTVL

SEQ ID NO: 499 - protein, 107 aa, synthetic construct
  REGION 1..107: note = Synthetic polypeptide
EIVLTQSPSSLSASVGDRVTITCQASQDISNFLNWYQQKPGKAPKLLIYDASSLETGVPSRFSGSGSGTDFTFTISSLQPEDIATYYCQQYDNLPLTFGGGTKLEIK

SEQ ID NO: 500 - protein, 107 aa, synthetic construct
  REGION 1..107: note = Synthetic polypeptide
DIRLTQSPSTLSASVGDRVTVTCRASQNINTYLAWYQQIPGKAPRLLIYRASTLESGVPSRFSGSGSGTEFTLTINSLQPDDYATYYCQHYETYSVRFGQGTKVEIK

SEQ ID NO: 501 - protein, 107 aa, synthetic construct
  REGION 1..107: note = Synthetic polypeptide
DIQVTQSPSSLSASVGDRVTITCRASQGISNYLAWYQQKPGKVPKLLIFAASTLRSGVPSRFRGSGSGTDFTLTISSLQPEDVATYYCQKYNSAPLTFGGGTKVEIK

SEQ ID NO: 502 - protein, 107 aa, synthetic construct
  REGION 1..107: note = Synthetic polypeptide
DIVMTQTPATLSLSPGERATLSCRASQSVSSYLAWYQQKPGQAPRLLIYGASNRATGIPARFSGSGSGTDFTLTISSLEPEDFAVYYCLQRTNWPFTFGPGTKVEIK

SEQ ID NO: 503 - protein, 107 aa, synthetic construct
  REGION 1..107: note = Synthetic polypeptide
DIVLTQSPSTLSASVGDRVTVTCRASQNINTYLAWYQQIPGKAPRLLIYRASSLESGVPSRFSGSGSGTEFTLTISSLQPDDFATYYCQHYNSFSVKFGQGTKVEIK

SEQ ID NO: 504 - protein, 109 aa, synthetic construct
  REGION 1..109: note = Synthetic polypeptide
SYELTQPPSVSVAPGQTARITCGGHNVGSKSVHWYQQKPGQAPVLVVYDDSDRPSGIPERFSGSNSGNTATLTISRVEAGDEADYYCQVWDSSSDHPWVFGGGTKVTVL

SEQ ID NO: 505 - protein, 111 aa, synthetic construct
  REGION 1..111: note = Synthetic polypeptide
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDTSLSAGRVFGGGTKLTVL

SEQ ID NO: 506 - protein, 109 aa, synthetic construct
  REGION 1..109: note = Synthetic polypeptide
QPVLTQPPSVSVAPGQTARITCGGNNIGSKSVHWYQQKPGQAPMLVIYSNSDRPSGIPERFSGSNSGITATLTISRVEAGDEADYHCQVWDTSIDHHWVFGGGTKLTVL

SEQ ID NO: 507 - protein, 111 aa, synthetic construct
  REGION 1..111: note = Synthetic polypeptide
QSVLIQPPSASGSPGQSVTISCTGTSSDVGGYNYVSWYQQHPGKAPKLMIYEVSKRPSGVPDRFSGSKSGNTASLTVSGLQAEDGADYYCSSYAGSNNWVVFGGGTKLTVL

SEQ ID NO: 508 - protein, 110 aa, synthetic construct
  REGION 1..110: note = Synthetic polypeptide
QPVLTQPASVSGSPGQSITISCTGTSTDVGGYNYVSWYQQYPGKAPKLIIYDVTNRPSGVSHRFSGSKSGNTASLTISGLQAEDEADYYCSSYTTTSLVIFGGGTKLTVL

SEQ ID NO: 509 - protein, 113 aa, synthetic construct
  REGION 1..113: note = Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPRTFGQGTRLEIK

SEQ ID NO: 510 - protein, 107 aa, synthetic construct
  REGION 1..107: note = Synthetic polypeptide
DIQVTQSPSSLSASVGGRVTITCRASQGIRNDLGWYQRKPGKAPKRLIYAASSLQSGVPSRFSGSGSGTEFTLTISSLQPEDFATYYCLQHNSYPLTFGGGTKVDIK

SEQ ID NO: 511 - protein, 107 aa, synthetic construct
  REGION 1..107: note = Synthetic polypeptide
DIQLTQSPSTLSASVGDRVTITCRASQSISTWLAWYQQKPGKAPKLLIYRASSLESGVPSRFSASGSGTEFTLSISSLQPDDFATYYCKQYNRNPYTFGQGTKVEIK

SEQ ID NO: 512 - protein, 107 aa, synthetic construct
  REGION 1..107: note = Synthetic polypeptide
DIQMTQSPSSLSASVGDRVTITCRASQGISSYLAWFQQKPGKVPKLLIYAASTLQSGVPSRFSGSGSGTDFTLTISSLQPEDVATYYCQKYNSAPQTFGQGTKVDIK

SEQ ID NO: 513 - protein, 108 aa, synthetic construct
  REGION 1..108: note = Synthetic polypeptide
EIVMTQSPATLSVSPGERATLSCRASQSVSFNLAWYQQKPGQAPRLLISRASTRAAGVPARFSGSGSGTEFTLTISSLQSEDFAVYYCQQYNNWPPLTFGGGTKLEIK

SEQ ID NO: 514 - protein, 113 aa, synthetic construct
  REGION 1..113: note = Synthetic polypeptide
DIQMTQSPDSLTVSLGERATINCKSSQSVLYSSNNKNSLAWYQQKPGQPPKLLIYWASTRESGVPDRFSGSGSGTDFTLTISSLQAADVAVYYCQQYYRTPWTFGQGTKVEIK

SEQ ID NO: 515 - protein, 110 aa, synthetic construct
  REGION 1..110: note = Synthetic polypeptide
QSVLTQPPSVSAAPGQKVTISCSGSSSNIGNNYVSWYQQLPGTAPKVLIYDNNKRPSGIPDRFSGSKSGTSATLGITGLQTGDEADYYCGTWDSALGAAVFGGGTKLTVL

SEQ ID NO: 516 - protein, 107 aa, synthetic construct
  REGION 1..107: note = Synthetic polypeptide
DIQLTQSPSTLSASVGDRVTITCRASQSVSSWLAWYQQKPGKAPRLLIYRASSLESGVPSRFSGSGSGTEFTLTISSLQPDDFAAYYCQQYNRDPYTFGQGTKVEIK

SEQ ID NO: 517 - protein, 105 aa, synthetic construct
  REGION 1..105: note = Synthetic polypeptide
SYELTQLPSVSVSPGQTARVTCSGDALQYVYWYQQKPGQAPVVVIYKDTERPSGIPERFSGSSSGTTVTLTITGVQAEDEADYYCQSADRSGSVIFGGGTKVTVL

SEQ ID NO: 518 - protein, 106 aa, synthetic construct
  REGION 1..106: note = Synthetic polypeptide
DIVMTQSPATLSLSPGERATLSCRASQSVSSYLAWYQQKPGQAPRLLIYDASNRATGIPARFSGSGSGTDFTLTISSLEPEDFAVYYCQQRSNWPSFGQGTKLEIK

SEQ ID NO: 519 - protein, 107 aa, synthetic construct
  REGION 1..107: note = Synthetic polypeptide
DIRLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 520 - protein, 108 aa, synthetic construct
  REGION 1..108: note = Synthetic polypeptide
ETTLTQSPGTLSLSPGERATLSCRASRSVSGNYLAWYQQKPGQAPRLLIYAASSRATGIPDRFSGGGSGTHFTLTISRLEPEDFAVYYCQQYGSSPRAFGQGTKVEIK

SEQ ID NO: 521 - protein, 107 aa, synthetic construct
  REGION 1..107: note = Synthetic polypeptide
EIVMTQSPSSLSASVGDRVTITCRASQSIRSYLNWYQQKPGKAPKLLIYAASSLQSGVPLRFSGSGSGTDFTLTISSLQPEDFATYYCQQSSITPPTFGQGTKLEIK

SEQ ID NO: 522 - protein, 107 aa, synthetic construct
  REGION 1..107: note = Synthetic polypeptide
DIQMTQSPSTLSASVGDRVTITCRASQSISSWLAWYQQKPGKAPKLLIYQASSLESGVPSRFSGSESGTEFTLTISSLQPDDFATYYCQQYNSFPFTFGPGTKVEIK

SEQ ID NO: 523 - protein, 107 aa, synthetic construct
  REGION 1..107: note = Synthetic polypeptide
DIVLTQSPSSLSASVGDRVTITCRASQSINNYLNWYQQKPGKAPNLLIFGASTLQSGVPSRFTGSGSGTVFTLTISSLQRDDFVIYYCQQTYSASGSFGQGTKVEIK

SEQ ID NO: 524 - protein, 107 aa, synthetic construct
  REGION 1..107: note = Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

SEQ ID NO: 525 - protein, 109 aa, synthetic construct
  REGION 1..109: note = Synthetic polypeptide
QSALIQPPSVSAAPGQKVTISCSGSSSNIGNNYVSWYQQLPGTAPKLLIYDNNKRPSGIPDRFSGSKSGTSATLGITGLQTGDEADYYCGTWDNSLGVVFGGGTQLTVL

SEQ ID NO: 526 - protein, 107 aa, synthetic construct
  REGION 1..107: note = Synthetic polypeptide
EIVLTQSPGTLSLSPGERATLSCRASQSVSSSYLAWYQQKPGQAPRLLIYGASSRATGIPDRFSGSGSGTDFTLSINRLEPEDFAVYYCQQYGSSPGFGQGTKVEIK

SEQ ID NO: 527 - protein, 107 aa, synthetic construct
  REGION 1..107: note = Synthetic polypeptide
DIVLTQSPSTLSASVGDRVTITCRASQSISDWLAWYQQKPGKAPGLLIYRASGLESGVPSRFSGSGSGTEFTLTISSLQPDDFATYYCHQYKDFPWTFGQGTKVDIK

SEQ ID NO: 528 - protein, 106 aa, synthetic construct
  REGION 1..106: note = Synthetic polypeptide
DIQMTQSPSTLSASVGDRVTITCRASQSISTWLAWYQLKPGKAPKLLIYKASNLQSGVPSRFSGSGSGTEFTLTISSLQPDDFATYYCQQYNSYSPWGQGTKLEIK

SEQ ID NO: 529 - protein, 107 aa, synthetic construct
  REGION 1..107: note = Synthetic polypeptide
EIVLTQSPGTLSLSPGERATLSCRASQSVSSRYLAWYRQKPGQAPRLLIYGASSRATGIPDRFSGSGSGTDFTLTISRLEPEDFAVYYCQQYGSSPFFGGGTKLEIK

SEQ ID NO: 530 - protein, 111 aa, synthetic construct
  REGION 1..111: note = Synthetic polypeptide
QSVLTQPASVSGSPGQSITISCTGTSSDVGGDKYVSWYQQHPGKAPKPMIYEVSNRPSGVSNRFSGSKSGNTASLTISGLQAEDEADYYCSSYTSSSTPVVFGGGTKLTVL

SEQ ID NO: 531 - protein, 110 aa, synthetic construct
  REGION 1..110: note = Synthetic polypeptide
QPVLTQPRSVSGSPGQSVTISCTGTSSDVGGYNYVSWYQQHPGKAPKLMISDVSKRPSGVPDRFSGSKSGNTASLTISGLQADDEADYYCCSYATNYGVVFGGGTKVTVL

SEQ ID NO: 532 - protein, 110 aa, synthetic construct
  REGION 1..110: note = Synthetic polypeptide
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVVFGGGTKVTVL

SEQ ID NO: 533 - protein, 111 aa, synthetic construct
  REGION 1..111: note = Synthetic polypeptide
QSVLTQPPSVSAAPGQKVTISCSGSSSNIGNNDVSWYQQLPGRAPKLLIYDNNKRPSGIPDRFSGSKSGTSATLGITGLQTGDEADYYCGAWDSSLSAHVVFGGGTKVTVL

SEQ ID NO: 534 - protein, 107 aa, synthetic construct
  REGION 1..107: note = Synthetic polypeptide
DIVMTQTPSTLSASVGDRVTVTCRASQSISDWLAWYQQKAGKAPKLLIYRASSLESGVPPRFSGSGSGTEFTLTISSLRPDDFATYYCQQYNRYPYTFGQGTKVDIK

SEQ ID NO: 535 - protein, 107 aa, synthetic construct
  REGION 1..107: note = Synthetic polypeptide
DIQVTQSPSSLSASVGDRVTITCRASQGIRNDLAWYQQRPGKAPKRLIYAASSLQSGVPSRFSGSGSGTEFTLTISSLQPEDFATYYCLQHHSYPWTFGQGTKVEIK

SEQ ID NO: 536 - protein, 107 aa, synthetic construct
  REGION 1..107: note = Synthetic polypeptide
DIRMTQSPSTLSASIGDRVTITCRASQSISDWLAWYLQKPGKAPSLLIYRASSLETGVPSRFSGRGSGTEFTLTISSLQPDDFGTYYCQQYNRDPYTFGQGTKVDIK

SEQ ID NO: 537 - protein, 107 aa, synthetic construct
  REGION 1..107: note = Synthetic polypeptide
DIQLTQSPSTLSASVGDRVTVTCRASQNVGGWLAWYQQKPGKAPKLLIFQASRLENGVPSRFSANASGTEFTLTIGSLQPDDFATYYCQQYNTYPYTFGQGTKVDIK

SEQ ID NO: 538 - protein, 107 aa, synthetic construct
  REGION 1..107: note = Synthetic polypeptide
DIQLTQSPSSLSASVGDRVTITCRASQSISQYLNWYQQKPGKAPKLLISPASSFQSGVPSRFSGSGSGTDFTLTITSLQPEDFATYYCQQSYSTPWTFGQGTKVDIK

SEQ ID NO: 539 - protein, 110 aa, synthetic construct
  REGION 1..110: note = Synthetic polypeptide
QSVLTQPASVSGSPGQSITISCTGTSSDVGGYNYVSWYQQHPGKAPKLMIYDVSNRPSGVSNRFSGSKSGNTASLTISGLQAEDEADYYCSSYTSSSTLVFGGGTQLTVL

SEQ ID NO: 540 - protein, 108 aa, synthetic construct
  REGION 1..108: note = Synthetic polypeptide
SYELTQPPSVSVAPGQTARIICGGNYIGGKSVHWYQQKPGQAPVLVVYNDNDRPSGIPERFSGSNSGNTATLTISRVAAGDEADYYCQVWDNSSDRRVFGGGTKLTVL

SEQ ID NO: 541 - protein, 106 aa, synthetic construct
  REGION 1..106: note = Synthetic polypeptide
DIRVTQSPATLSVSPGERATLSCRASQRVNSNLAWYQQKPGQAPRLLIYGASTRATGIPVRFSGSGSGTEFTLTISSLQSEDFAVYYCQQYNTWWTFGQGTKVEIK

SEQ ID NO: 542 - protein, 107 aa, synthetic construct
  REGION 1..107: note = Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIK

SEQ ID NO: 543 - protein, 107 aa, synthetic construct
  REGION 1..107: note = Synthetic polypeptide
DIVMTQSPATLSVSPGERATLSCRASQSVSSNLAWYQQKPGQAPRLLIYGASTRATGIPARFSGSGSGTEFTLTISSLQSEDFAVYSCQQYNTWPKTFGQGTKVEIK

SEQ ID NO: 544 - protein, 108 aa, synthetic construct
  REGION 1..108: note = Synthetic polypeptide
EIVLTQSPGTLSLSPGERATLSCRASQSVSSSFLAWYQQKPGQAPRLLIYGASSRATGIPDRFSGSGSGTDFTLTIRRLEPEDFAVYYCQQYGSSRRTFGQGTKVEIK

SEQ ID NO: 545 - protein, 107 aa, synthetic construct
  REGION 1..107: note = Synthetic polypeptide
DIRVTQSPSTLSASVGDRVTITCRASQSISSWLAWYQQKPGKAPKLLIYRASSLESGVPSRFSGSGSGTEFTLTISSLQPDDFATYYCQQYNRYPYTFGQGTKVEIK

SEQ ID NO: 546 - protein, 112 aa, synthetic construct
  REGION 1..112: note = Synthetic polypeptide
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTLDVVFGGGTKLTVL

SEQ ID NO: 547 - protein, 108 aa, synthetic construct
  REGION 1..108: note = Synthetic polypeptide
EIVMTQSPATLSVSPGERATLSCRASQSVSSNLAWYQQKPGQAPRLLIYGASTRATSIPARFSGSGSGTEFTLTISSLQSEDFAVYYCQQYNSWPPITFGQGTRLEIK

SEQ ID NO: 548 - protein, 107 aa, synthetic construct
  REGION 1..107: note = Synthetic polypeptide
DIRLTQSPSTLSASVGDRVSITCRASQSISDWLAWYQQKPGKAPKLLIYRASGLETGVPSRFSGSGSGTEFTLTISSLQPDDFATYYCQQYNRYPYTFGQGTKVDIK

SEQ ID NO: 549 - protein, 111 aa, synthetic construct
  REGION 1..111: note = Synthetic polypeptide
QPVLIQPPSASGTPGQRVTISCSGSSSNFGSNFVYWYQQLPGTAPKLLIYRVNQRPSGVPDRFSGSKSGTSASLAISGLRSEDEADYYCATWDVSLSNDVLFGGGTKLTVL

SEQ ID NO: 550 - protein, 109 aa, synthetic construct
  REGION 1..109: note = Synthetic polypeptide
DIVLTQSPATLSLSPGERATLSCRASQSVSSSYLSWYQQKPGQAPRLLIYGASSRATGIPDRFSGSGSGTDFTLTISRLEPEDFAVYYCQQYGSSPPITFGGGTKVEIK

SEQ ID NO: 551 - protein, 107 aa, synthetic construct
  REGION 1..107: note = Synthetic polypeptide
DIQMTQSPSSLSASVGDRVTITCRASQTITRYMNWYQQKPGEAPKLLIYATSSLQSGVPSRFSGSGSGTDFTLTITNLQPADFATYYCQQSSTTRWTFGQGTKVDIK

SEQ ID NO: 552 - protein, 107 aa, synthetic construct
  REGION 1..107: note = Synthetic polypeptide
DIRLTQSPSSLSASVGDRVTITCRASQDIRKFLNWYQQKLGKAPSLLIYGASSLQSGVPSRFSGSGSGTDFTLTISSLQPEDFAIYYCQHASTTPWTFGQGTKVEIK

SEQ ID NO: 553 - protein, 108 aa, synthetic construct
  REGION 1..108: note = Synthetic polypeptide
SYELTQPPSVSVSPGQTATITCSGDKLGYTYTCWYQQKPGQSPVLVIYQDTKRPSGIPERFSGSNSGNTATLTITGTQAMDEADYYCQAWDTTTAGGVFGGGTKLTVL

SEQ ID NO: 554 - protein, 108 aa, synthetic construct
  REGION 1..108: note = Synthetic polypeptide
DIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRAFGPGTKVEIK

SEQ ID NO: 555 - protein, 110 aa, synthetic construct
  REGION 1..110: note = Synthetic polypeptide
QSVLTQPPSASGSPGQSVTISCAGTRSDVGGYNFVSWYQQHPGKAPKLLIYEVNKRPSGVPDRFSGSKSANTASLTVSGLQAEDEAEYFCSSYGGNNDLVFGGGTKVTVL

SEQ ID NO: 556 - protein, 107 aa, synthetic construct
  REGION 1..107: note = Synthetic polypeptide
EIVMTQSPATLSLSPGERGTLSCRTSQSVSSFLAWYQQKPGQAPRLLMYDASNRATGIPARFSGSGSGTDFTLTISSLEPEDFAVYYCQQRSNWPYTFGQGTKVDIK

SEQ ID NO: 557 - protein, 106 aa, synthetic construct
  REGION 1..106: note = Synthetic polypeptide
GIQLTQSPSTLSASVGDRVTITCRASQSVSDWLAWYQQKPGRAPNLLIYRASSLQSGVPSRFSGSGSGTEFTLTINSLQPDDFATYYCQQYKTYWTFGQGTKVEIK

SEQ ID NO: 558 - protein, 110 aa, synthetic construct
  REGION 1..110: note = Synthetic polypeptide
QSVLTQPASVSGSPGQSITISCTGTSSDVGGYNYVSWYQQHPGKAPKLMIYEVSNRPSGVSNRFSGSKSGNTASLTISGLQAEDEADYYCSSYTSSGTNIFGTGTKLTVL

SEQ ID NO: 559 - protein, 108 aa, synthetic construct
  REGION 1..108: note = Synthetic polypeptide
DIVMTQTPATLSVSPGERATLSCRASQSVSSNVAWYQQKPGQAPRLLIHGASTRATGIPARFSGSGSGTEFTLTISSLQSEDFAVYYCQQYNNWPPLTFGGGTKLEIK

SEQ ID NO: 560 - protein, 108 aa, synthetic construct
  REGION 1..108: note = Synthetic polypeptide
SYELTQPPSVSVSPGQTARITCSGDALPKKYVYWFQQKSGQAPVLVIYEDRRGPSGIPERFSGSTSGTMATLTIRGAQVEDEADYFCYSTDSSGLLGVFGGGTKLTVL

SEQ ID NO: 561 - protein, 113 aa, synthetic construct
  REGION 1..113: note = Synthetic polypeptide
DIQMTQSPDSLAVSLGERATINCKSSQSVFYSSNSQNYLAWYQQKPGQPPKLLIYWASTRESGVPDRFSGSGSATDFSLTISSLQAEDVAVYYCQQFHSPPWTFGQGTKLEIK

SEQ ID NO: 562 - protein, 107 aa, synthetic construct
  REGION 1..107: note = Synthetic polypeptide
DIVMTQSPSTLSASVGDRVVITCRASQSISNWLAWYQQKSGKAPKLLIYKASRLESGVPSTFSGSGSGTEFTLTISSLQADDFASYYCQQYNDYPWTFGQGTKVEIK

SEQ ID NO: 563 - protein, 107 aa, synthetic construct
  REGION 1..107: note = Synthetic polypeptide
EIVMTQSPSSLSASVGDRVTITCRASQGIRNDLGWYQQKPGKAPKRLIYAASSLQSGVPSRFSGSGSGREFTLTISSLQPEDFATYYCLQHNTYPWTFGQGTKVEIK

SEQ ID NO: 564 - protein, 107 aa, synthetic construct
  REGION 1..107: note = Synthetic polypeptide
DIQVTQSPSTLSASVGDRVSITCRASQTISSWLAWYQQKPGKAPKLLMYKASNLQSGVPSRFTGSGSGTEFTLTISSLQPDDFATYYCQQYYSYPYTFGPGTKVDIK

SEQ ID NO: 565 - protein, 107 aa, synthetic construct
  REGION 1..107: note = Synthetic polypeptide
SYVLTQPPSVSVSPGQTARITCSGDALPKQYGYWYQQKPGQAPVLVIYKDSERPSGIPERFSGSSSGTTVTLTISGVQAEDEADYYCQSADRSGTVVFGGGTKLTVL

SEQ ID NO: 566 - protein, 110 aa, synthetic construct
  REGION 1..110: note = Synthetic polypeptide
QAVVTQPPSASGSPGQSVTISCTGTSSDVGGYNYVSWYQQHPGKAPKLMVYEVTKRPSGVPDRFSGSKSGNAASLTVSGLQAEDEAEYYCSSYAGSNALVFSGGTKLTVL

SEQ ID NO: 567 - protein, 110 aa, synthetic construct
  REGION 1..110: note = Synthetic polypeptide
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDSSNWVFGGGTKLTVL

SEQ ID NO: 568 - protein, 105 aa, synthetic construct
  REGION 1..105: note = Synthetic polypeptide
QPELTQPPSVSVSPGQTARITCSGDALSKQYAYWYQQKPGQAPVVVIYKDSERPSGIPERFSGSRSGTTVTLTISGVQAEDEADYYCHSPDSHVVFGGGTKLTVL

SEQ ID NO: 569 - protein, 108 aa, synthetic construct
  REGION 1..108: note = Synthetic polypeptide
SYELIQLPSASVAPGKTARITCGGNNIGSKSVHWYQQKPGQAPVLVVYDDSDRPSGIPERFSGSNSGNTATLTISRVEAGDEADYYCQVWDSSSDHEVFGGGTKLTVL

SEQ ID NO: 570 - protein, 103 aa, synthetic construct
  REGION 1..103: note = Synthetic polypeptide
DIQMTQSPSSLSASVGDRVTITCQASQDISNYLNWYQQKPGKAPKLLIYDVSKLKTGVPPRFSGSGSGTDFTFTISSLQPEDIATYYCQQWGTFGQGTKVDIK

SEQ ID NO: 571 - protein, 107 aa, synthetic construct
  REGION 1..107: note = Synthetic polypeptide
EIVLTQSPSTLSASVGDRVTITCRASQSISDWLAWYQQKPGKAPNLLIYRASSLESGVPSRFSGSGSGTEFTLTISSLQPDDFATYYCQQYNRYPYTFGQGTKVEIK

SEQ ID NO: 572 - protein, 107 aa, synthetic construct
  REGION 1..107: note = Synthetic polypeptide
DIQLTQSPSTLSASVGDRVTITCRASQSISDWLAWFQQKPGKAPKLLIYRASGLETGVPSRFSGSGSGTEFTLTISSLQPDDFATYYCQQYNRYSYTFGQGTKVEIK

SEQ ID NO: 573 - protein, 106 aa, synthetic construct
  REGION 1..106: note = Synthetic polypeptide
DIRLTQSPSTLSASVGDRVTITCRASQSISGWLAWYQQKPGKAPKLLIYKASILESGVPSRFSGSQSGTEFTLTISSLQPDDFATYYCQQYNNFWTFGQGTKLEIK

SEQ ID NO: 574 - protein, 111 aa, synthetic construct
  REGION 1..111: note = Synthetic polypeptide
QSVLTQPPSVSGAPGQRVTISCTGNSSNIGAGYEVHWYQQLPGTAPKLLIYGNNNRPSGVPDRFSGSKSGASGSLAVTGLRAEDEADYYCHSYDSNMSGSVFGGGTKVTVL

SEQ ID NO: 575 - protein, 108 aa, synthetic construct
  REGION 1..108: note = Synthetic polypeptide
EIVLTQSPSSLSASVGDRVTITCRASQGISNYLAWYQQKPGKAPKLLIYAASTLQSGVPSRFSGSGSGTDFILTISSLQPEDVATYYCQKYYSAPLITFGPGTKVEIK

SEQ ID NO: 576 - protein, 110 aa, synthetic construct
  REGION 1..110: note = Synthetic polypeptide
SYELTQPPSVSVSPGQTARITCSGDALPKQYAYWYQQKPGQAPVLVIYKDTERPSGIPERFSGSSSGTTVTLTISGVQAEDEADYYCQSADSSVADSSVVFGGGTKLTVL

SEQ ID NO: 577 - protein, 107 aa, synthetic construct
  REGION 1..107: note = Synthetic polypeptide
EIVLTQSPATLSLSPGERATLSCRASQSVSNYFAWYQQKPGQAPRLLIYGASNRATGVPARFSGSGSGTDFTLTISSLEPEDFAVYYCQQRSNWPYTFGQGTKVEIK

SEQ ID NO: 578 - protein, 110 aa, synthetic construct
  REGION 1..110: note = Synthetic polypeptide
NFMLTQPPSVSAAPGQKVTISCSGSNSNIGNNFVSWYQQLPGTAPKLLIYDNNERPSGIPDRFSGSKSVTSATLGITGLQTGDEADYYCGTWDNSLGMVVFGGGTKLTVL

SEQ ID NO: 579 - protein, 110 aa, synthetic construct
  REGION 1..110: note = Synthetic polypeptide
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTYVFGTGTKVTVL

SEQ ID NO: 580 - protein, 109 aa, synthetic construct
  REGION 1..109: note = Synthetic polypeptide
EIVLTQSPGTLALSPGERATLSCRASQSVSSYLAWYQQKPGQAPRLLIYDSSNRATGIPARFSGSGSGTDFTLTISSLEPEDFAVYYCQQPGNWPPAFTFGGGTKLEIK

SEQ ID NO: 581 - protein, 106 aa, synthetic construct
  REGION 1..106: note = Synthetic polypeptide
DIVMTQSPATLSVSPGERATLSCRASQSVTSKLAWYQQKPGQAPRLLIYGASTRATGIPARFSGSGSGTEFTLTISSLQSEDFAVYYCQQYNNWITFGQGTRLEIK

SEQ ID NO: 582 - protein, 107 aa, synthetic construct
  REGION 1..107: note = Synthetic polypeptide
DIQLTQSPSSVSASVGDRVTITCRASQGISSWLAWYQQKPGKAPKLLIYAASSLQSGVPSRFSGSGSGTDFTLTISSLQPEDFATYYCQQANSFPWTFGQGTKVDIK

SEQ ID NO: 583 - protein, 112 aa, synthetic construct
  REGION 1..112: note = Synthetic polypeptide
DIVMTQSPLSLPVTPGEPASISCRSSQSLLHSNGYNSLDWYLQKPGQSPQLLIYLGSNRASGVPDRFSGSGSGTDFTLKISRVEAEDVGVYYCMQALQTPYTFGQGTKLEIK

SEQ ID NO: 584 - protein, 110 aa, synthetic construct
  REGION 1..110: note = Synthetic polypeptide
QPVLTQPPSASGTPGQRVTISCSGSSSNIGSNTVHWYQQLPGTAPKLLIYSNNQRPSGVPDRLSGSRSGTSASLAISGLQSEDEAEYYCAAWDDNLIGVVFGGGTKLTVL

SEQ ID NO: 585 - protein, 111 aa, synthetic construct
  REGION 1..111: note = Synthetic polypeptide
QSALTQPPSASGSPGQSVTISCTGTSSDVGGYNYVSWYQQHPGKAPKLMIYEVSKRPSGVPDRFSGSKSGNTASLTVSGLQAEDEADYYCSSFAGSNNLYVFGTGTKVTVL

SEQ ID NO: 586 - protein, 110 aa, synthetic construct
  REGION 1..110: note = Synthetic polypeptide
QSVLTQPASVSGSPGQSITISCTGTSSDVGGYNYVSWYQQHPGKAPKLMIYDVTNRPSGVSNRFSGSRSGNTASLTISGLQAEDEADYYCSSYTRSSTRVFGGGTKLTVL

SEQ ID NO: 587 - protein, 109 aa, synthetic construct
  REGION 1..109: note = Synthetic polypeptide
QPVLTQPPSVSAAPGQKVTISCSGSSSNIGSNFVSWYQQFPGTAPKLLIYDDNKRPSGIPDRFSGSKSGTSATLGITGLQTGDEADYYCETWDSRLSVVFGGGTKLTVL

SEQ ID NO: 588 - protein, 110 aa, synthetic construct
  REGION 1..110: note = Synthetic polypeptide
QPVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKVTVL

SEQ ID NO: 589 - protein, 107 aa, synthetic construct
  REGION 1..107: note = Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVDIK

SEQ ID NO: 590 - protein, 107 aa, synthetic construct
  REGION 1..107: note = Synthetic polypeptide
DIRVTQSPSSLSASVGDRVTITSRASQSISSYLNWYQQKPGKAPKLLIYAASSLQSGVPSRFSGSGSGTDFTLTISSLQPEDFATYYCQQSYSTPRTFGGGTKVDIK

SEQ ID NO: 591 - protein, 111 aa, synthetic construct
  REGION 1..111: note = Synthetic polypeptide
QSVLTQPPSVSGAPGQRVTISCTGSSSDIGAGYDVHWYQQLPGTAPKLLIYGNTNRPSGVPDRFSGSKSGTSASLAITGLQAEDEADYYCQSYDSSLSGVVFGGGTKLTVL

SEQ ID NO: 592 - protein, 113 aa, synthetic construct
  REGION 1..113: note = Synthetic polypeptide
DIVLTQSPDSLAVSLGERAAINCKSSQSVFFSSDNKNYLAWYQQKPGQPPKLLIYWASTRESGVPDRFSGSGSGTDFTLTISSLQAEDVAVYYCQQFYTTPSTFGQGTKVEIK

SEQ ID NO: 593 - protein, 107 aa, synthetic construct
  REGION 1..107: note = Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGGGTKVEIK

SEQ ID NO: 594 - protein, 107 aa, synthetic construct
  REGION 1..107: note = Synthetic polypeptide
DIQLTQSPSSLSASVGDRVTITCRASQGIRNDLGWYQQKPGKAPKRLIYAASSLQSGVPSRFSGSGSGTEFTLTISSLQPEDFATYYCLQHNSYPLTFGGGTKVEIK

SEQ ID NO: 595 - protein, 110 aa, synthetic construct
  REGION 1..110: note = Synthetic polypeptide
NFMLTQPHSVSESPGNTVTISCTRSSGSIASTYVQWYQQRPGSAPSTVIYEDNQRPPGVPARFSGSIDSSSNSASLTISGLETEDEADYYCQSYDSTTVVFGGGTKVTVL

SEQ ID NO: 596 - protein, 111 aa, synthetic construct
  REGION 1..111: note = Synthetic polypeptide
SYVLTQPPSASGSPGQSVTISCTGTSSDFGGYNYVSWYQQHPGKAPKLMVYEVAKRPSGVPDRFSGSKSGNTASLTVSGLQAEDEADYYCSSYAGSNNFVVFGGGTKLTVL

SEQ ID NO: 597 - protein, 110 aa, synthetic construct
  REGION 1..110: note = Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAKVFGGGTKLTVL

SEQ ID NO: 598 - protein, 107 aa, synthetic construct
  REGION 1..107: note = Synthetic polypeptide
ETTLTQSPSTLSTSVGDRVTITCRASQSISSWLAWYQQKPGKAPKLLIYRASSLETEVPSRFSGSGSGTDFTLTISRLQPDDFATYFCQQYNRYPYTFGQGTKLEIK

SEQ ID NO: 599 - protein, 111 aa, synthetic construct
  REGION 1..111: note = Synthetic polypeptide
QPVLTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTFVLFGGGTKLTVL

SEQ ID NO: 600 - protein, 106 aa, synthetic construct
  REGION 1..106: note = Synthetic polypeptide
DIRVTQSPSSLSASVGDRVTISCRASESISIYLNWYQQKPGKAPNLLIYAASSLQRGVPSRFSGSGSGTDFTLTITSLQAEDFATYYCQQTFSIWTFGQGTKVEIK

SEQ ID NO: 601 - protein, 111 aa, synthetic construct
  REGION 1..111: note = Synthetic polypeptide
QPVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGKVFGGGTKLTVL

SEQ ID NO: 602 - protein, 110 aa, synthetic construct
  REGION 1..110: note = Synthetic polypeptide
QSALTQPPSASGSPGQSVTISCTGTSSDVGGFNYVSWYQQHPGRAPKLVIYEVNRRPSGVPDRFSGSKSGYTASLTVSGLQAEDEADYYCFSYAGSNNYVFGTGTKVTVL

SEQ ID NO: 603 - protein, 112 aa, synthetic construct
  REGION 1..112: note = Synthetic polypeptide
DIVMTQTPLSSPVTLGQPASISCRSSQSLVHSDGNTYLSWLQQRPGQPPRFLIYKISNRFSGVPDRFSGGGAGTDFTLKISRVEAEDVGVYYCMQASQFPLTFGGGTKVEIK

SEQ ID NO: 604 - protein, 108 aa, synthetic construct
  REGION 1..108: note = Synthetic polypeptide
EIVMTQSPSSLSASVGDRVTITCQASQDISNYLNWYQQKPGKAPKLLIYDASNLETGVPSRFSGSGSGTDFTFTISSLQPEDIATYYCQQYDNLPPVTFGQGTRLEIK

SEQ ID NO: 605 - protein, 107 aa, synthetic construct
  REGION 1..107: note = Synthetic polypeptide
QPVLTQPPSVSVSPGQTASITCSGDKLGDKYACWYQQKPGQSPVLVIYQDSKRPSGIPERFSGSNSGNTATLTISGTQAMDEADYYCQAWDSSTDVVFGGGTKVTVL

SEQ ID NO: 606 - protein, 107 aa, synthetic construct
  REGION 1..107: note = Synthetic polypeptide
DIQVTQSPSSLSASVGDRVTITCRASQGISNNLAWYQQKPGIFPKLLIYAASTLQSGVPSRFSGSGSGTDFILTISSLQPEDVATYYCQKYQSAPPTFGGGTKLEIK

SEQ ID NO: 607 - protein, 10 aa, synthetic construct
  REGION 1..10: note = Synthetic polypeptide
YSISSGFYWG

SEQ ID NO: 608 - protein, 16 aa, synthetic construct
  REGION 1..16: note = Synthetic polypeptide
SMYQSGITYYNPSLKS

SEQ ID NO: 609 - protein, 9 aa, synthetic construct
  REGION 1..9: note = Synthetic polypeptide
FTFSNYAMN

SEQ ID NO: 610 - protein, 17 aa, synthetic construct
  REGION 1..17: note = Synthetic polypeptide
AINRGGDSTYYADSVKG

SEQ ID NO: 611 - protein, 16 aa, synthetic construct
  REGION 1..16: note = Synthetic polypeptide
SMYHSGITYYNPSLKS

SEQ ID NO: 612 - protein, 9 aa, synthetic construct
  REGION 1..9: note = Synthetic polypeptide
FAFSSYGMH

SEQ ID NO: 613 - protein, 17 aa, synthetic construct
  REGION 1..17: note = Synthetic polypeptide
LIRFDGTIKYYADSVKG

SEQ ID NO: 614 - protein, 9 aa, synthetic construct
  REGION 1..9: note = Synthetic polypeptide
FTFNSHGMH

SEQ ID NO: 615 - protein, 17 aa, synthetic construct
  REGION 1..17: note = Synthetic polypeptide
VISYDGTKKYFADSVKG

SEQ ID NO: 616 - protein, 9 aa, synthetic construct
  REGION 1..9: note = Synthetic polypeptide
FTFRNYAMN

SEQ ID NO: 617 - protein, 17 aa, synthetic construct
  REGION 1..17: note = Synthetic polypeptide
GISGGGDSTNYADSVKG

SEQ ID NO: 618 - protein, 9 aa, synthetic construct
  REGION 1..9: note = Synthetic polypeptide
LIFRNYAMS

SEQ ID NO: 619 - protein, 17 aa, synthetic construct
  REGION 1..17: note = Synthetic polypeptide
SFSGSGGSAYYADSVKG

SEQ ID NO: 620 - protein, 17 aa, synthetic construct
  REGION 1..17: note = Synthetic polypeptide
GMRFDGTKIYYADSVKG

SEQ ID NO: 621 - protein, 10 aa, synthetic construct
  REGION 1..10: note = Synthetic polypeptide
GSISSDYWWS

SEQ ID NO: 622 - protein, 16 aa, synthetic construct
  REGION 1..16: note = Synthetic polypeptide
EIYHTGSTNYNPSLKS

SEQ ID NO: 623 - protein, 10 aa, synthetic construct
  REGION 1..10: note = Synthetic polypeptide
GSITSSNWWS

SEQ ID NO: 624 - protein, 16 aa, synthetic construct
  REGION 1..16: note = Synthetic polypeptide
DIYHSGSTSYNPSLKS

SEQ ID NO: 625 - protein, 10 aa, synthetic construct
  REGION 1..10: note = Synthetic polypeptide
DSISSNNWWS

SEQ ID NO: 626 - protein, 16 aa, synthetic construct
  REGION 1..16: note = Synthetic polypeptide
EIYHSGSTSYNPSLKS

SEQ ID NO: 627 - protein, 9 aa, synthetic construct
  REGION 1..9: note = Synthetic polypeptide
FTFGDYYMS

SEQ ID NO: 628 - protein, 17 aa, synthetic construct
  REGION 1..17: note = Synthetic polypeptide
YISSSGSSIYYTDSVRG

SEQ ID NO: 629 - protein, 10 aa, synthetic construct
  REGION 1..10: note = Synthetic polypeptide
GSISSSDWWS

SEQ ID NO: 630 - protein, 16 aa, synthetic construct
  REGION 1..16: note = Synthetic polypeptide
EIYHSGSTSYNPSVKS

SEQ ID NO: 631 - protein, 10 aa, synthetic construct
  REGION 1..10: note = Synthetic polypeptide
DSISSSHWWC

SEQ ID NO: 632 - protein, 10 aa, synthetic construct
  REGION 1..10: note = Synthetic polypeptide
GSISSSYWWS

SEQ ID NO: 633 - protein, 16 aa, synthetic construct
  REGION 1..16: note = Synthetic polypeptide
EVYHSGSTHYNPSLKS

SEQ ID NO: 634 - protein, 10 aa, synthetic construct
  REGION 1..10: note = Synthetic polypeptide
GSISSSNWWS

SEQ ID NO: 635 - protein, 16 aa, synthetic construct
  REGION 1..16: note = Synthetic polypeptide
DIYHTGSTSYNPSLKS

SEQ ID NO: 636 - protein, 9 aa, synthetic construct
  REGION 1..9: note = Synthetic polypeptide
FIFSDYYMN

SEQ ID NO: 637 - protein, 17 aa, synthetic construct
  REGION 1..17: note = Synthetic polypeptide
TISGSGKSIYYADSVKG

SEQ ID NO: 638 - protein, 9 aa, synthetic construct
  REGION 1..9: note = Synthetic polypeptide
FTFSDYYMS

SEQ ID NO: 639 - protein, 17 aa, synthetic construct
  REGION 1..17: note = Synthetic polypeptide
YITSSGNTKYYADSVKG

SEQ ID NO: 640 - protein, 9 aa, synthetic construct
  REGION 1..9: note = Synthetic polypeptide
FTFDDYAMH

SEQ ID NO: 641 - protein, 17 aa, synthetic construct
  REGION 1..17: note = Synthetic polypeptide
GISWNGGGIGYADSVKG

SEQ ID NO: 642 - protein, 9 aa, synthetic construct
  REGION 1..9: note = Synthetic polypeptide
FTFSSYAMS

SEQ ID NO: 643 - protein, 17 aa, synthetic construct
  REGION 1..17: note = Synthetic polypeptide
AISGSGGSTYYADSVKG

SEQ ID NO: 644 - protein, 10 aa, synthetic construct
  REGION 1..10: note = Synthetic polypeptide
GSISSNKWWS

SEQ ID NO: 645 - protein, 9 aa, synthetic construct
  REGION 1..9: note = Synthetic polypeptide
FTFSDDYMS

SEQ ID NO: 646 - protein, 17 aa, synthetic construct
  REGION 1..17: note = Synthetic polypeptide
YISGSGRAMYYADSVQG

SEQ ID NO: 647 - protein, 10 aa, synthetic construct
  REGION 1..10: note = Synthetic polypeptide
SSITSSHWWS

SEQ ID NO: 648 - protein, 16 aa, synthetic construct
  REGION 1..16: note = Synthetic polypeptide
DIYHSGGTTYNPSLKS

SEQ ID NO: 649 - protein, 9 aa, synthetic construct
  REGION 1..9: note = Synthetic polypeptide
FTFNNYYMR

SEQ ID NO: 650 - protein, 17 aa, synthetic construct
  REGION 1..17: note = Synthetic polypeptide
QISSSGSIKDYADSVKG

SEQ ID NO: 651 - protein, 9 aa, synthetic construct
  REGION 1..9: note = Synthetic polypeptide
FTLRSYGMH

SEQ ID NO: 652 - protein, 17 aa, synthetic construct
  REGION 1..17: note = Synthetic polypeptide
VSWYDGSNKHYADSVKG

SEQ ID NO: 653 - protein, 9 aa, synthetic construct
  REGION 1..9: note = Synthetic polypeptide
FTFSSYTMH

SEQ ID NO: 654 - protein, 17 aa, synthetic construct
  REGION 1..17: note = Synthetic polypeptide
VISYDGSNKYYADSVKG

SEQ ID NO: 655 - protein, 9 aa, synthetic construct
  REGION 1..9: note = Synthetic polypeptide
DSISVSYWS

SEQ ID NO: 656 - protein, 16 aa, synthetic construct
  REGION 1..16: note = Synthetic polypeptide
YIYNSGNANYNPSLES

SEQ ID NO: 657 - protein, 11 aa, synthetic construct
  REGION 1..11: note = Synthetic polypeptide
GSLSSDSHFWG

SEQ ID NO: 658 - protein, 16 aa, synthetic construct
  REGION 1..16: note = Synthetic polypeptide
YIYYSGNANYNPSLQS

SEQ ID NO: 659 - protein, 11 aa, synthetic construct
  REGION 1..11: note = Synthetic polypeptide
GSVSSGSYYWS

SEQ ID NO: 660 - protein, 16 aa, synthetic construct
  REGION 1..16: note = Synthetic polypeptide
YIYDSGNTNYNPSLKS

SEQ ID NO: 661 - protein, 17 aa, synthetic construct
  REGION 1..17: note = Synthetic polypeptide
CISSSGSMIYYADSVKG

SEQ ID NO: 662 - protein, 10 aa, synthetic construct
  REGION 1..10: note = Synthetic polypeptide
GSISTTDWWS

SEQ ID NO: 663 - protein, 16 aa, synthetic construct
  REGION 1..16: note = Synthetic polypeptide
EINQSGSTSYSPSFKS

SEQ ID NO: 664 - protein, 10 aa, synthetic construct
  REGION 1..10: note = Synthetic polypeptide
GSISSGNWWS

SEQ ID NO: 665 - protein, 16 aa, synthetic construct
  REGION 1..16: note = Synthetic polypeptide
EIYHSGSANYNPSLKS

SEQ ID NO: 666 - protein, 9 aa, synthetic construct
  REGION 1..9: note = Synthetic polypeptide
FTFTTYAMH

SEQ ID NO: 667 - protein, 17 aa, synthetic construct
  REGION 1..17: note = Synthetic polypeptide
AVSYDGNNKYYADSVKG

SEQ ID NO: 668 - protein, 10 aa, synthetic construct
  REGION 1..10: note = Synthetic polypeptide
DSISSTNWWS

SEQ ID NO: 669 - protein, 16 aa, synthetic construct
  REGION 1..16: note = Synthetic polypeptide
EIFHSGSTNYNPFLKS

SEQ ID NO: 670 - protein, 10 aa, synthetic construct
  REGION 1..10: note = Synthetic polypeptide
GSISSNNWWS

SEQ ID NO: 671 - protein, 16 aa, synthetic construct
  REGION 1..16: note = Synthetic polypeptide
DTYHSGSPSYNPSLKS

SEQ ID NO: 672 - protein, 9 aa, synthetic construct
  REGION 1..9: note = Synthetic polypeptide
FTFSNFGMH

SEQ ID NO: 673 - protein, 17 aa, synthetic construct
  REGION 1..17: note = Synthetic polypeptide
IISYDRSNKDYADSVKG

SEQ ID NO: 674 - protein, 10 aa, synthetic construct
  REGION 1..10: note = Synthetic polypeptide
ASISSNHWWT

SEQ ID NO: 675 - protein, 16 aa, synthetic construct
  REGION 1..16: note = Synthetic polypeptide
EIYHSGSPTYNPSLKS

SEQ ID NO: 676 - protein, 9 aa, synthetic construct
  REGION 1..9: note = Synthetic polypeptide
FTFSNSGMH

SEQ ID NO: 677 - protein, 17 aa, synthetic construct
  REGION 1..17: note = Synthetic polypeptide
LISYTGETKYYSDSLKA

SEQ ID NO: 678 - protein, 10 aa, synthetic construct
  REGION 1..10: note = Synthetic polypeptide
GSISSISWWS

SEQ ID NO: 679 - protein, 16 aa, synthetic construct
  REGION 1..16: note = Synthetic polypeptide
EINHSGSTVYNPSLKS

SEQ ID NO: 680 - protein, 9 aa, synthetic construct
  REGION 1..9: note = Synthetic polypeptide
FTFTDYYMS

SEQ ID NO: 681 - protein, 17 aa, synthetic construct
  REGION 1..17: note = Synthetic polypeptide
YISSSGNTRYYADSVKG

SEQ ID NO: 682 - protein, 10 aa, synthetic construct
  REGION 1..10: note = Synthetic polypeptide
GSITGSNWWS

SEQ ID NO: 683 - protein, 16 aa, synthetic construct
  REGION 1..16: note = Synthetic polypeptide
EIYHTGSTSYNPSLKS

SEQ ID NO: 684 - protein, 11 aa, synthetic construct
  REGION 1..11: note = Synthetic polypeptide
FSLSTSGVGVG

SEQ ID NO: 685 - protein, 16 aa, synthetic construct
  REGION 1..16: note = Synthetic polypeptide
LIYWDDDKRYSPSLKS

SEQ ID NO: 686 - protein, 17 aa, synthetic construct
  REGION 1..17: note = Synthetic polypeptide
VIWYDESNKYYADSVKG

SEQ ID NO: 687 - protein, 17 aa, synthetic construct
  REGION 1..17: note = Synthetic polypeptide
VISDSGGSTYYADSVKG

SEQ ID NO: 688 - protein, 9 aa, synthetic construct
  REGION 1..9: note = Synthetic polypeptide
FTFINYAMT

SEQ ID NO: 689 - protein, 17 aa, synthetic construct
  REGION 1..17: note = Synthetic polypeptide
AISGNGDGTYYADSVKG

SEQ ID NO: 690 - protein, 9 aa, synthetic construct
  REGION 1..9: note = Synthetic polypeptide
FTFSSYAMH

SEQ ID NO: 691 - protein, 17 aa, synthetic construct
  REGION 1..17: note = Synthetic polypeptide
VISHDGSNKYYADSVKG

SEQ ID NO: 692 - protein, 11 aa, synthetic construct
  REGION 1..11: note = Synthetic polypeptide
GSVRGGSHYWS

SEQ ID NO: 693 - protein, 16 aa, synthetic construct
  REGION 1..16: note = Synthetic polypeptide
YVYDSGSTNYNPSLKS

SEQ ID NO: 694 - protein, 9 aa, synthetic construct
  REGION 1..9: note = Synthetic polypeptide
FTFSNYAMS

SEQ ID NO: 695 - protein, 17 aa, synthetic construct
  REGION 1..17: note = Synthetic polypeptide
AISGSGDSTYYADSVKG

SEQ ID NO: 696 - protein, 17 aa, synthetic construct
  REGION 1..17: note = Synthetic polypeptide
YITSSGNTMYYADSVKG

SEQ ID NO: 697 - protein, 16 aa, synthetic construct
  REGION 1..16: note = Synthetic polypeptide
EIYHSGSTTYNPSLKS

SEQ ID NO: 698 - protein, 17 aa, synthetic construct
  REGION 1..17: note = Synthetic polypeptide
YISSSGNTIYYADSVKG

SEQ ID NO: 699 - protein, 9 aa, synthetic construct
  REGION 1..9: note = Synthetic polypeptide
FTFSNYGMH

SEQ ID NO: 700 - protein, 9 aa, synthetic construct
  REGION 1..9: note = Synthetic polypeptide
YTFNTYAMT

SEQ ID NO: 701 - protein, 17 aa, synthetic construct
  REGION 1..17: note = Synthetic polypeptide
WISTYNGNTVFGQKFQG

SEQ ID NO: 702 - protein, 9 aa, synthetic construct
  REGION 1..9: note = Synthetic polypeptide
FTFSTYWMS

SEQ ID NO: 703 - protein, 17 aa, synthetic construct
  REGION 1..17: note = Synthetic polypeptide
NIKQDGSEKYYVDSVKG

SEQ ID NO: 704 - protein, 17 aa, synthetic construct
  REGION 1..17: note = Synthetic polypeptide
VIWYDSRNQNYADSVKG

SEQ ID NO: 705 - protein, 17 aa, synthetic construct
  REGION 1..17: note = Synthetic polypeptide
TFSGRGGSTYYADFVKG

SEQ ID NO: 706 - protein, 9 aa, synthetic construct
  REGION 1..9: note = Synthetic polypeptide
FTFSSYGMH

SEQ ID NO: 707 - protein, 17 aa, synthetic construct
  REGION 1..17: note = Synthetic polypeptide
VISYDGSKKYSADSVKG

SEQ ID NO: 708 - protein, 9 aa, synthetic construct
  REGION 1..9: note = Synthetic polypeptide
FTFAEYAMH

SEQ ID NO: 709 - protein, 17 aa, synthetic construct
  REGION 1..17: note = Synthetic polypeptide
SISWNSGRIGYVDSVRG

SEQ ID NO: 710 - protein, 9 aa, synthetic construct
  REGION 1..9: note = Synthetic polypeptide
GSISSYYWS

SEQ ID NO: 711 - protein, 16 aa, synthetic construct
  REGION 1..16: note = Synthetic polypeptide
RIYTSGSGNYNPSLKR

SEQ ID NO: 712 - protein, 9 aa, synthetic construct
  REGION 1..9: note = Synthetic polypeptide
FTFSDYEMN

SEQ ID NO: 713 - protein, 17 aa, synthetic construct
  REGION 1..17: note = Synthetic polypeptide
HISSSGNIIYYADSVKG

SEQ ID NO: 714 - protein, 9 aa, synthetic construct
  REGION 1..9: note = Synthetic polypeptide
FTFSAYAMS

SEQ ID NO: 715 - protein, 17 aa, synthetic construct
  REGION 1..17: note = Synthetic polypeptide
AISGSDRRIYYADSVKG

SEQ ID NO: 716 - protein, 9 aa, synthetic construct
  REGION 1..9: note = Synthetic polypeptide
FTFSDHYMA

SEQ ID NO: 717 - protein, 19 aa, synthetic construct
  REGION 1..19: note = Synthetic polypeptide
RIRNKPNSYTTEYAASVKG

SEQ ID NO: 718 - protein, 9 aa, synthetic construct
  REGION 1..9: note = Synthetic polypeptide
YSFTTYGIS

SEQ ID NO: 719 - protein, 17 aa, synthetic construct
  REGION 1..17: note = Synthetic polypeptide
WISGYSGDTNYAQKVQG

SEQ ID NO: 720 - protein, 11 aa, synthetic construct
  REGION 1..11: note = Synthetic polypeptide
GSISSSSYYWG

SEQ ID NO: 721 - protein, 16 aa, synthetic construct
  REGION 1..16: note = Synthetic polypeptide
SIYYSGSTYYNPSLKS

SEQ ID NO: 722 - protein, 9 aa, synthetic construct
  REGION 1..9: note = Synthetic polypeptide
FTFSRYSMN

SEQ ID NO: 723 - protein, 17 aa, synthetic construct
  REGION 1..17: note = Synthetic polypeptide
SISHSGRYIYYADSEKG

SEQ ID NO: 724 - protein, 9 aa, synthetic construct
  REGION 1..9: note = Synthetic polypeptide
FTFSDHYMD

SEQ ID NO: 725 - protein, 19 aa, synthetic construct
  REGION 1..19: note = Synthetic polypeptide
RTRNKPNSHTTEYAASVKG

SEQ ID NO: 726 - protein, 9 aa, synthetic construct
  REGION 1..9: note = Synthetic polypeptide
FIYTNYAMY

SEQ ID NO: 727 - protein, 17 aa, synthetic construct
  REGION 1..17: note = Synthetic polypeptide
AISGSGGITYYADSVKG

SEQ ID NO: 728 - protein, 9 aa, synthetic construct
  REGION 1..9: note = Synthetic polypeptide
FIFSDYYMD

SEQ ID NO: 729 - protein, 19 aa, synthetic construct
  REGION 1..19: note = Synthetic polypeptide
RITNRPNSYTTEYAASVKG

SEQ ID NO: 730 - protein, 11 aa, synthetic construct
  REGION 1..11: note = Synthetic polypeptide
GSISSGSYYWS

SEQ ID NO: 731 - protein, 16 aa, synthetic construct
  REGION 1..16: note = Synthetic polypeptide
RIYTSGSTNYNPSLKS

SEQ ID NO: 732 - protein, 16 aa, synthetic construct
  REGION 1..16: note = Synthetic polypeptide
EIYHSESTNYNPSLKS

SEQ ID NO: 733 - protein, 17 aa, synthetic construct
  REGION 1..17: note = Synthetic polypeptide
GISWNSGSIGYADSVKG

SEQ ID NO: 734 - protein, 17 aa, synthetic construct
  REGION 1..17: note = Synthetic polypeptide
SIRSSGGRTEYADSVKG

SEQ ID NO: 735 - protein, 19 aa, synthetic construct
  REGION 1..19: note = Synthetic polypeptide
RIRNKPNSYATQYAASVKG

SEQ ID NO: 736 - protein, 9 aa, synthetic construct
  REGION 1..9: note = Synthetic polypeptide
FTFSSYSMN

SEQ ID NO: 737 - protein, 17 aa, synthetic construct
  REGION 1..17: note = Synthetic polypeptide
SISSRSSFMYYADSVKG

SEQ ID NO: 738 - protein, 17 aa, synthetic construct
  REGION 1..17: note = Synthetic polypeptide
LISYDGSNKYYADSVKG

SEQ ID NO: 739 - protein, 9 aa, synthetic construct
  REGION 1..9: note = Synthetic polypeptide
FTFSSLAMH

SEQ ID NO: 740 - protein, 17 aa, synthetic construct
  REGION 1..17: note = Synthetic polypeptide
TISYDVSNKYYADSVKG

SEQ ID NO: 741 - protein, 9 aa, synthetic construct
  REGION 1..9: note = Synthetic polypeptide
YTFTSYGIS

SEQ ID NO: 742 - protein, 17 aa, synthetic construct
  REGION 1..17: note = Synthetic polypeptide
WISAYNGNTNYAQKLQG

SEQ ID NO: 743 - protein, 11 aa, synthetic construct
  REGION 1..11: note = Synthetic polypeptide
GAISSGDYYWS

SEQ ID NO: 744 - protein, 16 aa, synthetic construct
  REGION 1..16: note = Synthetic polypeptide
YIHYSGTTYNNPSLKS

SEQ ID NO: 745 - protein, 17 aa, synthetic construct
  REGION 1..17: note = Synthetic polypeptide
VIRFDGSNTVYADSVKG

SEQ ID NO: 746 - protein, 17 aa, synthetic construct
  REGION 1..17: note = Synthetic polypeptide
VISYDGSYKWYADSVKG

SEQ ID NO: 747 - protein, 9 aa, synthetic construct
  REGION 1..9: note = Synthetic polypeptide
FSFSSHAMT

SEQ ID NO: 748 - protein, 17 aa, synthetic construct
  REGION 1..17: note = Synthetic polypeptide
SIRGSDRTTNYADSVKG

SEQ ID NO: 749 - protein, 9 aa, synthetic construct
  REGION 1..9: note = Synthetic polypeptide
FTFGTHAMS

SEQ ID NO: 750 - protein, 17 aa, synthetic construct
  REGION 1..17: note = Synthetic polypeptide
TFSGSGGRTYYADSVKG

SEQ ID NO: 751 - protein, 9 aa, synthetic construct
  REGION 1..9: note = Synthetic polypeptide
FTFSDYYMD

SEQ ID NO: 752 - protein, 19 aa, synthetic construct
  REGION 1..19: note = Synthetic polypeptide
GIRNKPNSYTTEYAASVKG

SEQ ID NO: 753 - protein, 9 aa, synthetic construct
  REGION 1..9: note = Synthetic polypeptide
ASIRSYLWS

SEQ ID NO: 754 - protein, 16 aa, synthetic construct
  REGION 1..16: note = Synthetic polypeptide
SIYHSGSTKYNPSLKS

SEQ ID NO: 755 - protein, 9 aa, synthetic construct
  REGION 1..9: note = Synthetic polypeptide
FTFSGNAMH

SEQ ID NO: 756 - protein, 17 aa, synthetic construct
  REGION 1..17: note = Synthetic polypeptide
VILYDGSNQYYADSVKG

SEQ ID NO: 757 - protein, 10 aa, synthetic construct
  REGION 1..10: note = Synthetic polypeptide
GSISSSNWWT

SEQ ID NO: 758 - protein, 16 aa, synthetic construct
  REGION 1..16: note = Synthetic polypeptide
EIYHSGSTNYNPSLES

SEQ ID NO: 759 - protein, 9 aa, synthetic construct
  REGION 1..9: note = Synthetic polypeptide
FTFTYYAMS

SEQ ID NO: 760 - protein, 17 aa, synthetic construct
  REGION 1..17: note = Synthetic polypeptide
GISGSGDSTYNADSVKG

SEQ ID NO: 761 - protein, 17 aa, synthetic construct
  REGION 1..17: note = Synthetic polypeptide
VISYDGSKKYFADSVKG

SEQ ID NO: 762 - protein, 17 aa, synthetic construct
  REGION 1..17: note = Synthetic polypeptide
VIWYDGSNKYYADSVKG

SEQ ID NO: 763 - protein, 11 aa, synthetic construct
  REGION 1..11: note = Synthetic polypeptide
GSISSGGYYWT

SEQ ID NO: 764 - protein, 16 aa, synthetic construct
  REGION 1..16: note = Synthetic polypeptide
YIYYTGSTYYNPSLKS

SEQ ID NO: 765 - protein, 9 aa, synthetic construct
  REGION 1..9: note = Synthetic polypeptide
FTFSSYAMT

SEQ ID NO: 766 - protein, 17 aa, synthetic construct
  REGION 1..17: note = Synthetic polypeptide
DMNHSGDRTNYADSVRG

SEQ ID NO: 767 - protein, 9 aa, synthetic construct
  REGION 1..9: note = Synthetic polypeptide
FIFSDHYMA

SEQ ID NO: 768 - protein, 19 aa, synthetic construct
  REGION 1..19: note = Synthetic polypeptide
RSRNRPNSYTTEYAASAKG

SEQ ID NO: 769 - protein, 9 aa, synthetic construct
  REGION 1..9: note = Synthetic polypeptide
YTFTGYYMH

SEQ ID NO: 770 - protein, 17 aa, synthetic construct
  REGION 1..17: note = Synthetic polypeptide
RINPNSGGTNYAQKFQG

SEQ ID NO: 771 - protein, 19 aa, synthetic construct
  REGION 1..19: note = Synthetic polypeptide
RIRNKPNGYTTEYAASVKG

SEQ ID NO: 772 - protein, 19 aa, synthetic construct
  REGION 1..19: note = Synthetic polypeptide
RSRNKPNSYITEYAASVKG

SEQ ID NO: 773 - protein, 11 aa, synthetic construct
  REGION 1..11: note = Synthetic polypeptide
FSLSNTKLGVS

SEQ ID NO: 774 - protein, 16 aa, synthetic construct
  REGION 1..16: note = Synthetic polypeptide
HIFSNAEKSSSKSLKS

SEQ ID NO: 775 - protein, 9 aa, synthetic construct
  REGION 1..9: note = Synthetic polypeptide
LTFSTYTLH

SEQ ID NO: 776 - protein, 17 aa, synthetic construct
  REGION 1..17: note = Synthetic polypeptide
VISSDGGNKYYADSVKG

SEQ ID NO: 777 - protein, 17 aa, synthetic construct
  REGION 1..17: note = Synthetic polypeptide
MMSYDGGDKNYADSVKG

SEQ ID NO: 778 - protein, 17 aa, synthetic construct
  REGION 1..17: note = Synthetic polypeptide
WISTYNGNTNYAQKLQG

SEQ ID NO: 779 - protein, 9 aa, synthetic construct
  REGION 1..9: note = Synthetic polypeptide
FTFSNYWMN

SEQ ID NO: 780 - protein, 19 aa, synthetic construct
  REGION 1..19: note = Synthetic polypeptide
RSTNKPNSYTTTYAASVRG

SEQ ID NO: 781 - protein, 9 aa, synthetic construct
  REGION 1..9: note = Synthetic polypeptide
GSFSGYYWS

SEQ ID NO: 782 - protein, 16 aa, synthetic construct
  REGION 1..16: note = Synthetic polypeptide
EINHRGSTDYNPSLKS

SEQ ID NO: 783 - protein, 9 aa, synthetic construct
  REGION 1..9: note = Synthetic polypeptide
FTFSHAWMT

SEQ ID NO: 784 - protein, 19 aa, synthetic construct
  REGION 1..19: note = Synthetic polypeptide
RIKSETDGGTANYAAPVKG

SEQ ID NO: 785 - protein, 9 aa, synthetic construct
  REGION 1..9: note = Synthetic polypeptide
GTFSSYVIS

SEQ ID NO: 786 - protein, 17 aa, synthetic construct
  REGION 1..17: note = Synthetic polypeptide
GIIPIFGTPNYAQKFQG

SEQ ID NO: 787 - protein, 9 aa, synthetic construct
  REGION 1..9: note = Synthetic polypeptide
FIFSSNSMH

SEQ ID NO: 788 - protein, 17 aa, synthetic construct
  REGION 1..17: note = Synthetic polypeptide
IISNDGRNKFYADAVKG

SEQ ID NO: 789 - protein, 19 aa, synthetic construct
  REGION 1..19: note = Synthetic polypeptide
RTRNKANSYTTKYAASVKG

SEQ ID NO: 790 - protein, 11 aa, synthetic construct
  REGION 1..11: note = Synthetic polypeptide
FSLSNTKMGVT

SEQ ID NO: 791 - protein, 16 aa, synthetic construct
  REGION 1..16: note = Synthetic polypeptide
HIFSNDEKSCNTSLKS

SEQ ID NO: 792 - protein, 19 aa, synthetic construct
  REGION 1..19: note = Synthetic polypeptide
RSRNKVNSYTTDYAASVKG

SEQ ID NO: 793 - protein, 16 aa, synthetic construct
  REGION 1..16: note = Synthetic polypeptide
YIYYSGSTNYNPSLKS

SEQ ID NO: 794 - protein, 9 aa, synthetic construct
  REGION 1..9: note = Synthetic polypeptide
FTFDDYDMH

SEQ ID NO: 795 - protein, 17 aa, synthetic construct
  REGION 1..17: note = Synthetic polypeptide
GISWNSGGRGYADSVKG

SEQ ID NO: 796 - protein, 17 aa, synthetic construct
  REGION 1..17: note = Synthetic polypeptide
VMSYDGSNKYYADSLKG

SEQ ID NO: 797 - protein, 9 aa, synthetic construct
  REGION 1..9: note = Synthetic polypeptide
FNFSSYGMH

SEQ ID NO: 798 - protein, 9 aa, synthetic construct
  REGION 1..9: note = Synthetic polypeptide
LTLSSSAMS

SEQ ID NO: 799 - protein, 17 aa, synthetic construct
  REGION 1..17: note = Synthetic polypeptide
GITGSGSDSSYAASVKG

SEQ ID NO: 800 - protein, 17 aa, synthetic construct
  REGION 1..17: note = Synthetic polypeptide
VISEDGNKDHYVDSVKG

SEQ ID NO: 801 - protein, 19 aa, synthetic construct
  REGION 1..19: note = Synthetic polypeptide
RSRNKVNSYITEYAASVKG

SEQ ID NO: 802 - protein, 9 aa, synthetic construct
  REGION 1..9: note = Synthetic polypeptide
FIFSDHYMD

SEQ ID NO: 803 - protein, 19 aa, synthetic construct
  REGION 1..19: note = Synthetic polypeptide
RIRNKPNSYTTDYAAYVKG

SEQ ID NO: 804 - protein, 9 aa, synthetic construct
  REGION 1..9: note = Synthetic polypeptide
FTLSSYVMH

SEQ ID NO: 805 - protein, 17 aa, synthetic construct
  REGION 1..17: note = Synthetic polypeptide
VISSDGTNKYYADSVKG

SEQ ID NO: 806 - protein, 10 aa, synthetic construct
  REGION 1..10: note = Synthetic polypeptide
GSISSDNWWS

SEQ ID NO: 807 - protein, 9 aa, synthetic construct
  REGION 1..9: note = Synthetic polypeptide
YTFTSYAMH

SEQ ID NO: 808 - protein, 17 aa, synthetic construct
  REGION 1..17: note = Synthetic polypeptide
WINAGNGNTKYSQKFQG

SEQ ID NO: 809 - protein, 19 aa, synthetic construct
  REGION 1..19: note = Synthetic polypeptide
HVGNKANTYTTEYAASVKG

SEQ ID NO: 810 - protein, 9 aa, synthetic construct
  REGION 1..9: note = Synthetic polypeptide
YTFTSYDIN

SEQ ID NO: 811 - protein, 17 aa, synthetic construct
  REGION 1..17: note = Synthetic polypeptide
WMNPNSGNTGYAQKFQG

SEQ ID NO: 812 - protein, 17 aa, synthetic construct
  REGION 1..17: note = Synthetic polypeptide
WINPNSGGTNYAQKFQG

SEQ ID NO: 813 - protein, 11 aa, synthetic construct
  REGION 1..11: note = Synthetic polypeptide
DSVSTNSAAWN

SEQ ID NO: 814 - protein, 18 aa, synthetic construct
  REGION 1..18: note = Synthetic polypeptide
RTYYRSKWYNDYALSVKS

SEQ ID NO: 815 - protein, 19 aa, synthetic construct
  REGION 1..19: note = Synthetic polypeptide
RARNRANSYTTEYAASVKG

SEQ ID NO: 816 - protein, 19 aa, synthetic construct
  REGION 1..19: note = Synthetic polypeptide
RIRNKVNSYTTEYAASVKG

SEQ ID NO: 817 - protein, 10 aa, synthetic construct
  REGION 1..10: note = Synthetic polypeptide
DSISSRSWWS

SEQ ID NO: 818 - protein, 16 aa, synthetic construct
  REGION 1..16: note = Synthetic polypeptide
EIYHSGTTTYSPSLKS

SEQ ID NO: 819 - protein, 16 aa, synthetic construct
  REGION 1..16: note = Synthetic polypeptide
EIYHSGSTNYNPSLKS

SEQ ID NO: 820 - protein, 9 aa, synthetic construct
  REGION 1..9: note = Synthetic polypeptide
YTFTSYAMN

SEQ ID NO: 821 - protein, 17 aa, synthetic construct
  REGION 1..17: note = Synthetic polypeptide
WINTNTGNPTYAQGFTG

SEQ ID NO: 822 - protein, 9 aa, synthetic construct
  REGION 1..9: note = Synthetic polypeptide
FTFSYSAIH

SEQ ID NO: 823 - protein, 10 aa, synthetic construct
  REGION 1..10: note = Synthetic polypeptide
ASISSNNWWS

SEQ ID NO: 824 - protein, 16 aa, synthetic construct
  REGION 1..16: note = Synthetic polypeptide
EIFHSGTTNYNPSLKS

SEQ ID NO: 825 - protein, 10 aa, synthetic construct
  REGION 1..10: note = Synthetic polypeptide
ISISSSNWWS

SEQ ID NO: 826 - protein, 16 aa, synthetic construct
  REGION 1..16: note = Synthetic polypeptide
EVYHSGSTKYNPSLKS

SEQ ID NO: 827 - protein, 9 aa, synthetic construct
  REGION 1..9: note = Synthetic polypeptide
FTFSTSPLH

SEQ ID NO: 828 - protein, 17 aa, synthetic construct
  REGION 1..17: note = Synthetic polypeptide
VSSFVATDKYYADSVKG

SEQ ID NO: 829 - protein, 17 aa, synthetic construct
  REGION 1..17: note = Synthetic polypeptide
SISSSSSYIYYADSVKG

SEQ ID NO: 830 - protein, 17 aa, synthetic construct
  REGION 1..17: note = Synthetic polypeptide
VIWYDGSNKNYADSVKG

SEQ ID NO: 831 - protein, 17 aa, synthetic construct
  REGION 1..17: note = Synthetic polypeptide
AISSSGGSTYYADSVKG

SEQ ID NO: 832 - protein, 9 aa, synthetic construct
  REGION 1..9: note = Synthetic polypeptide
FIFSNYWMS

SEQ ID NO: 833 - protein, 17 aa, synthetic construct
  REGION 1..17: note = Synthetic polypeptide
NIKPDGSEKYYVESVRG

SEQ ID NO: 834 - protein, 9 aa, synthetic construct
  REGION 1..9: note = Synthetic polypeptide
FTFSISGMH

SEQ ID NO: 835 - protein, 17 aa, synthetic construct
  REGION 1..17: note = Synthetic polypeptide
LIWYDGTKKYYADSVKG

SEQ ID NO: 836 - protein, 9 aa, synthetic construct
  REGION 1..9: note = Synthetic polypeptide
FSFGDYGMH

SEQ ID NO: 837 - protein, 17 aa, synthetic construct
  REGION 1..17: note = Synthetic polypeptide
VILFDGSKKFYADSVRG

SEQ ID NO: 838 - protein, 19 aa, synthetic construct
  REGION 1..19: note = Synthetic polypeptide
RTRNKANSYTTEYAASVKG

SEQ ID NO: 839 - protein, 9 aa, synthetic construct
  REGION 1..9: note = Synthetic polypeptide
FNLIDYAMH

SEQ ID NO: 840 - protein, 17 aa, synthetic construct
  REGION 1..17: note = Synthetic polypeptide
GISWNSRSIGYADSVKG

SEQ ID NO: 841 - protein, 13 aa, synthetic construct
  REGION 1..13: note = Synthetic polypeptide
SGSSSNIGNNYVS

SEQ ID NO: 842 - protein, 7 aa, synthetic construct
  REGION 1..7: note = Synthetic polypeptide
DNKKRPS

SEQ ID NO: 843 - protein, 11 aa, synthetic construct
  REGION 1..11: note = Synthetic polypeptide
RASQSVSSNLA

SEQ ID NO: 844 - protein, 7 aa, synthetic construct
  REGION 1..7: note = Synthetic polypeptide
DASNRAT

SEQ ID NO: 845 - protein, 13 aa, synthetic construct
  REGION 1..13: note = Synthetic polypeptide
SGSRSNIGTYTIN

SEQ ID NO: 846 - protein, 7 aa, synthetic construct
  REGION 1..7: note = Synthetic polypeptide
SNNRGPS

SEQ ID NO: 847 - protein, 13 aa, synthetic construct
  REGION 1..13: note = Synthetic polypeptide
SGSSSNIGNNYVA

SEQ ID NO: 848 - protein, 11 aa, synthetic construct
  REGION 1..11: note = Synthetic polypeptide
RASQTISVDLN

SEQ ID NO: 849 - protein, 7 aa, synthetic construct
  REGION 1..7: note = Synthetic polypeptide
AASTLQS

SEQ ID NO: 850 - protein, 11 aa, synthetic construct
  REGION 1..11: note = Synthetic polypeptide
RASQSIGSDLN

SEQ ID NO: 851 - protein, 7 aa, synthetic construct
  REGION 1..7: note = Synthetic polypeptide
AATGLQS

SEQ ID NO: 852 - protein, 13 aa, synthetic construct
  REGION 1..13: note = Synthetic polypeptide
SGSSSNIGTNTVS

SEQ ID NO: 853 - protein, 7 aa, synthetic construct
  REGION 1..7: note = Synthetic polypeptide
SRTQRPS

SEQ ID NO: 854 - protein, 13 aa, synthetic construct
  REGION 1..13: note = Synthetic polypeptide
SGSNSNIGNYYVS

SEQ ID NO: 855 - protein, 7 aa, synthetic construct
  REGION 1..7: note = Synthetic polypeptide
DNNKRPS

SEQ ID NO: 856 - protein, 13 aa, synthetic construct
  REGION 1..13: note = Synthetic polypeptide
SGSSSNIGNSYVS

SEQ ID NO: 857 - protein, 13 aa, synthetic construct
  REGION 1..13: note = Synthetic polypeptide
SGSSSNIGYSHVS

SEQ ID NO: 858 - protein, 7 aa, synthetic construct
  REGION 1..7: note = Synthetic polypeptide
DNDKRPS

SEQ ID NO: 859 - protein, 14 aa, synthetic construct
  REGION 1..14: note = Synthetic polypeptide
TGTSSDVGAYNFVS

SEQ ID NO: 860 - protein, 7 aa, synthetic construct
  REGION 1..7: note = Synthetic polypeptide
DVNKRPS

SEQ ID NO: 861 - protein, 13 aa, synthetic construct
  REGION 1..13: note = Synthetic polypeptide
SGSSSNIGSNYVS

SEQ ID NO: 862 - protein, 7 aa, synthetic construct
  REGION 1..7: note = Synthetic polypeptide
DNSKRPS

SEQ ID NO: 863 - protein, 11 aa, synthetic construct
  REGION 1..11: note = Synthetic polypeptide
RASRSVSSNLA

SEQ ID NO: 864 - protein, 7 aa, synthetic construct
  REGION 1..7: note = Synthetic polypeptide
GASTRAT

SEQ ID NO: 865 - protein, 11 aa, synthetic construct
  REGION 1..11: note = Synthetic polypeptide
RASQGISSWLA

SEQ ID NO: 866 - protein, 7 aa, synthetic construct
  REGION 1..7: note = Synthetic polypeptide
AASSLQS

SEQ ID NO: 867 - protein, 14 aa, synthetic construct
  REGION 1..14: note = Synthetic polypeptide
TGTSSDVGGYNYVS

SEQ ID NO: 868 - protein, 7 aa, synthetic construct
  REGION 1..7: note = Synthetic polypeptide
DVSNRPS

SEQ ID NO: 869 - protein, 14 aa, synthetic construct
  REGION 1..14: note = Synthetic polypeptide
TGTSSDIGAYNYVS

SEQ ID NO: 870 - protein, 7 aa, synthetic construct
  REGION 1..7: note = Synthetic polypeptide
DVTNRPS

SEQ ID NO: 871 - protein, 14 aa, synthetic construct
  REGION 1..14: note = Synthetic polypeptide
TGTGSDVGGYNFVS

SEQ ID NO: 872 - protein, 7 aa, synthetic construct
  REGION 1..7: note = Synthetic polypeptide
DVNNRPS

SEQ ID NO: 873 - protein, 11 aa, synthetic construct
  REGION 1..11: note = Synthetic polypeptide
RASQSISSYLN

SEQ ID NO: 874 - protein, 7 aa, synthetic construct
  REGION 1..7: note = Synthetic polypeptide
AASILQS

SEQ ID NO: 875 - protein, 13 aa, synthetic construct
  REGION 1..13: note = Synthetic polypeptide
TGSSSNIGAGYDV

SEQ ID NO: 876 - protein, 7 aa, synthetic construct
  REGION 1..7: note = Synthetic polypeptide
GNSNRPS

SEQ ID NO: 877 - protein, 11 aa, synthetic construct
  REGION 1..11: note = Synthetic polypeptide
RASQSVNSYLV

SEQ ID NO: 878 - protein, 11 aa, synthetic construct
  REGION 1..11: note = Synthetic polypeptide
RASQSVNRYLA

SEQ ID NO: 879 - protein, 11 aa, synthetic construct
  REGION 1..11: note = Synthetic polypeptide
RASQSVSNYLA

SEQ ID NO: 880 - protein, 7 aa, synthetic construct
  REGION 1..7: note = Synthetic polypeptide
DASSRAT

SEQ ID NO: 881 - protein, 14 aa, synthetic construct
  REGION 1..14: note = Synthetic polypeptide
TGTSSDIGGYNYVS

SEQ ID NO: 882 - protein, 13 aa, synthetic construct
  REGION 1..13: note = Synthetic polypeptide
SGRSSNIGNSDVS

SEQ ID NO: 883 - protein, 7 aa, synthetic construct
  REGION 1..7: note = Synthetic polypeptide
DNDERPS

SEQ ID NO: 884 - protein, 11 aa, synthetic construct
  REGION 1..11: note = Synthetic polypeptide
QASQDISNYLN

SEQ ID NO: 885 - protein, 7 aa, synthetic construct
  REGION 1..7: note = Synthetic polypeptide
DASNLER

SEQ ID NO: 886 - protein, 14 aa, synthetic construct
  REGION 1..14: note = Synthetic polypeptide
TGTSSDVGGDKYVS

SEQ ID NO: 887 - protein, 7 aa, synthetic construct
  REGION 1..7: note = Synthetic polypeptide
EVSNRPS

SEQ ID NO: 888 - protein, 13 aa, synthetic construct
  REGION 1..13: note = Synthetic polypeptide
SGSSSNIGNYYVS

SEQ ID NO: 889 - protein, 17 aa, synthetic construct
  REGION 1..17: note = Synthetic polypeptide
KSSQSVLFGSNQKSCLA

SEQ ID NO: 890 - protein, 7 aa, synthetic construct
  REGION 1..7: note = Synthetic polypeptide
WASTRES

SEQ ID NO: 891 - protein, 13 aa, synthetic construct
  REGION 1..13: note = Synthetic polypeptide
SGSSSNIGSNFVS

SEQ ID NO: 892 - protein, 13 aa, synthetic construct
  REGION 1..13: note = Synthetic polypeptide
SGSSSNFGNDYVS

SEQ ID NO: 893 - protein, 13 aa, synthetic construct
  REGION 1..13: note = Synthetic polypeptide
SGSSSNIGNDYVS

SEQ ID NO: 894 - protein, 14 aa, synthetic construct
  REGION 1..14: note = Synthetic polypeptide
TGTSSDVGGYKYVS

SEQ ID NO: 895 - protein, 11 aa, synthetic construct
  REGION 1..11: note = Synthetic polypeptide
QASQDISNFLN

SEQ ID NO: 896 - protein, 7 aa, synthetic construct
  REGION 1..7: note = Synthetic polypeptide
DASSLET

SEQ ID NO: 897 - protein, 11 aa, synthetic construct
  REGION 1..11: note = Synthetic polypeptide
RASQNINTYLA

SEQ ID NO: 898 - protein, 7 aa, synthetic construct
  REGION 1..7: note = Synthetic polypeptide
RASTLES

SEQ ID NO: 899 - protein, 11 aa, synthetic construct
  REGION 1..11: note = Synthetic polypeptide
RASQGISNYLA

SEQ ID NO: 900 - protein, 7 aa, synthetic construct
  REGION 1..7: note = Synthetic polypeptide
AASTLRS

SEQ ID NO: 901 - protein, 11 aa, synthetic construct
  REGION 1..11: note = Synthetic polypeptide
RASQSVSSYLA

SEQ ID NO: 902 - protein, 7 aa, synthetic construct
  REGION 1..7: note = Synthetic polypeptide
GASNRAT

SEQ ID NO: 903 - protein, 7 aa, synthetic construct
  REGION 1..7: note = Synthetic polypeptide
RASSLES

SEQ ID NO: 904 - protein, 11 aa, synthetic construct
  REGION 1..11: note = Synthetic polypeptide
GGHNVGSKSVH

SEQ ID NO: 905 - protein, 7 aa, synthetic construct
  REGION 1..7: note = Synthetic polypeptide
DDSDRPS

SEQ ID NO: 906 - protein, 11 aa, synthetic construct
  REGION 1..11: note = Synthetic polypeptide
GGNNIGSKSVH

SEQ ID NO: 907 - protein, 7 aa, synthetic construct
  REGION 1..7: note = Synthetic polypeptide
SNSDRPS

SEQ ID NO: 908 - protein, 7 aa, synthetic construct
  REGION 1..7: note = Synthetic polypeptide
EVSKRPS

SEQ ID NO: 909 - protein, 14 aa, synthetic construct
  REGION 1..14: note = Synthetic polypeptide
TGTSTDVGGYNYVS

SEQ ID NO: 910 - protein, 16 aa, synthetic construct
  REGION 1..16: note = Synthetic polypeptide
RSSQSLLHSNGYNYLD

SEQ ID NO: 911 - protein, 7 aa, synthetic construct
  REGION 1..7: note = Synthetic polypeptide
LGSNRAS

SEQ ID NO: 912 - protein, 11 aa, synthetic construct
  REGION 1..11: note = Synthetic polypeptide
RASQGIRNDLG

SEQ ID NO: 913 - protein, 11 aa, synthetic construct
  REGION 1..11: note = Synthetic polypeptide
RASQSISTWLA

SEQ ID NO: 914 - protein, 11 aa, synthetic construct
  REGION 1..11: note = Synthetic polypeptide
RASQGISSYLA

SEQ ID NO: 915 - protein, 11 aa, synthetic construct
  REGION 1..11: note = Synthetic polypeptide
RASQSVSFNLA

SEQ ID NO: 916 - protein, 7 aa, synthetic construct
  REGION 1..7: note = Synthetic polypeptide
RASTRAA

SEQ ID NO: 917 - protein, 17 aa, synthetic construct
  REGION 1..17: note = Synthetic polypeptide
KSSQSVLYSSNNKNSLA

SEQ ID NO: 918 - protein, 11 aa, synthetic construct
  REGION 1..11: note = Synthetic polypeptide
RASQSVSSWLA

SEQ ID NO: 919 - protein, 9 aa, synthetic construct
  REGION 1..9: note = Synthetic polypeptide
SGDALQYVY

SEQ ID NO: 920 - protein, 7 aa, synthetic construct
  REGION 1..7: note = Synthetic polypeptide
KDTERPS

SEQ ID NO: 921 - protein, 12 aa, synthetic construct
  REGION 1..12: note = Synthetic polypeptide
RASRSVSGNYLA

SEQ ID NO: 922 - protein, 7 aa, synthetic construct
  REGION 1..7: note = Synthetic polypeptide
AASSRAT

SEQ ID NO: 923 - protein, 11 aa, synthetic construct
  REGION 1..11: note = Synthetic polypeptide
RASQSIRSYLN

SEQ ID NO: 924 - protein, 11 aa, synthetic construct
  REGION 1..11: note = Synthetic polypeptide
RASQSISSWLA

SEQ ID NO: 925 - protein, 7 aa, synthetic construct
  REGION 1..7: note = Synthetic polypeptide
QASSLES

SEQ ID NO: 926 - protein, 11 aa, synthetic construct
  REGION 1..11: note = Synthetic polypeptide
RASQSINNYLN

SEQ ID NO: 927 - protein, 7 aa, synthetic construct
  REGION 1..7: note = Synthetic polypeptide
GASTLQS

SEQ ID NO: 928 - protein, 12 aa, synthetic construct
  REGION 1..12: note = Synthetic polypeptide
RASQSVSSSYLA

SEQ ID NO: 929 - protein, 7 aa, synthetic construct
  REGION 1..7: note = Synthetic polypeptide
GASSRAT

SEQ ID NO: 930 - protein, 11 aa, synthetic construct
  REGION 1..11: note = Synthetic polypeptide
RASQSISDWLA

SEQ ID NO: 931 - protein, 7 aa, synthetic construct
  REGION 1..7: note = Synthetic polypeptide
RASGLES

SEQ ID NO: 932 - protein, 7 aa, synthetic construct
  REGION 1..7: note = Synthetic polypeptide
KASNLQS

SEQ ID NO: 933 - protein, 12 aa, synthetic construct
  REGION 1..12: note = Synthetic polypeptide
RASQSVSSRYLA

SEQ ID NO: 934 - protein, 7 aa, synthetic construct
  REGION 1..7: note = Synthetic polypeptide
DVSKRPS

SEQ ID NO: 935 - protein, 13 aa, synthetic construct
  REGION 1..13: note = Synthetic polypeptide
TRSSGSIASNYVQ

SEQ ID NO: 936 - protein, 7 aa, synthetic construct
  REGION 1..7: note = Synthetic polypeptide
EDNQRPS

SEQ ID NO: 937 - protein, 13 aa, synthetic construct
  REGION 1..13: note = Synthetic polypeptide
SGSSSNIGNNDVS

SEQ ID NO: 938 - protein, 11 aa, synthetic construct
  REGION 1..11: note = Synthetic polypeptide
RASQGIRNDLA

SEQ ID NO: 939 - protein, 7 aa, synthetic construct
  REGION 1..7: note = Synthetic polypeptide
RASSLET

SEQ ID NO: 940 - protein, 11 aa, synthetic construct
  REGION 1..11: note = Synthetic polypeptide
RASQNVGGWLA

SEQ ID NO: 941 - protein, 7 aa, synthetic construct
  REGION 1..7: note = Synthetic polypeptide
QASRLEN

SEQ ID NO: 942 - protein, 11 aa, synthetic construct
  REGION 1..11: note = Synthetic polypeptide
RASQSISQYLN

SEQ ID NO: 943 - protein, 7 aa, synthetic construct
  REGION 1..7: note = Synthetic polypeptide
PASSFQS

SEQ ID NO: 944 - protein, 11 aa, synthetic construct
  REGION 1..11: note = Synthetic polypeptide
GGNYIGGKSVH

SEQ ID NO: 945 - protein, 7 aa, synthetic construct
  REGION 1..7: note = Synthetic polypeptide
NDNDRPS

SEQ ID NO: 946 - protein, 11 aa, synthetic construct
  REGION 1..11: note = Synthetic polypeptide
RASQRVNSNLA

SEQ ID NO: 947 - protein, 12 aa, synthetic construct
  REGION 1..12: note = Synthetic polypeptide
RASQSVSSSFLA

SEQ ID NO: 948 - protein, 7 aa, synthetic construct
  REGION 1..7: note = Synthetic polypeptide
RASGLET

SEQ ID NO: 949 - protein, 13 aa, synthetic construct
  REGION 1..13: note = Synthetic polypeptide
SGSSSNFGSNFVY

SEQ ID NO: 950 - protein, 7 aa, synthetic construct
  REGION 1..7: note = Synthetic polypeptide
RVNQRPS

SEQ ID NO: 951 - protein, 12 aa, synthetic construct
  REGION 1..12: note = Synthetic polypeptide
RASQSVSSSYLS

SEQ ID NO: 952 - protein, 11 aa, synthetic construct
  REGION 1..11: note = Synthetic polypeptide
RASQTITRYMN

SEQ ID NO: 953 - protein, 7 aa, synthetic construct
  REGION 1..7: note = Synthetic polypeptide
ATSSLQS

SEQ ID NO: 954 - protein, 11 aa, synthetic construct
  REGION 1..11: note = Synthetic polypeptide
RASQDIRKFLN

SEQ ID NO: 955 - protein, 7 aa, synthetic construct
  REGION 1..7: note = Synthetic polypeptide
GASSLQS

SEQ ID NO: 956 - protein, 11 aa, synthetic construct
  REGION 1..11: note = Synthetic polypeptide
SGDKLGYTYTC

SEQ ID NO: 957 - protein, 7 aa, synthetic construct
  REGION 1..7: note = Synthetic polypeptide
QDTKRPS

SEQ ID NO: 958 - protein, 14 aa, synthetic construct
  REGION 1..14: note = Synthetic polypeptide
AGTRSDVGGYNFVS

SEQ ID NO: 959 - protein, 7 aa, synthetic construct
  REGION 1..7: note = Synthetic polypeptide
EVNKRPS

SEQ ID NO: 960 - protein, 11 aa, synthetic construct
  REGION 1..11: note = Synthetic polypeptide
RTSQSVSSFLA

SEQ ID NO: 961 - protein, 11 aa, synthetic construct
  REGION 1..11: note = Synthetic polypeptide
RASQSVSDWLA

SEQ ID NO: 962 - protein, 7 aa, synthetic construct
  REGION 1..7: note = Synthetic polypeptide
RASSLQS

SEQ ID NO: 963 - protein, 11 aa, synthetic construct
  REGION 1..11: note = Synthetic polypeptide
RASQSVSSNVA

SEQ ID NO: 964 - protein, 11 aa, synthetic construct
  REGION 1..11: note = Synthetic polypeptide
SGDALPKKYVY

SEQ ID NO: 965 - protein, 7 aa, synthetic construct
  REGION 1..7: note = Synthetic polypeptide
EDRRGPS

SEQ ID NO: 966 - protein, 17 aa, synthetic construct
  REGION 1..17: note = Synthetic polypeptide
KSSQSVFYSSNSQNYLA

SEQ ID NO: 967 - protein, 11 aa, synthetic construct
  REGION 1..11: note = Synthetic polypeptide
RASQSISNWLA

SEQ ID NO: 968 - protein, 7 aa, synthetic construct
  REGION 1..7: note = Synthetic polypeptide
KASRLES

SEQ ID NO: 969 - protein, 11 aa, synthetic construct
  REGION 1..11: note = Synthetic polypeptide
RASQTISSWLA

SEQ ID NO: 970 - protein, 11 aa, synthetic construct
  REGION 1..11: note = Synthetic polypeptide
SGDALPKQYGY

SEQ ID NO: 971 - protein, 7 aa, synthetic construct
  REGION 1..7: note = Synthetic polypeptide
KDSERPS

SEQ ID NO: 972 - protein, 7 aa, synthetic construct
  REGION 1..7: note = Synthetic polypeptide
EVTKRPS

SEQ ID NO: 973 - protein, 11 aa, synthetic construct
  REGION 1..11: note = Synthetic polypeptide
SGDALSKQYAY

SEQ ID NO: 974 - protein, 7 aa, synthetic construct
  REGION 1..7: note = Synthetic polypeptide
DVSKLKT

SEQ ID NO: 975 - protein, 11 aa, synthetic construct
  REGION 1..11: note = Synthetic polypeptide
RASQSISGWLA

SEQ ID NO: 976 - protein, 7 aa, synthetic construct
  REGION 1..7: note = Synthetic polypeptide
KASILES

SEQ ID NO: 977 - protein, 14 aa, synthetic construct
  REGION 1..14: note = Synthetic polypeptide
TGNSSNIGAGYEVH

SEQ ID NO: 978 - protein, 7 aa, synthetic construct
  REGION 1..7: note = Synthetic polypeptide
GNNNRPS

SEQ ID NO: 979 - protein, 11 aa, synthetic construct
  REGION 1..11: note = Synthetic polypeptide
SGDALPKQYAY

SEQ ID NO: 980 - protein, 11 aa, synthetic construct
  REGION 1..11: note = Synthetic polypeptide
RASQSVSNYFA

SEQ ID NO: 981 - protein, 13 aa, synthetic construct
  REGION 1..13: note = Synthetic polypeptide
SGSNSNIGNNFVS

SEQ ID NO: 982 - protein, 7 aa, synthetic construct
  REGION 1..7: note = Synthetic polypeptide
DNNERPS

SEQ ID NO: 983 - protein, 7 aa, synthetic construct
  REGION 1..7: note = Synthetic polypeptide
DSSNRAT

SEQ ID NO: 984 - protein, 11 aa, synthetic construct
  REGION 1..11: note = Synthetic polypeptide
RASQSVTSKLA

SEQ ID NO: 985 - protein, 16 aa, synthetic construct
  REGION 1..16: note = Synthetic polypeptide
RSSQSLLHSNGYNSLD

SEQ ID NO: 986 - protein, 13 aa, synthetic construct
  REGION 1..13: note = Synthetic polypeptide
SGSSSNIGSNTVH

SEQ ID NO: 987 - protein, 7 aa, synthetic construct
  REGION 1..7: note = Synthetic polypeptide
SNNQRPS

SEQ ID NO: 988 - protein, 7 aa, synthetic construct
  REGION 1..7: note = Synthetic polypeptide
DDNKRPS

SEQ ID NO: 989 - protein, 14 aa, synthetic construct
  REGION 1..14: note = Synthetic polypeptide
TGSSSDIGAGYDVH

SEQ ID NO: 990 - protein, 7 aa, synthetic construct
  REGION 1..7: note = Synthetic polypeptide
GNTNRPS

SEQ ID NO: 991 - protein, 17 aa, synthetic construct
  REGION 1..17: note = Synthetic polypeptide
KSSQSVFFSSDNKNYLA

SEQ ID NO: 992 - protein, 13 aa, synthetic construct
  REGION 1..13: note = Synthetic polypeptide
TRSSGSIASTYVQ

SEQ ID NO: 993 - protein, 7 aa, synthetic construct
  REGION 1..7: note = Synthetic polypeptide
EDNQRPP

SEQ ID NO: 994 - protein, 14 aa, synthetic construct
  REGION 1..14: note = Synthetic polypeptide
TGTSSDFGGYNYVS

SEQ ID NO: 995 - protein, 7 aa, synthetic construct
  REGION 1..7: note = Synthetic polypeptide
EVAKRPS

SEQ ID NO: 996 - protein, 11 aa, synthetic construct
  REGION 1..11: note = Synthetic polypeptide
RASESISIYLN

SEQ ID NO: 997 - protein, 7 aa, synthetic construct
  REGION 1..7: note = Synthetic polypeptide
AASSLQR

SEQ ID NO: 998 - protein, 14 aa, synthetic construct
  REGION 1..14: note = Synthetic polypeptide
TGTSSDVGGFNYVS

SEQ ID NO: 999 - protein, 7 aa, synthetic construct
  REGION 1..7: note = Synthetic polypeptide
EVNRRPS

SEQ ID NO: 1000 - protein, 16 aa, synthetic construct
  REGION 1..16: note = Synthetic polypeptide
RSSQSLVHSDGNTYLS

SEQ ID NO: 1001 - protein, 7 aa, synthetic construct
  REGION 1..7: note = Synthetic polypeptide
KISNRFS

SEQ ID NO: 1002 - protein, 7 aa, synthetic construct
  REGION 1..7: note = Synthetic polypeptide
DASNLET

SEQ ID NO: 1003 - protein, 11 aa, synthetic construct
  REGION 1..11: note = Synthetic polypeptide
SGDKLGDKYAC

SEQ ID NO: 1004 - protein, 7 aa, synthetic construct
  REGION 1..7: note = Synthetic polypeptide
QDSKRPS

SEQ ID NO: 1005 - protein, 11 aa, synthetic construct
  REGION 1..11: note = Synthetic polypeptide
RASQGISNNLA

SEQ ID NO: 1006 - protein, 13 aa, synthetic construct
  REGION 1..13: note = Synthetic polypeptide
  VARIANT 5: note = S or T
  VARIANT 7: note = S or no amino acid
  VARIANT 8: note = L or P
  VARIANT 12..13: note = K, G, or R
  VARIANT 12: note = K, G, or R
GTWDXSXXSAGXV

SEQ ID NO: 1007 - protein, 14 aa, synthetic construct
  REGION 1..14: note = Synthetic polypeptide
  VARIANT 2: note = K or R
  VARIANT 3: note = Y, F, T, A, G, or Y
  VARIANT 7: note = S, N, or R
  VARIANT 8: note = A or G
  VARIANT 11: note = W or Y
  VARIANT 12: note = F, L, I, A, or E
  VARIANT 13: note = D, E, or H
  VARIANT 14: note = Y, H, or S
AXXYDSXXYYXXXX

SEQ ID NO: 1008 - protein, 7 aa, synthetic construct
  REGION 1..7: note = Synthetic polypeptide
  VARIANT 1: note = D, E, or R
  VARIANT 3: note = S, T, N, or A
  VARIANT 4: note = N, K, or Q
XVXXRPS

SEQ ID NO: 1009 - protein, 7 aa, synthetic construct
  REGION 1..7: note = Synthetic polypeptide
  VARIANT 1: note = R, Q, or K
  VARIANT 4: note = T, S, G, R, or I
  VARIANT 7: note = T or S
XASXLEX

SEQ ID NO: 1010 - protein, 16 aa, synthetic construct
  REGION 1..16: note = Synthetic polypeptide
  VARIANT 1: note = D, E, or S
  VARIANT 2: note = I or V
  VARIANT 3: note = F or Y
  VARIANT 5: note = X or T
  VARIANT 6: note = G or E
  VARIANT 7: note = S, G, or T
  VARIANT 8: note = T or A
  VARIANT 9: note = N, S, H, K, or T
  VARIANT 11: note = N or S
  VARIANT 13: note = S or F
  VARIANT 14: note = L or V
  VARIANT 15: note = K or E
XXXHXXXXXYXPXXXS

SEQ ID NO: 1011 - protein, 17 aa, synthetic construct
  REGION 1..17: note = Synthetic polypeptide
  VARIANT 1: note = V or L
  VARIANT 2: note = I or M
  VARIANT 3: note = S, W, or L
  VARIANT 4: note = F or Y
  VARIANT 6: note = E or G
  VARIANT 7: note = S or T
  VARIANT 8: note = K, N, or Y
  VARIANT 10: note = F, W, or Y
  VARIANT 11: note = Y or F
  VARIANT 15: note = V or L
  VARIANT 16: note = K or L
XXXXDXXXKXXADSXXG

SEQ ID NO: 1012 - protein, 13 aa, synthetic construct
  REGION 1..13: note = Synthetic polypeptide
  VARIANT 4: note = N or S
  VARIANT 7: note = I or F
  VARIANT 9: note = S or N
  VARIANT 10: note = N, Y, S, or D
  VARIANT 11: note = Y, F, or D
  VARIANT 13: note = S or A
SGSXSNXGXXXVX

SEQ ID NO: 1013 - protein, 14 aa, synthetic construct
  REGION 1..14: note = Synthetic polypeptide
  VARIANT 1: note = A or T
  VARIANT 4: note = S, G, or R
  VARIANT 5: note = S or T
  VARIANT 7: note = V, F, or I
  VARIANT 9: note = G or A
  VARIANT 10: note = Y, D, or F
  VARIANT 11: note = K or N
  VARIANT 12: note = Y or F
XGTXXDXGXXXXVS

SEQ ID NO: 1014 - protein, 9 aa, synthetic construct
  REGION 1..9: note = Synthetic polypeptide
  VARIANT 2: note = I or T
  VARIANT 6: note = H or Y
  VARIANT 9: note = A or D
FXFSDXYMX